(19) United States
(12) Patent Application Publication
(10) Pub. No.: US 2023/0332229 A1
(43) Pub. Date: Oct. 19, 2023

(54) METHODS AND SYSTEMS FOR DETERMINING A PREGNANCY-RELATED STATE OF A SUBJECT

(71) Applicant: Mirvie, Inc., South San Francisco, CA (US)

(72) Inventors: Maneesh Jain, South San Francisco, CA (US); Eugeni Namsaraev, South San Francisco, CA (US); Morten Rasmussen, South San Francisco, CA (US); Joan Camunas Soler, South San Francisco, CA (US); Farooq Siddiqui, South San Francisco, CA (US); Mitsu Reddy, South San Francisco, CA (US); Elaine Gee, Windsor, CA (US); Arkady Khodursky, Castro Valley, CA (US); Rory Nolan, Oakland, CA (US); Manfred Lee, South San Francisco, CA (US)

(21) Appl. No.: 18/167,322

(22) Filed: Feb. 10, 2023

Related U.S. Application Data
(63) Continuation of application No. PCT/US2021/045684, filed on Aug. 12, 2021.
(60) Provisional application No. 63/065,130, filed on Aug. 13, 2020.
(60) Provisional application No. 63/132,741, filed on Dec. 31, 2020.
(60) Provisional application No. 63/170,151, filed on Apr. 2, 2021.
(60) Provisional application No. 63/172,249, filed on Apr. 8, 2021.

(51) Int. Cl. C12Q 1/6876; G16B 40/00; G16H 50/20; G16H 50/30

(52) U.S. Cl. CPC C12Q 1/6876; G16B 40/00; G16H 50/20; G16H 50/30; C12Q 2600/158; G16H 20/10

(57) ABSTRACT

The present disclosure provides methods and systems directed to cell-free identification and/or monitoring of pregnancy-related states. A method for identifying or monitoring a presence or susceptibility of a pregnancy-related state of a subject may comprise assaying a cell-free biological sample derived from said subject to detect a set of biomarkers, and analyzing the set of biomarkers with a trained algorithm to determine the presence or susceptibility of the pregnancy-related state.

### CROSS-REFERENCE

This application is a continuation of International Application No. PCT/US2021/045684, filed Aug. 12, 2021, which claims the benefit of U.S. Patent Application No. 63/065,130, filed Aug. 13, 2020, U.S. Patent Application No. 63/132,741, filed Dec. 31, 2020, U.S. Patent Application No. 63/170,151, filed Apr. 2, 2021, and U.S. Patent Application No. 63/172,249, filed Apr. 8, 2021, each of which is incorporated by reference herein in its entirety.

### BACKGROUND

Every year, about 15 million pre-term births are reported globally, and over 300,000 women die of pregnancy related complications such as hemorrhage and hypertensive disorders like preeclampsia. Pre-term birth may affect as many as about 10% of pregnancies, of which the majority are spontaneous pre-term births. Pregnancy-related complications such as pre-term birth are a leading cause of neonatal death and of complications later in life. Further, such pregnancy-related complications can cause negative health effects on maternal health.

### SUMMARY

Currently, there may be a lack of meaningful, clinically actionable diagnostic screenings or tests available for many pregnancy-related complications such as pre-term birth. Thus, to make pregnancy as safe as possible, there exists a need for rapid, accurate methods for identifying and monitoring pregnancy-related states that are non-invasive and cost-effective, toward improving maternal and fetal health.

The present disclosure provides methods, systems, and kits for identifying or monitoring pregnancy-related states by processing cell-free biological samples obtained from or derived from subjects. Cell-free biological samples (e.g., plasma samples) obtained from subjects may be analyzed to identify the pregnancy-related state (which may include, e.g., measuring a presence, absence, or relative assessment of the pregnancy-related state). Such subjects may include subjects with one or more pregnancy-related states and subjects without pregnancy-related states. Pregnancy-related states may include, for example, pre-term birth, full-term birth, gestational age, due date (e.g., due date for an unborn baby or fetus of a subject), onset of labor, pregnancy-related hypertensive disorders (e.g., preeclampsia), eclampsia, gestational diabetes, a congenital disorder of a fetus of the subject, ectopic pregnancy, spontaneous abortion, stillbirth, post-partum complications (e.g., post-partum depression, hemorrhage or excessive bleeding, pulmonary embolism, cardiomyopathy, diabetes, anemia, and hypertensive disorders), hyperemesis gravidarum (morning sickness), hemorrhage or excessive bleeding during delivery, premature rupture of membrane, premature rupture of membrane in pre-term birth, placenta previa (placenta covering the cervix), intrauterine/fetal growth restriction, macrosomia (large fetus for gestational age), neonatal conditions (e.g., anemia, apnea, bradycardia and other heart defects, bronchopulmonary dysplasia or chronic lung disease, diabetes, gastroschisis, hydrocephaly, hyperbilirubinemia, hypocalcemia, hypoglycemia, intraventricular hemorrhage, jaundice, necrotizing enterocolitis, patent ductus arteriosis, periventricular leukomalacia, persistent pulmonary hypertension, polycythemia, respiratory distress syndrome, retinopathy of prematurity, and transient tachypnea), and fetal development stages or states (e.g., normal fetal organ function or development, and abnormal fetal organ function or development). For example, the fetal development stages or states may be related to normal fetal organ function or development and/or abnormal fetal organ function or development for a fetal organ selected from the group consisting of heart, large intestine, small intestine, retina, prefrontal cortex, midbrain, kidney, and esophagus.

In an aspect, the present disclosure provides a method for identifying a presence or susceptibility of a pregnancy-related state of a subject, comprising assaying transcripts and/or metabolites in a cell-free biological sample derived from the subject to detect a set of biomarkers, and analyzing the set of biomarkers with a trained algorithm to determine the presence or susceptibility of the pregnancy-related state. In some embodiments, the method comprises assaying the transcripts in the cell-free biological sample derived from the subject to detect the set of biomarkers. In some embodiments, the transcripts are assayed with nucleic acid sequencing. In some embodiments, the method comprises assaying the metabolites in the cell-free biological sample derived from the subject to detect the set of biomarkers. In some embodiments, the metabolites are assayed with a metabolomics assay.

In another aspect, the present disclosure provides a method for identifying a presence or susceptibility of a pregnancy-related state of a subject, comprising assaying a cell-free biological sample derived from the subject to detect a set of biomarkers, and analyzing the set of biomarkers with a trained algorithm to determine the presence or susceptibility of the pregnancy-related state among a set of at least three distinct pregnancy-related states at an accuracy of at least about 80%.

In some embodiments, the pregnancy-related state is selected from the group consisting of pre-term birth, full-term birth, gestational age, due date, onset of labor, pregnancy-related hypertensive disorders (e.g., preeclampsia), eclampsia, gestational diabetes, a congenital disorder of a fetus of the subject, ectopic pregnancy, spontaneous abortion, stillbirth, post-partum complications (e.g., post-partum depression, hemorrhage or excessive bleeding, pulmonary embolism, cardiomyopathy, diabetes, anemia, and hypertensive disorders), hyperemesis gravidarum (morning sickness), hemorrhage or excessive bleeding during delivery, premature rupture of membrane, premature rupture of membrane in pre-term birth, placenta previa (placenta covering the cervix), intrauterine/fetal growth restriction, macrosomia (large fetus for gestational age), neonatal conditions (e.g., anemia, apnea, bradycardia and other heart defects, bronchopulmonary dysplasia or chronic lung disease, diabetes, gastroschisis, hydrocephaly, hyperbilirubinemia, hypocalcemia, hypoglycemia, intraventricular hemorrhage, jaundice, necrotizing enterocolitis, patent ductus arteriosis, periventricular leukomalacia, persistent pulmonary hypertension, polycythemia, respiratory distress syndrome, retinopathy of prematurity, and transient tachypnea), and fetal development stages or states (e.g., normal fetal organ function or development, and abnormal fetal organ function or development). For example, the fetal development stages or states may be related to normal fetal organ function or development and/or abnormal fetal organ function or development for a fetal organ selected from the group consisting of heart, large intestine, small intestine, retina, prefrontal cortex, midbrain, kidney, and esophagus.

In some embodiments, the pregnancy-related state is a sub-type of pre-term birth, and the at least three distinct pregnancy-related states include at least two distinct sub-types of pre-term birth. In some embodiments, the sub-type of pre-term birth is a molecular sub-type of pre-term birth, and the at least two distinct sub-types of pre-term birth include at least two distinct molecular sub-types of pre-term birth. In some embodiments, the distinct molecular subtypes of pre-term birth comprise a molecular subtype of pre-term birth selected from the group consisting of presence or history of prior pre-term birth, presence or history of spontaneous pre-term birth, presence or history of late miscarriage, presence or history of receiving cervical surgery, presence or history of a uterine anomaly, presence or history of ethnicity specific pre-term birth risk (e.g., among an African-American population), and presence or history of pre-term premature rupture of membrane (PPROM).

In some embodiments, the pregnancy-related state is a sub-type of preeclampsia, and the at least three distinct pregnancy-related states include at least two distinct sub-types of preeclampsia. In some embodiments, the distinct molecular subtypes of preeclampsia comprise a molecular subtype of preeclampsia selected from the group consisting of: presence or history of chronic or pre-existing hypertension, presence or history of gestational hypertension, presence or history of mild preeclampsia (e.g., with delivery greater than 34 weeks gestational age), presence or history of severe preeclampsia (with delivery less than 34 weeks gestational age), presence or history of eclampsia, and presence or history of HELLP syndrome.

In some embodiments, the method further comprises identifying a clinical intervention for the subject based at least in part on the presence or susceptibility of the pregnancy-related state. In some embodiments, the clinical intervention is selected from a plurality of clinical interventions. In some embodiments, the method further comprises determining a likelihood of said determination of said susceptibility of said pregnancy-related state of said subject, after which subject can be provided with the clinical intervention. In some embodiments, the clinical intervention comprises a pharmacological, surgical, or procedural treatment to reduce severity, delay, or eliminate said future susceptibility pregnancy-related state of said subject (e.g., aspirin for preeclampsia and steroids for pre-term birth).

In some embodiments, the set of biomarkers comprises a genomic locus associated with due date, wherein the genomic locus is selected from the group consisting of genes listed in Table 1, Table 7, and Table 10. In some embodiments, the set of biomarkers comprises a genomic locus associated with gestational age, wherein the genomic locus is selected from the group consisting of genes listed in Table 2, genes listed in Table 3, genes listed in Table 4, genes listed in Table 23, genes listed in Table 24, genes listed in Table 25, and genes listed in Table 26. In some embodiments, the set of biomarkers comprises a genomic locus associated with pre-term birth, wherein the genomic locus is selected from the group consisting of genes listed in Table 5, genes listed in Table 6, genes listed in Table 8, RAB27B, RGS18, CLCN3, B3GNT2, COL24A1, CXCL8, and PTGS2. In some embodiments, the set of biomarkers comprises a genomic locus associated with pre-term birth, wherein the genomic locus is selected from the group consisting of genes listed in Table 12, genes listed in Table 14, genes listed in Table 20, genes listed in Table 21, genes listed in Table 34, genes listed in Table 40, genes listed in Table 41, genes listed in Table 42, genes listed in Table 43, genes listed in Table 44, genes listed in Table 45, genes listed in Table 46, and genes listed in Table 47. In some embodiments, the panel of said one or more genomic loci comprises a genomic locus associated with preeclampsia, wherein the genomic locus is selected from the group consisting of genes listed in Table 15, genes listed in Table 17, genes listed in Table 18, genes listed in Table 19, genes listed in Table 27, genes listed in Table 33, CLDN7, PAPPA2, SNORD14A, PLEKHH1, MAGEA10, TLE6, and FABP1. In some embodiments, the panel of said one or more genomic loci comprises a genomic locus associated with fetal organ development, wherein the genomic locus is selected from the group of genes listed in Table 29. In some embodiments, the set of biomarkers comprises a genomic locus associated with gestational diabetes mellitus, wherein the genomic locus is selected from the group consisting of genes listed in Table 36, genes listed in Table 37, genes listed in Table 38, and genes listed in Table 39.

In some embodiments, the set of biomarkers comprises at least 5 distinct genomic loci. In some embodiments, the set of biomarkers comprises at least 10 distinct genomic loci. In some embodiments, the set of biomarkers comprises at least 25 distinct genomic loci. In some embodiments, the set of biomarkers comprises at least 50 distinct genomic loci. In some embodiments, the set of biomarkers comprises at least 100 distinct genomic loci. In some embodiments, the set of biomarkers comprises at least 150 distinct genomic loci.

In another aspect, the present disclosure provides a method comprising assaying a cell-free biological sample derived from a subject; identifying said subject as having or at risk of having preeclampsia; and upon identifying said subject as having or at risk of having preeclampsia, administering an anti-hypertensive drug to said subject.

In another aspect, the present disclosure provides a method for identifying or monitoring a presence or susceptibility of a pregnancy-related state of a subject, comprising: (a) using a first assay to process a cell-free biological sample derived from said subject to generate a first dataset; (b) using a second assay to process a vaginal or cervical biological sample derived from said subject to generate a second dataset comprising a microbiome profile of said vaginal or cervical biological sample; (c) using an algorithm (e.g., a trained algorithm) to process at least said first dataset and said second dataset to determine said presence or susceptibility of said pregnancy-related state, which trained algorithm has an accuracy of at least about 80% over 50 independent samples; and (d) electronically outputting a report indicative of said presence or susceptibility of the pregnancy-related state of said subject.

In another aspect, the present disclosure provides a method for identifying or monitoring a presence or susceptibility of a pregnancy-related state of a subject, comprising: (a) using a first assay to process a cell-free biological sample derived from said subject to generate a first dataset; (b) using a second assay to process a second biological sample derived from said subject to generate a second dataset comprising a biomarker profile (e.g., DNA genetic profile, methylation profile, RNA transcriptomic profile, transcription product profile, proteomic profile, metabolome profile, and/or microbiome profile) of said second biological sample; (c) using an algorithm (e.g., a trained algorithm) to process at least said first dataset and said second dataset to determine said presence or susceptibility of said pregnancy-related state, which trained algorithm has an accuracy of at least about 80% over 50 independent samples; and (d) electronically outputting a report indicative of said presence or susceptibility of the pregnancy-related state of said subject.

In another aspect, the present disclosure provides a method for identifying or monitoring a presence or susceptibility of a pregnancy-related state of a subject, comprising: (a) using a first assay to process a cell-free biological sample derived from said subject to generate a first dataset; (b) using a second dataset comprising clinical data from a medical record of the subject; (c) using an algorithm (e.g., a trained algorithm) to process at least said first dataset and said second dataset to determine said presence or susceptibility of said pregnancy-related state, which trained algorithm has an accuracy of at least about 80% over 50 independent samples; and (d) electronically outputting a report indicative of said presence or susceptibility of the pregnancy-related state of said subject.

In some embodiments, said first assay comprises using cell-free ribonucleic acid (cfRNA) molecules derived from said cell-free biological sample to generate transcriptomic data, using transcription products (e.g., messenger RNA, transfer RNA, or ribosomal RNA) derived from said cell-free biological sample to generate transcription product data, using cell-free deoxyribonucleic acid (cfDNA) molecules derived from said cell-free biological sample to generate genomic data and/or methylation data, using proteins (e.g., pregnancy-associated proteins corresponding to pregnancy-associated genomic loci or genes) derived from said cell-free biological sample to generate proteomic data, or using metabolites derived from said cell-free biological sample to generate metabolomic data. In some embodiments, said cell-free biological sample is from a blood of said subject. In some embodiments, said cell-free biological sample is from a urine of said subject. In some embodiments, said first assay comprises using cell-free ribonucleic acid (cfRNA) molecules derived from said cell-free biological sample to generate transcriptomic data, and said second assay comprises using proteins (e.g., pregnancy-associated proteins corresponding to pregnancy-associated genomic loci or genes) derived from said cell-free biological sample to generate proteomic data. In some embodiments, said first assay comprises using cell-free deoxyribonucleic acid (cfDNA) molecules derived from said cell-free biological sample to generate genomic data and/or methylation data, and said second assay comprises using proteins (e.g., pregnancy-associated proteins corresponding to pregnancy-associated genomic loci or genes) derived from said cell-free biological sample to generate proteomic data.

In some embodiments, said first dataset comprises a first set of biomarkers associated with said pregnancy-related state. In some embodiments, said second dataset comprises a second set of biomarkers associated with said pregnancy-related state. In some embodiments, said second set of biomarkers is different from said first set of biomarkers.

In some embodiments, said pregnancy-related state is selected from the group consisting of pre-term birth, full-term birth, gestational age, due date, onset of labor, pregnancy-related hypertensive disorders, preeclampsia, eclampsia, gestational diabetes, a congenital disorder of a fetus of the subject, ectopic pregnancy, spontaneous abortion, stillbirth, post-partum complications, hyperemesis gravidarum (morning sickness), hemorrhage or excessive bleeding during delivery, premature rupture of membrane, premature rupture of membrane in pre-term birth, placenta previa (placenta covering the cervix), intrauterine/fetal growth restriction, macrosomia (large fetus for gestational age), neonatal conditions, and fetal development stages or states.

In some embodiments, said pregnancy-related state comprises pre-term birth. In some embodiments, said pregnancy-related state comprises gestational age. In some embodiments, said pregnancy-related state comprises preeclampsia.

In some embodiments, said cell-free biological sample is selected from the group consisting of cell-free ribonucleic acid (cfRNA), cell-free deoxyribonucleic acid (cfDNA), cell-free fetal DNA (cffDNA), plasma, serum, urine, saliva, amniotic fluid, and derivatives thereof. In some embodiments, said cell-free biological sample is obtained or derived from said subject using an ethylenediaminetetraacetic acid (EDTA) collection tube, a cell-free RNA collection tube, or a cell-free DNA collection tube. In some embodiments, the method further comprises fractionating a whole blood sample of said subject to obtain said cell-free biological sample.

In some embodiments, said first assay comprises a cfRNA assay or a metabolomics assay. In some embodiments, said metabolomics assay comprises targeted mass spectroscopy (MS) or an immune assay. In some embodiments, said cell-free biological sample comprises cfRNA or urine. In some embodiments, said first assay or said second assay comprises quantitative polymerase chain reaction (qPCR). In some embodiments, said first assay or said second assay comprises a home use test configured to be performed in a home setting.

In some embodiments, said trained algorithm determines said presence or susceptibility of said pregnancy-related state of said subject at a sensitivity of at least about 80%. In some embodiments, said trained algorithm determines said presence or susceptibility of said pregnancy-related state of said subject at a sensitivity of at least about 90%. In some embodiments, said trained algorithm determines said presence or susceptibility of said pregnancy-related state of said subject at a sensitivity of at least about 95%.

In some embodiments, said trained algorithm determines said presence or susceptibility of said pregnancy-related state of said subject at a positive predictive value (PPV) of at least about 70%. In some embodiments, said trained algorithm determines said presence or susceptibility of said pregnancy-related state of said subject at a positive predictive value (PPV) of at least about 80%. In some embodiments, said trained algorithm determines said presence or susceptibility of said pregnancy-related state thereof of said subject at a positive predictive value (PPV) of at least about 90%.

In some embodiments, said trained algorithm determines said presence or susceptibility of said pregnancy-related state of said subject with an Area Under Curve (AUC) of at least about 0.90. In some embodiments, said trained algorithm determines said presence or susceptibility of said pregnancy-related state of said subject with an Area Under Curve (AUC) of at least about 0.95. In some embodiments, said trained algorithm determines said presence or susceptibility of said pregnancy-related state of said subject with an Area Under Curve (AUC) of at least about 0.99.

In some embodiments, said subject is asymptomatic for one or more of: pre-term birth, onset of labor, pregnancy-related hypertensive disorders, preeclampsia, eclampsia, gestational diabetes, a congenital disorder of a fetus of the subject, ectopic pregnancy, spontaneous abortion, stillbirth, post-partum complications, hyperemesis gravidarum (morning sickness), hemorrhage or excessive bleeding during delivery, premature rupture of membrane, premature rupture of membrane in pre-term birth, placenta previa (placenta covering the cervix), intrauterine/fetal growth restriction, macrosomia (large fetus for gestational age), neonatal conditions, and abnormal fetal development stages or states. For example, the fetal development stages or states may be related to normal fetal organ function or development and/or abnormal fetal organ function or development for a fetal organ selected from the group consisting of heart, large intestine, small intestine, retina, prefrontal cortex, midbrain, kidney, and esophagus.

In some embodiments, said cell-free biological sample is collected from said subject within a given gestational age interval for detection of a pregnancy-related state. In some embodiments, said given gestational age interval is within about 1 day, about 2 days, about 3 days, about 4 days, about 5 days, about 6 days about 7 days, about 8 days, about 9 days, about 10 days, about 11 days, about 12 days, about 13 days, about 14 days, about 3 weeks, or about 4 weeks from a given gestational age. In some embodiments, said given gestational age is about 0 weeks, about 1 week, about 2 weeks, about 3 weeks, about 4 weeks, about 5 weeks, about 6 weeks, about 7 weeks, about 8 weeks, about 9 weeks, about 10 weeks, about 11 week, about 12 weeks, about 13 weeks, about 14 weeks, about 15 weeks, about 16 weeks, about 17 weeks, about 18 weeks, about 19 weeks, about 20 weeks, about 21 week, about 22 weeks, about 23 weeks, about 24 weeks, about 25 weeks, about 26 weeks, about 27 weeks, about 28 weeks, about 29 weeks, about 30 weeks, about 31 week, about 32 weeks, about 33 weeks, about 34 weeks, about 35 weeks, about 36 weeks, about 37 weeks, about 38 weeks, about 39 weeks, about 40 weeks, about 41 weeks, about 42 weeks, about 43 weeks, about 44 weeks, or about 45 weeks. In some embodiments, said pregnancy-related state comprises one or more of: pre-term birth, onset of labor, pregnancy-related hypertensive disorders, preeclampsia, eclampsia, gestational diabetes, a congenital disorder of a fetus of the subject, ectopic pregnancy, spontaneous abortion, stillbirth, post-partum complications, hyperemesis gravidarum (morning sickness), hemorrhage or excessive bleeding during delivery, premature rupture of membrane, premature rupture of membrane in pre-term birth, placenta previa (placenta covering the cervix), intrauterine/fetal growth restriction, macrosomia (large fetus for gestational age), neonatal conditions, and abnormal fetal development stages or states. For example, the fetal development stages or states may be related to normal fetal organ function or development and/or abnormal fetal organ function or development for a fetal organ selected from the group consisting of heart, large intestine, small intestine, retina, prefrontal cortex, midbrain, kidney, and esophagus.

In some embodiments, said trained algorithm is trained using at least about 10 independent training samples associated with said presence or susceptibility of said pregnancy-related state. In some embodiments, said trained algorithm is trained using no more than about 100 independent training samples associated with said presence or susceptibility of said pregnancy-related state. In some embodiments, said trained algorithm is trained using a first set of independent training samples associated with a presence or susceptibility of said pregnancy-related state and a second set of independent training samples associated with an absence or no susceptibility of said pregnancy-related state. In some embodiments, the method further comprises using said trained algorithm to process a set of clinical health data of said subject to determine said presence or susceptibility of said pregnancy-related state.

In some embodiments, (a) comprises (i) subjecting said cell-free biological sample to conditions that are sufficient to isolate, enrich, or extract a set of ribonucleic (RNA) molecules, deoxyribonucleic acid (DNA) molecules, transcription products (e.g., messenger RNA, transfer RNA, or ribosomal RNA), proteins (e.g., pregnancy-associated proteins corresponding to pregnancy-associated genomic loci or genes), or metabolites, and (ii) analyzing said set of RNA molecules, DNA molecules, proteins, or metabolites using said first assay to generate said first dataset. In some embodiments, the method further comprises extracting a set of nucleic acid molecules from said cell-free biological sample, and subjecting said set of nucleic acid molecules to sequencing to generate a set of sequencing reads, wherein said first dataset comprises said set of sequencing reads. In some embodiments, (b) comprises (i) subjecting said vaginal or cervical biological sample to conditions that are sufficient to isolate, enrich, or extract a population of microbes, and (ii) analyzing said population of microbes using said second assay to generate said second dataset.

In some embodiments, said sequencing is massively parallel sequencing. In some embodiments, said sequencing comprises nucleic acid amplification. In some embodiments, said nucleic acid amplification comprises polymerase chain reaction (PCR). In some embodiments, said sequencing comprises use of simultaneous reverse transcription (RT) and polymerase chain reaction (PCR). In some embodiments, the method further comprises using probes configured to selectively enrich said set of nucleic acid molecules corresponding to a panel of one or more genomic loci. In some embodiments, said probes are nucleic acid primers. In some embodiments, said probes have sequence complementarity with nucleic acid sequences of said panel of said one or more genomic loci.

In some embodiments, said panel of said one or more genomic loci comprises at least one genomic locus selected from the group consisting of ACTB, ADAM12, ALPP, ANXA3, APLF, ARG1, AVPR1A, CAMP, CAPN6, CD180, CGA, CGB, CLCN3, CPVL, CSH1, CSH2, CSHL1, CYP3A7, DAPP1, DCX, DEFA4, DGCR14, ELANE, ENAH, EPB42, FABP1, FAM212B-AS1, FGA, FGB, FRMD4B, FRZB, FSTL3, GH2, GNAZ, HAL, HSD17B1, HSD3B1, HSPB8, Immune, ITIH2, KLF9, KNG1, KRT8, LGALS14, LTF, LYPLAL1, MAP3K7CL, MEF2C, MMD, MMP8, MOB1B, NFATC2, OTC, P2RY12, PAPPA, PGLYRP1, PKHD1L1, PKHD1L1, PLAC1, PLAC4, POLE2, PPBP, PSG1, PSG4, PSG7, PTGER3, RAB11A, RAB27B, RAP1GAP, RGS18, RPL23AP7, S100A8, S100A9, S100P, SERPINA7, SLC2A2, SLC38A4, SLC4A1, TBC1D15, VCAN, VGLL1, B3GNT2, COL24A1, CXCL8, and PTGS2.

In some embodiments, said panel of said one or more genomic loci comprises at least 5 distinct genomic loci. In some embodiments, said panel of said one or more genomic loci comprises at least 10 distinct genomic loci.

In some embodiments, said panel of said one or more genomic loci comprises a genomic locus associated with pre-term birth, wherein said genomic locus is selected from the group consisting of ADAM12, ANXA3, APLF, AVPR1A, CAMP, CAPN6, CD180, CGA, CGB, CLCN3, CPVL, CSH2, CSHL1, CYP3A7, DAPP1, DGCR14, ELANE, ENAH, FAM212B-AS1, FRMD4B, GH2, HSPB8, Immune, KLF9, KRT8, LGALS14, LTF, LYPLAL1, MAP3K7CL, MMD, MOB1B, NFATC2, P2RY12, PAPPA, PGLYRP1, PKHD1L1, PKHD1L1, PLAC1, PLAC4, POLE2, PPBP, PSG1, PSG4, PSG7, RAB11A, RAB27B, RAP1GAP, RGS18, RPL23AP7, TBC1D15, VCAN, VGLL1, B3GNT2, COL24A1, CXCL8, and PTGS2.

In some embodiments, said panel of said one or more genomic loci comprises a genomic locus associated with gestational age, wherein said genomic locus is selected from the group consisting of ACTB, ADAM12, ALPP, ANXA3, ARG1, CAMP, CAPN6, CGA, CGB, CSH1, CSH2, CSHL1, CYP3A7, DCX, DEFA4, EPB42, FABP1, FGA, FGB, FRZB, FSTL3, GH2, GNAZ, HAL, HSD17B1, HSD3B1, HSPB8, ITIH2, KNG1, LGALS14, LTF, MEF2C, MMP8, OTC, PAPPA, PGLYRP1, PLAC1, PLAC4, PSG1, PSG4, PSG7, PTGER3, S100A8, S100A9, S100P, SERPINA7, SLC2A2, SLC38A4, SLC4A1, VGLL1, RAB27B, RGS18, CLCN3, B3GNT2, COL24A1, CXCL8, and PTGS2.

In some embodiments, the panel of said one or more genomic loci comprises a genomic locus associated with due date, wherein the genomic locus is selected from the group consisting of genes listed in Table 1, Table 7, and Table 10. In some embodiments, the panel of said one or more genomic loci comprises a genomic locus associated with gestational age, wherein the genomic locus is selected from the group of genes listed in Table 2, genes listed in Table 3, genes listed in Table 4, genes listed in Table 23, genes listed in Table 24, genes listed in Table 25, and genes listed in Table 26 In some embodiments, the panel of said one or more genomic loci comprises a genomic locus associated with pre-term birth, wherein the genomic locus is selected from the group consisting of genes listed in Table 5, genes listed in Table 6, genes listed in Table 8, genes listed in Table 12, genes listed in Table 14, genes listed in Table 20, genes listed in Table 21, genes listed in Table 34, genes listed in Table 40, genes listed in Table 41, genes listed in Table 42, genes listed in Table 43, genes listed in Table 44, genes listed in Table 45, genes listed in Table 46, genes listed in Table 47, RAB27B, RGS18, CLCN3, B3GNT2, COL24A1, CXCL8, and PTGS2. In some embodiments, the panel of said one or more genomic loci comprises a genomic locus associated with preeclampsia, wherein the genomic locus is selected from the group consisting of genes listed in Table 15, genes listed in Table 17, genes listed in Table 18, genes listed in Table 19, genes listed in Table 27, genes listed in Table 33, CLDN7, PAPPA2, SNORD14A, PLEKHH1, MAGEA10, TLE6, and FABP1. In some embodiments, the panel of said one or more genomic loci comprises a genomic locus associated with fetal organ development, wherein the genomic locus is selected from the group of genes listed in Table 29. In some embodiments, the set of biomarkers comprises a genomic locus associated with gestational diabetes mellitus, wherein the genomic locus is selected from the group consisting of genes listed in Table 36, genes listed in Table 37, genes listed in Table 38, and genes listed in Table 39. In some embodiments, the panel of the one or more genomic loci comprises at least 5 distinct genomic loci. In some embodiments, the panel of the one or more genomic loci comprises at least 10 distinct genomic loci. In some embodiments, the panel of the one or more genomic loci comprises at least 25 distinct genomic loci. In some embodiments, the panel of the one or more genomic loci comprises at least 50 distinct genomic loci. In some embodiments, the panel of the one or more genomic loci comprises at least 100 distinct genomic loci. In some embodiments, the panel of the one or more genomic loci comprises at least 150 distinct genomic loci.

In some embodiments, said cell-free biological sample is processed without nucleic acid isolation, enrichment, or extraction.

In some embodiments, said report is presented on a graphical user interface of an electronic device of a user. In some embodiments, said user is said subject.

In some embodiments, the method further comprises determining a likelihood of said determination of said presence or susceptibility of said pregnancy-related state of said subject.

In some embodiments, said trained algorithm comprises a supervised machine learning algorithm. In some embodiments, said supervised machine learning algorithm comprises a deep learning algorithm, a support vector machine (SVM), a neural network, or a Random Forest. In some embodiments, said trained algorithm comprises a differential expression algorithm. In some embodiments, said differential expression algorithm comprises a use comparison of stochastic models, generalized Poisson (GPseq), mixed Poisson (TSPM), Poisson log-linear (PoissonSeq), negative binomial (edgeR, DESeq, baySeq, NBPSeq), linear model fit by MAANOVA, or a combination thereof.

In some embodiments, the method further comprises providing said subject with a therapeutic intervention for said presence or susceptibility of said pregnancy-related state. In some embodiments, said therapeutic intervention comprises hydroxyprogesterone caproate, a vaginal progesterone, a natural progesterone IVR product, an prostaglandin F2 alpha receptor antagonist, or a beta2-adrenergic receptor agonist.

In some embodiments, the method further comprises monitoring said presence or susceptibility of said pregnancy-related state, wherein said monitoring comprises assessing said presence or susceptibility of said pregnancy-related state of said subject at a plurality of time points, wherein said assessing is based at least on said presence or susceptibility of said pregnancy-related state determined in (d) at each of said plurality of time points.

In some embodiments, a difference in said assessment of said presence or susceptibility of said pregnancy-related state of said subject among said plurality of time points is indicative of one or more clinical indications selected from the group consisting of: (i) a diagnosis of said presence or susceptibility of said pregnancy-related state of said subject, (ii) a prognosis of said presence or susceptibility of said pregnancy-related state of said subject, and (iii) an efficacy or non-efficacy of a course of treatment for treating said presence or susceptibility of said pregnancy-related state of said subject.

In some embodiments, the method further comprises stratifying said pre-term birth by using said trained algorithm to determine a molecular sub-type of said pre-term birth from among a plurality of distinct molecular subtypes of pre-term birth. In some embodiments, the plurality of distinct molecular subtypes of pre-term birth comprises a molecular subtype of pre-term birth selected from the group consisting of presence or history of prior pre-term birth, presence or history of spontaneous pre-term birth, presence or history of late miscarriage, presence or history of receiving cervical surgery, presence or history of a uterine anomaly, presence or history of ethnicity specific pre-term birth risk (e.g., among an African-American population), and presence or history of pre-term premature rupture of membrane (PPROM).

In some embodiments, the method further comprises stratifying said preeclampsia by using said trained algorithm to determine a molecular sub-type of said preeclampsia from among a plurality of distinct molecular subtypes of preeclampsia comprise a molecular subtype of preeclampsia selected from the group consisting of history of chronic/pre-existing hypertension, gestational hypertension, mild preeclampsia (with delivery >34 weeks), severe preeclampsia (with delivery <34 weeks), eclampsia, HELLP syndrome.

In another aspect, the present disclosure provides a computer-implemented method for predicting a risk of pre-term birth of a subject, comprising: (a) receiving clinical health data of said subject, wherein said clinical health data comprises a plurality of quantitative or categorical measures of said subject; (b) using an algorithm (e.g., a trained algorithm) to process said clinical health data of said subject to determine a risk score indicative of said risk of pre-term birth of said subject; and (c) electronically outputting a report indicative of said risk score indicative of said risk of pre-term birth of said subject.

In another aspect, the present disclosure provides a computer-implemented method for predicting a risk of preeclampsia of a subject, comprising: (a) receiving clinical health data of said subject, wherein said clinical health data comprises a plurality of quantitative or categorical measures of said subject; (b) using an algorithm (e.g., a trained algorithm) to process said clinical health data of said subject to determine a risk score indicative of said risk of preeclampsia of said subject; and (c) electronically outputting a report indicative of said risk score indicative of said risk of preeclampsia of said subject.

In some embodiments, said clinical health data comprises one or more quantitative measures selected from the group consisting of age, weight, height, body mass index (BMI), blood pressure, heart rate, glucose levels, number of previous pregnancies, and number of previous births. In some embodiments, said clinical health data comprises one or more categorical measures selected from the group consisting of race, ethnicity, history of medication or other clinical treatment, history of tobacco use, history of alcohol consumption, daily activity or fitness level, genetic test results, blood test results, imaging results, and fetal screening results.

In some embodiments, said trained algorithm determines said risk of pre-term birth of said subject at a sensitivity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, said trained algorithm determines said risk of pre-term birth of said subject at a specificity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, said trained algorithm determines said risk of pre-term birth of said subject at a positive predictive value (PPV) of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, said trained algorithm determines said risk of pre-term birth of said subject at a negative predictive value (NPV) of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, said trained algorithm determines said risk of pre-term birth of said subject with an Area Under Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, or at least about 0.99.

In some embodiments, said trained algorithm determines said risk of preeclampsia of said subject at a sensitivity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, said trained algorithm determines said risk of preeclampsia of said subject at a specificity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, said trained algorithm determines said risk of preeclampsia of said subject at a positive predictive value (PPV) of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, said trained algorithm determines said risk of preeclampsia of said subject at a negative predictive value (NPV) of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, said trained algorithm determines said risk of preeclampsia of said subject with an Area Under Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, or at least about 0.99.

In some embodiments, said subject is asymptomatic for one or more of: pre-term birth, onset of labor, pregnancy-related hypertensive disorders, preeclampsia, eclampsia, gestational diabetes, a congenital disorder of a fetus of said subject, ectopic pregnancy, spontaneous abortion, stillbirth, post-partum complications, hyperemesis gravidarum (morning sickness), hemorrhage or excessive bleeding during delivery, premature rupture of membrane, premature rupture of membrane in pre-term birth, placenta previa (placenta covering the cervix), intrauterine/fetal growth restriction, macrosomia (large fetus for gestational age), neonatal conditions, and abnormal fetal development stages or states. For example, the fetal development stages or states may be related to normal fetal organ function or development and/or abnormal fetal organ function or development for a fetal organ selected from the group consisting of heart, large intestine, small intestine, retina, prefrontal cortex, midbrain, kidney, and esophagus.

In some embodiments, said trained algorithm is trained using at least about 10 independent training samples associated with pre-term birth. In some embodiments, said trained algorithm is trained using no more than about 100 independent training samples associated with pre-term birth. In some embodiments, said trained algorithm is trained using a first set of independent training samples associated with a presence of pre-term birth and a second set of independent training samples associated with an absence of pre-term birth.

In some embodiments, said trained algorithm is trained using at least about 10 independent training samples associated with preeclampsia. In some embodiments, said trained algorithm is trained using no more than about 100 independent training samples associated with preeclampsia In some embodiments, said trained algorithm is trained using a first set of independent training samples associated with a presence of preeclampsia and a second set of independent training samples associated with an absence of preeclampsia.

In some embodiments, said report is presented on a graphical user interface of an electronic device of a user. In some embodiments, said user is said subject.

In some embodiments, said trained algorithm comprises a supervised machine learning algorithm. In some embodiments, said supervised machine learning algorithm comprises a deep learning algorithm, a support vector machine (SVM), a neural network, or a Random Forest. In some embodiments, said trained algorithm comprises a differential expression algorithm. In some embodiments, said differential expression algorithm comprises a use comparison of stochastic models, generalized Poisson (GPseq), mixed Poisson (TSPM), Poisson log-linear (PoissonSeq), negative binomial (edgeR, DESeq, baySeq, NBPSeq), linear model fit by MAANOVA, or a combination thereof.

In some embodiments, the method further comprises providing said subject with a therapeutic intervention based at least in part on said risk score indicative of said risk of pre-term birth. In some embodiments, said therapeutic intervention comprises hydroxyprogesterone caproate, a vaginal progesterone, a natural progesterone IVR product, an prostaglandin F2 alpha receptor antagonist, or a beta2-adrenergic receptor agonist.

In some embodiments, the method further comprises providing said subject with a therapeutic intervention based at least in part on said risk score indicative of said risk of preeclampsia. In some embodiments, said therapeutic intervention comprises antihypertensive drug therapy (such as but not limited to hydralazine, labetalol, nifedipine, and sodium nitroprusside), management or prevention of seizures (such as but not limited to magnesium sulfate, phenytoin, and diazepam), or prevention by low-dose aspirin therapy (e.g., 100 mg per day or less) to reduce the incidence of preeclampsia

In some embodiments, the method further comprises monitoring said risk of pre-term birth, wherein said monitoring comprises assessing said risk of pre-term birth of said subject at a plurality of time points, wherein said assessing is based at least on said risk score indicative of said risk of pre-term birth determined in (b) at each of said plurality of time points.

In some embodiments, the method further comprises monitoring said risk of preeclampsia, wherein said monitoring comprises assessing said risk of preeclampsia of said subject at a plurality of time points, wherein said assessing is based at least on said risk score indicative of said risk of preeclampsia determined in (b) at each of said plurality of time points.

In some embodiments, the method further comprises refining said risk score indicative of said risk of pre-term birth of said subject by performing one or more subsequent clinical tests for said subject, and processing results from said one or more subsequent clinical tests using a trained algorithm to determine an updated risk score indicative of said risk of pre-term birth of said subject. In some embodiments, said one or more subsequent clinical tests comprise an ultrasound imaging or a blood test. In some embodiments, said risk score comprises a likelihood of said subject having a pre-term birth within a pre-determined duration of time.

In some embodiments, the method further comprises refining said risk score indicative of said risk of preeclampsia of said subject by performing one or more subsequent clinical tests for said subject, and processing results from said one or more subsequent clinical tests using a trained algorithm to determine an updated risk score indicative of said risk of preeclampsia of said subject. In some embodiments, said one or more subsequent clinical tests comprise an ultrasound imaging or a blood test. In some embodiments, said risk score comprises a likelihood of said subject having a preeclampsia within a pre-determined duration of time.

In some embodiments, said pre-determined duration of time is about 1 hour, about 2 hours, about 4 hours, about 6 hours, about 8 hours, about 10 hours, about 12 hours, about 14 hours, about 16 hours, about 18 hours, about 20 hours, about 22 hours, about 24 hours, about 1.5 days, about 2 days, about 2.5 days, about 3 days, about 3.5 days, about 4 days, about 4.5 days, about 5 days, about 5.5 days, about 6 days, about 6.5 days, about 7 days, about 8 days, about 9 days, about 10 days, about 12 days, about 14 days, about 3 weeks, about 4 weeks, about 5 weeks, about 6 weeks, about 7 weeks, about 8 weeks, about 9 weeks, about 10 weeks, about 11 weeks, about 12 weeks, about 13 weeks, or more than about 13 weeks.

In another aspect, the present disclosure provides a computer system for predicting a risk of pre-term birth of a subject, comprising: a database that is configured to store clinical health data of said subject, wherein said clinical health data comprises a plurality of quantitative or categorical measures of said subject; and one or more computer processors operatively coupled to said database, wherein said one or more computer processors are individually or collectively programmed to: (i) use an algorithm (e.g., a trained algorithm) to process said clinical health data of said subject to determine a risk score indicative of said risk of pre-term birth of said subject; and (ii) electronically output a report indicative of said risk score indicative of said risk of pre-term birth of said subject.

In another aspect, the present disclosure provides a computer system for predicting a risk of preeclampsia of a subject, comprising: a database that is configured to store clinical health data of said subject, wherein said clinical health data comprises a plurality of quantitative or categorical measures of said subject; and one or more computer processors operatively coupled to said database, wherein said one or more computer processors are individually or collectively programmed to: (i) use an algorithm (e.g., a trained algorithm) to process said clinical health data of said subject to determine a risk score indicative of said risk of preeclampsia of said subject; and (ii) electronically output a report indicative of said risk score indicative of said risk of preeclampsia of said subject.

In some embodiments, the computer system further comprises an electronic display operatively coupled to said one or more computer processors, wherein said electronic display comprises a graphical user interface that is configured to display said report.

In another aspect, the present disclosure provides a non-transitory computer readable medium comprising machine-executable code that, upon execution by one or more computer processors, implements a method for predicting a risk of pre-term birth of a subject, said method comprising: (a) receiving clinical health data of said subject, wherein said clinical health data comprises a plurality of quantitative or categorical measures of said subject; (b) using an algorithm (e.g., a trained algorithm) to process said clinical health data of said subject to determine a risk score indicative of said risk of pre-term birth of said subject; and (c) electronically outputting a report indicative of said risk score indicative of said risk of pre-term birth of said subject.

In another aspect, the present disclosure provides a non-transitory computer readable medium comprising machine-executable code that, upon execution by one or more computer processors, implements a method for predicting a risk of preeclampsia of a subject, said method comprising: (a) receiving clinical health data of said subject, wherein said clinical health data comprises a plurality of quantitative or categorical measures of said subject; (b) using an algorithm (e.g., a trained algorithm) to process said clinical health data of said subject to determine a risk score indicative of said risk of preeclampsia of said subject; and (c) electronically outputting a report indicative of said risk score indicative of said risk of preeclampsia of said subject.

In another aspect, the present disclosure provides a method for determining a due date, due date range, or gestational age of a fetus of a pregnant subject, comprising assaying a cell-free biological sample derived from said pregnant subject to detect a set of biomarkers, and analyzing said set of biomarkers with a trained algorithm to determine said due date, due date range, or gestational age of said fetus.

In some embodiments, the method further comprises analyzing an estimated due date of said fetus of said pregnant subject using said trained algorithm, wherein said estimated due date is generated from ultrasound measurements of said fetus. In some embodiments, said set of biomarkers comprises a genomic locus associated with due date, wherein said genomic locus is selected from the group of genes listed in Table 1, Table 7, and Table 10.

In some embodiments, said set of biomarkers comprises at least 5 distinct genomic loci. In some embodiments, said set of biomarkers comprises at least 10 distinct genomic loci. In some embodiments, said set of biomarkers comprises at least 25 distinct genomic loci. In some embodiments, said set of biomarkers comprises at least 50 distinct genomic loci. In some embodiments, said set of biomarkers comprises at least 100 distinct genomic loci. In some embodiments, said set of biomarkers comprises at least 150 distinct genomic loci.

In some embodiments, the method further comprises identifying a clinical intervention for said pregnant subject based at least in part on said determined due date. In some embodiments, said clinical intervention is selected from a plurality of clinical interventions. In some embodiments, the method further comprises determining a likelihood of said determination of said susceptibility of said pregnancy-related state of said subject, after which subject can be provided with the clinical intervention. In some embodiments, the clinical intervention comprises a pharmacological, surgical, or procedural treatment to reduce severity, delay, or eliminate said future susceptibility pregnancy-related state of said subject (e.g., aspirin for PE and steroids for PTB).

In some embodiments, said time-to-delivery is less than 7.5 weeks. In some embodiments, said genomic locus is selected from ACKR2, AKAP3, ANO5, Clorf21, C2orf42, CARNS1, CASC15, CCDC102B, CDC45, CDIPT, CMTM1, COPS8, CTD-2267D19.3, CTD-2349P21.9, CXorf65, DDX11L1, DGUOK, DPAGT1, EIF4A1P2, FANK1, FERMT1, FKRP, GAMT, GOLGA6L4, KLLN, LINC01347, LTA, MAPK12, METRN, MKRN4P, MPC2, MYL12BP1, NME4, NPM1P30, PCLO, PIF1, PTP4A3, RIMKLB, RP13-88F20.1, S100B, SIGLEC14, SLAIN1, SPATA33, TFAP2C, TMSB4XP8, TRGV10, and ZNF124.

In some embodiments, said time-to-delivery is less than 5 weeks. In some embodiments, said genomic locus is selected from C2orf68, CACNB3, CD40, CDKL5, CTBS, CTD-2272G21.2, CXCL8, DHRS7B, EIF5A2, IFITM3, MIR24-2, MTSS1, MYSM1, NCK1-AS1, NR1H4, PDE1C, PEMT, PEX7, PIF1, PPP2R3A, RABIF, SIGLEC14, SLC25A53, SPANXN4, SUPT3H, ZC2HC1C, ZMYM1, and ZNF124.

In some embodiments, said time-to-delivery is less than 7.5 weeks. In some embodiments, said genomic locus is selected from ACKR2, AKAP3, ANO5, Clorf21, C2orf42, CARNS1, CASC15, CCDC102B, CDC45, CDIPT, CMTM1, collectionga, COPS8, CTD-2267D19.3, CTD-2349P21.9, DDX11L1, DGUOK, DPAGT1, EIF4A1P2, FANK1, FERMT1, FKRP, GAMT, GOLGA6L4, KLLN, LINC01347, LTA, MAPK12, METRN, MPC2, MYL12BP1, NME4, NPM1P30, PCLO, PIF1, PTP4A3, RIMKLB, RP13-88F20.1, S100B, SIGLEC14, SLAIN1, SPATA33, STAT1, TFAP2C, TMEM94, TMSB4XP8, TRGV10, ZNF124, and ZNF713.

In some embodiments, said time-to-delivery is less than 5 weeks. In some embodiments, said genomic locus is selected from ATP6V1E1P1, ATP8A2, C2orf68, CACNB3, CD40, CDKL4, CDKL5, CEP152, CLEC4D, COL18A1, collectionga, COX16, CTBS, CTD-2272G21.2, CXCL2, CXCL8, DHRS7B, DPPA4, EIF5A2, FERMT1, GNB1L, IFITM3, KATNAL1, LRCH4, MBD6, MIR24-2, MTSS1, MYSM1, NCK1-AS1, NPIPB4, NR1H4, PDE1C, PEMT, PEX7, PIF1, PPP2R3A, PXDN, RABIF, SERTAD3, SIGLEC14, SLC25A53, SPANXN4, SSH3, SUPT3H, TMEM150C, TNFAIP6, UPP1, XKR8, ZC2HC1C, ZMYM1, and ZNF124.

In some embodiments, said time-to-delivery is within about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days about 7 days, about 8 days, about 9 days, about 10 days, about 11 days, about 12 days, about 13 days, about 14 days, or about 3 weeks.

In some embodiments, said trained algorithm comprises a linear regression model or an ANOVA model. In some embodiments, said ANOVA model determines a maximum-likelihood time window corresponding to said due date from among a plurality of time windows. In some embodiments, said maximum-likelihood time window corresponds to a time-to-delivery of 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, or 20 weeks. In some embodiments, said ANOVA model determines a probability or likelihood of a time window corresponding to said due date from among a plurality of time windows. In some embodiments, said ANOVA model calculates a probability-weighted average across said plurality of time windows to determine an average or expected time window distance.

In another aspect, the present disclosure provides a method for identifying or monitoring a presence or susceptibility of a pregnancy-related state of a subject, comprising: (a) using a first assay to process a first cell-free biological sample derived from the subject to generate a first dataset; (b) based at least in part on the first dataset generated in (a), using a second assay different from the first assay to process a second cell-free biological sample derived from the subject to generate a second dataset indicative of the presence or susceptibility of the pregnancy-related state at a specificity greater than the first dataset; (c) using a trained algorithm to process at least the second dataset to determine the presence or susceptibility of the pregnancy-related state, which trained algorithm has an accuracy of at least about 80% over 50 independent samples; and (d) electronically outputting a report indicative of the presence or susceptibility of the pregnancy-related state of the subject.

In some embodiments, the first assay comprises using cell-free ribonucleic acid (cfRNA) molecules derived from the first cell-free biological sample to generate transcriptomic data, using transcription products (e.g., messenger RNA, transfer RNA, or ribosomal RNA) derived from said cell-free biological sample to generate transcription product data, using cell-free deoxyribonucleic acid (cfDNA) molecules derived from the first cell-free biological sample to generate genomic data and/or methylation data, using proteins (e.g., pregnancy-associated proteins corresponding to pregnancy-associated genomic loci or genes) derived from the first cell-free biological sample to generate proteomic data, or using metabolites derived from the first cell-free biological sample to generate metabolomic data. In some embodiments, the first cell-free biological sample is from a blood of the subject. In some embodiments, the first cell-free biological sample is from a urine of the subject. In some embodiments, the first dataset comprises a first set of biomarkers associated with the pregnancy-related state. In some embodiments, the second dataset comprises a second set of biomarkers associated with the pregnancy-related state. In some embodiments, the second set of biomarkers is different from the first set of biomarkers.

In some embodiments, the pregnancy-related state is selected from the group consisting of pre-term birth, full-term birth, gestational age, due date, onset of labor, pregnancy-related hypertensive disorders (e.g., preeclampsia), eclampsia, gestational diabetes, a congenital disorder of a fetus of the subject, ectopic pregnancy, spontaneous abortion, stillbirth, post-partum complications (e.g., post-partum depression, hemorrhage or excessive bleeding, pulmonary embolism, cardiomyopathy, diabetes, anemia, and hypertensive disorders), hyperemesis gravidarum (morning sickness), hemorrhage or excessive bleeding during delivery, premature rupture of membrane, premature rupture of membrane in pre-term birth, placenta previa (placenta covering the cervix), intrauterine/fetal growth restriction, macrosomia (large fetus for gestational age), neonatal conditions (e.g., anemia, apnea, bradycardia and other heart defects, bronchopulmonary dysplasia or chronic lung disease, diabetes, gastroschisis, hydrocephaly, hyperbilirubinemia, hypocalcemia, hypoglycemia, intraventricular hemorrhage, jaundice, necrotizing enterocolitis, patent ductus arteriosis, periventricular leukomalacia, persistent pulmonary hypertension, polycythemia, respiratory distress syndrome, retinopathy of prematurity, and transient tachypnea), and fetal development stages or states (e.g., normal fetal organ function or development, and abnormal fetal organ function or development). For example, the fetal development stages or states may be related to normal fetal organ function or development and/or abnormal fetal organ function or development for a fetal organ selected from the group consisting of heart, large intestine, small intestine, retina, prefrontal cortex, midbrain, kidney, and esophagus. In some embodiments, the pregnancy-related state comprises pre-term birth. In some embodiments, the pregnancy-related state comprises gestational age.

In some embodiments, the cell-free biological sample is selected from the group consisting of cell-free ribonucleic acid (cfRNA), cell-free deoxyribonucleic acid (cfDNA), cell-free fetal DNA (cffDNA), plasma, serum, urine, saliva, amniotic fluid, and derivatives thereof. In some embodiments, the first cell-free biological sample or the second cell-free biological sample is obtained or derived from the subject using an ethylenediaminetetraacetic acid (EDTA) collection tube, a cell-free RNA collection tube, or a cell-free DNA collection tube. In some embodiments, the method further comprises fractionating a whole blood sample of the subject to obtain the first cell-free biological sample or the second cell-free biological sample. In some embodiments, (i) the first assay comprises a cfRNA assay and the second assay comprises a metabolomics assay, or (ii) the first assay comprises a metabolomics assay and the second assay comprises a cfRNA assay. In some embodiments, (i) the first cell-free biological sample comprises cfRNA and the second cell-free biological sample comprises urine, or (ii) the first cell-free biological sample comprises urine and the second cell-free biological sample comprises cfRNA. In some embodiments, the first assay or the second assay comprises quantitative polymerase chain reaction (qPCR). In some embodiments, the first assay or the second assay comprises a home use test configured to be performed in a home setting. In some embodiments, the first assay or the second assay comprises a metabolomics assay. In some embodiments, the metabolomics assay comprises targeted mass spectroscopy (MS) or an immune assay.

In some embodiments, the first dataset is indicative of the presence or susceptibility of the pregnancy-related state at a sensitivity of at least about 80%. In some embodiments, the first dataset is indicative of the presence or susceptibility of the pregnancy-related state at a sensitivity of at least about 90%. In some embodiments, the first dataset is indicative of the presence or susceptibility of the pregnancy-related state at a sensitivity of at least about 95%. In some embodiments, the first dataset is indicative of the presence or susceptibility of the pregnancy-related state at a positive predictive value (PPV) of at least about 70%. In some embodiments, the first dataset is indicative of the presence or susceptibility of the pregnancy-related state at a positive predictive value (PPV) of at least about 80%. In some embodiments, the first dataset is indicative of the presence or susceptibility of the pregnancy-related state at a positive predictive value (PPV) of at least about 90%. In some embodiments, the second dataset is indicative of the presence or susceptibility of the pregnancy-related state at a specificity of at least about 90%. In some embodiments, the second dataset is indicative of the presence or susceptibility of the pregnancy-related state at a specificity of at least about 95%. In some embodiments, the second dataset is indicative of the presence or susceptibility of the pregnancy-related state at a specificity of at least about 99%. In some embodiments, the second dataset is indicative of the presence or susceptibility of the pregnancy-related state at a negative predictive value (NPV) of at least about 90%. In some embodiments, the second dataset is indicative of the presence or susceptibility of the pregnancy-related state at a negative predictive value (NPV) of at least about 95%. In some embodiments, the second dataset is indicative of the presence or susceptibility of the pregnancy-related state at a negative predictive value (NPV) of at least about 99%. In some embodiments, the trained algorithm determines the presence or susceptibility of the pregnancy-related state of the subject with an Area Under Curve (AUC) of at least about 0.90. In some embodiments, the trained algorithm determines the presence or susceptibility of the pregnancy-related state of the subject with an Area Under Curve (AUC) of at least about 0.95. In some embodiments, the trained algorithm determines the presence or susceptibility of the pregnancy-related state of the subject with an Area Under Curve (AUC) of at least about 0.99.

In some embodiments, the subject is asymptomatic for one or more of: pre-term birth, onset of labor, pregnancy-related hypertensive disorders (e.g., preeclampsia), eclampsia, gestational diabetes, a congenital disorder of a fetus of the subject, ectopic pregnancy, spontaneous abortion, stillbirth, post-partum complications (e.g., post-partum depression, hemorrhage or excessive bleeding, pulmonary embolism, cardiomyopathy, diabetes, anemia, and hypertensive disorders), hyperemesis gravidarum (morning sickness), hemorrhage or excessive bleeding during delivery, premature rupture of membrane, premature rupture of membrane in pre-term birth, placenta previa (placenta covering the cervix), intrauterine/fetal growth restriction, macrosomia (large fetus for gestational age), neonatal conditions (e.g., anemia, apnea, bradycardia and other heart defects, bronchopulmonary dysplasia or chronic lung disease, diabetes, gastroschisis, hydrocephaly, hyperbilirubinemia, hypocalcemia, hypoglycemia, intraventricular hemorrhage, jaundice, necrotizing enterocolitis, patent ductus arteriosis, periventricular leukomalacia, persistent pulmonary hypertension, polycythemia, respiratory distress syndrome, retinopathy of prematurity, and transient tachypnea), and abnormal fetal development stages or states (e.g., abnormal fetal organ function or development). For example, the fetal development stages or states may be related to normal fetal organ function or development and/or abnormal fetal organ function or development for a fetal organ selected from the group consisting of heart, large intestine, small intestine, retina, prefrontal cortex, midbrain, kidney, and esophagus.

In some embodiments, the trained algorithm is trained using at least about 10 independent training samples associated with the pregnancy-related state. In some embodiments, the trained algorithm is trained using no more than about 100 independent training samples associated with the pregnancy-related state. In some embodiments, the trained algorithm is trained using a first set of independent training samples associated with a presence of the pregnancy-related state and a second set of independent training samples associated with an absence of the pregnancy-related state. In some embodiments, the method further comprises using the trained algorithm to process the first dataset to determine the presence or susceptibility of the pregnancy-related state. In some embodiments, the method further comprises using the trained algorithm to process a set of clinical health data of the subject to determine the presence or susceptibility of the pregnancy-related state.

In some embodiments, (a) comprises (i) subjecting the first cell-free biological sample to conditions that are sufficient to isolate, enrich, or extract a first set of ribonucleic acid (RNA) molecules, deoxyribonucleic acid (DNA) molecules, proteins (e.g., pregnancy-associated proteins corresponding to pregnancy-associated genomic loci or genes), or metabolites, and (ii) analyzing the first set of RNA molecules, DNA molecules, proteins, or metabolites using the first assay to generate the first dataset. In some embodiments, the method further comprises extracting a first set of nucleic acid molecules from the first cell-free biological sample, and subjecting the first set of nucleic acid molecules to sequencing to generate a first set of sequencing reads, wherein the first dataset comprises the first set of sequencing reads. In some embodiments, the method further comprises extracting a first set of metabolites from the first cell-free biological sample, and assaying the first set of metabolites to generate the first dataset In some embodiments, (b) comprises (i) subjecting the second cell-free biological sample to conditions that are sufficient to isolate, enrich, or extract a second set of ribonucleic acid (RNA) molecules, deoxyribonucleic acid (DNA) molecules, proteins (e.g., pregnancy-associated proteins corresponding to pregnancy-associated genomic loci or genes), or metabolites, and (ii) analyzing the second set of RNA molecules, DNA molecules, proteins, or metabolites using the second assay to generate the second dataset. In some embodiments, the method further comprises extracting a second set of nucleic acid molecules from the second cell-free biological sample, and subjecting the second set of nucleic acid molecules to sequencing to generate a second set of sequencing reads, wherein the second dataset comprises the second set of sequencing reads. In some embodiments, the method further comprises extracting a second set of metabolites from the second cell-free biological sample, and assaying the second set of metabolites to generate the second dataset. In some embodiments, the sequencing is massively parallel sequencing. In some embodiments, the sequencing comprises nucleic acid amplification. In some embodiments, the nucleic acid amplification comprises polymerase chain reaction (PCR). In some embodiments, the sequencing comprises use of simultaneous reverse transcription (RT) and polymerase chain reaction (PCR).

In some embodiments, the method further comprises using probes configured to selectively enrich the first set of nucleic acid molecules or the second set of nucleic acid molecules corresponding to a panel of one or more genomic loci. In some embodiments, the probes are nucleic acid primers. In some embodiments, the probes have sequence complementarity with nucleic acid sequences of the panel of the one or more genomic loci. In some embodiments, the panel of the one or more genomic loci comprises at least one genomic locus selected from the group consisting of ACTB, ADAM12, ALPP, ANXA3, APLF, ARG1, AVPR1A, CAMP, CAPN6, CD180, CGA, CGB, CLCN3, CPVL, CSH1, CSH2, CSHL1, CYP3A7, DAPP1, DCX, DEFA4, DGCR14, ELANE, ENAH, EPB42, FABP1, FAM212B-AS1, FGA, FGB, FRMD4B, FRZB, FSTL3, GH2, GNAZ, HAL, HSD17B1, HSD3B1, HSPB8, Immune, ITIH2, KLF9, KNG1, KRT8, LGALS14, LTF, LYPLAL1, MAP3K7CL, MEF2C, MMD, MMP8, MOB1B, NFATC2, OTC, P2RY12, PAPPA, PGLYRP1, PKHD1L1, PKHD1L1, PLAC1, PLAC4, POLE2, PPBP, PSG1, PSG4, PSG7, PTGER3, RAB11A, RAB27B, RAP1GAP, RGS18, RPL23AP7, S100A8, S100A9, S100P, SERPINA7, SLC2A2, SLC38A4, SLC4A1, TBC1D15, VCAN, VGLL1, B3GNT2, COL24A1, CXCL8, and PTGS2.

In some embodiments, the panel of the one or more genomic loci comprises at least 5 distinct genomic loci. In some embodiments, the panel of the one or more genomic loci comprises at least 10 distinct genomic loci. In some embodiments, the panel of the one or more genomic loci comprises a genomic locus associated with pre-term birth, wherein said genomic locus is selected from the group consisting of ADAM12, ANXA3, APLF, AVPR1A, CAMP, CAPN6, CD180, CGA, CGB, CLCN3, CPVL, CSH2, CSHL1, CYP3A7, DAPP1, DGCR14, ELANE, ENAH, FAM212B-AS1, FRMD4B, GH2, HSPB8, Immune, KLF9, KRT8, LGALS14, LTF, LYPLAL1, MAP3K7CL, MMD, MOB1B, NFATC2, P2RY12, PAPPA, PGLYRP1, PKHD1L1, PKHD1L1, PLAC1, PLAC4, POLE2, PPBP, PSG1, PSG4, PSG7, RAB11A, RAB27B, RAP1GAP, RGS18, RPL23AP7, TBC1D15, VCAN, VGLL1, B3GNT2, COL24A1, CXCL8, and PTGS2. In some embodiments, the panel of the one or more genomic loci comprises a genomic locus associated with gestational age, wherein said genomic locus is selected from the group consisting of ACTB, ADAM12, ALPP, ANXA3, ARG1, CAMP, CAPN6, CGA, CGB, CSH1, CSH2, CSHL1, CYP3A7, DCX, DEFA4, EPB42, FABP1, FGA, FGB, FRZB, FSTL3, GH2, GNAZ, HAL, HSD17B1, HSD3B1, HSPB8, ITIH2, KNG1, LGALS14, LTF, MEF2C, MMP8, OTC, PAPPA, PGLYRP1, PLAC1, PLAC4, PSG1, PSG4, PSG7, PTGER3, S100A8, S100A9, S100P, SERPINA7, SLC2A2, SLC38A4, SLC4A1, VGLL1, B3GNT2, COL24A1, CXCL8, and PTGS2. In some embodiments, the panel of said one or more genomic loci comprises a genomic locus associated with due date, wherein the genomic locus is selected from the group of genes listed in Table 1, Table 7, and Table 10. In some embodiments, the panel of said one or more genomic loci comprises a genomic locus associated with gestational age, wherein the genomic locus is selected from the group of genes listed in Table 2, genes listed in Table 3, genes listed in Table 4, genes listed in Table 23, genes listed in Table 24, gene listed in Table 25, and genes listed in Table 26 In some embodiments, the panel of said one or more genomic loci comprises a genomic locus associated with pre-term birth, wherein the genomic locus is selected from the group of genes listed in Table 5, genes listed in Table 6, genes listed in Table 8, genes listed in Table 12, genes listed in Table 14, genes listed in Table 20, genes listed in Table 21, genes listed in Table 34, genes listed in Table 40, genes listed in Table 41, genes listed in Table 42, genes listed in Table 43, genes listed in Table 44, genes listed in Table 45, genes listed in Table 46, genes listed in Table 47, RAB27B, RGS18, CLCN3, B3GNT2, COL24A1, CXCL8, and PTGS2. In some embodiments, the panel of said one or more genomic loci comprises a genomic locus associated with preeclampsia, wherein the genomic locus is selected from the group consisting of genes listed in Table 15, genes listed in Table 17, genes listed in Table 18, genes listed in Table 19, genes listed in Table 27, genes listed in Table 33, CLDN7, PAPPA2, SNORD14A, PLEKHH1, MAGEA10, TLE6, and FABP1. In some embodiments, the panel of said one or more genomic loci comprises a genomic locus associated with fetal organ development, wherein the genomic locus is selected from the group of genes listed in Table 29. In some embodiments, the set of biomarkers comprises a genomic locus associated with gestational diabetes mellitus, wherein the genomic locus is selected from the group consisting of genes listed in Table 36, genes listed in Table 37, genes listed in Table 38, and genes listed in Table 39.

In some embodiments, the panel of the one or more genomic loci comprises at least 5 distinct genomic loci. In some embodiments, the panel of the one or more genomic loci comprises at least 10 distinct genomic loci. In some embodiments, the panel of the one or more genomic loci comprises at least 25 distinct genomic loci. In some embodiments, the panel of the one or more genomic loci comprises at least 50 distinct genomic loci. In some embodiments, the panel of the one or more genomic loci comprises at least 100 distinct genomic loci. In some embodiments, the panel of the one or more genomic loci comprises at least 150 distinct genomic loci. In some embodiments, the first cell-free biological sample or the second cell-free biological sample is processed without nucleic acid isolation, enrichment, or extraction. In some embodiments, the report is presented on a graphical user interface of an electronic device of a user. In some embodiments, the user is the subject.

In some embodiments, the method further comprises determining a likelihood of the determination of the presence or susceptibility of the pregnancy-related state of the subject. In some embodiments, the trained algorithm comprises a supervised machine learning algorithm. In some embodiments, the supervised machine learning algorithm comprises a deep learning algorithm, a support vector machine (SVM), a neural network, or a Random Forest. In some embodiments, said trained algorithm comprises a differential expression algorithm. In some embodiments, said differential expression algorithm comprises a use comparison of stochastic models, generalized Poisson (GPseq), mixed Poisson (TSPM), Poisson log-linear (PoissonSeq), negative binomial (edgeR, DESeq, baySeq, NBPSeq), linear model fit by MAANOVA, or a combination thereof. In some embodiments, the method further comprises providing the subject with a therapeutic intervention for the presence or susceptibility of the pregnancy-related state. In some embodiments, therapeutic intervention comprises a progesterone treatment such as hydroxyprogesterone caproate (e.g., 17-alpha hydroxyprogesterone caproate (17-P), LPCN 1107 from Lipocine, Makena from AMAG Pharma), a vaginal progesterone, or a natural progesterone IVR product (e.g., DARE-FRT1 (JNP-0301) from Juniper Pharma); a prostaglandin F2 alpha receptor antagonist (e.g., OBE022 from ObsEva); or a beta2-adrenergic receptor agonist (e.g., bedoradrine sulfate (MN-221) from MediciNova). Therapeutic interventions may be described by, for example, “WHO Recommendations on Interventions to Improve Preterm Birth Outcomes,” ISBN 9789241508988, World Health Organization, 2015, which is hereby incorporated by reference in its entirety. In some embodiments, the method further comprises monitoring the presence or susceptibility of the pregnancy-related state, wherein the monitoring comprises assessing the presence or susceptibility of the pregnancy-related state of the subject at a plurality of time points, wherein the assessing is based at least on the presence or susceptibility of the pregnancy-related state determined in (d) at each of the plurality of time points. In some embodiments, a difference in the assessment of the presence or susceptibility of the pregnancy-related state of the subject among the plurality of time points is indicative of one or more clinical indications selected from the group consisting of: (i) a diagnosis of the presence or susceptibility of the pregnancy-related state of the subject, (ii) a prognosis of the presence or susceptibility of the pregnancy-related state of the subject, and (iii) an efficacy or non-efficacy of a course of treatment for treating the presence or susceptibility of the pregnancy-related state of the subject.

In some embodiments, the method further comprises stratifying the pre-term birth by using the trained algorithm to determine a molecular sub-type of the pre-term birth from among a plurality of distinct molecular subtypes of pre-term birth. In some embodiments, the plurality of distinct molecular subtypes of pre-term birth comprises a molecular subtype of pre-term birth selected from the group consisting of presence or history of prior pre-term birth, presence or history of spontaneous pre-term birth, presence or history of late miscarriage, presence or history of receiving cervical surgery, presence or history of a uterine anomaly, presence or history of ethnicity specific pre-term birth risk (e.g., among an African-American population), and presence or history of pre-term premature rupture of membrane (PPROM).

In some embodiments, the method further comprises stratifying the preeclampsia by using said trained algorithm to determine a molecular sub-type of said preeclampsia from among a plurality of distinct molecular subtypes of preeclampsia. In some embodiments, the plurality of distinct molecular subtypes of preeclampsia comprises a molecular subtype of preeclampsia selected from the group consisting of: presence or history of chronic or pre-existing hypertension, presence or history of gestational hypertension, presence or history of mild preeclampsia (e.g., with delivery greater than 34 weeks gestational age), presence or history of severe preeclampsia (with delivery less than 34 weeks gestational age), presence or history of eclampsia, and presence or history of HELLP syndrome.

In another aspect, the present disclosure provides a computer system for identifying or monitoring a presence or susceptibility of the pregnancy-related state of a subject, comprising: a database that is configured to store a first dataset and a second dataset, wherein the second dataset is indicative of the presence or susceptibility of the pregnancy-related state at a specificity greater than the first dataset; and one or more computer processors operatively coupled to the database, wherein the one or more computer processors are individually or collectively programmed to: (i) use a trained algorithm to process at least the second dataset to determine the presence or susceptibility of the pregnancy-related state, which trained algorithm has an accuracy of at least about 80% over 50 independent samples; and (ii) electronically output a report indicative of the presence or susceptibility of the pregnancy-related state of the subject.

In some embodiments, the computer system further comprises an electronic display operatively coupled to the one or more computer processors, wherein the electronic display comprises a graphical user interface that is configured to display the report.

In another aspect, the present disclosure provides a non-transitory computer readable medium comprising machine-executable code that, upon execution by one or more computer processors, implements a method for identifying or monitoring a presence or susceptibility of the pregnancy-related state of a subject, the method comprising: (a) obtaining a first dataset, and a second dataset, wherein the second dataset is indicative of the presence or susceptibility of the pregnancy-related state at a specificity greater than the first dataset; (b) using a trained algorithm to process at least the second dataset to determine the pregnancy-related state, which trained algorithm has an accuracy of at least about 80% over 50 independent samples; and (c) electronically outputting a report indicative of the presence or susceptibility of the pregnancy-related state of the subject.

In another aspect, the present disclosure provides a method for identifying a presence or susceptibility of pregnancy-related state of a subject, comprising (i) assaying a first cell-free biological sample derived from the subject with a first assay to generate a first dataset, (ii) assaying a second cell-free biological sample derived from the subject with a second assay to generate a second dataset that is indicative of the presence or susceptibility of the pregnancy-related state at a specificity greater than the first dataset, and (iii) using a trained algorithm to process at least the second dataset to determine the presence or susceptibility of the pregnancy-related state at an accuracy of at least about 80%. In some embodiments, the accuracy is at least about 90%. In some embodiments, the pregnancy-related state is selected from the group consisting of pre-term birth, full-term birth, gestational age, due date, onset of labor, pregnancy-related hypertensive disorders (e.g., preeclampsia), eclampsia, gestational diabetes, a congenital disorder of a fetus of the subject, ectopic pregnancy, spontaneous abortion, stillbirth, post-partum complications (e.g., post-partum depression, hemorrhage or excessive bleeding, pulmonary embolism, cardiomyopathy, diabetes, anemia, and hypertensive disorders), hyperemesis gravidarum (morning sickness), hemorrhage or excessive bleeding during delivery, premature rupture of membrane, premature rupture of membrane in pre-term birth, placenta previa (placenta covering the cervix), intrauterine/fetal growth restriction, macrosomia (large fetus for gestational age), neonatal conditions (e.g., anemia, apnea, bradycardia and other heart defects, bronchopulmonary dysplasia or chronic lung disease, diabetes, gastroschisis, hydrocephaly, hyperbilirubinemia, hypocalcemia, hypoglycemia, intraventricular hemorrhage, jaundice, necrotizing enterocolitis, patent ductus arteriosis, periventricular leukomalacia, persistent pulmonary hypertension, polycythemia, respiratory distress syndrome, retinopathy of prematurity, and transient tachypnea), and fetal development stages or states (e.g., normal fetal organ function or development, and abnormal fetal organ function or development). For example, the fetal development stages or states may be related to normal fetal organ function or development and/or abnormal fetal organ function or development for a fetal organ selected from the group consisting of heart, large intestine, small intestine, retina, prefrontal cortex, midbrain, kidney, and esophagus.

In another aspect, the present disclosure provides a method for determining that a subject is at risk of pre-term birth, comprising assaying a cell-free biological sample derived from the subject to generate a dataset that is indicative of the pre-term birth risk at a specificity of at least 80%, and using a trained algorithm that is trained on samples independent of the cell-free biological sample to determine that the subject is at risk of pre-term birth at an accuracy of at least about 80%. In some embodiments, the accuracy is at least about 90%.

In another aspect, the present disclosure provides a method for determining that a subject is at risk of preeclampsia, comprising assaying a cell-free biological sample derived from the subject to generate a dataset that is indicative of the preeclampsia risk at a specificity of at least 80%, and using a trained algorithm that is trained on samples independent of the cell-free biological sample to determine that the subject is at risk of preeclampsia at an accuracy of at least about 80%. In some embodiments, the accuracy is at least about 90%.

In another aspect, the present disclosure provides a method for detecting a presence or risk of a prenatal metabolic genetic disease of a fetus of a pregnant subject, comprising: assaying ribonucleic acid (RNA) in a cell-free biological sample derived from said pregnant subject to detect a set of biomarkers; and analyzing said set of biomarkers with an algorithm (e.g., a trained algorithm) to detect said presence or risk of said prenatal metabolic genetic disease.

In another aspect, the present disclosure provides a method for detecting at least two health or physiological conditions of a fetus of a pregnant subject or of said pregnant subject, comprising: assaying a first cell-free biological sample obtained or derived from said pregnant subject at a first time point and a second cell-free biological sample obtained or derived from said pregnant subject at a second time point, to detect a first set of biomarkers at said first time point and a second set of biomarkers at said second time point, and analyzing said first set of biomarkers or said second set of biomarkers with a trained algorithm to detect said at least two health or physiological conditions.

In some embodiments, said at least two health or physiological conditions are selected from the group consisting of pre-term birth, full-term birth, gestational age, due date, onset of labor, a pregnancy-related hypertensive disorder, eclampsia, gestational diabetes, a congenital disorder of a fetus of said subject, ectopic pregnancy, spontaneous abortion, stillbirth, a post-partum complication, hyperemesis gravidarum, hemorrhage or excessive bleeding during delivery, premature rupture of membrane, premature rupture of membrane in pre-term birth, placenta previa, intrauterine/fetal growth restriction, macrosomia, a neonatal condition, and a fetal development stage or state. In some embodiments, said set of biomarkers comprises a genomic locus associated with due date, wherein said genomic locus is selected from the group consisting of genes listed in Table 1, Table 7, and Table 10. In some embodiments, said set of biomarkers comprises a genomic locus associated with gestational age, wherein said genomic locus is selected from the group consisting of genes listed in Table 2, genes listed in Table 3, genes listed in Table 4, genes listed in Table 23, genes listed in Table 24, genes listed in Table 25, and genes listed in Table 26. In some embodiments, said set of biomarkers comprises a genomic locus associated with pre-term birth, wherein said genomic locus is selected from the group consisting of genes listed in Table 5, genes listed in Table 6, genes listed in Table 8, genes listed in Table 12, genes listed in Table 14, genes listed in Table 20, genes listed in Table 21, genes listed in Table 34, genes listed in Table 40, genes listed in Table 41, genes listed in Table 42, genes listed in Table 43, genes listed in Table 44, genes listed in Table 45, genes listed in Table 46, genes listed in Table 47, RAB27B, RGS18, CLCN3, B3GNT2, COL24A1, CXCL8, and PTGS2. In some embodiments, said set of biomarkers comprises at least 5 distinct genomic loci. In some embodiments, the panel of said one or more genomic loci comprises a genomic locus associated with preeclampsia, wherein the genomic locus is selected from the group consisting of genes listed in Table 15, genes listed in Table 17, genes listed in Table 18, genes listed in Table 19, genes listed in Table 27, genes listed in Table 33, CLDN7, PAPPA2, SNORD14A, PLEKHH1, MAGEA10, TLE6, and FABP1. In some embodiments, the panel of said one or more genomic loci comprises a genomic locus associated with fetal organ development, wherein the genomic locus is selected from the group of genes listed in Table 29. In some embodiments, the set of biomarkers comprises a genomic locus associated with gestational diabetes mellitus, wherein the genomic locus is selected from the group consisting of genes listed in Table 36, genes listed in Table 37, genes listed in Table 38, and genes listed in Table 39.

In another aspect, the present disclosure provides a method comprising: assaying one or more cell-free biological samples obtained or derived from a pregnant subject to detect a set of biomarkers; and analyzing said set of biomarkers to identify (1) a due date or a range thereof of a fetus of said pregnant subject and (2) a health or physiological condition of said fetus of said pregnant subject or of said pregnant subject.

In some embodiments, the method further comprises analyzing said set of biomarkers with a trained algorithm. In some embodiments, said health or physiological condition is selected from the group consisting of pre-term birth, full-term birth, gestational age, due date, onset of labor, a pregnancy-related hypertensive disorder, eclampsia, gestational diabetes, a congenital disorder of a fetus of said subject, ectopic pregnancy, spontaneous abortion, stillbirth, a post-partum complication, hyperemesis gravidarum, hemorrhage or excessive bleeding during delivery, premature rupture of membrane, premature rupture of membrane in pre-term birth, placenta previa, intrauterine/fetal growth restriction, macrosomia, a neonatal condition, and a fetal development stage or state. In some embodiments, said set of biomarkers comprises a genomic locus associated with due date, wherein said genomic locus is selected from the group consisting of genes listed in Table 1, Table 7, and Table 10. In some embodiments, said set of biomarkers comprises a genomic locus associated with gestational age, wherein said genomic locus is selected from the group consisting of genes listed in Table 2, genes listed in Table 3, genes listed in Table 4, genes listed in Table 23, genes listed in Table 24, genes listed in Table 25, and genes listed in Table 26. In some embodiments, said set of biomarkers comprises a genomic locus associated with pre-term birth, wherein said genomic locus is selected from the group consisting of genes listed in Table 5, genes listed in Table 6, genes listed in Table 8, genes listed in Table 12, genes listed in Table 14, genes listed in Table 20, genes listed in Table 21, genes listed in Table 34, genes listed in Table 40, genes listed in Table 41, genes listed in Table 42, genes, listed in Table 43, genes listed in Table 44, genes listed in Table 45, genes listed in Table 46, genes listed in Table 47, RAB27B, RGS18, CLCN3, B3GNT2, COL24A1, CXCL8, and PTGS2. In some embodiments, said set of biomarkers comprises at least 5 distinct genomic loci. In some embodiments, the panel of said one or more genomic loci comprises a genomic locus associated with preeclampsia, wherein the genomic locus is selected from the group consisting of genes listed in Table 15, genes listed in Table 17, genes listed in Table 18, genes listed in Table 19, genes listed in Table 27, genes listed in Table 33, CLDN7, PAPPA2, SNORD14A, PLEKHH1, MAGEA10, TLE6, and FABP1. In some embodiments, the panel of said one or more genomic loci comprises a genomic locus associated with fetal organ development, wherein the genomic locus is selected from the group of genes listed in Table 29. In some embodiments, the set of biomarkers comprises a genomic locus associated with gestational diabetes mellitus, wherein the genomic locus is selected from the group consisting of genes listed in Table 36, genes listed in Table 37, genes listed in Table 38, and genes listed in Table 39.

In some embodiments, the method further comprises selecting a therapeutic intervention for said health or physiological condition of said fetus of said pregnant subject or of said pregnant subject, based at least in part on said set of biomarkers. In some embodiments, said therapeutic intervention is selected from among a plurality of therapeutic interventions. In some embodiments, said therapeutic intervention is selected based at least in part on a molecular subtype of said health or physiological condition determined based at least in part on said set of biomarkers.

In some embodiments, said health or physiological condition comprises preeclampsia. In some embodiments, said therapeutic intervention for said preeclampsia comprises a drug, a supplement, or a lifestyle recommendation. In some embodiments, said drug is selected from the group consisting of aspirin, progesterone, magnesium sulfate, a cholesterol medication (such as pravastatin), a heartburn medication (such as esomeprazole), an angiotensin II receptor antagonist (such as losartan), a calcium channel blocker (such as nifedipine), a diabetes medication (such as myo-inositol, metformin, glucovance, and liraglutide), and an erectile dysfunction medication (such as sildenafil citrate). In some embodiments, said supplement is selected from the group consisting of calcium, vitamin D, vitamin B3, and DHA. In some embodiments, said lifestyle recommendation is selected from the group consisting of exercise, nutrition counseling, meditation, stress relief, weight loss or maintenance, and improving sleep quality. In some embodiments, said therapeutic intervention for said preeclampsia is selected from a therapeutic intervention (e.g., treatment or prophylaxis) as disclosed in “WHO recommendations: Prevention and treatment of pre-eclampsia and eclampsia,” World Health Organization, ISBN 9789241548335, World Health Organization, 2011, which is incorporated by reference herein in its entirety. In some embodiments, said therapeutic intervention for said preeclampsia is selected from a therapeutic intervention (e.g., treatment or prophylaxis) as disclosed in “Summary of recommendations: Prevention and treatment of pre-eclampsia and eclampsia,” World Health Organization, WHO reference number WHO/RHR/11.30, World Health Organization, 2011, which is incorporated by reference herein in its entirety. In some embodiments, said therapeutic intervention for said preeclampsia is selected from a therapeutic intervention (e.g., treatment or prophylaxis) as disclosed in “WHO recommendations: Drug treatment for severe hypertension in pregnancy,” World Health Organization, ISBN 9789241550437, World Health Organization, 2018, which is incorporated by reference herein in its entirety.

In some embodiments, said health or physiological condition comprises pre-term birth. In some embodiments, said therapeutic intervention for said pre-term birth comprises a drug, a supplement, a lifestyle recommendation, a cervical cerclage, a cervical pessary, or electrical contraction inhibition. In some embodiments, said drug is selected from the group consisting of progesterone, erythromycin, a tocolytic medication (such as indomethacin), a corticosteroid, a vaginal flora (such as clindamycin and metronidazole), and an antioxidant (such as N-acetylcysteine). In some embodiments, said supplement is selected from the group consisting of calcium, vitamin D, and a probiotic (such as lactobacillus). In some embodiments, said lifestyle recommendation is selected from the group consisting of exercise, nutrition counseling, meditation, stress relief, weight loss or maintenance, and improving sleep quality. In some embodiments, said therapeutic intervention for said pre-term birth is selected from a therapeutic intervention (e.g., treatment or prophylaxis) as disclosed “WHO Recommendations on Interventions to Improve Preterm Birth Outcomes,” ISBN 9789241508988, World Health Organization, 2015, which is incorporated by reference herein in its entirety.

In some embodiments, said health or physiological condition comprises gestational diabetes mellitus (GDM). In some embodiments, said therapeutic intervention for said GDM comprises a drug, a supplement, or a lifestyle recommendation. In some embodiments, said drug is selected from the group consisting of insulin and a diabetes medication (such as myo-inositol, metformin, glucovance, and liraglutide). In some embodiments, said supplement is selected from the group consisting of vitamin D, choline, probiotics, and DHA. In some embodiments, said lifestyle recommendation is selected from the group consisting of exercise, nutrition counseling, meditation, stress relief, weight loss or maintenance, and improving sleep quality. In some embodiments, said therapeutic intervention for said gestational diabetes mellitus (GDM) is selected from a therapeutic intervention (e.g., treatment or prophylaxis) as disclosed “Diagnostic criteria and classification of hyperglycaemia first detected in pregnancy,” WHO reference number WHO/NMH/MND/13.2, World Health Organization, 2013, which is incorporated by reference herein in its entirety.

In another aspect, the present disclosure provides a method comprising: assaying one or more cell-free biological samples obtained or derived from a pregnant subject to detect a set of nucleic acids of non-human origin; and analyzing said set of nucleic acids of non-human origin to detect a health or physiological condition of a fetus of said pregnant subject or of said pregnant subject. In some embodiments, the nucleic acids of non-human origin comprise DNA or RNA of a non-human organism. In some embodiments, the non-human organism is a bacteria, a virus, or a parasite. In some embodiments, the method further comprises analyzing said set of nucleic acids of non-human origin using a trained algorithm.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates an example workflow of a method for identifying or monitoring a pregnancy-related state of a subject, in accordance with disclosed embodiments.

FIG. 2 illustrates a computer system that is programmed or otherwise configured to implement methods provided herein.

FIG. 3A shows a first cohort of subjects (e.g., pregnant women) that was established (with patient identification numbers shown on the x-axis), from which one or more biological samples (e.g., 2 or 3 each) were collected and assayed at different time points corresponding to an estimated gestational age (shown on the y-axis, in increasing order of estimated gestational age at delivery) of a fetus of each subject, in accordance with disclosed embodiments.

FIG. 3B shows a distribution of participants in the first cohort based on each participant's age at the time of medical record abstraction, in accordance with disclosed embodiments.

FIG. 3C shows a distribution of 100 participants in the first cohort based on each participant's race, in accordance with disclosed embodiments.

FIG. 3D shows a distribution of collected samples in the gestational age cohort based on each participant's estimated gestational age and trimester at the time of collection of each sample, in accordance with disclosed embodiments.

FIG. 3E shows a distribution of 225 collected samples in the first cohort based on the study sample type of the collected samples, in accordance with disclosed embodiments.

FIG. 4A shows a second cohort of subjects (e.g., pregnant women) that was established (with patient identification numbers shown on the x-axis), from which one or more biological samples (e.g., 1, 2, or 3 each) were collected and assayed at different time points corresponding to an estimated gestational age (shown on the y-axis, in increasing order of estimated gestational age at delivery) of a fetus of each subject, in accordance with disclosed embodiments.

FIG. 4B shows a distribution of participants in the second cohort based on each participant's age at the time of medical record abstraction, in accordance with disclosed embodiments.

FIG. 4C shows a distribution of 128 participants in the second cohort based on each participant's race, in accordance with disclosed embodiments.

FIG. 4D shows a distribution of collected samples in the second cohort based on each participant's estimated gestational age and trimester at the time of collection of each sample, in accordance with disclosed embodiments.

FIG. 4E shows a distribution of 160 collected samples in the second cohort based on the study sample type of the collected samples, in accordance with disclosed embodiments.

FIG. 5A shows a due date cohort of subjects (e.g., pregnant women) that was established (with patient identification numbers shown on the x-axis), from which one or more biological samples (e.g., 1 or 2 each) were collected and assayed at different time points corresponding to an estimated gestational age (shown on the y-axis, in increasing order of estimated gestational age at delivery) of a fetus of each subject, in accordance with disclosed embodiments.

FIG. 5B shows a distribution of collected samples in the due date cohort based on the time between the date of sample collection and the date of delivery (time to delivery), in accordance with disclosed embodiments.

FIG. 5C is a Venn diagram showing the overlap of genes used in the first and second predictive models of due date, in accordance with disclosed embodiments. The first predictive model had a total of 51 most predictive genes, and the second predictive model had a total of 49 most predictive genes; further, only 5 genes overlapped between the two predictive models.

FIG. 5D is a plot showing the concordance between a predicted time to delivery (in weeks) and the observed (actual) time to delivery (in weeks) for the subjects in the due date cohort, in accordance with disclosed embodiments.

FIG. 5E shows a summary of the predictive models for predicting due date, including a predictive model using samples with a time-to-delivery of less than 5 weeks and predictive model using samples with a time-to-delivery of less than 7.5 weeks; different predictive models were generated with estimated due date information (e.g., determined using estimated gestational age from ultrasound measurements) and without the estimated due date information.

FIG. 6A shows a gestational age cohort of subjects (e.g., pregnant women) that was established (with patient identification numbers shown on the x-axis), from which one or more biological samples (e.g., 1 or 2 each) were collected and assayed at different time points corresponding to an estimated gestational age (shown on the y-axis, in increasing order of estimated gestational age at delivery) of a fetus of each subject, in accordance with disclosed embodiments.

FIG. 6B is a visual model showing mutual information of the whole transcriptome, where expression of a plurality of gestational age-associated genes varies with gestational age throughout the course of a pregnancy, in accordance with disclosed embodiments.

FIG. 6C is a plot showing the concordance between a predicted gestational age (in weeks) and the measured gestational age (in weeks) for the subjects in the gestational age cohort, in accordance with disclosed embodiments. The subjects are stratified in the plot by major race (e.g., white, non-black Hispanic, Asian, Afro-American, Native American, mixed race (e.g., two or more races), or unknown).

FIGS. 7A-7B show results for a pre-term birth (PTB) cohort of subjects (e.g., pregnant women), which included a set of pre-term case samples (e.g., from women having pre-term births) and a set of pre-term control samples (e.g., from women having full-term births), in accordance with disclosed embodiments. Across the pre-term case samples and pre-term control samples, the distributions of gestational age at time of collection were similar (FIG. 7A), while the distributions of gestational age at delivery were clearly distinguishable to a statistically significant extent (FIG. 7B).

FIGS. 7C-7E show differential gene expression of the B3GNT2, BPI, and ELANE genes, respectively, between the pre-term case samples (left) and pre-term control samples (right), in accordance with disclosed embodiments.

FIG. 7F shows a legend for the results from pre-term case samples and pre-term control samples shown in FIGS. 7C-7E, in accordance with disclosed embodiments.

FIG. 7G shows a receiver-operating characteristic (ROC) curve showing the performance of the predictive model for pre-term delivery across the 10-fold cross-validation, in accordance with disclosed embodiments.

FIG. 8 shows an example of a distribution of vaginal singleton births by obstetrician-estimated gestational age in the U.S.

FIG. 9A-9E show different methods of predicting due date for a fetus of a pregnant subject, including predicting an actual day (with error) (FIG. 9A), predicting a week (or other window) of delivery (FIG. 9B), predicting whether a delivery is expected to occur before or after a certain time boundary (FIG. 9C), predicting in which bin among a plurality of bins (e.g., 6 bins) a delivery is expected to occur (FIG. 9D), and predicting a relative risk or relative likelihood of an early delivery or a late delivery (FIG. 9E).

FIG. 10 shows a data workflow that is performed to develop a due date prediction model (e.g., classifier).

FIGS. 11A-11B show prediction error of a due date prediction model that is trained on 270 and 310 patients, respectively.

FIG. 12 shows a receiver-operator characteristic ROC) curve for a pre-term birth prediction model, using a set of 22 genes for a set of 79 samples obtained from a cohort of Caucasian subjects. The mean area-under-the-curve (AUC) for the ROC curve was 0.91±0.10.

FIG. 13A shows a receiver-operator characteristic ROC) curve for a pre-term birth prediction model, using a set of genes for a set of 45 samples obtained from a cohort of subjects having African or African-American ancestries (AA cohort). The mean area-under-the-curve (AUC) for the ROC curve was 0.82±0.08.

FIG. 13B shows a gene panel for a pre-term birth prediction model for three different AA cohorts (cohort 1, cohort 2, and cohort 3), including RAB27B, RGS18, CLCN3, B3GNT2, COL24A1, CXCL8, and PTGS2.

FIG. 14A shows a workflow for performing multiple assays for assessment of a plurality of pregnancy-related conditions using a single bodily sample (e.g., a single blood draw) obtained from a pregnant subject.

FIG. 14B shows a combination of conditions which can be tested from a single blood draw along a pregnancy progression of a pregnant subject.

FIG. 15A shows a Discovery 1 cohort of 310 mixed race subjects (e.g., pregnant women) that was established (with patient identification numbers shown on the x-axis), from which biological samples were collected and assayed at different time points corresponding to an estimated gestational age (shown on the y-axis, in increasing order of estimated gestational age at delivery) of a fetus of each subject, in accordance with disclosed embodiments.

FIG. 15B shows a Discovery 2 cohort of 86 Caucasian subjects, respectively, that was established (with patient identification numbers shown on the x-axis), from which biological samples were collected and assayed at different time points corresponding to an estimated gestational age (shown on the y-axis, in increasing order of estimated gestational age at delivery) of a fetus of each subject, in accordance with disclosed embodiments.

FIG. 15C shows a distribution of participants in the Discovery 1 mixed race cohort based on blood sample collection gestation.

FIG. 15D shows a distribution of participants in the Discovery 2 Caucasian cohort, respectively, based on blood sample collection gestation.

FIG. 15E shows a distribution of samples collected in the Discovery 1 mixed race cohort by weeks before birth.

FIG. 15F shows a distribution of participants in the Discovery 2 Caucasian cohort by weeks before birth.

FIG. 16A shows expression trends and significant abundance level separation for a set of top 4 genes (EFHD1, ADCY6, HTR1, and PAPPA2) between samples collected at 1 week before birth.

FIG. 16B shows correlation p-value significance of log₁₀(p-value) exceeds a threshold of 1 for 3 genes (HTRA1, PAPPA2, and EFHD1) in several discovery and validation cohorts.

FIG. 17A shows a first cohort of 192 subjects (e.g., pregnant women) that was established (with patient identification numbers shown on the x-axis), from which biological samples were collected and assayed at different time points corresponding to an estimated gestational age (shown on the y-axis, in increasing order of estimated gestational age at delivery) of a fetus of each subject, in accordance with disclosed embodiments.

FIG. 17B shows a first cohort distribution of participants in case (upper graph) and control (lower graph) group based on each participant's age at the time of medical record abstraction, in accordance with disclosed embodiments.

FIG. 17C shows a first cohort distribution of participants in case (left graph) and control (right graph) group based on each participant's race, in accordance with disclosed embodiments.

FIG. 17D shows a distribution of 192 collected samples in the first cohort based on the study sample type of the collected samples.

FIG. 18A shows a second cohort of 76 subjects (e.g., pregnant women) that was established (with patient identification numbers shown on the x-axis), from which biological samples were collected and assayed at different time points corresponding to an estimated gestational age (shown on the y-axis, in increasing order of estimated gestational age at delivery) of a fetus of each subject, in accordance with disclosed embodiments.

FIG. 18B shows a second cohort distribution of participants in case (left graph) and control (right graph) group based on each participant's race, in accordance with disclosed embodiments.

FIG. 18C shows a distribution of 76 collected samples (25 pre-term samples and 51 full-term controls) in the second cohort based on the study sample type of the collected samples.

FIG. 19A shows a quantile-quantile (QQ) plot for a signal in pre-term birth-associated genes in the first cohort.

FIG. 19B shows a receiver-operator characteristic (ROC) curve for the high pre-term birth prediction model, using all differentially expressed genes in the first cohort. The mean area-under-the-curve (AUC) for the ROC curve was 0.75±0.08.

FIG. 19C shows a receiver-operator characteristic (ROC) curve for a set of top 9 genes (EFHD1, ABI3BP, NEAT1, HSD17B1, CDR1-AS, GCM1, DAPK2, ZCCHC7, COL3A1, and AKR7A2) in the first cohort. The mean area-under-the-curve (AUC) for the ROC curve was 0.80±0.07, with relative contributions from each gene.

FIG. 20A shows a distribution of demographic statistics for this subset of early PTB samples and controls in the second cohort that were included in the analysis.

FIG. 20B shows a quantile-quantile (QQ) plot for a differential expression signal in pre-term birth-associated genes in the second cohort.

FIG. 20C shows boxplots and significant abundance level separation for the top 12 differentially expressed genes (ANGPTL3, NPM1P26, HIST1H4F, CRY1, BHMT, C2orf49, OASL, SELE, CHD4, IFIT1, DHX38, and DNASE1) for early PTB in the second cohort.

FIG. 21 shows a first cohort of 18 subjects (e.g., pregnant women) that was established (with patient identification numbers shown on the x-axis), from which biological samples were collected and assayed at different time points corresponding to an estimated gestational age (shown on the y-axis, in increasing order of estimated gestational age at delivery) of a fetus of each subject, in accordance with disclosed embodiments.

FIG. 22A shows a second cohort of 130 subjects (pregnant women) that was established (with patient identification numbers shown on the x-axis), from which 144 biological samples were collected and assayed at different time points corresponding to an estimated gestational age (shown on the y-axis, in increasing order of estimated gestational age at delivery) of a fetus of each subject, in accordance with disclosed embodiments.

FIG. 22B shows a second cohort distribution of 130 participants in case (left graph) and control (right graph) group based on each participant's race, in accordance with disclosed embodiments.

FIG. 22C shows a distribution of 144 collected samples in the second cohort based on the study sample type of the collected samples.

FIG. 23 shows a significant abundance level separation between cases and healthy controls for the top 20 differentially expressed genes for preeclampsia (PE) in the first cohort.

FIG. 24A shows a distribution of demographic statistics for the subset of PE samples and controls in the second cohort.

FIG. 24B shows a quantile-quantile (QQ) plot for a differential expression signal in preeclampsia-associated genes in the second cohort.

FIG. 24C show boxplots and significant abundance level separation in a set of top 12 genes for preeclampsia in the second cohort (AGAP9, ANKRD1, CIS, CCDC181, CIAPIN1, EPS8L1, FBLN1, FUNDC2P2, KISS1, MLF1, PAPPA2, and TFPI2).

FIG. 25A shows a cohort of 351 subjects (pregnant women) that was established (with patient identification numbers shown on the x-axis), from which 351 biological samples were collected and assayed at different time points corresponding to an estimated gestational age (shown on the y-axis, in increasing order of estimated gestational age at delivery) of a fetus of each subject, in accordance with disclosed embodiments.

FIG. 25B shows quantile-quantile (QQ) plots for a differential expression signal in preeclampsia-associated genes in the analyses with and without chronic hypertension control subjects.

FIG. 25C shows a receiver-operator characteristic (ROC) curve for a training cohort (Example 9) and a test (Example 10) cohort for a preeclampsia prediction model, using all differentially expressed genes in the Example 9 cohort. The mean area-under-the-curve (AUC) for the ROC curve was 0.75 and 0.66 for the training cohort and the test cohort, respectively.

FIG. 25D shows a receiver-operator characteristic (ROC) curve for combined cohorts. The mean area-under-the-curve (AUC) for the ROC curve was 0.76.

FIG. 26A shows a combined data set for pre-term birth cohorts from Example 4 and Example 8, and an additional cohort based on blood collection and delivery gestational age.

FIG. 26B shows a cohort of 281 subjects (pregnant women) that was established (with patient identification numbers shown on the x-axis), from which 281 biological samples were collected and assayed at different time points corresponding to an estimated gestational age (shown on the y-axis, in increasing order of estimated gestational age at delivery) of a fetus of each subject, in accordance with disclosed embodiments.

FIG. 26C shows a quantile-quantile (QQ) plot for a differential expression signal in pre-term birth cases with delivery between 28 to 35 weeks for blood samples collected from subjects at between 20 to 28 weeks of gestation age.

FIG. 27A shows a combined data set for combined cohorts based on blood collection and delivery gestational age, which comprises different races of maternal donors.

FIG. 27B is a plot showing the relationship between a predicted gestational age (in weeks) and the measured gestational age (in weeks) for the subjects in the gestational age cohort in held-out test data. Gray bands represent one and two standard deviations. 494 genes were used for Lasso modeling.

FIG. 27C is a plot showing the concordance between a predicted gestational age (in weeks) and the measured gestational age (in weeks) for the subjects in the gestational age cohort in held-out test data. 57 transcriptomic features were used for Lasso modeling.

FIG. 27D is a plot showing the concordance between a predicted gestational age (in weeks) and the measured gestational age (in weeks) for the subjects in the gestational age cohort in the held-out testing data. 70 genes were used for the RFE method.

FIG. 27E is a plot showing the concordance between a predicted gestational age (in weeks) and the measured gestational age (in weeks) for the subjects in the gestational age cohort in held-out test data in first trimester modeling.

FIG. 28A shows a quantile-quantile (QQ) plot for differential expression between preeclampsia and control for genes across the whole transcriptome in one of the outer training sets. FABP1 is labeled to highlight its relative ranking among the differentially expressed genes.

FIG. 28B shows the distribution of the area-under-the-curve (AUC) across the one hundred held-out outer testing sets for a preeclampsia prediction linear model based on FABP1. The mean AUC across the outer testing sets is 0.67.

FIG. 28C shows the distribution of the area-under-the-curve (AUC) across the one hundred held-out outer testing sets for a preeclampsia prediction linear model based on PAPPA2 in combination with the nine abundant genes with significant differential expression (adjusted p-value<0.05) between preeclampsia cases and controls. The nine abundant genes include FABP1, CDCA2, HMGB3, ELANE, CDC20, SHCBP1, OLFM4, S100A9, S100A12. The mean AUC across the outer testing sets is 0.73.

FIG. 29A shows upward temporal profiles of fetal organ developmental signatures of fetal small intestine, developing hearts, and fetal retina gene sets in training cohort. Plasma transcriptome fractions for 3 top upregulated embryonic gene sets were averaged across all samples in a given collection window with error bars corresponding to 95% confidence interval around the mean.

FIG. 29B shows upward trends for fetal organ developmental signatures of fetal small intestine, developing hearts, and fetal retina gene sets in the training and holdout cohorts as a linear function of gestational age.

FIG. 29C shows the verification modeling of the top three downward trending gene sets with gestation age (kidney nephron progenitor cells, esophagus C4 epithelial cells, and prefrontal cortex (PFC) brain C4 cells in training (H) and held out test cohorts (A, B, G).

FIG. 30 shows plasma sampling and cohort overview by gestational age. Different cohorts labeled are A-H. Circles represent plasma samples from liquid biopsies. Maternal donors are of different races.

FIGS. 31A-31C show gestational age modeling in full term pregnancies. FIG. 31A: Model predictions from held-out test cfRNA transcript data in Lasso linear model versus ultrasound predicted gestational age. Dark gray zone is 1 standard deviation, light gray zone is 2 standard deviations. FIG. 31B: Variance explained from ANOVA. FIG. 31C: Learning curve for gestational age modeling. Model for gestational age is trained with increasing sample size, error is plotted for both training set (Cross-validated) and held-out test set. Error bars are 1 standard deviation.

FIGS. 32A-32C show temporal profiles of developmental signatures from embryonic gene sets. Maternal plasma transcriptome fractions for gene set averaged across all samples in a given collection window. FIG. 32A: Fetal small intestine gene set. FIG. 32B: Developing heart gene set. FIG. 32C: Nephron progenitor gene set. Error bars correspond to 95% confidence interval around the mean. CPM, counts per million. N=91 for each timepoint and gene set.

FIGS. 33A-33B show features and model performance for prediction of preeclampsia. FIG. 33A: Quantile-quantile plot ranked Spearman p-values for preeclamptic women versus controls. p-values are calculated from Spearman correlations on cohort corrected data for each gene. Genes used in model are labeled. Black dotted line is expectation. FIG. 33B: Receiver operating characteristic curve (mean and 95% confidence intervals) for logistic regression model for preeclampsia without the intermediate risk group.

FIG. 34 shows principal components analysis of all samples used in the gestational age model.

FIGS. 35A-35B show temporal profiles of pregnancy-related endocrine signatures during pregnancy. Seven pregnancy-related gene ontology term signatures identified as highly significantly enriched (α=0.01) were profiled across collection times using cumulative CPM. Plasma transcriptome fractions for each gene set were averaged across all samples in a given collection window with error bars corresponding to 95% confidence interval around the mean. Panels correspond to different ranges of CPM, for the ease of comparison. CPM, counts per million. N=91 for each timepoint and gene set.

FIG. 36 shows validation of gene set signature across all cohorts with longitudinal samples. Linear fits of transcriptome fractions for all samples across corresponding gestational ages recorded at the collection times. The band around the solid line corresponds to the 95% CI. a, Fetal small intestine gene set. b, Developing heart gene set. c, Nephron progenitor gene set. All slopes for the gestational age coefficient are distinct from 0 at a confidence level of 0.05, except for the “Nephron progenitor” set in cohort G.

FIG. 37 shows temporal structure in the data determines the trends. For each of the significantly enriched gene sets, the trends were evaluated by bootstrapping (B=1,000) the original data (blue lines) and the time-scrambled data obtained by reshuffling collection times (grey lines). a, Fetal small intestine gene set. b, Developing heart gene set. c, Nephron progenitor gene set.

FIGS. 38A-38B show gene set enrichment analysis for gene ontology sets. a, Top-20 upregulated gene sets. b, Top-20 downregulated gene sets. ES, enrichment score. −ES, negative enrichment score. Color gradient for adjusted p-value.

FIG. 39 shows a quantile-quantile (QQ) plot for a differential expression signal in a QQ plot for differential expression in ePTB cases.

FIG. 40 shows a quantile-quantile (QQ) plot for a differential expression signal in a QQ plot for differential expression in gestational diabetes mellitus (GDM) cases, including the top 4 differentially expressed genes.

FIG. 41 shows a clinical intervention care plan algorithm to improve early pre-term birth outcomes following results of predictive tests administered in the second trimester.

FIG. 42 shows a clinical intervention care plan algorithm to improve preeclampsia outcomes following results of predictive tests administered in the second trimester.

FIG. 43 shows a clinical intervention care plan algorithm to improve gestational diabetes mellitus (GDM) outcomes based on prediction test administered in the second trimester.

FIG. 44A shows a combined data set for pre-term birth cohorts from Examples 4, 8, and 11, and an additional cohort based on blood collection and delivery gestational age.

FIG. 44B shows a cohort of 150 subjects (pregnant women) that was established (with patient identification numbers shown on the x-axis), from which 150 biological samples were collected and assayed at different time points corresponding to an estimated gestational age (shown on the y-axis, in increasing order of estimated gestational age at delivery) of a fetus of each subject.

FIG. 44C shows a quantile-quantile (QQ) plot for a differential expression signal in a QQ plot for differentially expressed genes in pre-term birth cases for samples collected between 17 and 28 weeks of gestation.

FIG. 44D shows a quantile-quantile (QQ) plot for a differential expression signal in a QQ plot for differentially expressed genes in pre-term birth cases for samples collected between 23 and 26 weeks of gestation.

FIG. 44E shows a quantile-quantile (QQ) plot for a differential expression signal in a QQ plot for differentially expressed genes in pre-term birth cases for samples collected between 17 and 23 weeks of gestation.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used in the specification and claims, the singular form “a”, “an”, and “the” include plural references unless the context clearly dictates otherwise. For example, the term “a nucleic acid” includes a plurality of nucleic acids, including mixtures thereof.

As used herein, the term “subject,” generally refers to an entity or a medium that has testable or detectable genetic information. A subject can be a person, individual, or patient. A subject can be a vertebrate, such as, for example, a mammal. Non-limiting examples of mammals include humans, simians, farm animals, sport animals, rodents, and pets. A subject can be a pregnant female subject. The subject can be a woman having a fetus (or multiple fetuses) or suspected of having the fetus (or multiple fetuses). The subject can be a person that is pregnant or is suspected of being pregnant. The subject may be displaying a symptom(s) indicative of a health or physiological state or condition of the subject, such as a pregnancy-related health or physiological state or condition of the subject. As an alternative, the subject can be asymptomatic with respect to such health or physiological state or condition.

The term “pregnancy-related state,” as used herein, generally refers to any health, physiological, and/or biochemical state or condition of a subject that is pregnant or is suspected of being pregnant, or of a fetus (or multiple fetuses) of the subject. Examples of pregnancy-related states include, without limitation, pre-term birth, full-term birth, gestational age, due date, onset of labor, pregnancy-related hypertensive disorders (e.g., preeclampsia), eclampsia, gestational diabetes, a congenital disorder of a fetus of the subject, ectopic pregnancy, spontaneous abortion, stillbirth, post-partum complications (e.g., post-partum depression, hemorrhage or excessive bleeding, pulmonary embolism, cardiomyopathy, diabetes, anemia, and hypertensive disorders), hyperemesis gravidarum (morning sickness), hemorrhage or excessive bleeding during delivery, premature rupture of membrane, premature rupture of membrane in pre-term birth, placenta previa (placenta covering the cervix), intrauterine/fetal growth restriction, macrosomia (large fetus for gestational age), neonatal conditions (e.g., anemia, apnea, bradycardia and other heart defects, bronchopulmonary dysplasia or chronic lung disease, diabetes, gastroschisis, hydrocephaly, hyperbilirubinemia, hypocalcemia, hypoglycemia, intraventricular hemorrhage, jaundice, necrotizing enterocolitis, patent ductus arteriosis, periventricular leukomalacia, persistent pulmonary hypertension, polycythemia, respiratory distress syndrome, retinopathy of prematurity, and transient tachypnea), and fetal development stages or states (e.g., normal fetal organ function or development, and abnormal fetal organ function or development). For example, the fetal development stages or states may be related to normal fetal organ function or development and/or abnormal fetal organ function or development for a fetal organ selected from the group consisting of heart, large intestine, small intestine, retina, prefrontal cortex, midbrain, kidney, and esophagus. In some situations, the pregnancy-related state is not associated with the health or physiological state or condition of a fetus (or multiple fetuses) of the subject.

As used herein, the term “sample,” generally refers to a biological sample obtained from or derived from one or more subjects. Biological samples may be cell-free biological samples or substantially cell-free biological samples, or may be processed or fractionated to produce cell-free biological samples. For example, cell-free biological samples may include cell-free ribonucleic acid (cfRNA), cell-free deoxyribonucleic acid (cfDNA), cell-free fetal DNA (cffDNA), plasma, serum, urine, saliva, amniotic fluid, and derivatives thereof. Cell-free biological samples may be obtained or derived from subjects using an ethylenediaminetetraacetic acid (EDTA) collection tube, a cell-free RNA collection tube (e.g., Streck), or a cell-free DNA collection tube (e.g., Streck). Cell-free biological samples may be derived from whole blood samples by fractionation. Biological samples or derivatives thereof may contain cells. For example, a biological sample may be a blood sample or a derivative thereof (e.g., blood collected by a collection tube or blood drops), a vaginal sample (e.g., a vaginal swab), or a cervical sample (e.g., a cervical swab).

As used herein, the term “nucleic acid” generally refers to a polymeric form of nucleotides of any length, either deoxyribonucleotides (dNTPs) or ribonucleotides (rNTPs), or analogs thereof. Nucleic acids may have any three-dimensional structure, and may perform any function, known or unknown. Non-limiting examples of nucleic acids include deoxyribonucleic (DNA), ribonucleic acid (RNA), coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or after assembly of the nucleic acid. The sequence of nucleotides of a nucleic acid may be interrupted by non-nucleotide components. A nucleic acid may be further modified after polymerization, such as by conjugation or binding with a reporter agent.

As used herein, the term “target nucleic acid” generally refers to a nucleic acid molecule in a starting population of nucleic acid molecules having a nucleotide sequence whose presence, amount, and/or sequence, or changes in one or more of these, are desired to be determined. A target nucleic acid may be any type of nucleic acid, including DNA, RNA, and analogs thereof. As used herein, a “target ribonucleic acid (RNA)” generally refers to a target nucleic acid that is RNA. As used herein, a “target deoxyribonucleic acid (DNA)” generally refers to a target nucleic acid that is DNA.

As used herein, the terms “amplifying” and “amplification” generally refer to increasing the size or quantity of a nucleic acid molecule. The nucleic acid molecule may be single-stranded or double-stranded. Amplification may include generating one or more copies or “amplified product” of the nucleic acid molecule. Amplification may be performed, for example, by extension (e.g., primer extension) or ligation. Amplification may include performing a primer extension reaction to generate a strand complementary to a single-stranded nucleic acid molecule, and in some cases generate one or more copies of the strand and/or the single-stranded nucleic acid molecule. The term “DNA amplification” generally refers to generating one or more copies of a DNA molecule or “amplified DNA product.” The term “reverse transcription amplification” generally refers to the generation of deoxyribonucleic acid (DNA) from a ribonucleic acid (RNA) template via the action of a reverse transcriptase.

Every year, about 15 million pre-term births are reported globally. Pre-term birth may affect as many as about 10% of pregnancies, of which the majority are spontaneous pre-term births. Currently, there may be no meaningful, clinically actionable diagnostic screenings or tests available for many pregnancy-related complications such as pre-term birth. However, pregnancy-related complications such as pre-term birth are a leading cause of neonatal death and of complications later in life. Further, such pregnancy-related complications can cause negative health effects on maternal health. Thus, to make pregnancy as safe as possible, there exists a need for rapid, accurate methods for identifying and monitoring pregnancy-related states that are non-invasive and cost-effective, toward improving maternal and fetal health.

Current tests for prenatal care may be in inaccessible and incomplete. For cases in which pregnancies progress without pregnancy-related complications, limited methods of pregnancy monitoring may be available for a pregnancy subject, such as molecular tests, ultrasound imaging, and estimation of gestational age and/or due date using the last menstrual period. However, such monitoring methods may be complex, expensive, and unreliable. For example, molecular tests cannot predict gestational age, ultrasound imaging is expensive and best performed during the first trimester of pregnancy, and estimation of gestational age and/or due date using the last menstrual period can be unreliable. Further, for cases in which pregnancies progress with pregnancy-related complications such as risk of spontaneous pre-term delivery, the clinical utility of molecular tests, ultrasound imaging, and demographic factors may be limited. For example, molecular tests may have a limited BMI (body mass index) range, a limited gestational age and/or due date range (about 2 weeks), and a low positive predictive value (PPV); ultrasound imaging may be expensive and have low PPV and specificity; and the use of demographic factors to predict risk of pregnancy-related complications may be unreliable. Therefore, there exists an urgent clinical need for accurate and affordable non-invasive diagnostic methods for detection and monitoring of pregnancy-related states (e.g., estimation of gestational age, due date, and/or onset of labor, and prediction of pregnancy-related complications such as pre-term birth) toward clinically actionable outcomes.

The present disclosure provides methods, systems, and kits for identifying or monitoring pregnancy-related states by processing cell-free biological samples obtained from or derived from subjects (e.g., pregnancy female subjects). Cell-free biological samples (e.g., plasma samples) obtained from subjects may be analyzed to identify the pregnancy-related state (which may include, e.g., measuring a presence, absence, or quantitative assessment (e.g., risk) of the pregnancy-related state). Such subjects may include subjects with one or more pregnancy-related states and subjects without pregnancy-related states. Pregnancy-related states may include, for example, pre-term birth, full-term birth, gestational age, due date, onset of labor, pregnancy-related hypertensive disorders (e.g., preeclampsia), eclampsia, gestational diabetes, a congenital disorder of a fetus of the subject, ectopic pregnancy, spontaneous abortion, stillbirth, post-partum complications (e.g., post-partum depression, hemorrhage or excessive bleeding, pulmonary embolism, cardiomyopathy, diabetes, anemia, and hypertensive disorders), hyperemesis gravidarum (morning sickness), hemorrhage or excessive bleeding during delivery, premature rupture of membrane, premature rupture of membrane in pre-term birth, placenta previa (placenta covering the cervix), intrauterine/fetal growth restriction, and macrosomia (large fetus for gestational age). In some embodiments, pregnancy-related states are not associated with the health of a fetus. In some embodiments, pregnancy-related states include neonatal conditions (e.g., anemia, apnea, bradycardia and other heart defects, bronchopulmonary dysplasia or chronic lung disease, diabetes, gastroschisis, hydrocephaly, hyperbilirubinemia, hypocalcemia, hypoglycemia, intraventricular hemorrhage, jaundice, necrotizing enterocolitis, patent ductus arteriosis, periventricular leukomalacia, persistent pulmonary hypertension, polycythemia, respiratory distress syndrome, retinopathy of prematurity, and transient tachypnea) and fetal development stages or states (e.g., normal fetal organ function or development, and abnormal fetal organ function or development). For example, the fetal development stages or states may be related to normal fetal organ function or development and/or abnormal fetal organ function or development for a fetal organ selected from the group consisting of heart, large intestine, small intestine, retina, prefrontal cortex, midbrain, kidney, and esophagus.

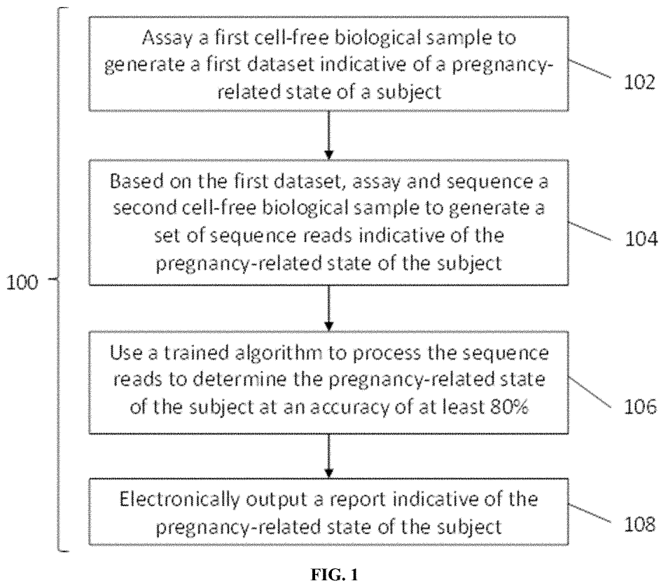

FIG. 1 illustrates an example workflow of a method for identifying or monitoring a pregnancy-related state of a subject, in accordance with disclosed embodiments. In an aspect, the present disclosure provides a method 100 for identifying or monitoring a pregnancy-related state of a subject. The method 100 may comprise using a first assay to process a first cell-free biological sample derived from said subject to generate a first dataset (as in operation 102). Next, based at least in part on the first dataset generated, the method 100 may optionally comprise using a second assay (e.g., different from the first assay) to process a second cell-free biological sample derived from the subject to generate a second dataset indicative of the pregnancy-related state at a specificity greater than the first dataset. For example, ribonucleic acid (RNA) molecules extracted from a second cell-free plasma sample may be sequenced to generate a set of sequence reads indicative of a pregnancy-related state of the subject (as in operation 104). In some embodiments, a first cell-free biological sample can be obtained from a subject at a first time point for processing with a first assay. Then, optionally a second cell-free biological sample can be obtained from the same subject at a second time point for processing with a second assay. In some embodiments, a cell-free biological sample can be obtained from a subject and then aliquoted to produce a first cell-free biological sample and a second cell-free biological sample, which are then processed with a first assay and a second assay, respectively. Next, a trained algorithm may be used to process the first dataset and/or the second dataset to determine the pregnancy-related state of the subject (as in operation 106). The trained algorithm may be configured to identify the pregnancy-related state at an accuracy of at least about 80% over 50 independent samples. A report may then be electronically outputted that is indicative of (e.g., identifies or provides an indication of) presence or susceptibility of the pregnancy-related state of the subject (as in operation 108).

### Assaying Cell-Free Biological Samples

The cell-free biological samples may be obtained or derived from a human subject (e.g., a pregnant female subject). The cell-free biological samples may be stored in a variety of storage conditions before processing, such as different temperatures (e.g., at room temperature, under refrigeration or freezer conditions, at 25° C., at 4° C., at −18° C., −20° C., or at −80° C.) or different suspensions (e.g., EDTA collection tubes, cell-free RNA collection tubes, or cell-free DNA collection tubes).

The cell-free biological sample may be obtained from a subject with a pregnancy-related state (e.g., a pregnancy-related complication), from a subject that is suspected of having a pregnancy-related state (e.g., a pregnancy-related complication), or from a subject that does not have or is not suspected of having the pregnancy-related state (e.g., a pregnancy-related complication). The pregnancy-related state may comprise a pregnancy-related complication, such as pre-term birth, pregnancy-related hypertensive disorders (e.g., preeclampsia), eclampsia, gestational diabetes, a congenital disorder of a fetus of the subject, ectopic pregnancy, spontaneous abortion, stillbirth, post-partum complications (e.g., post-partum depression, hemorrhage or excessive bleeding, pulmonary embolism, cardiomyopathy, diabetes, anemia, and hypertensive disorders), hyperemesis gravidarum (morning sickness), hemorrhage or excessive bleeding during delivery, premature rupture of membrane, premature rupture of membrane in pre-term birth, placenta previa (placenta covering the cervix), intrauterine/fetal growth restriction, macrosomia (large fetus for gestational age), neonatal conditions (e.g., anemia, apnea, bradycardia and other heart defects, bronchopulmonary dysplasia or chronic lung disease, diabetes, gastroschisis, hydrocephaly, hyperbilirubinemia, hypocalcemia, hypoglycemia, intraventricular hemorrhage, jaundice, necrotizing enterocolitis, patent ductus arteriosis, periventricular leukomalacia, persistent pulmonary hypertension, polycythemia, respiratory distress syndrome, retinopathy of prematurity, and transient tachypnea), and abnormal fetal development stages or states (e.g., abnormal fetal organ function or development). The pregnancy-related state may comprise a full-term birth, normal fetal development stages or states (e.g., normal fetal organ function or development), or absence of a pregnancy-related complication (e.g., pre-term birth, pregnancy-related hypertensive disorders (e.g., preeclampsia), eclampsia, gestational diabetes, a congenital disorder of a fetus of the subject, ectopic pregnancy, spontaneous abortion, stillbirth, post-partum complications (e.g., post-partum depression, hemorrhage or excessive bleeding, pulmonary embolism, cardiomyopathy, diabetes, anemia, and hypertensive disorders), hyperemesis gravidarum (morning sickness), hemorrhage or excessive bleeding during delivery, premature rupture of membrane, premature rupture of membrane in pre-term birth, placenta previa (placenta covering the cervix), intrauterine/fetal growth restriction, macrosomia (large fetus for gestational age), neonatal conditions (e.g., anemia, apnea, bradycardia and other heart defects, bronchopulmonary dysplasia or chronic lung disease, diabetes, gastroschisis, hydrocephaly, hyperbilirubinemia, hypocalcemia, hypoglycemia, intraventricular hemorrhage, jaundice, necrotizing enterocolitis, patent ductus arteriosis, periventricular leukomalacia, persistent pulmonary hypertension, polycythemia, respiratory distress syndrome, retinopathy of prematurity, and transient tachypnea), and abnormal fetal development stages or states (e.g., abnormal fetal organ function or development)). The pregnancy-related state may comprise a quantitative assessment of pregnancy such as gestational age (e.g., measured in days, weeks or months) or due date (e.g., expressed as a predicted or estimated calendar date or range of calendar dates). The pregnancy-related state may comprise a quantitative assessment of a pregnancy-related complication such as a likelihood, a susceptibility, or a risk (e.g., expressed as a probability, a relative probability, an odds ratio, or a risk score or risk index) of the pregnancy-related complication (e.g., pre-term birth, onset of labor, pregnancy-related hypertensive disorders (e.g., preeclampsia), eclampsia, gestational diabetes, a congenital disorder of a fetus of the subject, ectopic pregnancy, spontaneous abortion, stillbirth, post-partum complications (e.g., post-partum depression, hemorrhage or excessive bleeding, pulmonary embolism, cardiomyopathy, diabetes, anemia, and hypertensive disorders), hyperemesis gravidarum (morning sickness), hemorrhage or excessive bleeding during delivery, premature rupture of membrane, premature rupture of membrane in pre-term birth, placenta previa (placenta covering the cervix), intrauterine/fetal growth restriction, macrosomia (large fetus for gestational age), neonatal conditions (e.g., anemia, apnea, bradycardia and other heart defects, bronchopulmonary dysplasia or chronic lung disease, diabetes, gastroschisis, hydrocephaly, hyperbilirubinemia, hypocalcemia, hypoglycemia, intraventricular hemorrhage, jaundice, necrotizing enterocolitis, patent ductus arteriosis, periventricular leukomalacia, persistent pulmonary hypertension, polycythemia, respiratory distress syndrome, retinopathy of prematurity, and transient tachypnea), and abnormal fetal development stages or states (e.g., abnormal fetal organ function or development)). For example, the pregnancy-related state may comprise a likelihood or susceptibility of an onset of labor in the future (e.g., within about 1 hour, about 2 hours, about 4 hours, about 6 hours, about 8 hours, about 10 hours, about 12 hours, about 14 hours, about 16 hours, about 18 hours, about 20 hours, about 22 hours, about 24 hours, about 1.5 days, about 2 days, about 2.5 days, about 3 days, about 3.5 days, about 4 days, about 4.5 days, about 5 days, about 5.5 days, about 6 days, about 6.5 days, about 7 days, about 8 days, about 9 days, about 10 days, about 12 days, about 14 days, about 3 weeks, about 4 weeks, about 5 weeks, about 6 weeks, about 7 weeks, about 8 weeks, about 9 weeks, about 10 weeks, about 11 weeks, about 12 weeks, about 13 weeks, or more than about 13 weeks). For example, the fetal development stages or states may be related to normal fetal organ function or development and/or abnormal fetal organ function or development for a fetal organ selected from the group consisting of heart, large intestine, small intestine, retina, prefrontal cortex, midbrain, kidney, and esophagus.

The cell-free biological sample may be taken before and/or after treatment of a subject with the pregnancy-related complication. Cell-free biological samples may be obtained from a subject during a treatment or a treatment regime. Multiple cell-free biological samples may be obtained from a subject to monitor the effects of the treatment over time. The cell-free biological sample may be taken from a subject known or suspected of having a pregnancy-related state (e.g., pregnancy-related complication) for which a definitive positive or negative diagnosis is not available via clinical tests. The sample may be taken from a subject suspected of having a pregnancy-related complication. The cell-free biological sample may be taken from a subject experiencing unexplained symptoms, such as fatigue, nausea, weight loss, aches and pains, weakness, or bleeding. The cell-free biological sample may be taken from a subject having explained symptoms. The cell-free biological sample may be taken from a subject at risk of developing a pregnancy-related complication due to factors such as familial history, age, hypertension or pre-hypertension, diabetes or pre-diabetes, overweight or obesity, environmental exposure, lifestyle risk factors (e.g., smoking, alcohol consumption, or drug use), or presence of other risk factors.

The cell-free biological sample may contain one or more analytes capable of being assayed, such as cell-free ribonucleic acid (cfRNA) molecules suitable for assaying to generate transcriptomic data, using transcription products (e.g., messenger RNA, transfer RNA, or ribosomal RNA) derived from said cell-free biological sample to generate transcription product data, cell-free deoxyribonucleic acid (cfDNA) molecules suitable for assaying to generate genomic data and/or methylation data, proteins (e.g., pregnancy-associated proteins corresponding to pregnancy-associated genomic loci or genes) suitable for assaying to generate proteomic data, metabolites suitable for assaying to generate metabolomic data, or a mixture or combination thereof. One or more such analytes (e.g., cfRNA molecules, cfDNA molecules, proteins, or metabolites) may be isolated or extracted from one or more cell-free biological samples of a subject for downstream assaying using one or more suitable assays.

After obtaining a cell-free biological sample from the subject, the cell-free biological sample may be processed to generate datasets indicative of a pregnancy-related state of the subject. For example, a presence, absence, or quantitative assessment of nucleic acid molecules of the cell-free biological sample at a panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins (e.g., corresponding to pregnancy-associated genomic loci or genes), and/or metabolome data comprising quantitative measures of a panel of pregnancy-related state-associated metabolites may be indicative of a pregnancy-related state. Processing the cell-free biological sample obtained from the subject may comprise (i) subjecting the cell-free biological sample to conditions that are sufficient to isolate, enrich, or extract a plurality of nucleic acid molecules, proteins (e.g., pregnancy-associated proteins corresponding to pregnancy-associated genomic loci or genes), and/or metabolites, and (ii) assaying the plurality of nucleic acid molecules, proteins, and/or metabolites to generate the dataset.

In some embodiments, a plurality of nucleic acid molecules is extracted from the cell-free biological sample and subjected to sequencing to generate a plurality of sequencing reads. The nucleic acid molecules may comprise ribonucleic acid (RNA) or deoxyribonucleic acid (DNA). The nucleic acid molecules (e.g., RNA or DNA) may be extracted from the cell-free biological sample by a variety of methods, such as a FastDNA Kit protocol from MP Biomedicals, a QIAamp DNA cell-free biological mini kit from Qiagen, or a cell-free biological DNA isolation kit protocol from Norgen Biotek. The extraction method may extract all RNA or DNA molecules from a sample. Alternatively, the extract method may selectively extract a portion of RNA or DNA molecules from a sample. Extracted RNA molecules from a sample may be converted to DNA molecules by reverse transcription (RT).

The sequencing may be performed by any suitable sequencing methods, such as massively parallel sequencing (MPS), paired-end sequencing, high-throughput sequencing, next-generation sequencing (NGS), shotgun sequencing, single-molecule sequencing, nanopore sequencing, semiconductor sequencing, pyrosequencing, sequencing-by-synthesis (SBS), sequencing-by-ligation, sequencing-by-hybridization, and RNA-Seq (Illumina).

The sequencing may comprise nucleic acid amplification (e.g., of RNA or DNA molecules). In some embodiments, the nucleic acid amplification is polymerase chain reaction (PCR). A suitable number of rounds of PCR (e.g., PCR, qPCR, reverse-transcriptase PCR, digital PCR, etc.) may be performed to sufficiently amplify an initial amount of nucleic acid (e.g., RNA or DNA) to a desired input quantity for subsequent sequencing. In some cases, the PCR may be used for global amplification of target nucleic acids. This may comprise using adapter sequences that may be first ligated to different molecules followed by PCR amplification using universal primers. PCR may be performed using any of a number of commercial kits, e.g., provided by Life Technologies, Affymetrix, Promega, Qiagen, etc. In other cases, only certain target nucleic acids within a population of nucleic acids may be amplified. Specific primers, possibly in conjunction with adapter ligation, may be used to selectively amplify certain targets for downstream sequencing. The PCR may comprise targeted amplification of one or more genomic loci, such as genomic loci associated with pregnancy-related states. The sequencing may comprise use of simultaneous reverse transcription (RT) and polymerase chain reaction (PCR), such as a OneStep RT-PCR kit protocol by Qiagen, NEB, Thermo Fisher Scientific, or Bio-Rad.

RNA or DNA molecules isolated or extracted from a cell-free biological sample may be tagged, e.g., with identifiable tags, to allow for multiplexing of a plurality of samples. Any number of RNA or DNA samples may be multiplexed. For example a multiplexed reaction may contain RNA or DNA from at least about 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more than 100 initial cell-free biological samples. For example, a plurality of cell-free biological samples may be tagged with sample barcodes such that each DNA molecule may be traced back to the sample (and the subject) from which the DNA molecule originated. Such tags may be attached to RNA or DNA molecules by ligation or by PCR amplification with primers.

After subjecting the nucleic acid molecules to sequencing, suitable bioinformatics processes may be performed on the sequence reads to generate the data indicative of the presence, absence, or relative assessment of the pregnancy-related state. For example, the sequence reads may be aligned to one or more reference genomes (e.g., a genome of one or more species such as a human genome). The aligned sequence reads may be quantified at one or more genomic loci to generate the datasets indicative of the pregnancy-related state. For example, quantification of sequences corresponding to a plurality of genomic loci associated with pregnancy-related states may generate the datasets indicative of the pregnancy-related state.

The cell-free biological sample may be processed without any nucleic acid extraction. For example, the pregnancy-related state may be identified or monitored in the subject by using probes configured to selectively enrich nucleic acid (e.g., RNA or DNA) molecules corresponding to the plurality of pregnancy-related state-associated genomic loci. The probes may be nucleic acid primers. The probes may have sequence complementarity with nucleic acid sequences from one or more of the plurality of pregnancy-related state-associated genomic loci or genomic regions. The plurality of pregnancy-related state-associated genomic loci or genomic regions may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, or more distinct pregnancy-related state-associated genomic loci or genomic regions. The plurality of pregnancy-related state-associated genomic loci or genomic regions may comprise one or more members (e.g., 1, 2, 3, 4, 5, 6, 7, 8, 9, 10, 11, 12, 13, 14, 15, 16, 17, 18, 19, 20, about 25, about 30, about 35, about 40, about 45, about 50, about 55, about 60, about 65, about 70, about 75, about 80, or more) selected from the group consisting of ACTB, ADAM12, ALPP, ANXA3, APLF, ARG1, AVPR1A, CAMP, CAPN6, CD180, CGA, CGB, CLCN3, CPVL, CSH1, CSH2, CSHL1, CYP3A7, DAPP1, DCX, DEFA4, DGCR14, ELANE, ENAH, EPB42, FABP1, FAM212B-AS1, FGA, FGB, FRMD4B, FRZB, FSTL3, GH2, GNAZ, HAL, HSD17B1, HSD3B1, HSPB8, Immune, ITIH2, KLF9, KNG1, KRT8, LGALS14, LTF, LYPLAL1, MAP3K7CL, MEF2C, MMD, MMP8, MOB1B, NFATC2, OTC, P2RY12, PAPPA, PGLYRP1, PKHD1L1, PKHD1L1, PLAC1, PLAC4, POLE2, PPBP, PSG1, PSG4, PSG7, PTGER3, RAB11A, RAB27B, RAP1GAP, RGS18, RPL23AP7, S100A8, S100A9, S100P, SERPINA7, SLC2A2, SLC38A4, SLC4A1, TBC1D15, VCAN, VGLL1, B3GNT2, COL24A1, CXCL8, and PTGS2. The pregnancy-related state-associated genomic loci or genomic regions may be associated with gestational age, pre-term birth, due date, onset of labor, or other pregnancy-related states or complications, such as the genomic loci described by, for example, Ngo et al. (“Noninvasive blood tests for fetal development predict gestational age and preterm delivery,” Science, 360(6393), pp. 1133-1136, 8 Jun. 2018), which is hereby incorporated by reference in its entirety.

The probes may be nucleic acid molecules (e.g., RNA or DNA) having sequence complementarity with nucleic acid sequences (e.g., RNA or DNA) of the one or more genomic loci (e.g., pregnancy-related state-associated genomic loci). These nucleic acid molecules may be primers or enrichment sequences. The assaying of the cell-free biological sample using probes that are selective for the one or more genomic loci (e.g., pregnancy-related state-associated genomic loci) may comprise use of array hybridization (e.g., microarray-based), polymerase chain reaction (PCR), or nucleic acid sequencing (e.g., RNA sequencing or DNA sequencing). In some embodiments, DNA or RNA may be assayed by one or more of: isothermal DNA/RNA amplification methods (e.g., loop-mediated isothermal amplification (LAMP), helicase dependent amplification (HDA), rolling circle amplification (RCA), recombinase polymerase amplification (RPA)), immunoassays, electrochemical assays, surface-enhanced Raman spectroscopy (SERS), quantum dot (QD)-based assays, molecular inversion probes, droplet digital PCR (ddPCR), CRISPR/Cas-based detection (e.g., CRISPR-typing PCR (ctPCR), specific high-sensitivity enzymatic reporter un-locking (SHERLOCK), DNA endonuclease targeted CRISPR trans reporter (DETECTR), and CRISPR-mediated analog multi-event recording apparatus (CAMERA)), and laser transmission spectroscopy (LTS).

The assay readouts may be quantified at one or more genomic loci (e.g., pregnancy-related state-associated genomic loci) to generate the data indicative of the pregnancy-related state. For example, quantification of array hybridization or polymerase chain reaction (PCR) corresponding to a plurality of genomic loci (e.g., pregnancy-related state-associated genomic loci) may generate data indicative of the pregnancy-related state. Assay readouts may comprise quantitative PCR (qPCR) values, digital PCR (dPCR) values, digital droplet PCR (ddPCR) values, fluorescence values, etc., or normalized values thereof. The assay may be a home use test configured to be performed in a home setting.

In some embodiments, multiple assays are used to process cell-free biological samples of a subject. For example, a first assay may be used to process a first cell-free biological sample obtained or derived from the subject to generate a first dataset; and based at least in part on the first dataset, a second assay different from said first assay may be used to process a second cell-free biological sample obtained or derived from the subject to generate a second dataset indicative of said pregnancy-related state. The first assay may be used to screen or process cell-free biological samples of a set of subjects, while the second or subsequent assays may be used to screen or process cell-free biological samples of a smaller subset of the set of subjects. The first assay may have a low cost and/or a high sensitivity of detecting one or more pregnancy-related states (e.g., pregnancy-related complication), that is amenable to screening or processing cell-free biological samples of a relatively large set of subjects. The second assay may have a higher cost and/or a higher specificity of detecting one or more pregnancy-related states (e.g., pregnancy-related complication), that is amenable to screening or processing cell-free biological samples of a relatively small set of subjects (e.g., a subset of the subjects screened using the first assay). The second assay may generate a second dataset having a specificity (e.g., for one or more pregnancy-related states such as pregnancy-related complications) greater than the first dataset generated using the first assay. As an example, one or more cell-free biological samples may be processed using a cfRNA assay on a large set of subjects and subsequently a metabolomics assay on a smaller subset of subjects, or vice versa. The smaller subset of subjects may be selected based at least in part on the results of the first assay.

Alternatively, multiple assays may be used to simultaneously process cell-free biological samples of a subject. For example, a first assay may be used to process a first cell-free biological sample obtained or derived from the subject to generate a first dataset indicative of the pregnancy-related state; and a second assay different from the first assay may be used to process a second cell-free biological sample obtained or derived from the subject to generate a second dataset indicative of the pregnancy-related state. Any or all of the first dataset and the second dataset may then be analyzed to assess the pregnancy-related state of the subject. For example, a single diagnostic index or diagnosis score can be generated based on a combination of the first dataset and the second dataset. As another example, separate diagnostic indexes or diagnosis scores can be generated based on the first dataset and the second dataset.

The cell-free biological samples may be processed to identify a set of biomarker RNA transcripts that are indicative of a set of corresponding biomarker proteins (e.g., pregnancy-associated proteins corresponding to pregnancy-associated genomic loci or genes), pathways, and/or metabolites. For example, a given biomarker RNA transcript may be expected to be translated into a corresponding given biomarker protein or a gene regulator for a corresponding given biomarker protein. Therefore, identifying a presence or absence of the given biomarker RNA transcript in a biological sample may be indicative of a presence or absence of a corresponding biomarker protein. As another example, a given biomarker RNA transcript may be expected to correlate with a corresponding given pathway. Therefore, identifying a presence or absence of the given biomarker RNA transcript in a biological sample may be indicative of a presence or absence of the corresponding pathway activity. As another example, a given biomarker RNA transcript may be expected to correlate with a corresponding given biomarker metabolite. Therefore, identifying a presence or absence of the given biomarker RNA transcript in a biological sample may be indicative of a presence or absence of the corresponding biomarker metabolite. In some embodiments, the set of corresponding biomarker proteins, pathways, and/or metabolites comprises pregnancy-related state-associated proteins (e.g., corresponding to pregnancy-associated genomic loci or genes), pathways, and/or metabolites. In some embodiments, the set of corresponding biomarker proteins, pathways, and/or metabolites comprises placental proteins, pathways, and/or metabolites. For example, identifying a presence or absence of the PAPPA gene may be indicative of a presence or absence of the PAPPA protein analog.

The cell-free biological samples may be processed using a metabolomics assay. For example, a metabolomics assay can be used to identify a quantitative measure (e.g., indicative of a presence, absence, or relative amount) of each of a plurality of pregnancy-related state-associated metabolites in a cell-free biological sample of the subject. The metabolomics assay may be configured to process cell-free biological samples such as a blood sample or a urine sample (or derivatives thereof) of the subject. A quantitative measure (e.g., indicative of a presence, absence, or relative amount) of pregnancy-related state-associated metabolites in the cell-free biological sample may be indicative of one or more pregnancy-related states. The metabolites in the cell-free biological sample may be produced (e.g., as an end product or a byproduct) as a result of one or more metabolic pathways corresponding to pregnancy-related state-associated genes. Assaying one or more metabolites of the cell-free biological sample may comprise isolating or extracting the metabolites from the cell-free biological sample. The metabolomics assay may be used to generate datasets indicative of the quantitative measure (e.g., indicative of a presence, absence, or relative amount) of each of a plurality of pregnancy-related state-associated metabolites in the cell-free biological sample of the subject.

The metabolomics assay may analyze a variety of metabolites in the cell-free biological sample, such as small molecules, lipids, amino acids, peptides, nucleotides, hormones and other signaling molecules, cytokines, minerals and elements, polyphenols, fatty acids, dicarboxylic acids, alcohols and polyols, alkanes and alkenes, keto acids, glycolipids, carbohydrates, hydroxy acids, purines, prostanoids, catecholamines, acyl phosphates, phospholipids, cyclic amines, amino ketones, nucleosides, glycerolipids, aromatic acids, retinoids, amino alcohols, pterins, steroids, carnitines, leukotrienes, indoles, porphyrins, sugar phosphates, coenzyme A derivatives, glucuronides, ketones, sugar phosphates, inorganic ions and gases, sphingolipids, bile acids, alcohol phosphates, amino acid phosphates, aldehydes, quinones, pyrimidines, pyridoxals, tricarboxylic acids, acyl glycines, cobalamin derivatives, lipoamides, biotin, and polyamines.

The metabolomics assay may comprise, for example, one or more of: mass spectroscopy (MS), targeted MS, gas chromatography (GC), high performance liquid chromatography (HPLC), capillary electrophoresis (CE), nuclear magnetic resonance (NMR) spectroscopy, ion-mobility spectrometry, Raman spectroscopy, electrochemical assay, or immune assay.

The cell-free biological samples may be processed using a methylation-specific assay. For example, a methylation-specific assay can be used to identify a quantitative measure (e.g., indicative of a presence, absence, or relative amount) of methylation each of a plurality of pregnancy-related state-associated genomic loci in a cell-free biological sample of the subject. The methylation-specific assay may be configured to process cell-free biological samples such as a blood sample or a urine sample (or derivatives thereof) of the subject. A quantitative measure (e.g., indicative of a presence, absence, or relative amount) of methylation of pregnancy-related state-associated genomic loci in the cell-free biological sample may be indicative of one or more pregnancy-related states. The methylation-specific assay may be used to generate datasets indicative of the quantitative measure (e.g., indicative of a presence, absence, or relative amount) of methylation of each of a plurality of pregnancy-related state-associated genomic loci in the cell-free biological sample of the subject.

The methylation-specific assay may comprise, for example, one or more of: a methylation-aware sequencing (e.g., using bisulfite treatment), pyrosequencing, methylation-sensitive single-strand conformation analysis (MS-SSCA), high-resolution melting analysis (HRM), methylation-sensitive single-nucleotide primer extension (MS-SnuPE), base-specific cleavage/MALDI-TOF, microarray-based methylation assay, methylation-specific PCR, targeted bisulfite sequencing, oxidative bisulfite sequencing, mass spectroscopy-based bisulfite sequencing, or reduced representation bisulfite sequence (RRBS).

The cell-free biological samples may be processed using a proteomics assay. For example, a proteomics assay can be used to identify a quantitative measure (e.g., indicative of a presence, absence, or relative amount) of each of a plurality of pregnancy-related state-associated proteins (e.g., corresponding to pregnancy-associated genomic loci or genes) or polypeptides in a cell-free biological sample of the subject. The proteomics assay may be configured to process cell-free biological samples such as a blood sample or a urine sample (or derivatives thereof) of the subject. A quantitative measure (e.g., indicative of a presence, absence, or relative amount) of pregnancy-related state-associated proteins (e.g., corresponding to pregnancy-associated genomic loci or genes) or polypeptides in the cell-free biological sample may be indicative of one or more pregnancy-related states. The proteins or polypeptides in the cell-free biological sample may be produced (e.g., as an end product, an intermediate product, or a byproduct) as a result of one or more biochemical pathways corresponding to pregnancy-related state-associated genes. Assaying one or more proteins or polypeptides of the cell-free biological sample may comprise isolating or extracting the proteins or polypeptides from the cell-free biological sample. The proteomics assay may be used to generate datasets indicative of the quantitative measure (e.g., indicative of a presence, absence, or relative amount) of each of a plurality of pregnancy-related state-associated proteins or polypeptides in the cell-free biological sample of the subject.

The proteomics assay may analyze a variety of proteins (e.g., pregnancy-associated proteins corresponding to pregnancy-associated genomic loci or genes) or polypeptides in the cell-free biological sample, such as proteins made under different cellular conditions (e.g., development, cellular differentiation, or cell cycle). The proteomics assay may comprise, for example, one or more of: an antibody-based immunoassay, an Edman degradation assay, a mass spectrometry-based assay (e.g., matrix-assisted laser desorption/ionization (MALDI) and electrospray ionization (ESI)), a top-down proteomics assay, a bottom-up proteomics assay, a mass spectrometric immunoassay (MSIA), a stable isotope standard capture with anti-peptide antibodies (SISCAPA) assay, a fluorescence two-dimensional differential gel electrophoresis (2-D DIGE) assay, a quantitative proteomics assay, a protein microarray assay, or a reverse-phased protein microarray assay. The proteomics assay may detect post-translational modifications of proteins or polypeptides (e.g., phosphorylation, ubiquitination, methylation, acetylation, glycosylation, oxidation, and nitrosylation). The proteomics assay may identify or quantify one or more proteins or polypeptides from a database (e.g., Human Protein Atlas, PeptideAtlas, and UniProt).

### Kits

The present disclosure provides kits for identifying or monitoring a pregnancy-related state of a subject. A kit may comprise probes for identifying a quantitative measure (e.g., indicative of a presence, absence, or relative amount) of sequences at each of a plurality of pregnancy-related state-associated genomic loci in a cell-free biological sample of the subject. A quantitative measure (e.g., indicative of a presence, absence, or relative amount) of sequences at each of a plurality of pregnancy-related state-associated genomic loci in the cell-free biological sample may be indicative of one or more pregnancy-related states. The probes may be selective for the sequences at the plurality of pregnancy-related state-associated genomic loci in the cell-free biological sample. A kit may comprise instructions for using the probes to process the cell-free biological sample to generate datasets indicative of a quantitative measure (e.g., indicative of a presence, absence, or relative amount) of sequences at each of the plurality of pregnancy-related state-associated genomic loci in a cell-free biological sample of the subject.

The probes in the kit may be selective for the sequences at the plurality of pregnancy-related state-associated genomic loci in the cell-free biological sample. The probes in the kit may be configured to selectively enrich nucleic acid (e.g., RNA or DNA) molecules corresponding to the plurality of pregnancy-related state-associated genomic loci. The probes in the kit may be nucleic acid primers. The probes in the kit may have sequence complementarity with nucleic acid sequences from one or more of the plurality of pregnancy-related state-associated genomic loci or genomic regions. The plurality of pregnancy-related state-associated genomic loci or genomic regions may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or more distinct pregnancy-related state-associated genomic loci or genomic regions. The plurality of pregnancy-related state-associated genomic loci or genomic regions may comprise one or more members selected from the group consisting of ACTB, ADAM12, ALPP, ANXA3, APLF, ARG1, AVPR1A, CAMP, CAPN6, CD180, CGA, CGB, CLCN3, CPVL, CSH1, CSH2, CSHL1, CYP3A7, DAPP1, DCX, DEFA4, DGCR14, ELANE, ENAH, EPB42, FABP1, FAM212B-AS1, FGA, FGB, FRMD4B, FRZB, FSTL3, GH2, GNAZ, HAL, HSD17B1, HSD3B1, HSPB8, Immune, ITIH2, KLF9, KNG1, KRT8, LGALS14, LTF, LYPLAL1, MAP3K7CL, MEF2C, MMD, MMP8, MOB1B, NFATC2, OTC, P2RY12, PAPPA, PGLYRP1, PKHD1L1, PKHD1L1, PLAC1, PLAC4, POLE2, PPBP, PSG1, PSG4, PSG7, PTGER3, RAB11A, RAB27B, RAP1GAP, RGS18, RPL23AP7, S100A8, S100A9, S1OOP, SERPINA7, SLC2A2, SLC38A4, SLC4A1, TBC1D15, VCAN, VGLL1, B3GNT2, COL24A1, CXCL8, and PTGS2.

The instructions in the kit may comprise instructions to assay the cell-free biological sample using the probes that are selective for the sequences at the plurality of pregnancy-related state-associated genomic loci in the cell-free biological sample. These probes may be nucleic acid molecules (e.g., RNA or DNA) having sequence complementarity with nucleic acid sequences (e.g., RNA or DNA) from one or more of the plurality of pregnancy-related state-associated genomic loci. These nucleic acid molecules may be primers or enrichment sequences. The instructions to assay the cell-free biological sample may comprise introductions to perform array hybridization, polymerase chain reaction (PCR), or nucleic acid sequencing (e.g., DNA sequencing or RNA sequencing) to process the cell-free biological sample to generate datasets indicative of a quantitative measure (e.g., indicative of a presence, absence, or relative amount) of sequences at each of the plurality of pregnancy-related state-associated genomic loci in the cell-free biological sample. A quantitative measure (e.g., indicative of a presence, absence, or relative amount) of sequences at each of a plurality of pregnancy-related state-associated genomic loci in the cell-free biological sample may be indicative of one or more pregnancy-related states.

The instructions in the kit may comprise instructions to measure and interpret assay readouts, which may be quantified at one or more of the plurality of pregnancy-related state-associated genomic loci to generate the datasets indicative of a quantitative measure (e.g., indicative of a presence, absence, or relative amount) of sequences at each of the plurality of pregnancy-related state-associated genomic loci in the cell-free biological sample. For example, quantification of array hybridization or polymerase chain reaction (PCR) corresponding to the plurality of pregnancy-related state-associated genomic loci may generate the datasets indicative of a quantitative measure (e.g., indicative of a presence, absence, or relative amount) of sequences at each of the plurality of pregnancy-related state-associated genomic loci in the cell-free biological sample. Assay readouts may comprise quantitative PCR (qPCR) values, digital PCR (dPCR) values, digital droplet PCR (ddPCR) values, fluorescence values, etc., or normalized values thereof.

A kit may comprise a metabolomics assay for identifying a quantitative measure (e.g., indicative of a presence, absence, or relative amount) of each of a plurality of pregnancy-related state-associated metabolites in a cell-free biological sample of the subject. A quantitative measure (e.g., indicative of a presence, absence, or relative amount) of pregnancy-related state-associated metabolites in the cell-free biological sample may be indicative of one or more pregnancy-related states. The metabolites in the cell-free biological sample may be produced (e.g., as an end product or a byproduct) as a result of one or more metabolic pathways corresponding to pregnancy-related state-associated genes. A kit may comprise instructions for isolating or extracting the metabolites from the cell-free biological sample and/or for using the metabolomics assay to generate datasets indicative of the quantitative measure (e.g., indicative of a presence, absence, or relative amount) of each of a plurality of pregnancy-related state-associated metabolites in the cell-free biological sample of the subject.

### Trained Algorithms

After using one or more assays to process one or more cell-free biological samples derived from the subject to generate one or more datasets indicative of the pregnancy-related state or pregnancy-related complication, a trained algorithm may be used to process one or more of the datasets (e.g., at each of a plurality of pregnancy-related state-associated genomic loci) to determine the pregnancy-related state. For example, the trained algorithm may be used to determine quantitative measures of sequences at each of the plurality of pregnancy-related state-associated genomic loci in the cell-free biological samples. The trained algorithm may be configured to identify the pregnancy-related state with an accuracy of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or more than 99% for at least about 25, at least about 50, at least about 100, at least about 150, at least about 200, at least about 250, at least about 300, at least about 350, at least about 400, at least about 450, at least about 500, or more than about 500 independent samples.

The trained algorithm may comprise a supervised machine learning algorithm. The trained algorithm may comprise a classification and regression tree (CART) algorithm. The supervised machine learning algorithm may comprise, for example, a Random Forest, a support vector machine (SVM), a neural network, or a deep learning algorithm. The trained algorithm may comprise a differential expression algorithm. The differential expression algorithm may comprise a use comparison of stochastic models, generalized Poisson (GPseq), mixed Poisson (TSPM), Poisson log-linear (PoissonSeq), negative binomial (edgeR, DESeq, baySeq, NBPSeq), linear model fit by MAANOVA, or a combination thereof. The trained algorithm may comprise an unsupervised machine learning algorithm.

The trained algorithm may be configured to accept a plurality of input variables and to produce one or more output values based on the plurality of input variables. The plurality of input variables may comprise one or more datasets indicative of a pregnancy-related state. For example, an input variable may comprise a number of sequences corresponding to or aligning to each of the plurality of pregnancy-related state-associated genomic loci. The plurality of input variables may also include clinical health data of a subject.

The trained algorithm may comprise a classifier, such that each of the one or more output values comprises one of a fixed number of possible values (e.g., a linear classifier, a logistic regression classifier, etc.) indicating a classification of the cell-free biological sample by the classifier. The trained algorithm may comprise a binary classifier, such that each of the one or more output values comprises one of two values (e.g., {0, 1}, {positive, negative}, or {high-risk, low-risk}) indicating a classification of the cell-free biological sample by the classifier. The trained algorithm may be another type of classifier, such that each of the one or more output values comprises one of more than two values (e.g., {0, 1, 2}, {positive, negative, or indeterminate}, or {high-risk, intermediate-risk, or low-risk}) indicating a classification of the cell-free biological sample by the classifier. The output values may comprise descriptive labels, numerical values, or a combination thereof. Some of the output values may comprise descriptive labels. Such descriptive labels may provide an identification or indication of the disease or disorder state of the subject, and may comprise, for example, positive, negative, high-risk, intermediate-risk, low-risk, or indeterminate. Such descriptive labels may provide an identification of a treatment for the subject's pregnancy-related state, and may comprise, for example, a therapeutic intervention, a duration of the therapeutic intervention, and/or a dosage of the therapeutic intervention suitable to treat a pregnancy-related condition. Such descriptive labels may provide an identification of secondary clinical tests that may be appropriate to perform on the subject, and may comprise, for example, an imaging test, a blood test, a computed tomography (CT) scan, a magnetic resonance imaging (MRI) scan, an ultrasound scan, a chest X-ray, a positron emission tomography (PET) scan, a PET-CT scan, a cell-free biological cytology, an amniocentesis, a non-invasive prenatal test (NIPT), or any combination thereof. For example, such descriptive labels may provide a prognosis of the pregnancy-related state of the subject. As another example, such descriptive labels may provide a relative assessment of the pregnancy-related state (e.g., an estimated gestational age in number of days, weeks, or months) of the subject. Some descriptive labels may be mapped to numerical values, for example, by mapping “positive” to 1 and “negative” to 0.

Some of the output values may comprise numerical values, such as binary, integer, or continuous values. Such binary output values may comprise, for example, {0, 1},{positive, negative}, or {high-risk, low-risk}. Such integer output values may comprise, for example, {0, 1, 2}. Such continuous output values may comprise, for example, a probability value of at least 0 and no more than 1. Such continuous output values may comprise, for example, an un-normalized probability value of at least 0. Such continuous output values may indicate a prognosis of the pregnancy-related state of the subject. Some numerical values may be mapped to descriptive labels, for example, by mapping 1 to “positive” and 0 to “negative.”

Some of the output values may be assigned based on one or more cutoff values. For example, a binary classification of samples may assign an output value of “positive” or 1 if the sample indicates that the subject has at least a 50% probability of having a pregnancy-related state (e.g., pregnancy-related complication). For example, a binary classification of samples may assign an output value of “negative” or 0 if the sample indicates that the subject has less than a 50% probability of having a pregnancy-related state (e.g., pregnancy-related complication). In this case, a single cutoff value of 50% is used to classify samples into one of the two possible binary output values. Examples of single cutoff values may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, and about 99%.

As another example, a classification of samples may assign an output value of “positive” or 1 if the sample indicates that the subject has a probability of having a pregnancy-related state (e.g., pregnancy-related complication) of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more. The classification of samples may assign an output value of “positive” or 1 if the sample indicates that the subject has a probability of having a pregnancy-related state (e.g., pregnancy-related complication) of more than about 50%, more than about 55%, more than about 60%, more than about 65%, more than about 70%, more than about 75%, more than about 80%, more than about 85%, more than about 90%, more than about 91%, more than about 92%, more than about 93%, more than about 94%, more than about 95%, more than about 96%, more than about 97%, more than about 98%, or more than about 99%.

The classification of samples may assign an output value of “negative” or 0 if the sample indicates that the subject has a probability of having a pregnancy-related state (e.g., pregnancy-related complication) of less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1%. The classification of samples may assign an output value of “negative” or 0 if the sample indicates that the subject has a probability of having a pregnancy-related state (e.g., pregnancy-related complication) of no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%.

The classification of samples may assign an output value of “indeterminate” or 2 if the sample is not classified as “positive”, “negative”, 1, or 0. In this case, a set of two cutoff values is used to classify samples into one of the three possible output values. Examples of sets of cutoff values may include {1%, 99%}, {2%, 98%}, {5%, 95%}, {10%, 90%}, {15%, 85%}, {20%, 80%}, {25%, 75%}, {30%, 70%}, {35%, 65%}, {40%, 60%}, and {45%, 55%}. Similarly, sets of n cutoff values may be used to classify samples into one of n+1 possible output values, where n is any positive integer.

The trained algorithm may be trained with a plurality of independent training samples. Each of the independent training samples may comprise a cell-free biological sample from a subject, associated datasets obtained by assaying the cell-free biological sample (as described elsewhere herein), and one or more known output values corresponding to the cell-free biological sample (e.g., a clinical diagnosis, prognosis, absence, or treatment efficacy of a pregnancy-related state of the subject). Independent training samples may comprise cell-free biological samples and associated datasets and outputs obtained or derived from a plurality of different subjects. Independent training samples may comprise cell-free biological samples and associated datasets and outputs obtained at a plurality of different time points from the same subject (e.g., on a regular basis such as weekly, biweekly, or monthly). Independent training samples may be associated with presence of the pregnancy-related state (e.g., training samples comprising cell-free biological samples and associated datasets and outputs obtained or derived from a plurality of subjects known to have the pregnancy-related state). Independent training samples may be associated with absence of the pregnancy-related state (e.g., training samples comprising cell-free biological samples and associated datasets and outputs obtained or derived from a plurality of subjects who are known to not have a previous diagnosis of the pregnancy-related state or who have received a negative test result for the pregnancy-related state).

The trained algorithm may be trained with at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The independent training samples may comprise cell-free biological samples associated with presence of the pregnancy-related state and/or cell-free biological samples associated with absence of the pregnancy-related state. The trained algorithm may be trained with no more than about 500, no more than about 450, no more than about 400, no more than about 350, no more than about 300, no more than about 250, no more than about 200, no more than about 150, no more than about 100, or no more than about 50 independent training samples associated with presence of the pregnancy-related state. In some embodiments, the cell-free biological sample is independent of samples used to train the trained algorithm.

The trained algorithm may be trained with a first number of independent training samples associated with presence of the pregnancy-related state and a second number of independent training samples associated with absence of the pregnancy-related state. The first number of independent training samples associated with presence of the pregnancy-related state may be no more than the second number of independent training samples associated with absence of the pregnancy-related state. The first number of independent training samples associated with presence of the pregnancy-related state may be equal to the second number of independent training samples associated with absence of the pregnancy-related state. The first number of independent training samples associated with presence of the pregnancy-related state may be greater than the second number of independent training samples associated with absence of the pregnancy-related state.

The trained algorithm may be configured to identify the pregnancy-related state at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more; for at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The accuracy of identifying the pregnancy-related state by the trained algorithm may be calculated as the percentage of independent test samples (e.g., subjects known to have the pregnancy-related state or subjects with negative clinical test results for the pregnancy-related state) that are correctly identified or classified as having or not having the pregnancy-related state.

The trained algorithm may be configured to identify the pregnancy-related state with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the pregnancy-related state using the trained algorithm may be calculated as the percentage of cell-free biological samples identified or classified as having the pregnancy-related state that correspond to subjects that truly have the pregnancy-related state.

The trained algorithm may be configured to identify the pregnancy-related state with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the pregnancy-related state using the trained algorithm may be calculated as the percentage of cell-free biological samples identified or classified as not having the pregnancy-related state that correspond to subjects that truly do not have the pregnancy-related state.

The trained algorithm may be configured to identify the pregnancy-related state with a clinical sensitivity at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the pregnancy-related state using the trained algorithm may be calculated as the percentage of independent test samples associated with presence of the pregnancy-related state (e.g., subjects known to have the pregnancy-related state) that are correctly identified or classified as having the pregnancy-related state.

The trained algorithm may be configured to identify the pregnancy-related state with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the pregnancy-related state using the trained algorithm may be calculated as the percentage of independent test samples associated with absence of the pregnancy-related state (e.g., subjects with negative clinical test results for the pregnancy-related state) that are correctly identified or classified as not having the pregnancy-related state.

The trained algorithm may be configured to identify the pregnancy-related state with an Area-Under-Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more. The AUC may be calculated as an integral of the Receiver Operator Characteristic (ROC) curve (e.g., the area under the ROC curve) associated with the trained algorithm in classifying cell-free biological samples as having or not having the pregnancy-related state.

The trained algorithm may be adjusted or tuned to improve one or more of the performance, accuracy, PPV, NPV, clinical sensitivity, clinical specificity, or AUC of identifying the pregnancy-related state. The trained algorithm may be adjusted or tuned by adjusting parameters of the trained algorithm (e.g., a set of cutoff values used to classify a cell-free biological sample as described elsewhere herein, or weights of a neural network). The trained algorithm may be adjusted or tuned continuously during the training process or after the training process has completed.

After the trained algorithm is initially trained, a subset of the inputs may be identified as most influential or most important to be included for making high-quality classifications. For example, a subset of the plurality of pregnancy-related state-associated genomic loci may be identified as most influential or most important to be included for making high-quality classifications or identifications of pregnancy-related states (or sub-types of pregnancy-related states). The plurality of pregnancy-related state-associated genomic loci or a subset thereof may be ranked based on classification metrics indicative of each genomic locus's influence or importance toward making high-quality classifications or identifications of pregnancy-related states (or sub-types of pregnancy-related states). Such metrics may be used to reduce, in some cases significantly, the number of input variables (e.g., predictor variables) that may be used to train the trained algorithm to a desired performance level (e.g., based on a desired minimum accuracy, PPV, NPV, clinical sensitivity, clinical specificity, AUC, or a combination thereof). For example, if training the trained algorithm with a plurality comprising several dozen or hundreds of input variables in the trained algorithm results in an accuracy of classification of more than 99%, then training the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality can yield decreased but still acceptable accuracy of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%). The subset may be selected by rank-ordering the entire plurality of input variables and selecting a predetermined number (e.g., no more than about 5, no more than about 10, no more than about 15, no more than about 20, no more than about 25, no more than about 30, no more than about 35, no more than about 40, no more than about 45, no more than about 50, or no more than about 100) of input variables with the best classification metrics.

### Identifying or Monitoring a Pregnancy-Related State

After using a trained algorithm to process the dataset, the pregnancy-related state or pregnancy-related complication may be identified or monitored in the subject. The identification may be based at least in part on quantitative measures of sequence reads of the dataset at a panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins, and/or metabolome data comprising quantitative measures of a panel of pregnancy-related state-associated metabolites.

The pregnancy-related state may be identified in the subject at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The accuracy of identifying the pregnancy-related state by the trained algorithm may be calculated as the percentage of independent test samples (e.g., subjects known to have the pregnancy-related state or subjects with negative clinical test results for the pregnancy-related state) that are correctly identified or classified as having or not having the pregnancy-related state.

The pregnancy-related state may be identified in the subject with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the pregnancy-related state using the trained algorithm may be calculated as the percentage of cell-free biological samples identified or classified as having the pregnancy-related state that correspond to subjects that truly have the pregnancy-related state.

The pregnancy-related state may be identified in the subject with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the pregnancy-related state using the trained algorithm may be calculated as the percentage of cell-free biological samples identified or classified as not having the pregnancy-related state that correspond to subjects that truly do not have the pregnancy-related state.

The pregnancy-related state may be identified in the subject with a clinical sensitivity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the pregnancy-related state using the trained algorithm may be calculated as the percentage of independent test samples associated with presence of the pregnancy-related state (e.g., subjects known to have the pregnancy-related state) that are correctly identified or classified as having the pregnancy-related state.

The pregnancy-related state may be identified in the subject with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the pregnancy-related state using the trained algorithm may be calculated as the percentage of independent test samples associated with absence of the pregnancy-related state (e.g., subjects with negative clinical test results for the pregnancy-related state) that are correctly identified or classified as not having the pregnancy-related state.

In an aspect, the present disclosure provides a method for determining that a subject is at risk of pre-term birth, comprising assaying a cell-free biological sample derived from the subject to generate a dataset that is indicative of said pre-term birth risk at a specificity of at least 80%, and using a trained algorithm that is trained on samples independent of the cell-free biological sample to determine that the subject is at risk of pre-term birth at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more.

After the pregnancy-related state is identified in a subject, a sub-type of the pregnancy-related state (e.g., selected from among a plurality of sub-types of the pregnancy-related state) may further be identified. The sub-type of the pregnancy-related state may be determined based at least in part on the quantitative measures of sequence reads of the dataset at a panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins, and/or metabolome data comprising quantitative measures of a panel of pregnancy-related state-associated metabolites. For example, the subject may be identified as being at risk of a sub-type of pre-term birth (e.g., selected from among a plurality of sub-types of pre-term birth). After identifying the subject as being at risk of a sub-type of pre-term birth, a clinical intervention for the subject may be selected based at least in part on the sub-type of pre-term birth for which the subject is identified as being at risk. In some embodiments, the clinical intervention is selected from a plurality of clinical interventions (e.g., clinically indicated for different sub-types of pre-term birth).

In some embodiments, the trained algorithm may determine that the subject is at risk of pre-term birth of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more.

The trained algorithm may determine that the subject is at risk of pre-term birth at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more.

Upon identifying the subject as having the pregnancy-related state, the subject may be optionally provided with a therapeutic intervention (e.g., prescribing an appropriate course of treatment to treat the pregnancy-related state of the subject). The therapeutic intervention may comprise a prescription of an effective dose of a drug, a further testing or evaluation of the pregnancy-related state, a further monitoring of the pregnancy-related state, an induction or inhibition of labor, or a combination thereof. If the subject is currently being treated for the pregnancy-related state with a course of treatment, the therapeutic intervention may comprise a subsequent different course of treatment (e.g., to increase treatment efficacy due to non-efficacy of the current course of treatment).

The therapeutic intervention may comprise recommending the subject for a secondary clinical test to confirm a diagnosis of the pregnancy-related state. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a cell-free biological cytology, an amniocentesis, a non-invasive prenatal test (NIPT), or any combination thereof.

The quantitative measures of sequence reads of the dataset at the panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins, and/or metabolome data comprising quantitative measures of a panel of pregnancy-related state-associated metabolites may be assessed over a duration of time to monitor a patient (e.g., subject who has pregnancy-related state or who is being treated for pregnancy-related state). In such cases, the quantitative measures of the dataset of the patient may change during the course of treatment. For example, the quantitative measures of the dataset of a patient with decreasing risk of the pregnancy-related state due to an effective treatment may shift toward the profile or distribution of a healthy subject (e.g., a subject without a pregnancy-related complication). Conversely, for example, the quantitative measures of the dataset of a patient with increasing risk of the pregnancy-related state due to an ineffective treatment may shift toward the profile or distribution of a subject with higher risk of the pregnancy-related state or a more advanced pregnancy-related state.

The pregnancy-related state of the subject may be monitored by monitoring a course of treatment for treating the pregnancy-related state of the subject. The monitoring may comprise assessing the pregnancy-related state of the subject at two or more time points. The assessing may be based at least on the quantitative measures of sequence reads of the dataset at a panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins, and/or metabolome data comprising quantitative measures of a panel of pregnancy-related state-associated metabolites determined at each of the two or more time points.

In some embodiments, a difference in the quantitative measures of sequence reads of the dataset at a panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins, and/or metabolome data comprising quantitative measures of a panel of pregnancy-related state-associated metabolites determined between the two or more time points may be indicative of one or more clinical indications, such as (i) a diagnosis of the pregnancy-related state of the subject, (ii) a prognosis of the pregnancy-related state of the subject, (iii) an increased risk of the pregnancy-related state of the subject, (iv) a decreased risk of the pregnancy-related state of the subject, (v) an efficacy of the course of treatment for treating the pregnancy-related state of the subject, and (vi) a non-efficacy of the course of treatment for treating the pregnancy-related state of the subject.

In some embodiments, a difference in the quantitative measures of sequence reads of the dataset at a panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins, and/or metabolome data comprising quantitative measures of a panel of pregnancy-related state-associated metabolites determined between the two or more time points may be indicative of a diagnosis of the pregnancy-related state of the subject. For example, if the pregnancy-related state was not detected in the subject at an earlier time point but was detected in the subject at a later time point, then the difference is indicative of a diagnosis of the pregnancy-related state of the subject. A clinical action or decision may be made based on this indication of diagnosis of the pregnancy-related state of the subject, such as, for example, prescribing a new therapeutic intervention for the subject. The clinical action or decision may comprise recommending the subject for a secondary clinical test to confirm the diagnosis of the pregnancy-related state. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a cell-free biological cytology, an amniocentesis, a non-invasive prenatal test (NIPT), or any combination thereof.

In some embodiments, a difference in the quantitative measures of sequence reads of the dataset at a panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins, and/or metabolome data comprising quantitative measures of a panel of pregnancy-related state-associated metabolites determined between the two or more time points may be indicative of a prognosis of the pregnancy-related state of the subject.

In some embodiments, a difference in the quantitative measures of sequence reads of the dataset at a panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins, and/or metabolome data comprising quantitative measures of a panel of pregnancy-related state-associated metabolites determined between the two or more time points may be indicative of the subject having an increased risk of the pregnancy-related state. For example, if the pregnancy-related state was detected in the subject both at an earlier time point and at a later time point, and if the difference is a negative difference (e.g., the quantitative measures of sequence reads of the dataset at a panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins, and/or metabolome data comprising quantitative measures of a panel of pregnancy-related state-associated metabolites increased from the earlier time point to the later time point), then the difference may be indicative of the subject having an increased risk of the pregnancy-related state. A clinical action or decision may be made based on this indication of the increased risk of the pregnancy-related state, e.g., prescribing a new therapeutic intervention or switching therapeutic interventions (e.g., ending a current treatment and prescribing a new treatment) for the subject. The clinical action or decision may comprise recommending the subject for a secondary clinical test to confirm the increased risk of the pregnancy-related state. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a cell-free biological cytology, an amniocentesis, a non-invasive prenatal test (NIPT), or any combination thereof.

In some embodiments, a difference in the quantitative measures of sequence reads of the dataset at a panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins, and/or metabolome data comprising quantitative measures of a panel of pregnancy-related state-associated metabolites determined between the two or more time points may be indicative of the subject having a decreased risk of the pregnancy-related state. For example, if the pregnancy-related state was detected in the subject both at an earlier time point and at a later time point, and if the difference is a positive difference (e.g., the quantitative measures of sequence reads of the dataset at a panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins, and/or metabolome data comprising quantitative measures of a panel of pregnancy-related state-associated metabolites decreased from the earlier time point to the later time point), then the difference may be indicative of the subject having a decreased risk of the pregnancy-related state. A clinical action or decision may be made based on this indication of the decreased risk of the pregnancy-related state (e.g., continuing or ending a current therapeutic intervention) for the subject. The clinical action or decision may comprise recommending the subject for a secondary clinical test to confirm the decreased risk of the pregnancy-related state. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a cell-free biological cytology, an amniocentesis, a non-invasive prenatal test (NIPT), or any combination thereof.

In some embodiments, a difference in the quantitative measures of sequence reads of the dataset at a panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins, and/or metabolome data comprising quantitative measures of a panel of pregnancy-related state-associated metabolites determined between the two or more time points may be indicative of an efficacy of the course of treatment for treating the pregnancy-related state of the subject. For example, if the pregnancy-related state was detected in the subject at an earlier time point but was not detected in the subject at a later time point, then the difference may be indicative of an efficacy of the course of treatment for treating the pregnancy-related state of the subject. A clinical action or decision may be made based on this indication of the efficacy of the course of treatment for treating the pregnancy-related state of the subject, e.g., continuing or ending a current therapeutic intervention for the subject. The clinical action or decision may comprise recommending the subject for a secondary clinical test to confirm the efficacy of the course of treatment for treating the pregnancy-related state. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a cell-free biological cytology, an amniocentesis, a non-invasive prenatal test (NIPT), or any combination thereof.

In some embodiments, a difference in the quantitative measures of sequence reads of the dataset at a panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins, and/or metabolome data comprising quantitative measures of a panel of pregnancy-related state-associated metabolites determined between the two or more time points may be indicative of a non-efficacy of the course of treatment for treating the pregnancy-related state of the subject. For example, if the pregnancy-related state was detected in the subject both at an earlier time point and at a later time point, and if the difference is a negative or zero difference (e.g., the quantitative measures of sequence reads of the dataset at a panel of pregnancy-related state-associated genomic loci (e.g., quantitative measures of RNA transcripts or DNA at the pregnancy-related state-associated genomic loci), proteomic data comprising quantitative measures of proteins of the dataset at a panel of pregnancy-related state-associated proteins, and/or metabolome data comprising quantitative measures of a panel of pregnancy-related state-associated metabolites increased or remained at a constant level from the earlier time point to the later time point), and if an efficacious treatment was indicated at an earlier time point, then the difference may be indicative of a non-efficacy of the course of treatment for treating the pregnancy-related state of the subject. A clinical action or decision may be made based on this indication of the non-efficacy of the course of treatment for treating the pregnancy-related state of the subject, e.g., ending a current therapeutic intervention and/or switching to (e.g., prescribing) a different new therapeutic intervention for the subject. The clinical action or decision may comprise recommending the subject for a secondary clinical test to confirm the non-efficacy of the course of treatment for treating the pregnancy-related state. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a cell-free biological cytology, an amniocentesis, a non-invasive prenatal test (NIPT), or any combination thereof.

In another aspect, the present disclosure provides a computer-implemented method for predicting a risk of pre-term birth of a subject, comprising: (a) receiving clinical health data of the subject, wherein the clinical health data comprises a plurality of quantitative or categorical measures of said subject; (b) using a trained algorithm to process the clinical health data of the subject to determine a risk score indicative of the risk of pre-term birth of the subject; and (c) electronically outputting a report indicative of the risk score indicative of the risk of pre-term birth of the subject.

In some embodiments, for example, the clinical health data comprises one or more quantitative measures of the subject, such as age, weight, height, body mass index (BMI), blood pressure, heart rate, glucose levels, number of previous pregnancies, and number of previous births. As another example, the clinical health data can comprise one or more categorical measures, such as race, ethnicity, history of medication or other clinical treatment, history of tobacco use, history of alcohol consumption, daily activity or fitness level, genetic test results, blood test results, imaging results, and fetal screening results.

In some embodiments, the computer-implemented method for predicting a risk of pre-term birth of a subject is performed using a computer or mobile device application. For example, a subject can use a computer or mobile device application to input her own clinical health data, including quantitative and/or categorical measures. The computer or mobile device application can then use a trained algorithm to process the clinical health data to determine a risk score indicative of the risk of pre-term birth of the subject. The computer or mobile device application can then display a report indicative of the risk score indicative of the risk of pre-term birth of the subject.

In some embodiments, the risk score indicative of the risk of pre-term birth of the subject can be refined by performing one or more subsequent clinical tests for the subject. For example, the subject can be referred by a physician for one or more subsequent clinical tests (e.g., an ultrasound imaging or a blood test) based on the initial risk score. Next, the computer or mobile device application may process results from the one or more subsequent clinical tests using a trained algorithm to determine an updated risk score indicative of the risk of pre-term birth of the subject.

In some embodiments, the risk score comprises a likelihood of the subject having a pre-term birth within a pre-determined duration of time. For example, the pre-determined duration of time may be about 1 hour, about 2 hours, about 4 hours, about 6 hours, about 8 hours, about 10 hours, about 12 hours, about 14 hours, about 16 hours, about 18 hours, about 20 hours, about 22 hours, about 24 hours, about 1.5 days, about 2 days, about 2.5 days, about 3 days, about 3.5 days, about 4 days, about 4.5 days, about 5 days, about 5.5 days, about 6 days, about 6.5 days, about 7 days, about 8 days, about 9 days, about 10 days, about 12 days, about 14 days, about 3 weeks, about 4 weeks, about 5 weeks, about 6 weeks, about 7 weeks, about 8 weeks, about 9 weeks, about 10 weeks, about 11 weeks, about 12 weeks, about 13 weeks, or more than about 13 weeks.

### Outputting a Report of the Pregnancy-Related State

After the pregnancy-related state is identified or an increased risk of the pregnancy-related state is monitored in the subject, a report may be electronically outputted that is indicative of (e.g., identifies or provides an indication of) the pregnancy-related state of the subject. The subject may not display a pregnancy-related state (e.g., is asymptomatic of the pregnancy-related state such as a pregnancy-related complication). The report may be presented on a graphical user interface (GUI) of an electronic device of a user. The user may be the subject, a caretaker, a physician, a nurse, or another health care worker.

The report may include one or more clinical indications such as (i) a diagnosis of the pregnancy-related state of the subject, (ii) a prognosis of the pregnancy-related state of the subject, (iii) an increased risk of the pregnancy-related state of the subject, (iv) a decreased risk of the pregnancy-related state of the subject, (v) an efficacy of the course of treatment for treating the pregnancy-related state of the subject, and (vi) a non-efficacy of the course of treatment for treating the pregnancy-related state of the subject. The report may include one or more clinical actions or decisions made based on these one or more clinical indications. Such clinical actions or decisions may be directed to therapeutic interventions, induction or inhibition of labor, or further clinical assessment or testing of the pregnancy-related state of the subject.

For example, a clinical indication of a diagnosis of the pregnancy-related state of the subject may be accompanied with a clinical action of prescribing a new therapeutic intervention for the subject. As another example, a clinical indication of an increased risk of the pregnancy-related state of the subject may be accompanied with a clinical action of prescribing a new therapeutic intervention or switching therapeutic interventions (e.g., ending a current treatment and prescribing a new treatment) for the subject. As another example, a clinical indication of a decreased risk of the pregnancy-related state of the subject may be accompanied with a clinical action of continuing or ending a current therapeutic intervention for the subject. As another example, a clinical indication of an efficacy of the course of treatment for treating the pregnancy-related state of the subject may be accompanied with a clinical action of continuing or ending a current therapeutic intervention for the subject. As another example, a clinical indication of a non-efficacy of the course of treatment for treating the pregnancy-related state of the subject may be accompanied with a clinical action of ending a current therapeutic intervention and/or switching to (e.g., prescribing) a different new therapeutic intervention for the subject.

### Computer Systems

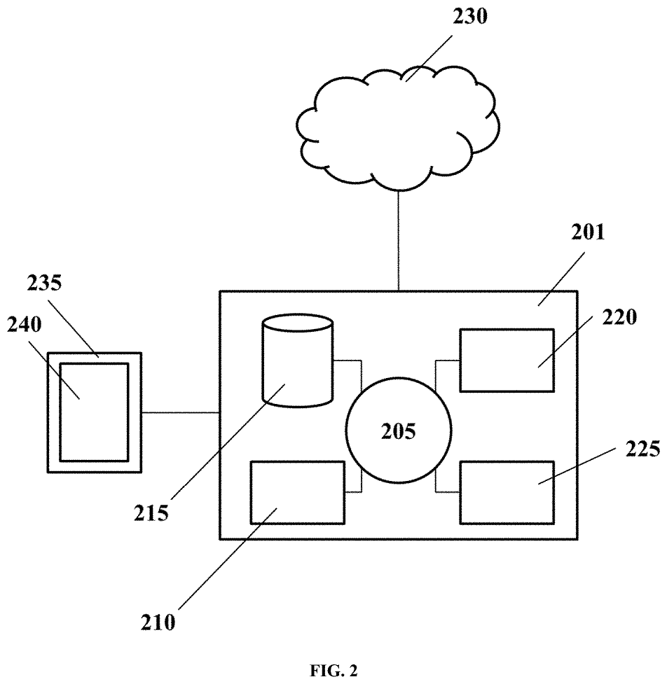

The present disclosure provides computer systems that are programmed to implement methods of the disclosure. FIG. 2 shows a computer system 201 that is programmed or otherwise configured to, for example, (i) train and test a trained algorithm, (ii) use the trained algorithm to process data to determine a pregnancy-related state of a subject, (iii) determine a quantitative measure indicative of a pregnancy-related state of a subject, (iv) identify or monitor the pregnancy-related state of the subject, and (v) electronically output a report that indicative of the pregnancy-related state of the subject.

The computer system 201 can regulate various aspects of analysis, calculation, and generation of the present disclosure, such as, for example, (i) training and testing a trained algorithm, (ii) using the trained algorithm to process data to determine a pregnancy-related state of a subject, (iii) determining a quantitative measure indicative of a pregnancy-related state of a subject, (iv) identifying or monitoring the pregnancy-related state of the subject, and (v) electronically outputting a report that indicative of the pregnancy-related state of the subject. The computer system 201 can be an electronic device of a user or a computer system that is remotely located with respect to the electronic device. The electronic device can be a mobile electronic device.

The computer system 201 includes a central processing unit (CPU, also “processor” and “computer processor” herein) 205, which can be a single core or multi core processor, or a plurality of processors for parallel processing. The computer system 201 also includes memory or memory location 210 (e.g., random-access memory, read-only memory, flash memory), electronic storage unit 215 (e.g., hard disk), communication interface 220 (e.g., network adapter) for communicating with one or more other systems, and peripheral devices 225, such as cache, other memory, data storage and/or electronic display adapters. The memory 210, storage unit 215, interface 220 and peripheral devices 225 are in communication with the CPU 205 through a communication bus (solid lines), such as a motherboard. The storage unit 215 can be a data storage unit (or data repository) for storing data. The computer system 201 can be operatively coupled to a computer network (“network”) 230 with the aid of the communication interface 220. The network 230 can be the Internet, an internet and/or extranet, or an intranet and/or extranet that is in communication with the Internet.

The network 230 in some cases is a telecommunication and/or data network. The network 230 can include one or more computer servers, which can enable distributed computing, such as cloud computing. For example, one or more computer servers may enable cloud computing over the network 230 (“the cloud”) to perform various aspects of analysis, calculation, and generation of the present disclosure, such as, for example, (i) training and testing a trained algorithm, (ii) using the trained algorithm to process data to determine a pregnancy-related state of a subject, (iii) determining a quantitative measure indicative of a pregnancy-related state of a subject, (iv) identifying or monitoring the pregnancy-related state of the subject, and (v) electronically outputting a report that indicative of the pregnancy-related state of the subject. Such cloud computing may be provided by cloud computing platforms such as, for example, Amazon Web Services (AWS), Microsoft Azure, Google Cloud Platform, and IBM cloud. The network 230, in some cases with the aid of the computer system 201, can implement a peer-to-peer network, which may enable devices coupled to the computer system 201 to behave as a client or a server.

The CPU 205 may comprise one or more computer processors and/or one or more graphics processing units (GPUs). The CPU 205 can execute a sequence of machine-readable instructions, which can be embodied in a program or software. The instructions may be stored in a memory location, such as the memory 210. The instructions can be directed to the CPU 205, which can subsequently program or otherwise configure the CPU 205 to implement methods of the present disclosure. Examples of operations performed by the CPU 205 can include fetch, decode, execute, and writeback.

The CPU 205 can be part of a circuit, such as an integrated circuit. One or more other components of the system 201 can be included in the circuit. In some cases, the circuit is an application specific integrated circuit (ASIC).

The storage unit 215 can store files, such as drivers, libraries and saved programs. The storage unit 215 can store user data, e.g., user preferences and user programs. The computer system 201 in some cases can include one or more additional data storage units that are external to the computer system 201, such as located on a remote server that is in communication with the computer system 201 through an intranet or the Internet.

The computer system 201 can communicate with one or more remote computer systems through the network 230. For instance, the computer system 201 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 201 via the network 230.

Methods as described herein can be implemented by way of machine (e.g., computer processor) executable code stored on an electronic storage location of the computer system 201, such as, for example, on the memory 210 or electronic storage unit 215. The machine executable or machine readable code can be provided in the form of software. During use, the code can be executed by the processor 205. In some cases, the code can be retrieved from the storage unit 215 and stored on the memory 210 for ready access by the processor 205. In some situations, the electronic storage unit 215 can be precluded, and machine-executable instructions are stored on memory 210.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 201 can include or be in communication with an electronic display 235 that comprises a user interface (UI) 240 for providing, for example, (i) a visual display indicative of training and testing of a trained algorithm, (ii) a visual display of data indicative of a pregnancy-related state of a subject, (iii) a quantitative measure of a pregnancy-related state of a subject, (iv) an identification of a subject as having a pregnancy-related state, or (v) an electronic report indicative of the pregnancy-related state of the subject. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 205. The algorithm can, for example, (i) train and test a trained algorithm, (ii) use the trained algorithm to process data to determine a pregnancy-related state of a subject, (iii) determine a quantitative measure indicative of a pregnancy-related state of a subject, (iv) identify or monitor the pregnancy-related state of the subject, and (v) electronically output a report that indicative of the pregnancy-related state of the subject.

### EXAMPLES

### Example 1: Cohorts of Subjects

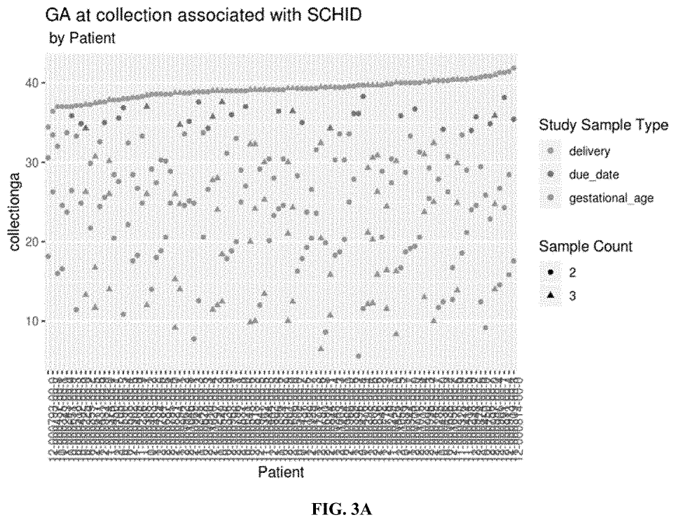

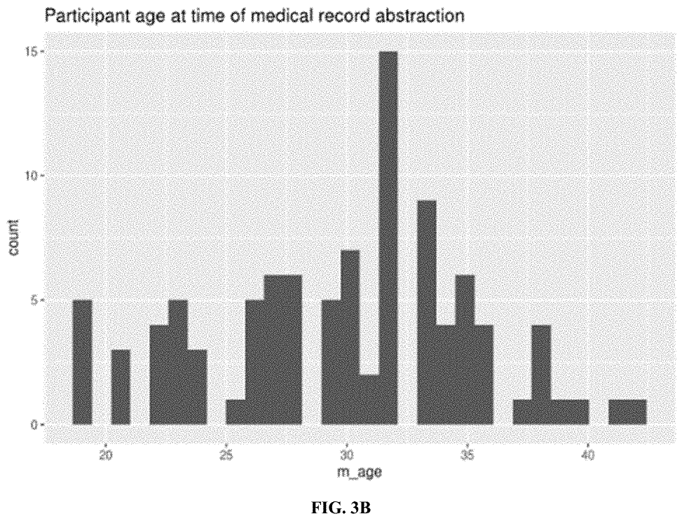

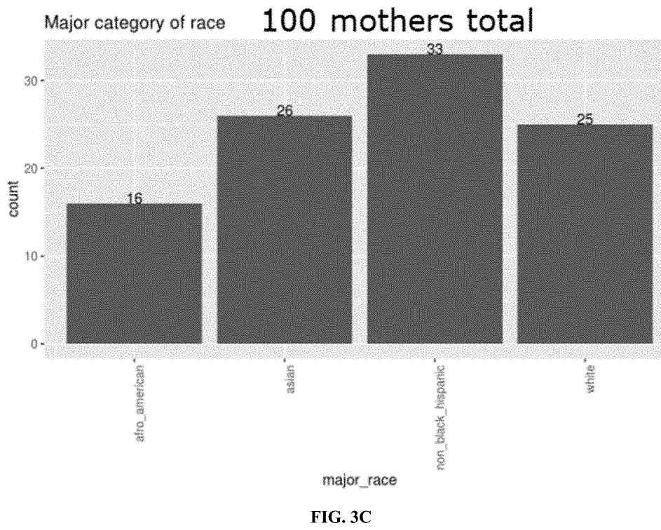

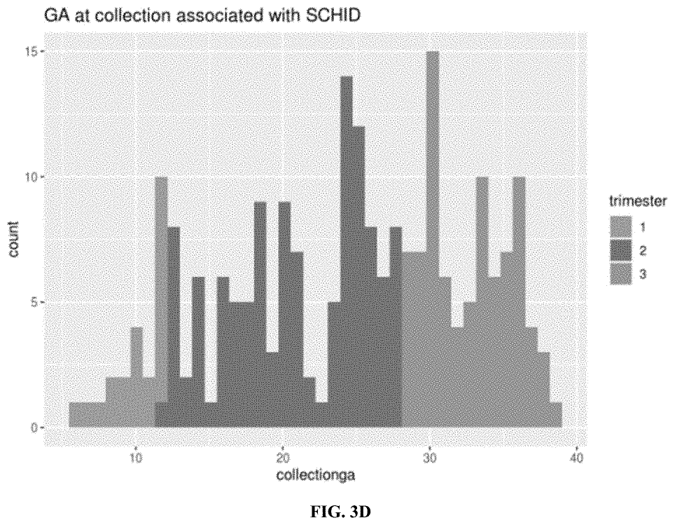

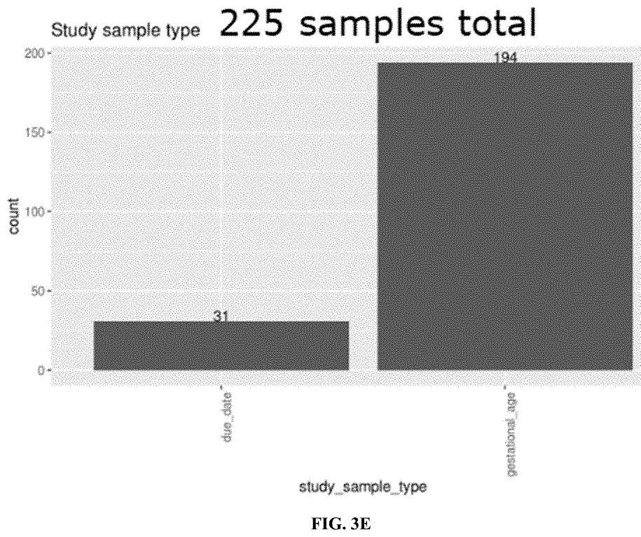

As shown in FIG. 3A, a first cohort of subjects (e.g., pregnant women) was established (with patient identification numbers shown on the x-axis), from which one or more biological samples (e.g., 2 or 3 each) were collected and assayed at different time points corresponding to an estimated gestational age (shown on the y-axis, in increasing order of estimated gestational age at delivery) of a fetus of each subject, using methods and systems of the present disclosure. For example, the estimated gestational age (shown on the y-axis) may be determined using methods such as ultrasound imaging, a last menstrual period (LMP) date, or a combination thereof, and may range from 0 to about 42 weeks. The first cohort includes subjects from whom different sample types were collected for use in different studies, including studies for the prediction of delivery, prediction of due date, and prediction of actual gestational age of a fetus of each subject. FIG. 3B shows a distribution of participants in the first cohort based on each participant's age at the time of medical record abstraction. FIG. 3C shows a distribution of 100 participants in the first cohort based on each participant's race. FIG. 3D shows a distribution of collected samples in the gestational age cohort based on each participant's estimated gestational age and trimester at the time of collection of each sample. FIG. 3E shows a distribution of 225 collected samples in the first cohort based on the study sample type of the collected samples.

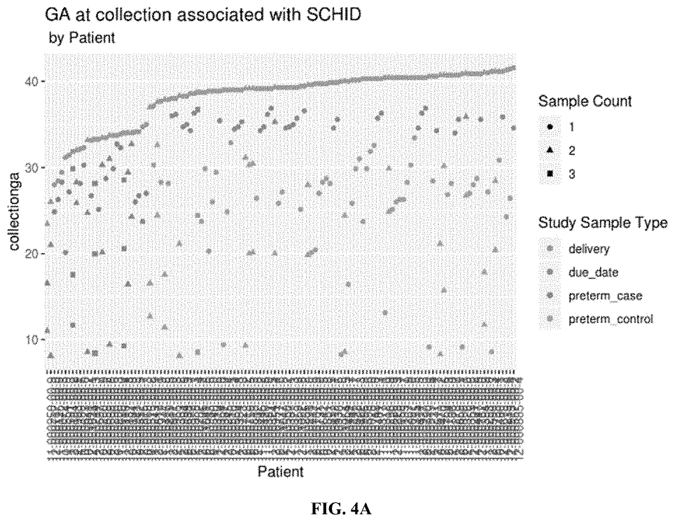

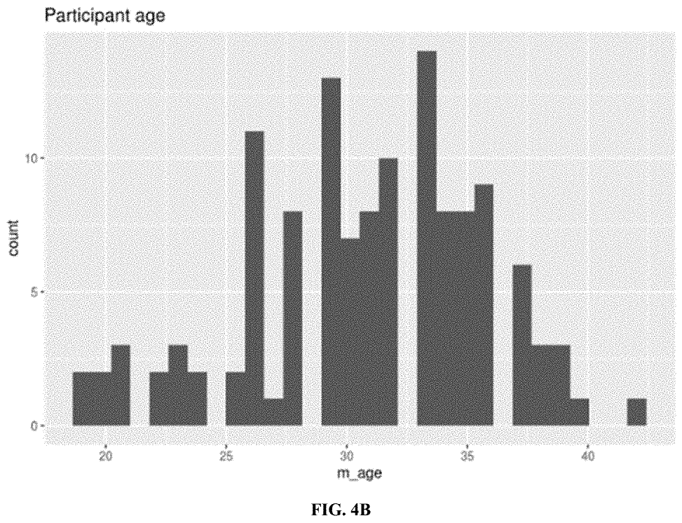

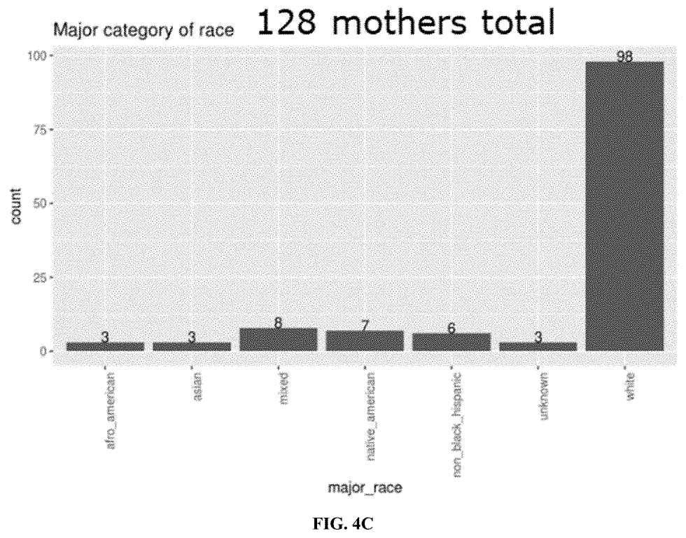

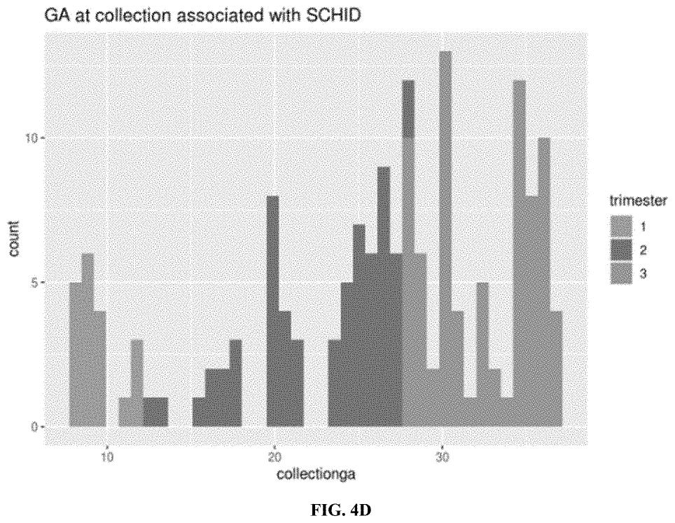

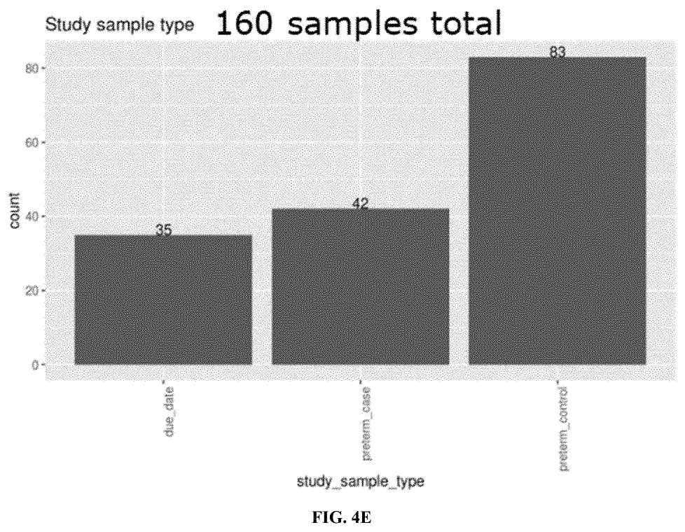

As shown in FIG. 4A, a second cohort of subjects (e.g., pregnant women) was established (with patient identification numbers shown on the x-axis), from which one or more biological samples (e.g., 1, 2, or 3 each) were collected and assayed at different time points corresponding to an estimated gestational age (shown on the y-axis, in increasing order of estimated gestational age at delivery) of a fetus of each subject, using methods and systems of the present disclosure. For example, the estimated gestational age (shown on the y-axis) may be determined using methods such as ultrasound imaging, a last menstrual period (LMP) date, or a combination thereof, and may range from 0 to about 42 weeks. The second cohort includes subjects from whom different sample types were collected for use in different studies, including studies for the prediction of pre-term birth, prediction of delivery, prediction of due date, and prediction of actual gestational age of a fetus of each subject. FIG. 4B shows a distribution of participants in the second cohort based on each participant's age at the time of medical record abstraction. FIG. 4C shows a distribution of 128 participants in the second cohort based on each participant's race. FIG. 4D shows a distribution of collected samples in the second cohort based on each participant's estimated gestational age and trimester at the time of collection of each sample. FIG. 4E shows a distribution of 160 collected samples in the second cohort based on the study sample type of the collected samples.

### Example 2: Prediction of Due Date

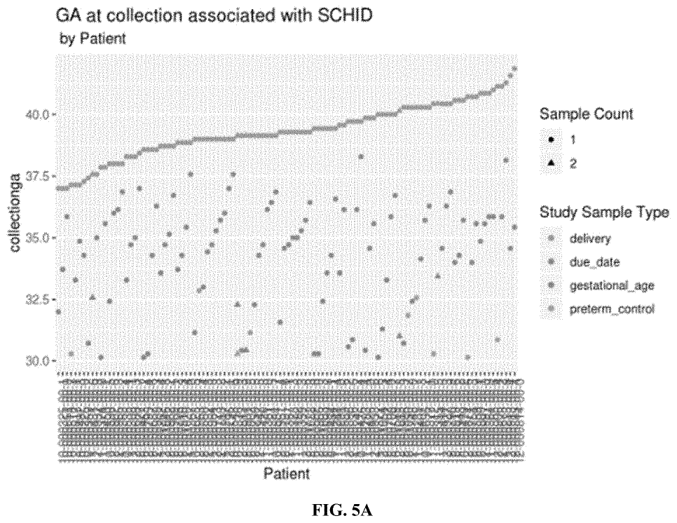

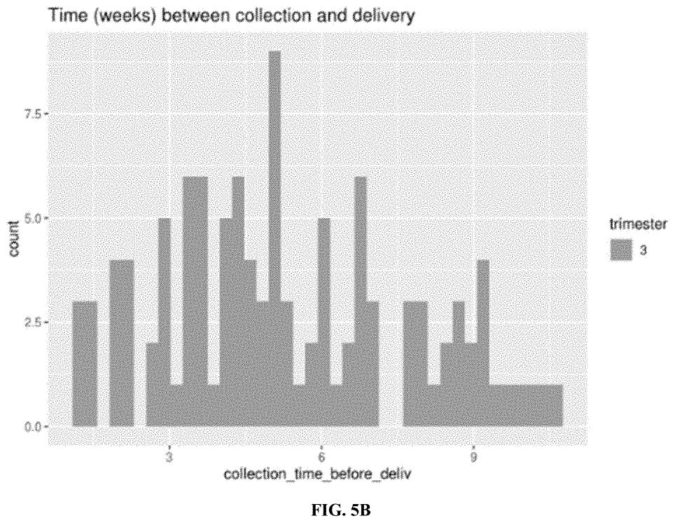

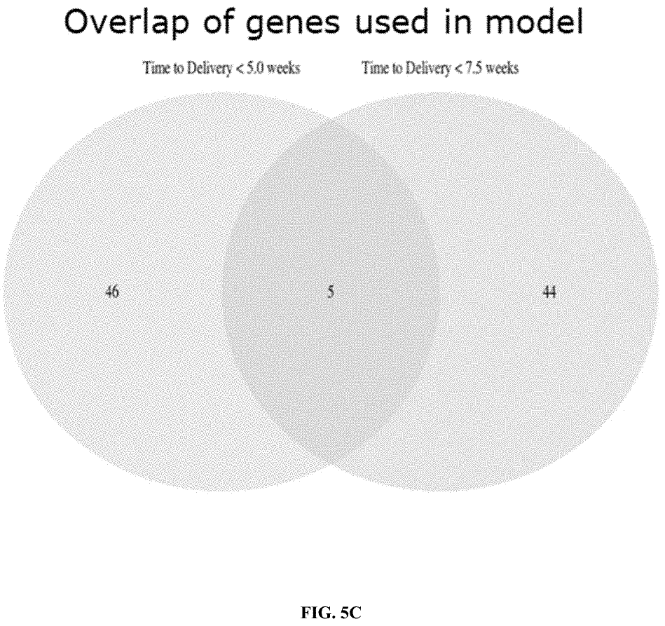

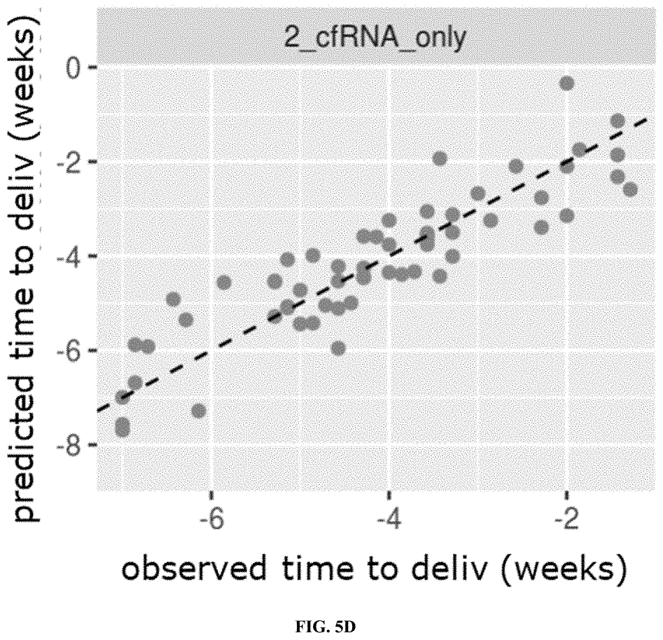

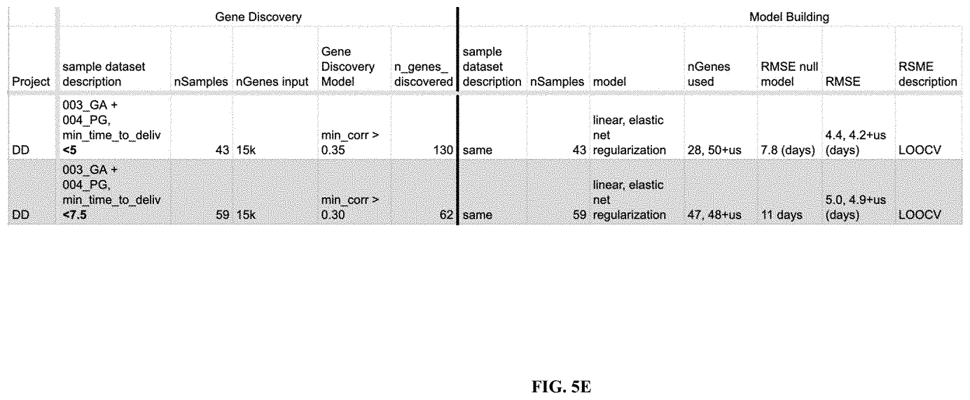

As shown in FIG. 5A, a due date cohort of subjects (e.g., pregnant women) was established (with patient identification numbers shown on the x-axis), from which one or more biological samples (e.g., 1 or 2 each) were collected and assayed at different time points corresponding to an estimated gestational age (shown on the y-axis, in increasing order of estimated gestational age at delivery) of a fetus of each subject, using methods and systems of the present disclosure. The due date cohort included subjects from the first cohort and second cohort, as described in Example 1. The due date cohort includes subjects from whom different sample types were collected for use in different studies, including studies for the prediction of pre-term birth (e.g., as controls), prediction of delivery, prediction of due date, and prediction of actual gestational age of a fetus of each subject.

FIG. 5B shows a distribution of collected samples in the due date cohort based on the time between the date of sample collection and the date of delivery (time to delivery). All samples were collected in the third trimester of pregnancy, less than 12 weeks before the date of delivery, of which 59 samples had a time-to-delivery of less than 7.5 weeks and 43 samples had a time-to-delivery of less than 5 weeks. Using systems and methods of the present disclosure, a first set of predictive models was generated from the 59 samples with a time-to-delivery of less than 7.5 weeks, and a second set of predictive models was generated from the 43 samples with a time-to-delivery of less than 5 weeks. The sets of predictive models included a predictive model generated with estimated due date information (e.g., determined using estimated gestational age from ultrasound measurements) and without the estimated due date information. Each of the predictive models comprised a linear regression model with elastic net regularization. The generation of the predictive models included identifying four sets of genes which had the highest correlation with (e.g., were most predictive of) due date (e.g., as measured by time to delivery) among the respective cohorts, including (1) less than 7.5 weeks time-to-delivery with estimated due date information, (2) less than 7.5 weeks time-to-delivery without estimated due date information, (3) less than 5 weeks time-to-delivery with estimated due date information, and (4) less than 5 weeks time-to-delivery without estimated due date information. These four sets of genes that are predictive for due date are listed in Table 1.

TABLE 1
Sets of Genes Predictive for Due Date by Cohort

| | Predictive Genes Included | Predictive Genes Not Included |
|---|---|---|
| Cohort | in Predictive Model | in Predictive Model |
| <7.5 weeks time-to-delivery | ACKR2, AKAP3, ANO5, | ADAMTS10, ADCY6, |
| with estimated due date info | C1orf21, C2orf42, CARNS1, | ATP9A, CCDC173, |
| | CASC15, CCDC102B, | CLIC4P1, CXorf65, |
| | CDC45, CDIPT, CMTM1, | KBTBD11, MKRN4P, |
| | collectionga, COPS8, CTD- | MKRN9P, NEXN-AS1, |
| | 2267D19.3, CTD-2349P21.9, | SMG1P2, ST13P3, XXbac- |
| | DDX11L1, DGUOK, | BPG252P9.9, ZNF114 |
| | DPAGT1, EIF4A1P2, | |
| | FANK1, FERMT1, FKRP, | |
| | GAMT, GOLGA6L4, KLLN, | |
| | LINC01347, LTA, MAPK12, | |
| | METRN, MPC2, MYL12BP1, | |
| | NME4, NPM1P30, PCLO, | |
| | PIF1, PTP4A3, RIMKLB, | |
| | RP13-88F20.1, S100B, | |
| | SIGLEC14, SLAIN1, | |
| | SPATA33, STAT1, TFAP2C, | |
| | TMEM94, TMSB4XP8, | |
| | TRGV10, ZNF124, ZNF713 | |
| <7.5 weeks time-to-delivery | ACKR2, AKAP3, ANO5, | ADAMTS10, ADCY6, |
| without estimated due date | C1orf21, C2orf42, CARNS1, | ATP9A, CCDC173, |
| info | CASC15, CCDC102B, | CLIC4P1, KBTBD11, |
| | CDC45, CDIPT, CMTM1, | MKRN9P, NEXN-AS1, |
| | COPS8, CTD-2267D19.3, | SMG1P2, ST13P3, STAT1, |
| | CTD-2349P21.9, CXorf65, | TMEM94, XXbac- |
| | DDX11L1, DGUOK, | BPG252P9.9, ZNF114, |
| | DPAGT1, EIF4A1P2, | ZNF713 |
| | FANK1, FERMT1, FKRP, | |
| | GAMT, GOLGA6L4, KLLN, | |
| | LINC01347, LTA, MAPK12, | |
| | METRN, MKRN4P, MPC2, | |
| | MYL12BP1, NME4, | |
| | NPM1P30, PCLO, PIF1, | |
| | PTP4A3, RIMKLB, RP13- | |
| | 88F20.1, S100B, SIGLEC14, | |
| | SLAIN1, SPATA33, | |
| | TFAP2C, TMSB4XP8, | |
| | TRGV10, ZNF124 | |
| <5 weeks time-to-delivery | ATP6V1E1P1, ATP8A2, | AB019441.29, AC004076.9, |
| with estimated due date info | C2orf68, CACNB3, CD40, | ACKR2, ADAMTS10, ADM, |
| | CDKL4, CDKL5, CEP152, | AP5B1, APOE, AQP9, |
| | CLEC4D, COL18A1, | ARHGEF40, BCL3, CA4, |
| | collectionga, COX16, CTBS, | CCDC84, CCR3, CD177, |
| | CTD-2272G21.2, CXCL2, | CDPF1, CFAP46, CHST7, |
| | CXCL8, DHRS7B, DPPA4, | CLYBL, CMTM1, CRADD, |
| | EIF5A2, FERMT1, GNB1L, | CSF3R, CXCL1, DAPK2, |
| | IFITM3, KATNAL1, LRCH4, | DLEC1, DPAGT1, ECHDC2, |
| | MBD6, MIR24-2, MTSS1, | ERP27, FCGR3B, FKRP, |
| | MYSM1, NCK1-AS1, | FUT7, GZMM, HAUS4, |
| | NPIPB4, NR1H4, PDE1C, | HKDC1, HMGB1P11, |
| | PEMT, PEX7, PIF1, | IGLV3-21, IL18R1, IRX3, |
| | PPP2R3A, PXDN, RABIF, | KBTBD11, KCNJ2, KDM6B, |
| | SERTAD3, SIGLEC14, | LEMD2, LINC00694, LIPE- |
| | SLC25A53, SPANXN4, | AS1, LMF2, LMLN-AS1, |
| | SSH3, SUPT3H, | LPCAT4, LRG1, MAP3K10, |
| | TMEM150C, TNFAIP6, | MAP3K6, MAPK12, |
| | UPP1, XKR8, ZC2HC1C, | METTL26, MGAM, |
| | ZMYM1, ZNF124 | MID1IP1, MIF-AS1, MME, |
| | | MRPL23, NAP1L4P3, |
| | | NLRP6, NPIPA5, NUP58, |
| | | OPRL1, PADI2, PGS1, POR, |
| | | RBKS, RNASET2, |
| | | SDCBPP2, SHE, SUMO2, |
| | | SUOX, SURF1, TATDN2, |
| | | TFE3, TMCC3, TMEM8A, |
| | | TMEM94, TOR1B, UNKL, |
| | | ZDHHC18, ZNF668 |
| <5 weeks time-to-delivery | C2orf68, CACNB3, CD40, | AB019441.29, AC004076.9, |
| without estimated due date | CDKL5, CTBS, CTD- | ACKR2, ADAMTS10, ADM, |
| info | 2272G21.2, CXCL8, | AP5B1, APOE, AQP9, |
| | DHRS7B, EIF5A2, IFITM3, | ARHGEF40, ATP6V1E1P1, |
| | MIR24-2, MTSS1, MYSM1, | ATP8A2, BCL3, CA4, |
| | NCK1-AS1, NR1H4, PDE1C, | CCDC84, CCR3, CD177, |
| | PEMT, PEX7, PIF1, | CDKL4, CDPF1, CEP152, |
| | PPP2R3A, RABIF, | CFAP46, CHST7, CLEC4D, |
| | SIGLEC14, SLC25A53, | CLYBL, CMTM1, COL18A1, |
| | SPANXN4, SUPT3H, | COX16, CRADD, CSF3R, |
| | ZC2HC1C, ZMYM1, ZNF124 | CXCL1, CXCL2, DAPK2, |
| | | DLEC1, DPAGT1, DPPA4, |
| | | ECHDC2, ERP27, FCGR3B, |
| | | FERMT1, FKRP, FUT7, |
| | | GNB1L, GZMM, HAUS4, |
| | | HKDC1, HMGB1P11, |
| | | IGLV3-21, IL18R1, IRX3, |
| | | KATNAL1, KBTBD11, |
| | | KCNJ2, KDM6B, LEMD2, |
| | | LINC00694, LIPE-AS1, |
| | | LMF2, LMLN-AS1, |
| | | LPCAT4, LRCH4, LRG1, |
| | | MAP3K10, MAP3K6, |
| | | MAPK12, MBD6, METTL26, |
| | | MGAM, MID1IP1, MIF-AS1, |
| | | MME, MRPL23, NAP1L4P3, |
| | | NLRP6, NPIPA5, NPIPB4, |
| | | NUP58, OPRL1, PADI2, |
| | | PGS1, POR, PXDN, RBKS, |
| | | RNASET2, SDCBPP2, |
| | | SERTAD3, SHE, SSH3, |
| | | SUMO2, SUOX, SURF1, |
| | | TATDN2, TFE3, TMCC3, |
| | | TMEM150C, TMEM8A, |
| | | TMEM94, TNFAIP6, |
| | | TOR1B, UNKL, UPP1, |
| | | XKR8, ZDHHC18, ZNF668 |

FIG. 5C is a Venn diagram showing the overlap of genes used in the first and second predictive models of due date. The first predictive model had a total of 51 most predictive genes, and the second predictive model had a total of 49 most predictive genes; further, only 5 genes overlapped between the two predictive models.

FIG. 5D is a plot showing the concordance between a predicted time to delivery (in weeks) and the observed (actual) time to delivery (in weeks) for the subjects in the due date cohort. The predicted time to delivery outcomes were generated using the respective predictive model based on the predictive genes listed in Table 1.

FIG. 5E shows a summary of the predictive models for predicting due date, including a predictive model using samples with a time-to-delivery of less than 5 weeks and predictive model using samples with a time-to-delivery of less than 7.5 weeks; different predictive models were generated with estimated due date information (e.g., determined using estimated gestational age from ultrasound measurements) and without the estimated due date information. A total of about 15,000 genes were evaluated for use in the predictive model (e.g., as part of the gene discovery process). Further, a total of 130 genes and 62 genes were identified as being predictive for due date among the “<5-week” and “<7.5-week” sample sets, respectively. A total of 28 and 47 genes were identified for inclusion in the predictive model for predicting due date without estimated due date information (e.g., from ultrasound) among the “<5-week” and “<7.5-week” sample sets, respectively. A total of 50 and 48 genes were identified for inclusion in the predictive model for predicting due date with estimated due date information (e.g., from ultrasound) among the “<5-week” and “<7.5-week” sample sets, respectively.

### Example 3: Prediction of Gestational Age (GA)

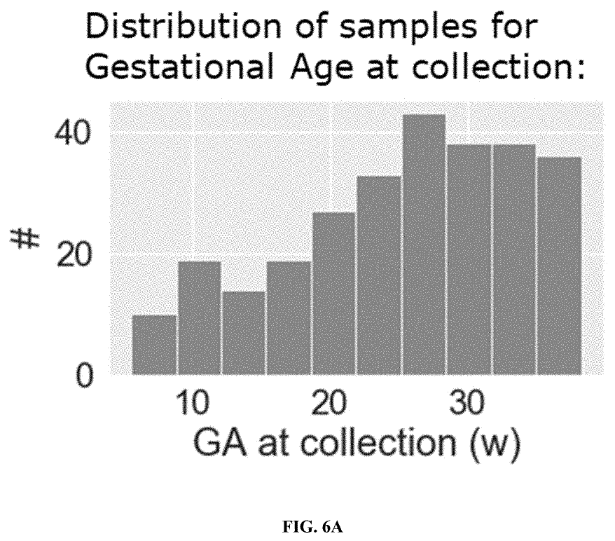

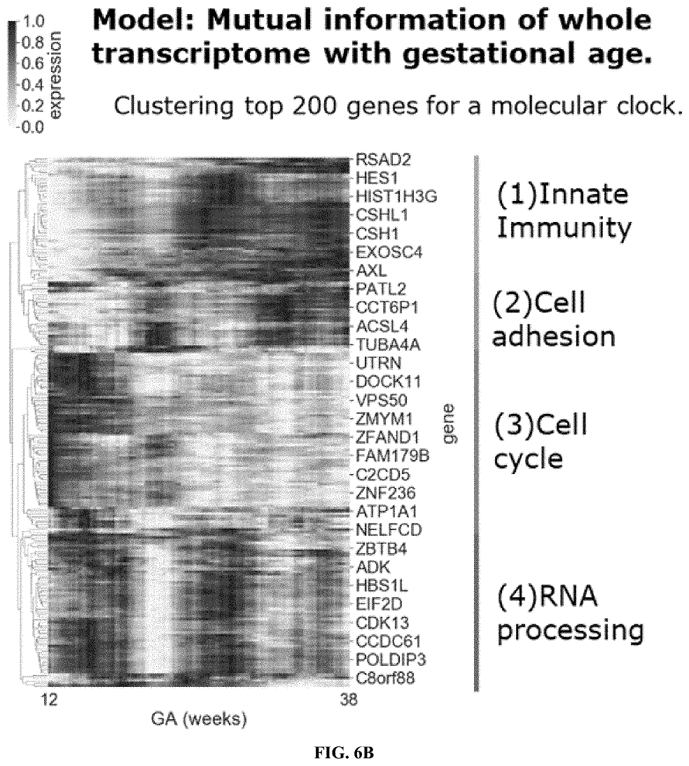

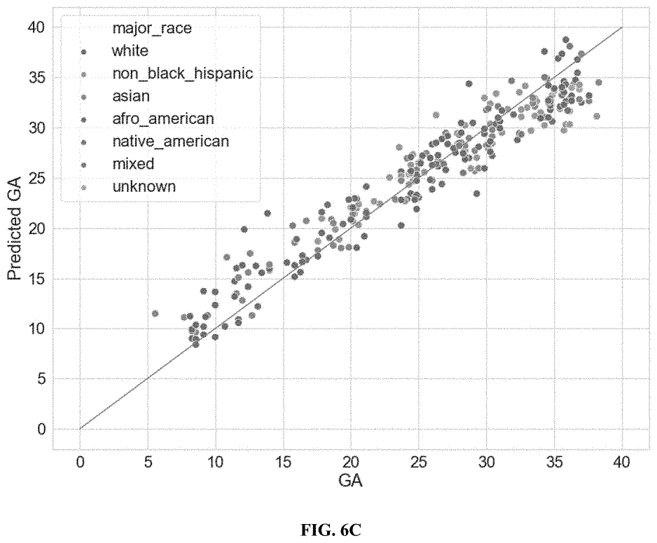

As shown in FIG. 6A, a gestational age cohort of subjects (e.g., pregnant women) was established, from which one or more biological samples (e.g., 1 or 2 each) were collected and assayed at different time points corresponding to an estimated gestational age of a fetus of each subject, using methods and systems of the present disclosure. The gestational age cohort included subjects from the first cohort, as described in Example 1. The gestational age cohort includes subjects from whom different sample types were collected for use in different studies, including studies for the prediction of delivery, prediction of due date, and prediction of actual gestational age of a fetus of each subject.

FIG. 6B is a visual model showing mutual information of the whole transcriptome, where expression of a plurality of gestational age-associated genes varies with gestational age throughout the course of a pregnancy. As shown in the figure, different clusters of genes exhibit fluctuations (e.g., increases and decreases) during different times (e.g., at different estimated gestational ages) throughout the course of a pregnancy. For example, genes associated with innate immunity (e.g., RSAD2, HES1, HIST1H3G, CSHL1, CSH1, EXOSC4, and AXL) and genes associated with cell adhesion (e.g., PATL2, CCT6P1, ACSL4, and TUBA4A) exhibited increased expression during the latter portion of pregnancy as compared to the earlier portion of pregnancy. As another example, genes associated with cell cycle (e.g., UTRN, DOCK11, VPS50, ZMYM1, ZFAND1, FAM179B, C2CD5, and ZNF236) exhibited increased expression during the earlier portion of pregnancy as compared to the latter portion of pregnancy. As another example, genes associated with RNA processing (e.g., ZBTB4, ADK, HBS1L, EIF2D, CDK13, CCDC61, POLDIP3, and C8orf88) exhibited increased expression during the earlier and middle portions of pregnancy as compared to the latter portion of pregnancy. Therefore, different sets or clusters of genes can be assayed for use as a “molecular clock” to track and predict different gestational ages of a fetus during the course of a pregnancy. These sets of genes that are predictive for gestational age are listed in Table 2. Further, pathways that are predictive for gestational age are listed in Table 3 by cluster.

TABLE 2
Sets of Genes Predictive for Gestational Age by Cluster

| Cluster | Genes |
|---|---|
| 1 | CSHL1, CAPN6, PAPPA, LGALS14, SVEP1, VGLL3, ARMCX6, EXPH5, HDGF, |
| | HSD3B1, OSBP2, BEX1, CSH2, HIST1H2AL, HCFC1R1, AL773572.7, ACTG1, |
| | MMP8, UBE2L6, CPNE2, EFHD1, CSH1, HES1, RSAD2, RNASE3, CARD16, |
| | S100A12, NDUFS5, LRIF1, EXOSC4, CYP19A1, NXF3, STAT1, G6PC3, TACC2, |
| | HIST1H3G, BCL7B, DEFA4, OLFM4, OXTR, IF16, RDX, CAT, PLAC4, |
| | FAM207A, AXL, PGLYRP1 |
| 2 | PATL2, NAPA, PRUNE1, ST20, ATF4, FAXDC2, BEX3, ZNF117, TCEAL3, |
| | EHD3, TUBA1B, GPR180, SUCNR1, OTUD5, ACSL4, PDIA3, ZBED5-AS1, VIL1, |
| | ITM2B, TUBA4A, CECR2, RPAP3, CCT6P1, KCNMB1 |
| 3 | SCAF8, SEC24B, MYCBP2, FNDC3A, C2CD5, FRA10AC1, KIAA0368, PLOD1, |
| | ZNF44, SLC12A2, RARS, AUP1, NARS2, GON4L, RBL1, SPG11, C3orf62, VPS50, |
| | AKAP7, CEP290, WAPL, RIC1, EXOC4, UTRN, BIRC6, FASTKD1, SNRNP48, |
| | CEP128, BPTF, RLF, ZNF236, MAP4K3, DYRK1A, ZMYM1, TTC13, RNF121, |
| | REPS1, CCDC141, DOCK11, DEK, CCNL1, ATP1A1, NSD1, MIPOL1, VCAN, |
| | ZNRF2, ITSN2, EZH1, CACUL1, MIS18BP1, USP48, KMT5B, MCCC1, TBC1D32, |
| | CCDC66, ENSG00000173088, SMAD4, ATAD5, FAM179B, KPNA5, ZFAND1, |
| | CARNMT1, ZDHHC5, TASP1, PCGF6, PHIP |
| 4 | CCDC61, POLDIP3, IKBKE, SIPA1L1, NOC2L, PLEC, PLXND1, MAP2K2, |
| | HIVEP3, FAM111A, AOAH, ARHGAP30, DOCK10, FAM217B, NBPF1, |
| | HNRNPA1, DTX2, MTBP, SLC26A2, LRRK1, NFATC1, FLNB, MARCKS, BRD9, |
| | SNRPA1, TAF3, MYO1G, ZNF557, CD53, HBS1L, NFKBIE, EIF2D, PARP14, |
| | NCL, VPS18, ADK, PSMG4, IMP3, SH2D1B, CHTOP, NELFCD, PABPC1, |
| | TSHZ1, ZNF383, SDCCAG3, CDK13, TTC39C, ZBTB4, PUM2, C1orf123, GCDH, |
| | SGTA, NOL4L, LMCD1, KLHL2 |
| 5 | GABARAPL2, RAB6C, RAB6A |
| 6 | MBNL3, MYL4, C8orf88, FTLP3, RAB2B |

TABLE 3
Pathways Predictive for Gestational Age by Cluster

| | | | | Entities False |
|---|---|---|---|---|
| | | | Entities | Detection |
| Cluster | Pathway Identifier | Pathway Name | p Value | Rate (FDR) |
| 1 | R-HSA-909733 | Interferon alpha/beta signaling | 1.16E−04 | 0.030180579 |
| 1 | R-HSA-913531 | Interferon Signaling | 2.08E−04 | 0.030180579 |
| 1 | R-HSA-9013508 | NOTCH3 Intracellular Domain Regulates | 4.72E−04 | 0.037300063 |
| | | Transcription | | |
| 1 | R-HSA-1280215 | Cytokine Signaling in Immune system | 5.18E−04 | 0.037300063 |
| 1 | R-HSA-196025 | Formation of annular gap junctions | 9.90E−04 | 0.056424803 |
| 1 | R-HSA-190873 | Gap junction degradation | 0.001175517 | 0.056424803 |
| 1 | R-HSA-437239 | Recycling pathway of L1 | 0.001591097 | 0.060736546 |
| 1 | R-HSA-8941856 | RUNX3 regulates NOTCH signaling | 0.002067719 | 0.060736546 |
| 1 | R-HSA-2197563 | NOTCH2 intracellular domain regulates | 0.002067719 | 0.060736546 |
| | | transcription | | |
| 1 | R-HSA-1059683 | Interleukin-6 signaling | 0.002328072 | 0.060736546 |
| 1 | R-HSA-9012852 | Signaling by NOTCH3 | 0.002336021 | 0.060736546 |
| 1 | R-HSA-446353 | Cell-extracellular matrix interactions | 0.002892685 | 0.060737316 |
| 1 | R-HSA-196071 | Metabolism of steroid hormones | 0.003139605 | 0.060737316 |
| 1 | R-HSA-210744 | Regulation of gene expression in late | 0.003196701 | 0.060737316 |
| | | stage (branching morphogenesis) | | |
| | | pancreatic bud precursor cells | | |
| 1 | R-HSA-193993 | Mineralocorticoid biosynthesis | 0.003196701 | 0.060737316 |
| 1 | R-HSA-6798695 | Neutrophil degranulation | 0.003621161 | 0.065180904 |
| 1 | R-HSA-9013695 | NOTCH4 Intracellular Domain Regulates | 0.005317217 | 0.085315773 |
| | | Transcription | | |
| 1 | R-HSA-194002 | Glucocorticoid biosynthesis | 0.005718941 | 0.085315773 |
| 1 | R-HSA193048 | Androgen biosynthesis | 0.005718941 | 0.085315773 |
| 1 | R-HSA-912694 | Regulation of IFNA signaling | 0.006134158 | 0.085315773 |
| 1 | R-HSA-982772 | Growth hormone receptor signaling | 0.006562752 | 0.085315773 |
| 1 | R-HSA-6783589 | Interleukin-6 family signaling | 0.00700461 | 0.091059924 |
| 1 | R-HSA-168256 | Immune System | 0.007818938 | 0.093827257 |
| 2 | R-HSA-8955332 | Carboxyterminal post-translational | 1.49E−04 | 0.01808342 |
| | | modifications of tubulin | | |
| 2 | R-HSA-983231 | Factors involved in megakaryocyte | 5.42E−04 | 0.01808342 |
| | | development and platelet production | | |
| 2 | R-HSA-190840 | Microtubule-dependent trafficking of | 8.77E−04 | 0.01808342 |
| | | connexons from Golgi to the plasma | | |
| | | membrane | | |
| 2 | R-HSA-190872 | Transport of connexons to the plasma | 9.58E−04 | 0.01808342 |
| | | membrane | | |
| 2 | R-HSA-389977 | Post-chaperonin tubulin folding pathway | 0.001128943 | 0.01808342 |
| 2 | R-HSA-6811434 | COPI-dependent Golgi-to-ER retrograde | 0.001205561 | 0.01808342 |
| | | traffic | | |
| 2 | R-HSA-6807878 | COPI-mediated anterograde transport | 0.001205561 | 0.01808342 |
| 2 | R-HSA-389960 | Formation of tubulin folding | 0.001615847 | 0.022621853 |
| | | intermediates by CCT/TriC | | |
| 2 | R-HSA-9619483 | Activation of AMPK downstream of | 0.002065423 | 0.024371102 |
| | | NMDARs | | |
| 2 | R-HSA-5626467 | RHO GTPases activate IQGAPs | 0.002309953 | 0.024371102 |
| 2 | R-HSA-389958 | Cooperation of Prefoldin and TriC/CCT | 0.00243711 | 0.024371102 |
| | | in actin and tubulin folding | | |
| 2 | R-HSA-190861 | Gap junction assembly | 0.002978066 | 0.024970608 |
| 2 | R-HSA-8856688 | Golgi-to-ER retrograde transport | 0.003023387 | 0.024970608 |
| 2 | R-HSA-381042 | PERK regulates gene expression | 0.003121326 | 0.024970608 |
| 2 | R-HSA-199977 | ER to Golgi Anterograde Transport | 0.004028523 | 0.027278879 |
| 2 | R-HSA-9609736 | Assembly and cell surface presentation of | 0.004047319 | 0.027278879 |
| | | NMDA receptors | | |
| 2 | R-HSA-190828 | Gap junction trafficking | 0.004727036 | 0.027278879 |
| 2 | R-HSA-437239 | Recycling pathway of L1 | 0.005269036 | 0.027278879 |
| 2 | R-HSA-5620924 | Intraflagellar transport | 0.005455776 | 0.027278879 |
| 2 | R-HSA-157858 | Gap junction trafficking and regulation | 0.005455776 | 0.027278879 |
| 2 | R-HSA-6811436 | COPI-independent Golgi-to-ER | 0.006846767 | 0.034233833 |
| | | retrograde traffic | | |
| 2 | R-HSA-983189 | Kinesins | 0.00792863 | 0.03517302 |
| 2 | R-HSA-3371497 | HSP90 chaperone cycle for steroid | 0.008381604 | 0.03517302 |
| | | hormone receptors (SHR) | | |
| 2 | R-HSA-6811442 | Intra-Golgi and retrograde Golgi-to-ER | 0.008817252 | 0.03517302 |
| | | traffic | | |
| 2 | R-HSA-446203 | Asparagine N-linked glycosylation | 0.00885181 | 0.03517302 |
| 2 | R-HSA-948021 | Transport to the Golgi and subsequent | 0.008927485 | 0.03517302 |
| | | modification | | |
| 2 | R-HSA-1445148 | Translocation of SLC2A4 (GLUT4) to the | 0.010560059 | 0.03517302 |
| | | plasma membrane | | |
| 2 | R-HSA-392499 | Metabolism of proteins | 0.0111176 | 0.03517302 |
| 2 | R-HSA-8852276 | The role of GTSE1 in G2/M progression | 0.011600388 | 0.03517302 |
| | | after G2 checkpoint | | |
| 2 | R-HSA-205025 | NADE modulates death signalling | 0.01172434 | 0.03517302 |
| 2 | R-HSA-438064 | Post NMDA receptor activation events | 0.01527754 | 0.045832619 |
| 2 | R-HSA-380320 | Recruitment of NuMA to mitotic | 0.015578704 | 0.046736112 |
| | | centrosomes | | |
| 2 | R-HSA-390466 | Chaperonin-mediated protein folding | 0.016497529 | 0.049492587 |
| 2 | R-HSA-434313 | Intracellular metabolism of fatty acids | 0.017536692 | 0.052610075 |
| | | regulates insulin secretion | | |
| 2 | R-HSA-391251 | Protein folding | 0.018403238 | 0.055209713 |
| 2 | R-HSA-1296052 | Ca2+ activated K+ channels | 0.019466807 | 0.056873842 |
| 2 | R-HSA-109582 | Hemostasis | 0.020531826 | 0.056873842 |
| 2 | R-HSA-442755 | Activation of NMDA receptors and | 0.020738762 | 0.056873842 |
| | | postsynaptic events | | |
| 2 | R-HSA-5610787 | Hedgehog ‘off’ state | 0.024645005 | 0.056873842 |
| 2 | R-HSA-373760 | L1CAM interactions | 0.026893295 | 0.056873842 |
| 2 | R-HSA-2500257 | Resolution of Sister Chromatid Cohesion | 0.028436921 | 0.056873842 |
| 2 | R-HSA-381183 | ATF6 (ATF6-alpha) activates chaperone | 0.029062665 | 0.05812533 |
| | | genes | | |
| 2 | R-HSA-381033 | ATF6 (ATF6-alpha) activates chaperones | 0.032875598 | 0.065751195 |
| 2 | R-HSA-2132295 | MHC class II antigen presentation | 0.034112102 | 0.068224205 |
| 2 | R-HSA-5663220 | RHO GTPases Activate Formins | 0.034533251 | 0.069066501 |
| 2 | R-HSA-418457 | cGMP effects | 0.034776645 | 0.069553291 |
| 2 | R-HSA-381119 | Unfolded Protein Response (UPR) | 0.037102976 | 0.074205952 |
| 2 | R-HSA-5358351 | Signaling by Hedgehog | 0.042915289 | 0.077519335 |
| 2 | R-HSA-400451 | Free fatty acids regulate insulin secretion | 0.051724699 | 0.077519335 |
| 2 | R-HSA-389957 | Prefoldin mediated transfer of substrate | 0.055451773 | 0.077519335 |
| | | to CCT/TriC | | |
| 2 | R-HSA-2467813 | Separation of Sister Chromatids | 0.055478287 | 0.077519335 |
| 2 | R-HSA-68877 | Mitotic Prometaphase | 0.062192558 | 0.077519335 |
| 2 | R-HSA-5617833 | Cilium Assembly | 0.062720246 | 0.077519335 |
| 2 | R-HSA-68882 | Mitotic Anaphase | 0.062720246 | 0.077519335 |
| 2 | R-HSA-2555396 | Mitotic Metaphase and Anaphase | 0.064312651 | 0.077519335 |
| 2 | R-HSA-380994 | ATF4 activates genes in response to | 0.064707762 | 0.077519335 |
| | | endoplasmic reticulum stress | | |
| 2 | R-HSA-69275 | G2/M Transition | 0.064846542 | 0.077519335 |
| 2 | R-HSA-453274 | Mitotic G2-G2/M phases | 0.06591891 | 0.077519335 |
| 2 | R-HSA-936440 | Negative regulators of DDX58/IFIH1 | 0.068385614 | 0.077519335 |
| | | signaling | | |
| 2 | R-HSA-112316 | Neuronal System | 0.07344898 | 0.077519335 |
| 2 | R-HSA-112314 | Neurotransmitter receptors and | 0.075836046 | 0.077519335 |
| | | postsynaptic signal transmission | | |
| 2 | R-HSA-901042 | Calnexin/calreticulin cycle | 0.077519335 | 0.077519335 |
| 2 | R-HSA-392154 | Nitric oxide stimulates guanylate cyclase | 0.077519335 | 0.077519335 |
| 2 | R-HSA-5689896 | Ovarian tumor domain proteases | 0.081148593 | 0.081148593 |
| 2 | R-HSA-597592 | Post-translational protein modification | 0.085097153 | 0.085097153 |
| 2 | R-HSA-6811438 | Intra-Golgi traffic | 0.090161601 | 0.090161601 |
| 2 | R-HSA-75876 | Synthesis of very long-chain fatty acyl- | 0.095528421 | 0.095528421 |
| | | CoAs | | |
| 2 | R-HSA-5683826 | Surfactant metabolism | 0.099089328 | 0.099089328 |
| 3 | R-HSA-1538133 | G0 and Early G1 | 8.71E−04 | 0.206527784 |
| 3 | R-HSA-1362277 | Transcription of E2F targets under | 0.006680493 | 0.291565226 |
| | | negative control by DREAM complex | | |
| 3 | R-HSA-453279 | Mitotic G1-G1/S phases | 0.010050075 | 0.291565226 |
| 3 | R-HSA-3304347 | Loss of Function of SMAD4 in Cancer | 0.014424835 | 0.291565226 |
| 3 | R-HSA-3311021 | SMAD4 MH2 Domain Mutants in Cancer | 0.014424835 | 0.291565226 |
| 3 | R-HSA-3315487 | SMAD2/3 MH2 Domain Mutants in | 0.014424835 | 0.291565226 |
| | | Cancer | | |
| 3 | R-HSA-2173796 | SMAD2/SMAD3:SMAD4 heterotrimer | 0.015567079 | 0.291565226 |
| | | regulates transcription | | |
| 3 | R-HSA-3214841 | PKMTs methylate histone lysines | 0.023826643 | 0.291565226 |
| 3 | R-HSA-8952158 | RUNX3 regulates BCL2L11 (BIM) | 0.028644567 | 0.291565226 |
| | | transcription | | |
| 3 | R-HSA-2173793 | Transcriptional activity of | 0.029469648 | 0.291565226 |
| | | SMAD2/SMAD3:SMAD4 heterotrimer | | |
| 3 | R-HSA-8941855 | RUNX3 regulates CDKN1A transcription | 0.038011863 | 0.291565226 |
| 3 | R-HSA-3304349 | Loss of Function of SMAD2/3 in Cancer | 0.038011863 | 0.291565226 |
| 3 | R-HSA-444821 | Relaxin receptors | 0.038011863 | 0.291565226 |
| 3 | R-HSA-9645135 | STATS Activation | 0.04266207 | 0.291565226 |
| 3 | R-HSA-3595174 | Defective CHST14 causes EDS, | 0.04266207 | 0.291565226 |
| | | musculocontractural type | | |
| 3 | R-HSA-3595172 | Defective CHST3 causes SEDCJD | 0.04266207 | 0.291565226 |
| 3 | R-HSA-3304351 | Signaling by TGF-beta Receptor Complex | 0.04266207 | 0.291565226 |
| | | in Cancer | | |
| 3 | R-HSA-379724 | tRNA Aminoacylation | 0.043286108 | 0.291565226 |
| 3 | R-HSA-1640170 | Cell Cycle | 0.04679213 | 0.291565226 |
| 3 | R-HSA-3595177 | Defective CHSY1 causes TPBS | 0.047290122 | 0.291565226 |
| 3 | R-HSA-2470946 | Cohesin Loading onto Chromatin | 0.047290122 | 0.291565226 |
| 3 | R-HSA-426117 | Cation-coupled Chloride cotransporters | 0.047290122 | 0.291565226 |
| 3 | R-HSA-3371599 | Defective HLCS causes multiple | 0.047290122 | 0.291565226 |
| | | carboxylase deficiency | | |
| 3 | R-HSA-351906 | Apoptotic cleavage of cell adhesion | 0.051896124 | 0.291565226 |
| | | proteins | | |
| 3 | R-HSA-176974 | Unwinding of DNA | 0.056480178 | 0.291565226 |
| 3 | R-HSA-3323169 | Defects in biotin (Btn) metabolism | 0.056480178 | 0.291565226 |
| 3 | R-HSA-1445148 | Translocation of SLC2A4 (GLUT4) to the | 0.056493106 | 0.291565226 |
| | | plasma membrane | | |
| 3 | R-HSA-69278 | Cell Cycle, Mitotic | 0.057847859 | 0.291565226 |
| 3 | R-HSA-2022923 | Dermatan sulfate biosynthesis | 0.061042388 | 0.291565226 |
| 3 | R-HSA-2468052 | Establishment of Sister Chromatid | 0.061042388 | 0.291565226 |
| | | Cohesion | | |
| 3 | R-HSA-170834 | Signaling by TGF-beta Receptor Complex | 0.064216491 | 0.291565226 |
| 3 | R-HSA-68884 | Mitotic Telophase/Cytokinesis | 0.070101686 | 0.291565226 |
| 3 | R-HSA-1502540 | Signaling by Activin | 0.070101686 | 0.291565226 |
| 3 | R-HSA-8983432 | Interleukin-15 signaling | 0.074598978 | 0.291565226 |
| 3 | R-HSA-196780 | Biotin transport and metabolism | 0.087962635 | 0.291565226 |
| 3 | R-HSA-1362300 | Transcription of E2F targets under | 0.092374782 | 0.291565226 |
| | | negative control by p107 (RBL1) and | | |
| | | p130 (RBL2) in complex with HDAC1 | | |
| 3 | R-HSA-3560783 | Defective B4GALT7 causes EDS, | 0.096765893 | 0.291565226 |
| | | progeroid type | | |
| 3 | R-HSA-4420332 | Defective B3GALT6 causes EDSP2 and | 0.096765893 | 0.291565226 |
| | | SEMDJL1 | | |
| 3 | R-HSA-6804114 | TP53 Regulates Transcription of Genes | 0.096765893 | 0.291565226 |
| | | Involved in G2 Cell Cycle Arrest | | |
| 4 | R-HSA-8953854 | Metabolism of RNA | 0.008040167 | 0.222786123 |
| 4 | R-HSA-9013508 | NOTCH3 Intracellular Domain Regulates | 0.011600797 | 0.222786123 |
| | | Transcription | | |
| 4 | R-HSA-3304347 | Loss of Function of SMAD4 in Cancer | 0.013386586 | 0.222786123 |
| 4 | R-HSA-3560792 | Defective 5LC26A2 causes | 0.013386586 | 0.222786123 |
| | | chondrodysplasias | | |
| 4 | R-HSA-3311021 | SMAD4 MH2 Domain Mutants in Cancer | 0.013386586 | 0.222786123 |
| 4 | R-HSA-3315487 | SMAD2/3 MH2 Domain Mutants in | 0.013386586 | 0.222786123 |
| | | Cancer | | |
| 4 | R-HSA-73857 | RNA Polymerase II Transcription | 0.014524942 | 0.222786123 |
| 4 | R-HSA-8952158 | RUNX3 regulates BCL2L11 (BIM) | 0.026596735 | 0.222786123 |
| | | transcription | | |
| 4 | R-HSA-72203 | Processing of Capped Intron-Containing | 0.028244596 | 0.222786123 |
| | | Pre-mRNA | | |
| 4 | R-HSA-72187 | mRNA 3′-end processing | 0.028277064 | 0.222786123 |
| 4 | R-HSA-74160 | Gene expression (Transcription) | 0.02961978 | 0.222786123 |
| 4 | R-HSA-9012852 | Signaling by NOTCH3 | 0.032891337 | 0.222786123 |

FIG. 6C is a plot showing the concordance between a predicted gestational age (in weeks) and the measured gestational age (in weeks) for the subjects in the gestational age cohort. The subjects are stratified in the plot by major race (e.g., white, non-black Hispanic, Asian, Afro-American, Native American, mixed race (e.g., two or more races), or unknown). It is noteworthy that the data shows that, unlike many biological phenotypes, the gestational biomarkers model (e.g., prediction of gestational age based on a set of gestational age-associated biomarker genes) is independent of race or ethnicity. This observation indicates that the underlying molecular clock of pregnancy is highly conserved across races/ethnicities, which has a practical implication of making a universal assay for gestational age feasible. The predicted gestational ages were generated using a predictive model for gestational age (a Lasso model generating with a 10-fold cross-validation) based on the predictive genes listed in Table 2 and/or the predictive pathways listed in Table 3. Further, the predictive model weights of genes that are predictive for gestational age are listed in Table 4.

TABLE 4
Predictive Model Weights of Genes Predictive for Gestational Age

| Gene | Weight |
|---|---|
| CGA | −2.3291809 |
| CSH1 | 2.0997422 |
| CAPN6 | 1.58718823 |
| UBE2L6 | 0.78006933 |
| CYP19A1 | 0.7495651 |
| MCEMP1 | 0.66188425 |
| STAT1 | 0.62796009 |
| ANGPT2 | −0.61766869 |
| SUCNR1 | 0.60439183 |
| EXPH5 | 0.55503889 |
| LRMP | −0.53240046 |
| RGS9 | 0.43352062 |
| NXF3 | 0.40263822 |
| DDI2 | −0.39475793 |
| PPP2CB | −0.34436392 |
| BBX | 0.34034586 |
| FCGR2A | 0.33904027 |
| NREP | 0.33265012 |
| BEX1 | 0.27078087 |
| RYR3 | −0.25427064 |
| IGHA1 | −0.24225842 |
| IL18BP | −0.22511377 |
| SLC7A11 | 0.21310441 |
| TCHH | 0.2115899 |
| SMAD5 | −0.19126152 |
| FAM114A1 | −0.18288572 |
| CCDC66 | −0.18079341 |
| PLS3 | −0.17781532 |
| BCAT1 | 0.17680457 |
| RECQL | 0.17503129 |
| CD96 | 0.15741167 |
| FAM214A | −0.15229302 |
| GCNT1 | 0.14693661 |
| DCAF17 | −0.14675868 |
| HIST1H2BB | 0.1407058 |
| CCT6B | 0.13180261 |
| FBXL20 | −0.12456705 |
| H19 | −0.12185332 |
| SKIL | 0.11799157 |
| ABCB10 | 0.11737993 |
| FARS2 | 0.11728322 |
| SERPINB10 | 0.11535642 |
| MCCC1 | −0.10689218 |
| FTH1P7 | 0.10503966 |
| SLC4A7 | −0.10328859 |
| TCN1 | 0.10244934 |
| ARHGAP42 | −0.10056675 |
| RAC1 | 0.09965553 |
| EED | −0.09795522 |
| RAB8B | 0.09392322 |
| SOX12 | −0.09281749 |
| UBE2G1 | −0.09063966 |
| CFAP70 | −0.09009795 |
| SPA17 | 0.08878255 |
| RASAL2 | −0.08386265 |
| RHAG | 0.07777724 |
| NQO2 | 0.07671752 |
| NKAPL | 0.07183955 |
| SORBS2 | 0.07127603 |
| BTRC | −0.07061876 |
| LAMTOR3 | 0.06135476 |
| RDX | 0.06114729 |
| APOL4 | 0.06043051 |
| SVEP1 | 0.06015624 |
| IGHV3-23 | −0.05726866 |
| PPCS | 0.05506125 |
| TNIP3 | 0.05448006 |
| WDSUB1 | −0.05228332 |
| TMEM14A | 0.0522635 |
| SEMA3C | 0.05196743 |
| SUZ12 | −0.04935669 |
| GATSL2 | −0.0426659 |
| TMEM109 | 0.03944985 |
| CPNE2 | 0.03713674 |
| REEP5 | 0.03492848 |
| GCSAML | 0.03481997 |
| LYRM9 | 0.03446721 |
| CENPV | −0.03301296 |
| NEK6 | 0.03186441 |
| PET100 | −0.03081952 |
| FAM221A | −0.0293719 |
| ZDHHC8 | −0.02866679 |
| IGSF21 | 0.02810308 |
| FAM63B | −0.0259032 |
| HABP4 | −0.02585663 |
| LEMD3 | −0.01949602 |
| WDR27 | −0.01899405 |
| AXL | 0.01873862 |
| SMARCA1 | 0.01789833 |
| GNPAT | 0.01659611 |
| IGHV3-7 | −0.01587266 |
| DYNC2LI1 | −0.01543354 |
| PROS2P | 0.01216718 |
| ATP9A | 0.01210078 |
| HBEGF | −0.01123074 |
| COMT | 0.01102531 |
| DYNLT3 | 0.00555317 |
| TBC1D32 | −0.00434216 |
| MYL12B | 0.0037807 |

### Example 4: Prediction of Pre-Term Birth (PTB)

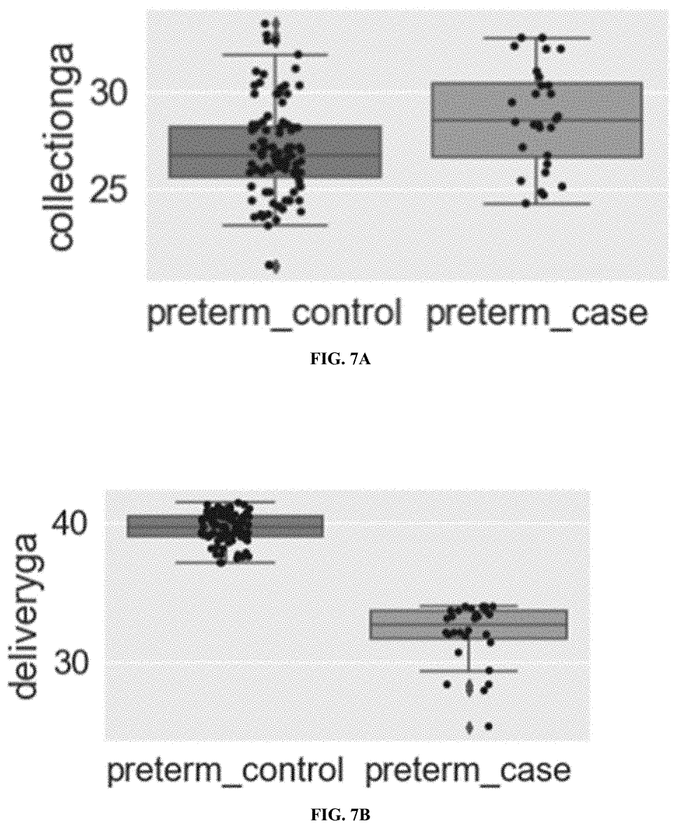

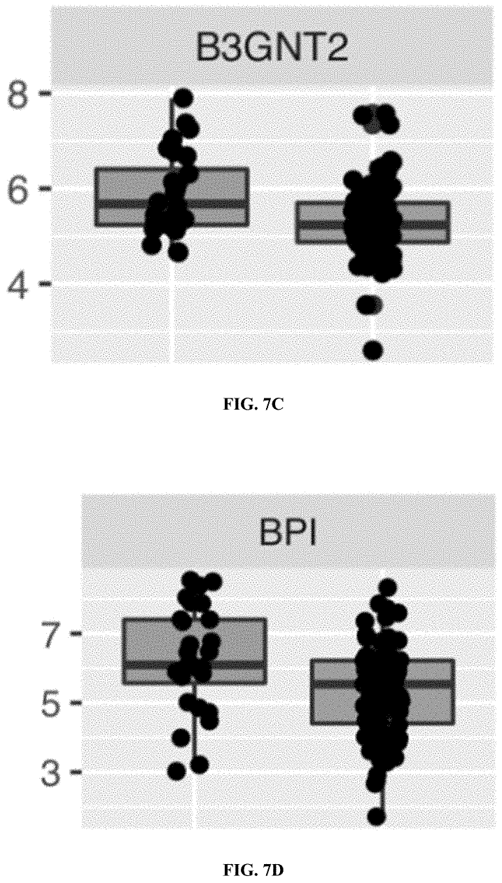

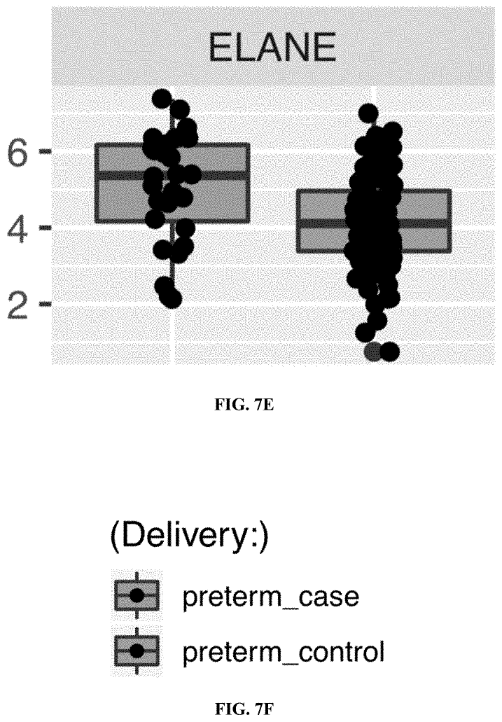

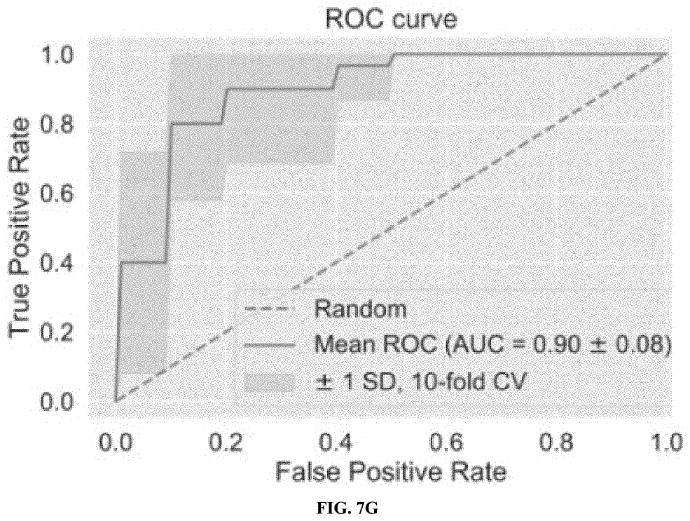

As shown in FIGS. 7A-7B, a pre-term birth (PTB) cohort of subjects (e.g., pregnant women) was established, from which one or more biological samples (e.g., 1, 2, 3, or more than 3 each) were collected and assayed at different time points corresponding to an estimated gestational age of a fetus of each subject, using methods and systems of the present disclosure. The pre-term birth cohort included subjects from the second cohort, as described in Example 1. The pre-term birth cohort includes subjects from whom different sample types were collected for use in different studies, including studies for the prediction of pre-term birth, prediction of delivery, prediction of due date, and prediction of actual gestational age of a fetus of each subject. As shown in the figure, a total of 160 samples from 128 pregnant subjects of the pre-term birth cohort were collected and assayed, of which 118 samples were collected from 100 pregnant subjects having full-term births and 42 samples were collected from 28 pregnant subjects having pre-term births (e.g., defined as occurring before an estimated gestational age of 37 weeks). The pre-term birth (PTB) cohort included a set of pre-term case samples (e.g., from women having pre-term births) and a set of pre-term control samples (e.g., from women having full-term births). Across the pre-term case samples and pre-term control samples, the distributions of gestational age at time of collection were similar (FIG. 7A), while the distributions of gestational age at delivery were clearly distinguishable to a statistically significant extent (FIG. 7B).

An analysis for differentially expressed genes between the pre-term case samples and pre-term control samples was performed, revealing that 151 genes were upregulated and 37 genes were downregulated. For example, FIGS. 7C-7E show differential gene expression of the B3GNT2, BP, and ELANE genes, respectively, between the pre-term case samples (left) and pre-term control samples (right). FIG. 7F shows a legend for the results from pre-term case samples and pre-term control samples shown in FIGS. 7C-7E. A set of genes that are predictive for pre-term birth (PTB) are listed in Table 5. Further, the predictive model weights of genes that are predictive for pre-term birth (PTB) are listed in Table 6.

TABLE 5
Set of Genes Predictive for Pre-Term Birth (PTB)

| Gene | BaseMean | Log2FoldChange | lfcSE | Stat | P Value | P_adj |
|---|---|---|---|---|---|---|
| MKI67 | 400.830667 | −0.601319668 | 0.108179231 | 32.84474216 | 9.98207E−09 | 9.05274E−05 |
| TPX2 | 65.5033344 | −0.581186144 | 0.110641746 | 29.0631565 | 7.00567E−08 | 0.000317672 |
| B3GNT2 | 50.6724879 | −0.811226454 | 0.166164856 | 24.85992629 | 6.16508E−07 | 0.001863703 |
| TOP2A | 216.98909 | −0.405447156 | 0.086617399 | 22.58819561 | 2.00714E−06 | 0.004550689 |
| CFAP45 | 124.955577 | −0.775232315 | 0.16837313 | 21.97718654 | 2.75911E−06 | 0.005004467 |
| RABEP1 | 589.967939 | 0.172443456 | 0.037329151 | 21.04101979 | 4.49555E−06 | 0.00502318 |
| SPAG5 | 23.1133858 | −0.653772557 | 0.145799452 | 20.86325357 | 4.93267E−06 | 0.00502318 |
| MRVI1 | 124.226298 | −0.680912281 | 0.155527024 | 20.7857985 | 5.13624E−06 | 0.00502318 |
| HIST1H2BB | 67.0856736 | −0.621390031 | 0.142395396 | 20.78222285 | 5.14584E−06 | 0.00502318 |
| IRX3 | 24.1768218 | −1.212908431 | 0.274268915 | 20.64129438 | 5.53885E−06 | 0.00502318 |
| PRC1 | 93.5892327 | −0.3611091 | 0.081976316 | 19.92418748 | 8.05745E−06 | 0.006094756 |
| ACSM3 | 27.2003668 | −0.716459154 | 0.169223045 | 19.92251129 | 8.06451E−06 | 0.006094756 |
| LTF | 95.8462149 | −1.197283648 | 0.285286547 | 19.21981298 | 1.16498E−05 | 0.008127079 |
| CLSPN | 101.400363 | −0.379383578 | 0.088756166 | 18.72100697 | 1.51306E−05 | 0.009801412 |
| ABCA13 | 28.4998585 | −1.147381421 | 0.276646667 | 18.52138019 | 1.68009E−05 | 0.009992992 |
| DAP3 | 276.946453 | 0.200259669 | 0.046325618 | 18.38293849 | 1.80668E−05 | 0.009992992 |
| CLPX | 260.222378 | 0.208245562 | 0.048240765 | 18.31405149 | 1.8732E−05 | 0.009992992 |
| PRDM4 | 73.7117025 | −0.280318521 | 0.068189159 | 17.43554082 | 2.97216E−05 | 0.014220995 |
| HJURP | 49.7967158 | −0.48470193 | 0.118013732 | 17.43093908 | 2.97937E−05 | 0.014220995 |
| CEACAM8 | 40.6294185 | −1.167910698 | 0.291855251 | 17.00860876 | 3.72107E−05 | 0.016873202 |
| WDR43 | 162.21835 | 0.201833504 | 0.048851646 | 16.90058186 | 3.93895E−05 | 0.01701064 |
| PHGDH | 64.6602039 | −1.038524899 | 0.272984761 | 16.10479806 | 5.9932E−05 | 0.024705606 |
| SPRY1 | 18.6318178 | −0.739453446 | 0.191408208 | 15.96857116 | 6.44028E−05 | 0.025394321 |
| COQ2 | 32.7210234 | −0.494334868 | 0.129086701 | 15.47489359 | 8.36084E−05 | 0.031168137 |
| SGO2 | 79.0913883 | −0.278147351 | 0.071596767 | 15.42336324 | 8.59194E−05 | 0.031168137 |
| FBN1 | 18.0266461 | −0.786173751 | 0.199134531 | 15.16720482 | 9.83976E−05 | 0.034321842 |
| GPSM2 | 63.6368478 | −0.305850326 | 0.079647479 | 15.04158139 | 0.000105168 | 0.034781625 |
| WASL | 69.0262558 | −0.314359854 | 0.082595598 | 15.00219484 | 0.000107386 | 0.034781625 |
| C10orf88 | 34.4590779 | −0.561281119 | 0.150387991 | 14.86051191 | 0.000115761 | 0.036201295 |
| MAPK10 | 62.7246279 | −0.787771018 | 0.214606489 | 14.75561567 | 0.000122382 | 0.036996225 |
| SDAD1 | 119.719558 | 0.323236991 | 0.083187212 | 14.62160832 | 0.000131399 | 0.038440635 |
| AP1AR | 52.9450923 | 0.296319236 | 0.07703744 | 14.44196908 | 0.000144545 | 0.039709576 |
| CEACAM6 | 17.6472741 | −1.040919908 | 0.28533353 | 14.37541601 | 0.000149745 | 0.039709576 |
| VPS9D1 | 31.4783536 | −0.64593929 | 0.173835235 | 14.35682089 | 0.000151231 | 0.039709576 |
| MEAF6 | 181.85469 | 0.234732787 | 0.061260932 | 14.3070259 | 0.000155284 | 0.039709576 |
| FOXM1 | 20.5441036 | −0.636516603 | 0.171727594 | 14.23388904 | 0.000161437 | 0.039709576 |
| SHCBP1 | 21.3472375 | −0.459928249 | 0.124085932 | 14.22723861 | 0.000162008 | 0.039709576 |
| CIT | 124.514777 | −0.328433636 | 0.088967509 | 13.99039883 | 0.000183747 | 0.043852559 |
| ACADVL | 137.011451 | −0.430868422 | 0.117813378 | 13.82728288 | 0.000200405 | 0.044288458 |
| BCORL1 | 111.923293 | −0.402393529 | 0.109550057 | 13.80336562 | 0.000202972 | 0.044288458 |
| HIST1H3F | 33.0009859 | −0.537748862 | 0.147682317 | 13.79931363 | 0.000203411 | 0.044288458 |
| ERI2 | 29.8917001 | −0.429671723 | 0.11865343 | 13.70904243 | 0.000213424 | 0.044288458 |
| ASPM | 108.467082 | −0.303317686 | 0.083048184 | 13.6994066 | 0.000214522 | 0.044288458 |
| LATS2 | 72.1128433 | −0.43419763 | 0.120730726 | 13.61286351 | 0.000224641 | 0.044288458 |
| P4HB | 308.144977 | −0.467363453 | 0.130617695 | 13.59109153 | 0.000227261 | 0.044288458 |
| RRM2 | 57.4816431 | −0.639528628 | 0.178697012 | 13.55808795 | 0.000231293 | 0.044288458 |
| HIST1H2AH | 39.7276884 | −0.738920384 | 0.209333866 | 13.55131997 | 0.000232128 | 0.044288458 |
| TBC1D7 | 20.8101265 | −0.491912362 | 0.137149751 | 13.53297652 | 0.000234408 | 0.044288458 |
| ZSCAN29 | 85.830534 | −0.403022474 | 0.113370078 | 13.47259044 | 0.000242074 | 0.044803426 |
| MRTO4 | 16.8779413 | 0.691948182 | 0.183119079 | 13.42031428 | 0.000248914 | 0.04514802 |
| ELANE | 29.9488832 | −0.86703039 | 0.248991041 | 13.32739769 | 0.000261556 | 0.045573275 |
| CCNA2 | 20.5346159 | −0.627654197 | 0.175281296 | 13.30323568 | 0.000264948 | 0.045573275 |
| NXF3 | 21.9931399 | −0.874037001 | 0.246746166 | 13.29345619 | 0.000266334 | 0.045573275 |
| C11orf24 | 39.2455928 | −0.422115026 | 0.118646242 | 13.24101829 | 0.000273889 | 0.045998149 |
| NUSAP1 | 163.110628 | −0.312315279 | 0.087355935 | 13.1574169 | 0.000286383 | 0.04722202 |
| CPNE2 | 98.1394967 | −0.412819488 | 0.115624299 | 13.1056335 | 0.000294409 | 0.047678502 |
| ENPP4 | 21.988534 | −0.702457326 | 0.199003539 | 13.00559611 | 0.000310561 | 0.049411963 |
| TADA3 | 384.86541 | −0.461754693 | 0.132540423 | 12.96637032 | 0.000317136 | 0.049588081 |
| CENPJ | 86.1330533 | −0.400578337 | 0.113794638 | 12.91463148 | 0.000326024 | 0.049862843 |
| BPI | 70.1177976 | −0.889016784 | 0.256224363 | 12.8843149 | 0.000331347 | 0.049862843 |
| FAM117B | 78.1729146 | 0.485833993 | 0.13119025 | 12.86163207 | 0.000335388 | 0.049862843 |
| HIBADH | 70.6973939 | 0.306490029 | 0.084559119 | 12.80182626 | 0.000346281 | 0.050537255 |
| DEFA3 | 67.2275316 | −1.117768363 | 0.327944883 | 12.7746206 | 0.000351354 | 0.050537255 |
| TAF1A | 25.0593769 | 0.374110248 | 0.103231417 | 12.74667933 | 0.000356642 | 0.050537255 |
| HIST1H1B | 194.721138 | −0.716085762 | 0.209616837 | 12.64672494 | 0.000376224 | 0.052491955 |
| NCAPG2 | 81.8608202 | −0.2529091 | 0.072071056 | 12.58777256 | 0.000388279 | 0.052889151 |
| MTG1 | 24.3831654 | 0.341740344 | 0.095511983 | 12.57598756 | 0.000390735 | 0.052889151 |
| CKAP2L | 58.9317012 | −0.343643101 | 0.098381001 | 12.52409347 | 0.000401738 | 0.053578821 |
| TRA2B | 676.542908 | −0.25572298 | 0.073568397 | 12.45496838 | 0.000416881 | 0.05479272 |
| ZBTB26 | 19.2710753 | −0.541284898 | 0.159692134 | 12.22219578 | 0.000472243 | 0.060690018 |
| ITGAE | 55.6496691 | −0.580656414 | 0.170762602 | 12.19638948 | 0.000478821 | 0.060690018 |
| TMEM204 | 24.0591736 | −0.617192385 | 0.182647993 | 12.18471832 | 0.000481826 | 0.060690018 |
| DNAJC9 | 194.988335 | −0.462822231 | 0.13578116 | 12.12914118 | 0.0004964 | 0.061483925 |
| ARG1 | 72.4908196 | −0.796757664 | 0.24170391 | 12.07453342 | 0.000511153 | 0.061483925 |
| TRA2A | 242.818114 | −0.370177056 | 0.10842455 | 12.05283964 | 0.000517135 | 0.061483925 |
| HIST1H2AG | 375.263091 | −0.293447479 | 0.085887285 | 12.04075155 | 0.0005205 | 0.061483925 |
| PPP2R5C | 408.606687 | 0.137459246 | 0.039387142 | 12.00514553 | 0.000530539 | 0.061483925 |
| UTP3 | 79.2980827 | 0.461692517 | 0.129523005 | 11.97005354 | 0.000540624 | 0.061483925 |
| BMS1 | 183.723177 | 0.241018859 | 0.068716246 | 11.95976754 | 0.000543617 | 0.061483925 |
| WHSC1 | 185.31172 | −0.226521785 | 0.066425648 | 11.92423415 | 0.000554084 | 0.061483925 |
| NUP133 | 110.269171 | 0.156526589 | 0.04522015 | 11.91679955 | 0.0005563 | 0.061483925 |
| SLC25A15 | 42.0037796 | −0.596960989 | 0.178414071 | 11.860334 | 0.000573423 | 0.061483925 |
| MYO1E | 88.9824676 | 0.404503129 | 0.114157332 | 11.84234693 | 0.000578988 | 0.061483925 |
| TLE1 | 22.5766189 | 0.54382872 | 0.153891879 | 11.84212637 | 0.000579057 | 0.061483925 |
| CENPF | 286.307473 | −0.601321328 | 0.18356237 | 11.81108262 | 0.000588792 | 0.061483925 |
| HNRNPM | 1750.4597 | 0.170158862 | 0.04909502 | 11.81061753 | 0.000588939 | 0.061483925 |
| CCNE2 | 19.1264461 | −0.354971369 | 0.104477344 | 11.77598515 | 0.000599998 | 0.061483925 |
| TNKS2 | 219.507656 | 0.158809062 | 0.046014002 | 11.7758489 | 0.000600041 | 0.061483925 |
| TYMS | 62.2905051 | −0.499118477 | 0.148971538 | 11.73008608 | 0.000614977 | 0.061483925 |
| ATP1B1 | 66.7258463 | −0.78171204 | 0.242172775 | 11.7283898 | 0.000615538 | 0.061483925 |
| HSPA4 | 603.817699 | 0.130939432 | 0.038066225 | 11.70951895 | 0.000621812 | 0.061483925 |
| KIF11 | 74.4096422 | −0.291879346 | 0.086082108 | 11.68479707 | 0.000630129 | 0.061483925 |
| GPR155 | 31.7649463 | −0.478814886 | 0.143773625 | 11.66861505 | 0.000635633 | 0.061483925 |
| KCTD18 | 81.6905015 | −0.494420831 | 0.149178602 | 11.66380216 | 0.00063728 | 0.061483925 |
| CHMP1A | 78.9514046 | −0.28448745 | 0.084366365 | 11.6295058 | 0.000649138 | 0.061968763 |
| CYB5R4 | 245.544953 | −0.240885249 | 0.071641203 | 11.58170704 | 0.000666038 | 0.062919751 |
| SURF4 | 39.7092905 | −0.423964499 | 0.127821348 | 11.55995935 | 0.000673873 | 0.063003677 |
| UBFD1 | 23.440026 | 0.51702477 | 0.1473821 | 11.49849634 | 0.000696525 | 0.064457005 |
| MS4A3 | 45.4722541 | −0.846596609 | 0.259710365 | 11.42078505 | 0.00072627 | 0.066474938 |
| ZNF100 | 72.7823971 | −0.313967903 | 0.093889894 | 11.40367192 | 0.000732991 | 0.066474938 |
| FBRSL1 | 157.84346 | −0.423476217 | 0.129442424 | 11.34208635 | 0.000757702 | 0.067456821 |
| HIST1H3B | 160.992723 | −0.563354995 | 0.172589487 | 11.33283675 | 0.000761485 | 0.067456821 |
| JMJD1C | 1173.54762 | −0.321356114 | 0.096927602 | 11.32153835 | 0.000766132 | 0.067456821 |
| HDGF | 1516.62537 | −0.320347942 | 0.097986788 | 11.29956087 | 0.000775254 | 0.067603661 |
| GFOD1 | 46.2615555 | −0.390620305 | 0.120574865 | 11.26119987 | 0.00079144 | 0.067733245 |
| ZNF347 | 56.7785617 | −0.483136357 | 0.147301017 | 11.24435006 | 0.000798658 | 0.067733245 |
| NT5C2 | 315.658417 | −0.288282573 | 0.087621237 | 11.24321471 | 0.000799146 | 0.067733245 |
| SERPINB10 | 30.1641459 | −0.91614822 | 0.286942518 | 11.16704123 | 0.000832633 | 0.069647542 |
| ADCY3 | 131.715381 | −0.755386896 | 0.235882849 | 11.15713403 | 0.000837091 | 0.069647542 |
| HDAC6 | 85.9990103 | −0.257845644 | 0.078305194 | 11.12402269 | 0.000852168 | 0.07025735 |
| FNBP1L | 688.822315 | −0.583258432 | 0.179846878 | 11.02494984 | 0.000898937 | 0.073445592 |
| CDCA2 | 27.9846514 | −0.351604469 | 0.106383011 | 10.96863027 | 0.000926672 | 0.074331571 |
| PKP2 | 59.0515065 | −0.5919732 | 0.185121482 | 10.93505182 | 0.000943618 | 0.074331571 |
| MAFG | 62.4155814 | −0.475736151 | 0.148504114 | 10.92588387 | 0.0009483 | 0.074331571 |
| HIST1H2AL | 100.449723 | −0.549602282 | 0.171209237 | 10.91134298 | 0.000955772 | 0.074331571 |
| CD109 | 226.319539 | −0.722114926 | 0.221290922 | 10.9069803 | 0.000958026 | 0.074331571 |
| MMP8 | 61.7414815 | −0.963025712 | 0.306340595 | 10.89073584 | 0.000966464 | 0.074331571 |
| ANLN | 115.731414 | −0.295842283 | 0.090850141 | 10.88941321 | 0.000967155 | 0.074331571 |
| MTMR10 | 733.404726 | −0.480452862 | 0.149333198 | 10.85233363 | 0.000986713 | 0.075197506 |
| PMPCB | 132.728427 | 0.238068066 | 0.071311803 | 10.80424715 | 0.001012675 | 0.076052074 |
| ZDHHC3 | 66.0394411 | −0.260252119 | 0.080306011 | 10.80055166 | 0.001014699 | 0.076052074 |
| STRN4 | 542.589927 | −0.403498387 | 0.125812989 | 10.75598871 | 0.001039424 | 0.077266708 |
| SLC30A1 | 41.582641 | −0.48709392 | 0.153134635 | 10.73638939 | 0.001050491 | 0.077454495 |
| THUMPD1 | 309.207619 | −0.406262264 | 0.127203679 | 10.67845738 | 0.001083904 | 0.079219698 |
| UNC13D | 448.751353 | −0.435984447 | 0.136240502 | 10.66273958 | 0.001093154 | 0.079219698 |
| COL6A3 | 229.356044 | −0.871540967 | 0.279680555 | 10.64316563 | 0.001104784 | 0.079219698 |
| DACH1 | 49.7307281 | −0.357313535 | 0.109906151 | 10.60586614 | 0.001127294 | 0.079219698 |
| PDZD8 | 154.486387 | −0.257891719 | 0.079851585 | 10.59729745 | 0.001132531 | 0.079219698 |
| MCM7 | 83.7976273 | −0.306443012 | 0.09451062 | 10.59553298 | 0.001133612 | 0.079219698 |
| H2AFX | 26.7167358 | −0.621633373 | 0.195620526 | 10.59232889 | 0.001135578 | 0.079219698 |
| PDLIM7 | 380.727424 | −0.505011238 | 0.160089466 | 10.53019631 | 0.001174397 | 0.080999672 |
| XRCC2 | 19.1233452 | −0.678008232 | 0.21669442 | 10.52303581 | 0.001178957 | 0.080999672 |
| HIST1H2AD | 97.3430238 | −0.34596932 | 0.108676691 | 10.44132953 | 0.001232265 | 0.083449616 |
| SNX2 | 647.453038 | 0.202977723 | 0.061821064 | 10.4402004 | 0.001233019 | 0.083449616 |
| CDK1 | 18.0714248 | −0.51816235 | 0.162355531 | 10.33963387 | 0.001302038 | 0.087226169 |
| CCDC71L | 37.33982 | −0.400919901 | 0.127802181 | 10.32455688 | 0.001312718 | 0.087226169 |
| CKLF | 37.8805589 | −0.462449877 | 0.14699266 | 10.29862805 | 0.001331292 | 0.087226169 |
| NBEAL2 | 340.162037 | −0.432033009 | 0.136441565 | 10.29489473 | 0.001333988 | 0.087226169 |
| BLK | 43.4801839 | 0.634035324 | 0.188877899 | 10.29085666 | 0.00133691 | 0.087226169 |
| TBC1D17 | 58.4749713 | −0.373545049 | 0.118601337 | 10.24113633 | 0.00137343 | 0.087484066 |
| LEF1 | 151.118851 | 0.643948384 | 0.191173884 | 10.23488179 | 0.001378094 | 0.087484066 |
| ZMIZ2 | 192.67977 | −0.414950646 | 0.133664118 | 10.22724077 | 0.001383815 | 0.087484066 |
| PROSC | 153.538309 | 0.198924963 | 0.061677357 | 10.22540842 | 0.001385191 | 0.087484066 |
| HBG2 | 345.124523 | −0.918493788 | 0.296215427 | 10.21880457 | 0.001390159 | 0.087484066 |
| G6PD | 636.863085 | −0.407286058 | 0.13130294 | 10.20745346 | 0.001398742 | 0.087484066 |
| SCAMP2 | 67.7773099 | −0.394249471 | 0.126956056 | 10.16850961 | 0.001428597 | 0.088739365 |
| ADSL | 225.751847 | 0.196671315 | 0.061110072 | 10.14454322 | 0.00144729 | 0.089288946 |
| TTC14 | 35.3500103 | −0.41643018 | 0.131587484 | 10.10593962 | 0.001477922 | 0.090562679 |
| SNX19 | 56.1029379 | −0.586594521 | 0.192975491 | 10.07305605 | 0.001504533 | 0.091574547 |
| SSH1 | 283.720048 | −0.430272183 | 0.139594448 | 10.01954535 | 0.001548877 | 0.092537718 |
| PUDP | 20.5130162 | 0.344091852 | 0.108081232 | 10.01828007 | 0.001549941 | 0.092537718 |
| MECP2 | 485.159305 | −0.330039312 | 0.106259251 | 10.01705997 | 0.001550968 | 0.092537718 |
| CD63 | 369.814694 | −0.370604322 | 0.119643987 | 9.97005192 | 0.00159107 | 0.093697832 |
| KCNMB1 | 50.8034229 | −0.621752932 | 0.205706399 | 9.966132454 | 0.001594461 | 0.093697832 |
| MAPKAPK5 | 123.545681 | 0.16432536 | 0.051688944 | 9.958128716 | 0.001601407 | 0.093697832 |
| GSN | 1142.9619 | −0.513473609 | 0.167530371 | 9.917485992 | 0.001637159 | 0.095175581 |
| LOXHD1 | 199.692968 | −0.731866353 | 0.24195628 | 9.90140628 | 0.001651525 | 0.095364629 |
| RSRC2 | 830.686621 | −0.262498114 | 0.084618777 | 9.890390225 | 0.001661441 | 0.095364629 |
| NLRX1 | 30.7233614 | −0.509357783 | 0.166698746 | 9.843889299 | 0.001703968 | 0.095988604 |
| SEPT1 | 110.886498 | 0.323262856 | 0.101511457 | 9.840581353 | 0.001707035 | 0.095988604 |
| CD69 | 38.0149845 | −0.674155226 | 0.219370446 | 9.834226717 | 0.001712943 | 0.095988604 |
| ZWINT | 24.8850687 | −0.39823044 | 0.128888897 | 9.819550962 | 0.001726665 | 0.095988604 |
| MPZL3 | 113.172834 | −0.654041276 | 0.209805319 | 9.802115693 | 0.001743112 | 0.095988604 |
| C19orf60 | 16.0678764 | 0.360656348 | 0.114692869 | 9.795694668 | 0.001749209 | 0.095988604 |
| DHRS7 | 141.576438 | −0.39952924 | 0.130352818 | 9.792485914 | 0.001752264 | 0.095988604 |
| HIST1H3D | 53.2585736 | −0.400948931 | 0.129905156 | 9.781128458 | 0.001763121 | 0.095988604 |
| URGCP | 27.7194428 | 0.340624969 | 0.106525549 | 9.762391628 | 0.00178118 | 0.095988604 |
| SLFN5 | 215.94271 | 0.480638388 | 0.148370925 | 9.739063308 | 0.001803928 | 0.095988604 |
| DENND5B | 61.3148853 | 0.314946804 | 0.099031435 | 9.735650377 | 0.001807281 | 0.095988604 |
| HDAC8 | 41.9432708 | −0.268324265 | 0.087630995 | 9.735604359 | 0.001807326 | 0.095988604 |
| MPO | 58.7414306 | −0.702404473 | 0.234008372 | 9.732980597 | 0.001809908 | 0.095988604 |
| LBR | 97.386483 | −0.388828754 | 0.12690985 | 9.718285563 | 0.001824436 | 0.096196585 |
| SLC25A17 | 26.6395003 | −0.435027079 | 0.141781328 | 9.693486997 | 0.001849223 | 0.096939895 |
| PHF10 | 89.6542661 | 0.211046689 | 0.067249255 | 9.670560543 | 0.001872442 | 0.097592955 |
| C5orf51 | 85.5546517 | −0.439052137 | 0.144932302 | 9.651442593 | 0.001892029 | 0.09763215 |
| LIMA1 | 90.6336708 | −0.243337275 | 0.079242036 | 9.61963325 | 0.001925082 | 0.09763215 |
| KIF4A | 42.6606646 | −0.303097287 | 0.099303103 | 9.597227403 | 0.001948714 | 0.09763215 |
| HOMER2 | 762.904045 | −0.64907536 | 0.218124585 | 9.596591311 | 0.001949389 | 0.09763215 |
| MYB | 80.830462 | −0.386211669 | 0.126466593 | 9.595490392 | 0.001950558 | 0.09763215 |
| NMT2 | 49.2941549 | 0.453745355 | 0.141576441 | 9.579588804 | 0.001967525 | 0.09763215 |
| ERICH1 | 445.217991 | −0.412096292 | 0.134791355 | 9.570673095 | 0.001977103 | 0.09763215 |
| LOX | 38.7753467 | −0.837609776 | 0.282800795 | 9.568551905 | 0.001979389 | 0.09763215 |
| EMC7 | 38.9232153 | −0.297068531 | 0.097179965 | 9.56836946 | 0.001979585 | 0.09763215 |
| RNF167 | 143.994981 | −0.28593229 | 0.094447548 | 9.567198302 | 0.001980849 | 0.09763215 |
| SVIL | 640.967988 | −0.425770686 | 0.139799407 | 9.551376014 | 0.001997996 | 0.097944996 |
| SGMS1 | 55.9206306 | −0.461626108 | 0.15425216 | 9.533346984 | 0.002017718 | 0.098380034 |
| IMPAD1 | 53.4291124 | −0.579371195 | 0.19336976 | 9.502711545 | 0.002051685 | 0.099376942 |
| MAPK6 | 287.705426 | −0.48667072 | 0.162417619 | 9.495218971 | 0.00206008 | 0.099376942 |

TABLE 6
Predictive Model Weights of Genes
Predictive for Pre-Term Birth (PTB)

| Gene | Weight |
|---|---|
| ELANE | 0.0989222 |
| ACSM3 | 0.07557269 |
| MAPK10 | 0.06882871 |
| IRX3 | 0.06702434 |
| SPAG5 | 0.06010713 |
| B3GNT2 | 0.05968447 |
| LOX | 0.05033319 |
| H2AFX | 0.04841582 |
| ITGAE | 0.03649107 |
| ARL4A | −0.0354448 |
| ZBTB26 | 0.03028558 |
| BEX1 | 0.02647277 |
| HBG2 | 0.02617242 |
| SNX19 | 0.0248166 |
| CCNA2 | 0.02240897 |
| TLE1 | −0.0213883 |
| TMEM204 | 0.01798467 |
| MRTO4 | −0.0124935 |
| PHGDH | 0.01168144 |
| IMPAD1 | 0.00555929 |
| KCNMB1 | 0.00518973 |
| ENPP4 | 0.00388786 |
| MMP8 | −0.0029393 |
| MPZL3 | 0.00211636 |
| NLRX1 | 0.00085898 |

FIG. 7G shows a receiver-operating characteristic (ROC) curve showing the performance of the predictive model for pre-term delivery across the 10-fold cross-validation. As shown in the figure, the predictive model for predicting pre-term delivery achieved a mean area under the curve (AUC) of 0.90±0.08, thereby demonstrating the excellent performance of the predictive model for predicting pre-term delivery.

### Example 5: Prediction of Due Date (DD)

Using systems and methods of the present disclosure, a prediction model is developed to predict a due date of a fetus of a pregnant subject. For example, the predicted due date can be a number of days (e.g., 1 day, 2 days, 3 days, 4 days, 5 days, 6 days, or 7 days) or weeks (e.g., 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, or 45 weeks) until an expected delivery of the fetus of the pregnant subject. As another example, the predicted due date can be a future date on which the delivery of the fetus of the pregnant subject is expected to occur.

The prediction model may be based on assaying a sample (e.g., a blood draw) of a pregnant subject at a given time point (e.g., at an estimated gestational age of 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, or 45 weeks).

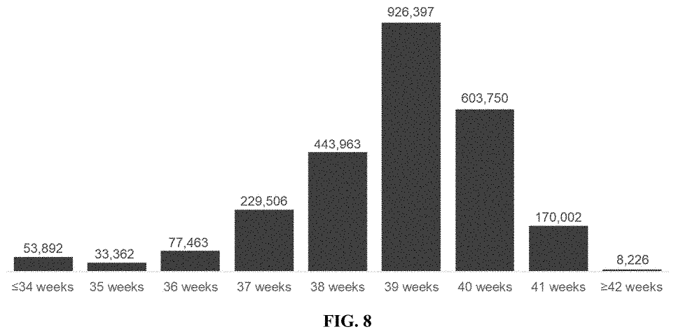

FIG. 8 shows an example of a distribution of vaginal singleton births by obstetrician-estimated gestational age in the U.S. This figure shows that only 23.7% of vaginal singleton births occur at an estimated gestational age of 40 weeks, and about 67% of vaginal singleton births occur at an estimated gestational age of 39-41 weeks. Therefore, such variation of time of delivery illustrates the need for a better predictor of delivery date that uses a molecular clock, using systems and methods of the present disclosure.

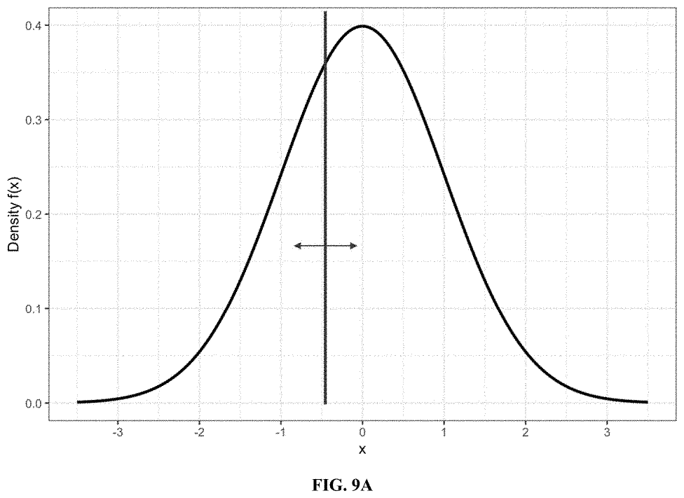

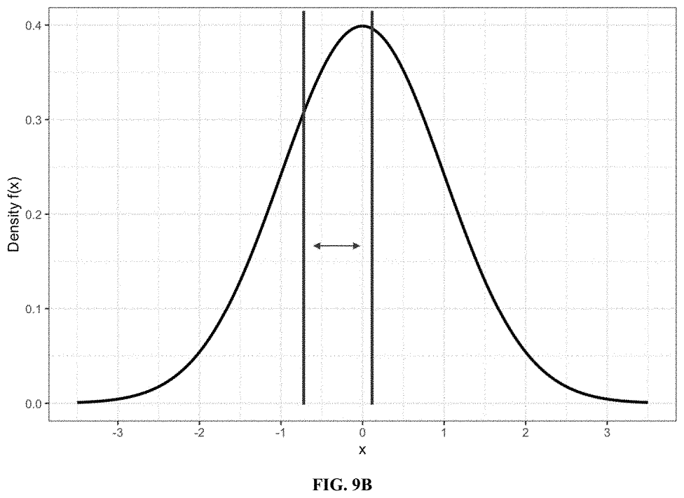

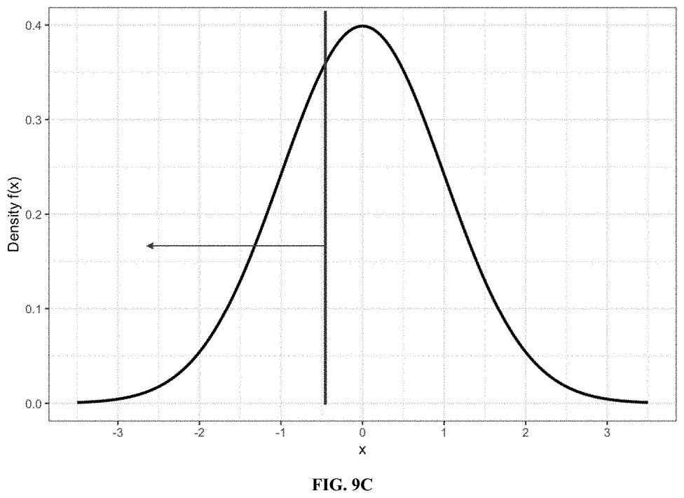

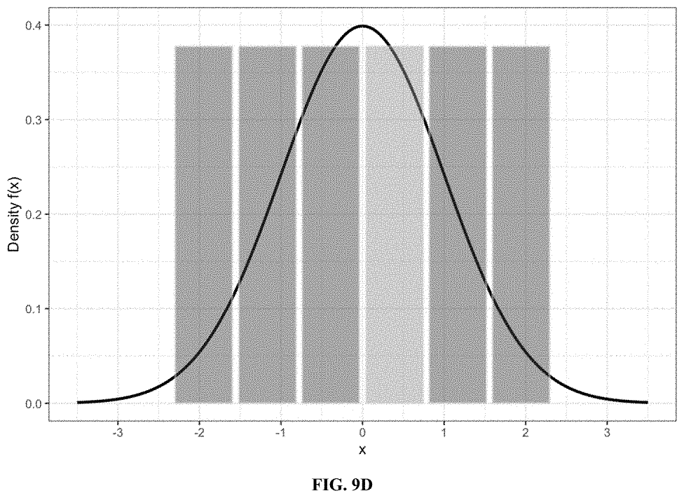

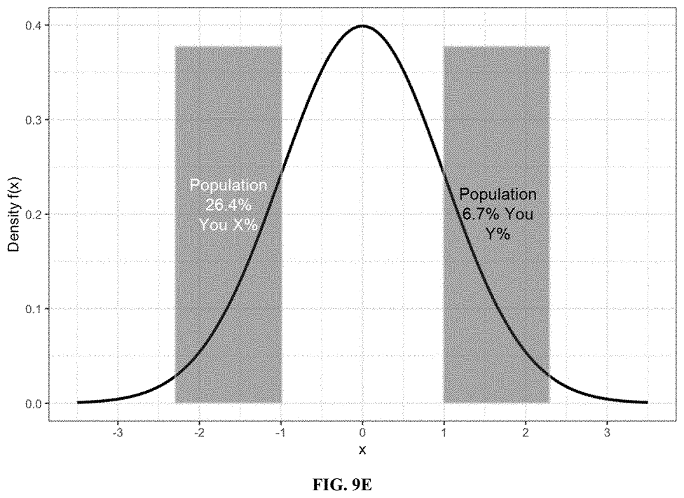

FIG. 9A-9E show different methods of predicting due date for a fetus of a pregnant subject, including predicting an actual day (with error) (FIG. 9A), predicting a week (or other window) of delivery (FIG. 9B), predicting whether a delivery is expected to occur before or after a certain time boundary (FIG. 9C), predicting in which bin among a plurality of bins (e.g., 6 bins) a delivery is expected to occur (FIG. 9D), and predicting a relative risk or relative likelihood of an early delivery or a late delivery (FIG. 9E).

For example, the due date prediction model may be used to predict an actual day (with error) (FIG. 9A). For example, the predicted due date may be a number of days (e.g., 1 day, 2 days, 3 days, 4 days, 5 days, 6 days, or 7 days) or weeks (e.g., 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, or 45 weeks) until an expected delivery of the fetus of the pregnant subject. As another example, the predicted due date may be a future date on which the delivery of the fetus of the pregnant subject is expected to occur. As another example, the predicted due date may be an estimated gestational age (e.g., 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, or 45 weeks) for which the delivery of the fetus of the pregnant subject is expected to occur. The predicted due date may be provided along with an error or confidence interval (e.g., 1 day, 2 days, 3 days, 4 days, 5 days, 6 days, 7 days, 2 weeks, 3 weeks, or 4 weeks) for the predicted due date. The predicted due date may be provided along with an estimated likelihood or confidence (e.g., about 50%, 55%, 60%, 65%, 70%, 75%, 80%, 85%, 90%, 91%, 92%, 93%, 94%, 95%, 96%, 97%, 98%, or 99%) for the predicted due date.

As another example, the due date prediction model may be used to predict a week (or other window) of delivery (FIG. 9B). For example, the predicted due date may be a number of weeks (e.g., 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, or 45 weeks) until an expected delivery of the fetus of the pregnant subject. As another example, the predicted due date may be a future week (e.g., a week on the calendar) on which the delivery of the fetus of the pregnant subject is expected to occur. As another example, the predicted due date may be an estimated gestational age (e.g., 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, or 45 weeks) for which the delivery of the fetus of the pregnant subject is expected to occur. The predicted due date may be provided along with an estimated likelihood or confidence (e.g., about 50%, 55%, 60%, 65%, 70%, 75%, 80%, 85%, 90%, 91%, 92%, 93%, 94%, 95%, 96%, 97%, 98%, or 99%) for the predicted due date.

As another example, the due date prediction model may be used to predict whether a delivery is expected to occur before or after a certain time boundary (FIG. 9C). For example, the time boundary may be a number of weeks (e.g., 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, or 45 weeks) of estimated gestational age. For example, the time boundary may be an estimated gestational age of 40 weeks.

As another example, the due date prediction model may be used to predict which bin among a plurality of bins (e.g., 6 bins) a delivery is expected to occur (FIG. 9D). For example, the bins (e.g., time windows) may be equal ranges of time (e.g., 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks; or 1 month, 2 months, 3 months, 4 months, or 5 months; or a trimester among the first, second, or third trimesters). The predicted due date may be provided along with an estimated likelihood or confidence (e.g., about 50%, 55%, 60%, 65%, 70%, 75%, 80%, 85%, 90%, 91%, 92%, 93%, 94%, 95%, 96%, 97%, 98%, or 99%) for the predicted due date bin or time window.

As another example, the due date prediction model may be used to predict a relative risk or relative likelihood of an early delivery or a late delivery (FIG. 9E). For example, the prediction may comprise a relative risk or relative likelihood of an early delivery or a late delivery of about 10%, 20%, 30%, 40%, 50%, 55%, 60%, 65%, 70%, 75%, 80%, 85%, 90%, 91%, 92%, 93%, 94%, 95%, 96%, 97%, 98%, or 99%. An early delivery may be defined as a due date at an estimated gestational age of less than 40 weeks, while a late delivery may be defined as a due date at an estimated gestational age of more than 40 weeks.

A due date prediction model was trained using samples collected from a gestational age (GA) cohort of pregnant subjects, all of whom had an estimated gestational age of a fetus of 34 weeks to 36 weeks. A training dataset was obtained using a cohort of 270 and 312 samples (about half of which was Caucasian and half of which was AA), of which 41 samples were designated as lab outliers and not used and 1 sample had an outlier low CPM. Further, a test dataset of 64 samples was obtained using a cohort (003_GA) of 19 samples (most of whom were Caucasian) and a cohort (009_VG) of 47 validation samples (all of whom had an estimated gestational age of a fetus of 34 weeks to 36 weeks, and most of whom were Caucasian).

Gene discovery was performed to develop the due date prediction model as follows. A set of 241 input genes, comprising candidate marker genes, was used. Using the training dataset, a subset of these candidate marker genes was identified as having a high median(log 2_CPM) value of greater than 0.5. An analysis of variance (ANOVA) was performed using a set of 248 genes (as shown in Table 7) for actual time to delivery for the training samples (e.g., −7 weeks vs. −2 weeks for the top 100 genes, and −6 weeks vs. −3 weeks for the top 100 genes). A Pearson linear correlation was performed to identify the top 100 genes among the candidate marker genes having the strongest statistical correlation to due date. A number of different prediction models were tested for prediction of time-to-delivery bins. First, the standard of care was used in which a predicted time to delivery was made based on a predicted due date at a gestational age of 40 weeks. Second, an estimated gestational age using ultrasound data only was used, using the collectionga cohort as an input to the elastic net prediction model. Third, an estimated gestational age using cfDNA only was used, using an input of log 2_CPMs of genes and confounders (e.g., parity, BMI, smoking status, etc.) as inputs to the elastic net prediction model. Fourth, an estimated gestational age using both cfDNA plus ultrasound was used, using an input of log 2 CPMs of genes, confounders, and collectionga input to the elastic net prediction model.

TABLE 7
Set of 248 Genes Used in ANOVA Model
Genes

ABCB1, AC010468.1, AC068657.2, AC078899.1, AC079250.1, AC114752.3,
ACOX1, ACTA2, ACTBP8, ACTG1P15, ADAM12, ADCK5, ADGRE1, ADGRG5,
ADGRL2, AKR1C1, AKR1E2, ALG1, ALS2, AMT, ANO5, ANP32AP1, ANP32C,
APBA3, ARFGEF3, ASMTL, ATAD3A, ATF4P3, ATP8B3, BBOF1, BBS4, BCAR3,
BCYRN1, C14orf119, C1orf228, C2orf42, C6orf106, C6orf47, C9orf3, CALM1P1,
CALM2, CAMK2D, CASC4P1, CD177, CD68, CDC27, CDC42P6, CDK5RAP2,
CFAP43, CFAP70, CHAC2, CHCHD4, CHKA, CKAP2, CLC, CLN5, CMTM3,
CNOT6LP1, CNTNAP2, COPA, CRH, CSRNP2, CSTF2, CTB-79E8.3, CXCR3,
CXXC4, CYP51A1, CYYR1, DAB2IP, DCUN1D1, DEPDC1B, DHCR24, DHTKD1,
DOCK9, DRAM1, DSC2, EEF1A1P16, EIF1AXP1, EIF3LP2, EIF4EBP3, ELMOD3,
ETFRF1, EVX2, EXO5, FAM120A, FBP1, FBXL14, FCGR3B, FGF2, FLII, FN1,
FTH1P3, FZD6, GABPA, GAS2, GATAD2B, GLIS2, GLRA4, GOLGA2, H2BFS,
HMGB1P11, HMGB3P22, HMGCS1, HNRNPKP1, HNRNPKP4, HP, HPCAL1,
HSPG2, ICAM4, ICMT, IKZF2, IL2RA, INHBA, INPP5K, INTS4, INTS6, ITGA3,
ITGB4, KCMF1, KCNK5, KIF3A, KLHDC8B, KLRC1, LRP5, MAGT1, MAPK1,
MAPK11, MAPK13, MCCC1, MCEMP1, MECP2,
Metazoa_SRP_ENSG00000278771, MGAT3, MIB1, MOB4, MORF4L1, MRRF, MT-TE,
MT-TP, MTDHP3, MUT, MYL12BP2, NAP1L1P1, NCOA1, NDUFV2P1, NEK6,
NEMP2, NRCAM, OASL, OGDH, PAK3, PAPPA, PAPPA2, PASK, PDZRN4, PERP,
PIGM, PMM1, PPIL1, PPM1H, PRICKLE4, PRKCZ, PSG9, PSMC3IP, PTMA,
RAB3GAP2, RAB43, RAP1BP1, RBBP4P1, RELL1, RFX2, RN7SL1, RN7SL396P,
RN7SL767P, RNA5SP355, RNY1, ROBO3, RP1-121G13.3, RP3-393E18.1,
RPL14P3, RPL15P2, RPL19P16, RPL5P5, RPTOR, RRN3P1, RSU1P1, SCAND1,
SEPT7P2, SERPINB9, SHISA5, SIRPG, SKOR1, SKP1P1, SLC43A1, SNRNP48,
SPCS2, SRGAP2C, SRP9P1, STAG3L2, STAT5B, STRAP, STX2, SVEP1, SYN2,
TAF6L, TANC1, TEK, TGDS, THOC3, THOC7, TIE1, TMA7, TMEM14A,
TMEM222, TMEM237, TMEM8A, TPI1P1, TRAV12-2, TRAV14DV4, TRIM36,
TTBK2, TTC28, UBE2R2, UQCRHL, VPS33B, WDR37, WDR77, WTH3DI,
Y_RNA_ENSG00000199303, Y_RNA_ENSG00000201412,
Y_RNA_ENSG00000202357, Y_RNA_ENSG00000202533,
Y_RNA_ENSG00000252891, YPEL2, ZBED5-AS1, ZBTB16, ZBTB20, ZEB2P1,
ZFY, ZNF148, ZNF319, ZNF563, ZNF696, ZNF714, ZSCAN16-AS1, ZSCAN22,
ZSCAN30

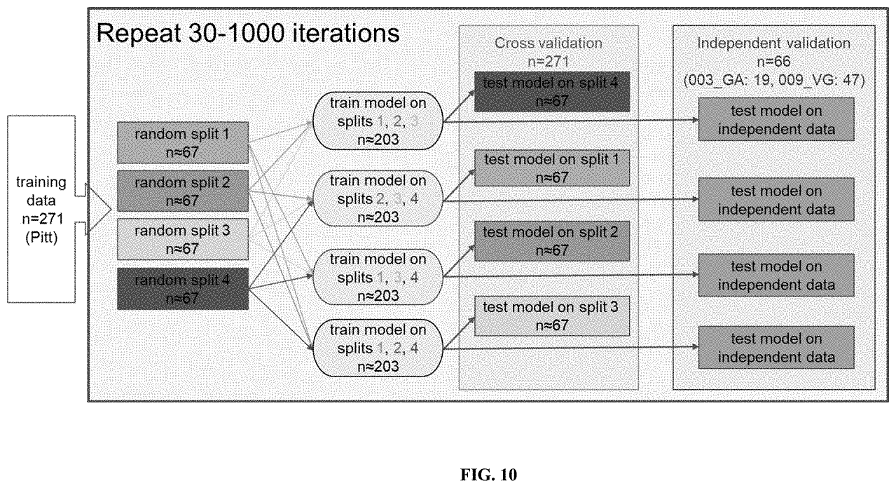

FIG. 10 shows a data workflow that is performed to develop a due date prediction model (e.g., classifier). First, the training data (n=271 samples) is randomly split up into 4 sets of 67 samples each. Next, the model is trained using different combinations of 3 of the 4 split sets that are creating by leaving out 1 split set at a time (e.g., a first combination of splits 1, 2, 3; a second combination of splits 2, 3, 4; a third combination of splits 1, 3, 4; and a fourth combination of splits 1, 2, 4; each having n=203 samples). Next, cross-validation is performed using the n=271 samples, where each of the 4 models are tested on the held-out split set (n=67 samples). Next, independent validation of each of the models is performed, whereby the models are tested on independent data (e.g., the testing dataset).

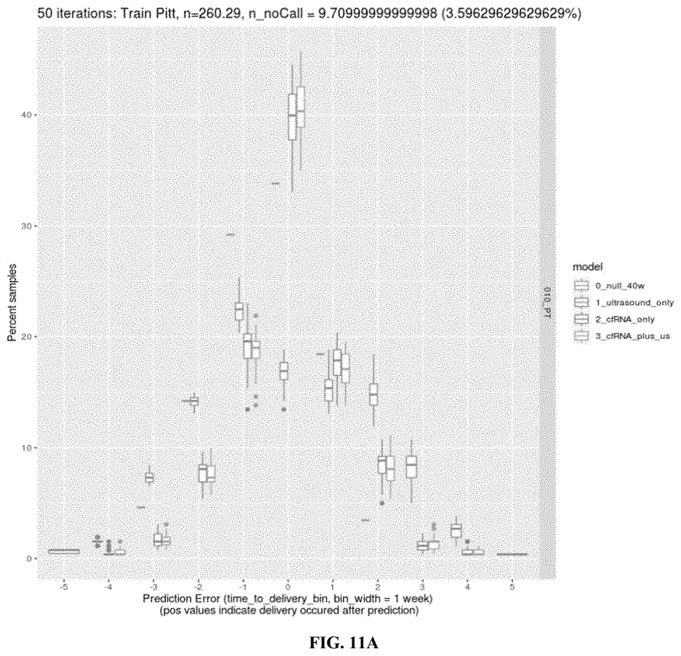

FIGS. 11A-11B show prediction error of a due date prediction model that is trained on 270 and 310 patients, respectively. The plot shows the percent of samples having a given prediction error (e.g., time to delivery bin, with a bin width of 1 week, where positive values indicate that delivery occurred after the predicted due date and negative values indicate that delivery occurred before the predicted due date). The figures show improved accuracy and lower error in due date prediction using the cfRNA-only model or the cfRNA-plus-ultrasound model, as compared to the standard-of-care (40 weeks) model and the ultrasound-only model.

### Example 6: Prediction of Pre-Term Birth (PTB)

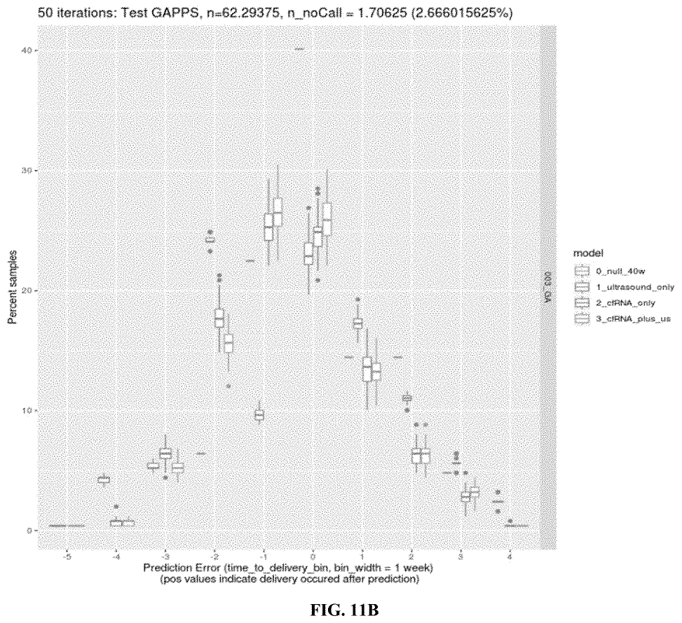

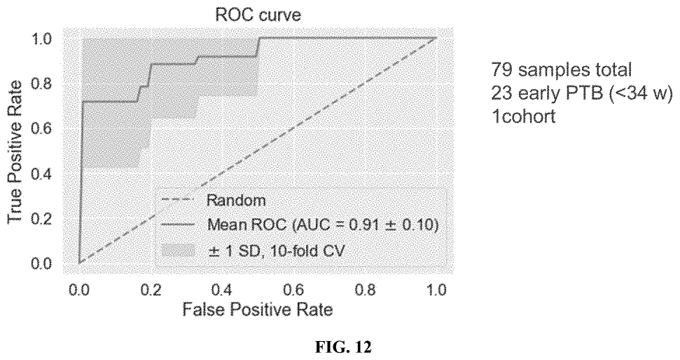

Using systems and methods of the present disclosure, a prediction model was developed to predict a risk of pre-term birth (PTB) of a pregnant subject. The dataset obtained from a cohort of Caucasian subjects (as described in Example 4) was re-analyzed with a modified gene list, as shown in Table 8. FIG. 12 shows a receiver-operator characteristic ROC) curve for the pre-term birth prediction model, using a set of 22 genes for a set of 79 samples obtained from a cohort of Caucasian subjects. Of the 79 total samples, 23 had early PTB (defined as delivery before 34 weeks of estimated gestational age). The mean area-under-the-curve (AUC) for the ROC curve was 0.91±0.10.

TABLE 8
Genes Predictive for Pre-Term Birth (PTB) (Caucasian)
Gene

SLC2A5
ESPN
LOX
IRX3
SPDYC
BEX1
ANK3
MTRNR2L12
MAPK10
B3GNT2
COL6A3
DDX11L10
NBPF3
U2AF1
MT1X
PHGDH
HBG2
RPL23AP7
CTD-3092A11.1
HLA-G
COL4A2
GSTM5

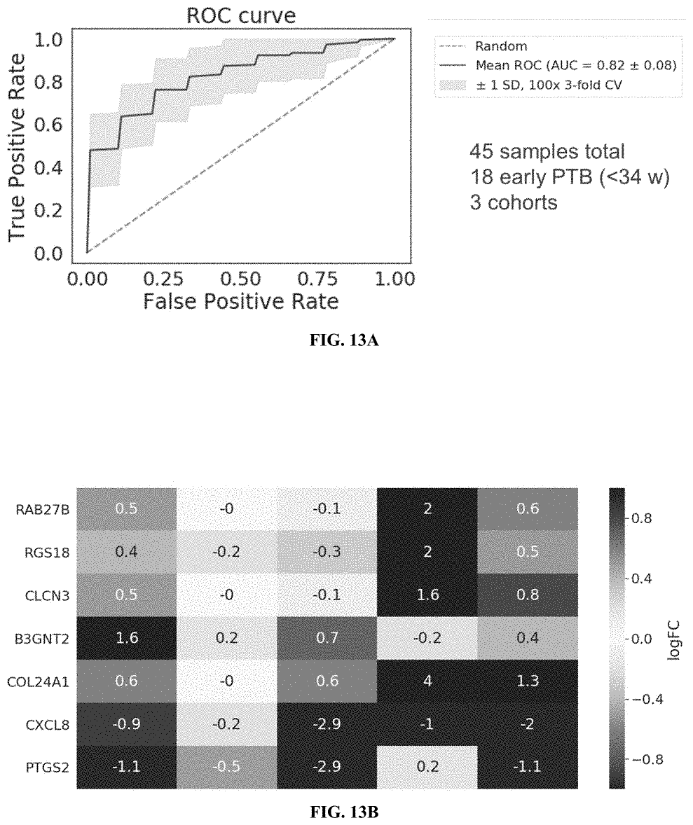

Further, FIG. 13A shows a receiver-operator characteristic ROC) curve for a pre-term birth prediction model, using a set of genes for a set of 45 samples obtained from a cohort of subjects having African or African-American ancestries (AA cohort). Of the 45 total samples, 18 had early PTB (defined as delivery before 34 weeks of estimated gestational age). The mean area-under-the-curve (AUC) for the ROC curve was 0.82±0.08.

FIG. 13B shows a gene panel for a pre-term birth prediction model for three different AA cohorts (cohort 1, cohort 2, and cohort 3), including RAB27B, RGS18, CLCN3, B3GNT2, COL24A1, CXCL8, and PTGS2.

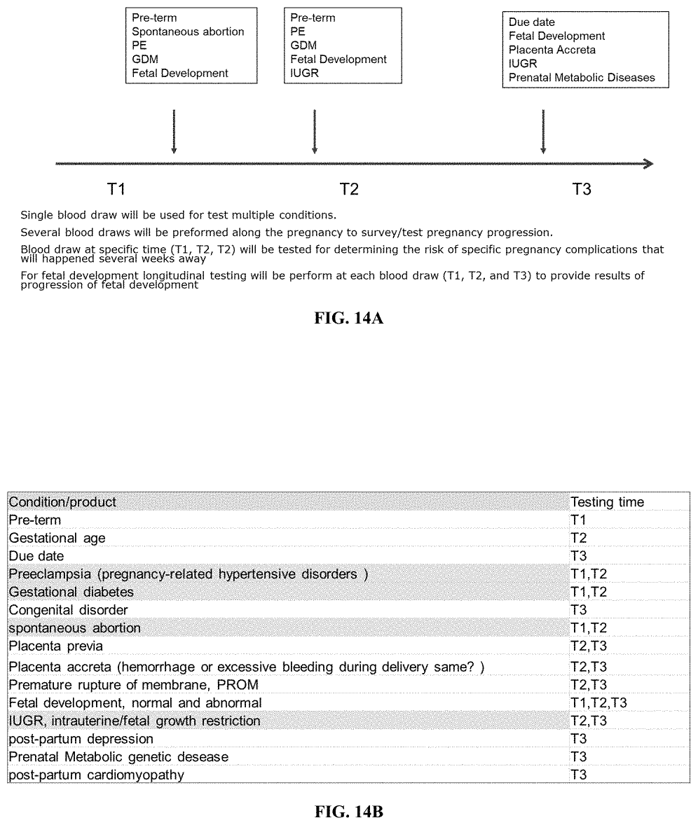

FIG. 14A shows a workflow for performing multiple assays for assessment of a plurality of pregnancy-related conditions using a single bodily sample (e.g., a single blood draw) obtained from a pregnant subject. Several blood draws can be performed along the pregnancy to survey and test the pregnancy progression. Blood draws obtained at specific time points (e.g., T1, T2, and T3) are tested for determining the risk of specific pregnancy-related complications that may happen several weeks away. For fetal development, longitudinal testing is performed at each blood draw (T1, T2, and T3) to provide results of the progression of fetal development. For example, a first blood sample may be obtained from a pregnant subject at time T1 (e.g., during the first trimester of pregnancy), a second blood sample may be obtained from the pregnant subject at time T2 (e.g., during the second trimester of pregnancy), and a third blood sample may be obtained from the pregnant subject at time T3 (e.g., during the third trimester of pregnancy). The blood sample obtained at time T1 may be used for assaying for pregnancy-related conditions that may be detectable or predictable in early-stage pregnancy or the first trimester of pregnancy, such as pre-term birth, spontaneous abortion, PE, GDM, and fetal development. The blood sample obtained at time T2 may be used for assaying for pregnancy-related conditions that may be detectable or predictable in mid-stage pregnancy or the second trimester of pregnancy, such as pre-term birth, PE, GDM, fetal development, and IUGR. The blood sample obtained at time T3 may be used for assaying for pregnancy-related conditions that may be detectable or predictable in late-stage pregnancy or the third trimester of pregnancy, such as due date, fetal development, placenta accreta, IUGR, prenatal metabolic diseases, and neonatal metabolic genetic diseases from RNA.

FIG. 14B shows a combination of conditions which can be tested from a single blood draw along a pregnancy progression of a pregnant subject. The blood sample obtained at time T1 may be used for assaying for pregnancy-related conditions that may be detectable or predictable in early-stage pregnancy or the first trimester of pregnancy, such as pre-term birth, preeclampsia (pregnancy-related hypertensive disorders), gestational diabetes, spontaneous abortion, and fetal development (normal and abnormal). The blood sample obtained at time T2 may be used for assaying for pregnancy-related conditions that may be detectable or predictable in mid-stage pregnancy or the second trimester of pregnancy, such as gestational age, preeclampsia (pregnancy-related hypertensive disorders), gestational diabetes, spontaneous abortion, placenta previa, placenta accreta (hemorrhage or excessive bleeding delivery), premature rupture of membrane (PROM), fetal development (normal and abnormal), and intrauterine/fetal growth restriction (IUGR). The blood sample obtained at time T3 may be used for assaying for pregnancy-related conditions that may be detectable or predictable in late-stage pregnancy or the third trimester of pregnancy, such as due date, congenital disorders, placenta previa, placenta accreta (hemorrhage or excessive bleeding delivery), premature rupture of membrane (PROM), fetal development (normal and abnormal), and intrauterine/fetal growth restriction (IUGR), post-partum depression, prenatal metabolic genetic disease, post-partum cardiomyopathy, and neonatal metabolic genetic diseases from RNA.

### Example 7: Prediction of Imminent Birth

Using systems and methods of the present disclosure, a prediction model was developed to detect or predict a risk of imminent birth of a pregnant subject. For example, a birth that occurs or is predicted to occur within the next 1 to 3 weeks may be considered as an imminent birth. The prediction model development comprised obtaining a cohort of subjects and training the prediction model on a training dataset corresponding to the cohort of subjects.

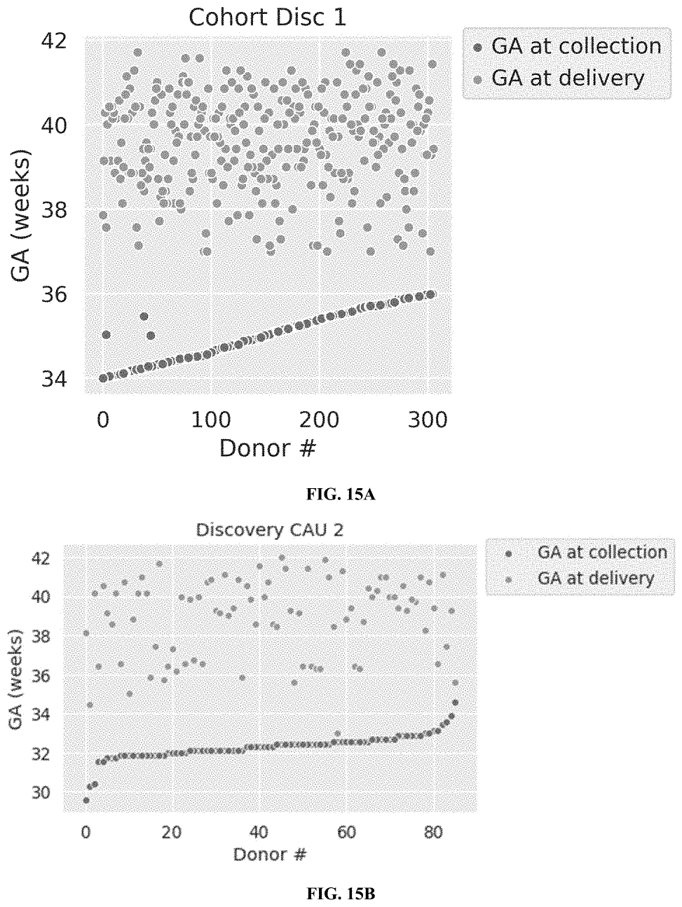

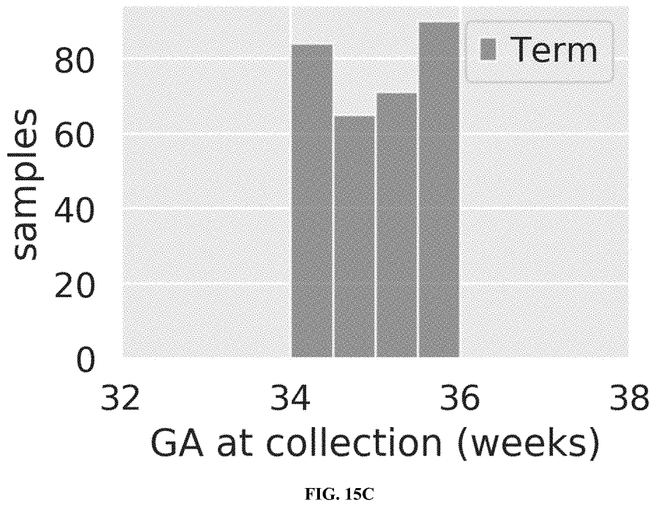

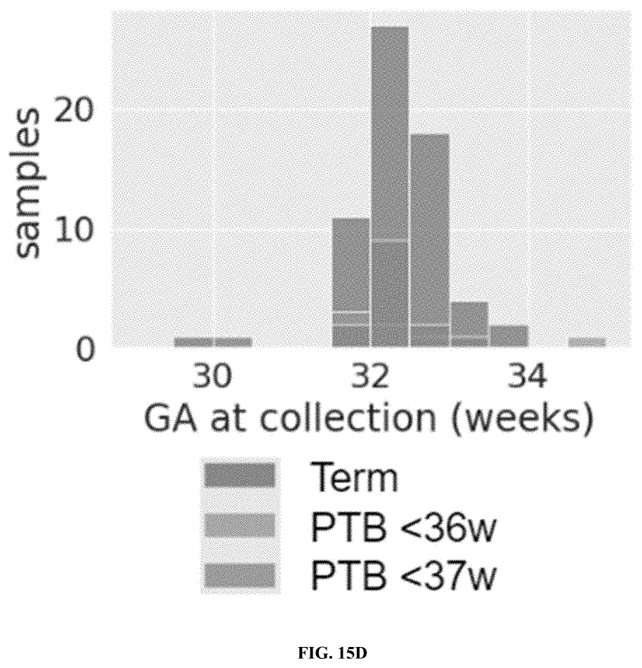

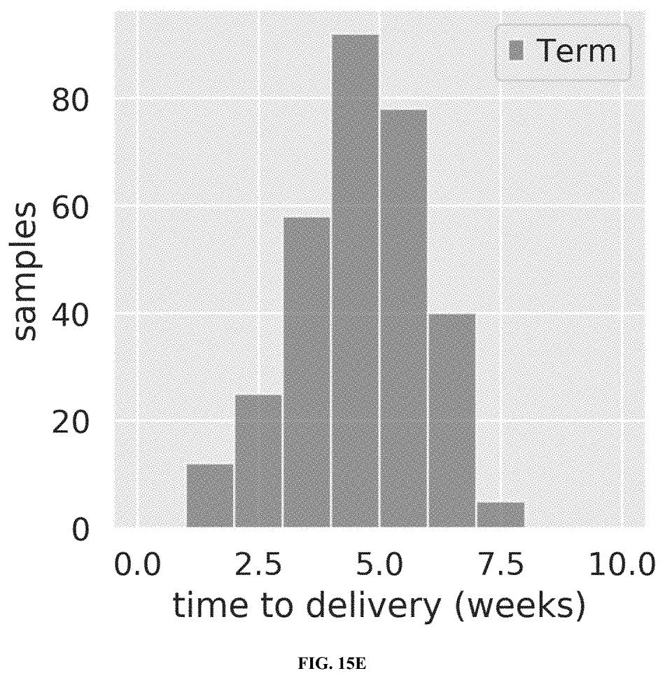

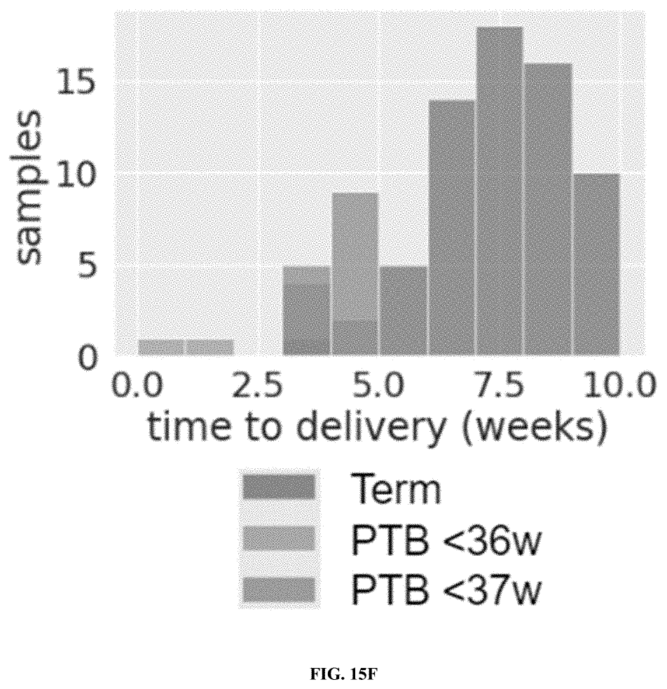

The cohort of subjects was obtained as follows. As shown in FIGS. 15A-15B, a Discovery 1 cohort of 310 mixed race subjects (e.g., pregnant women) and a Discovery 2 cohort of 86 Caucasian subjects, respectively, were established (with patient identification numbers shown on the x-axis). From these cohorts, one or more biological samples (e.g., 1 or 2) were collected and assayed at different time points corresponding to an estimated gestational age (shown on the y-axis, in increasing order of estimated gestational age at delivery) of a fetus of each subject, using methods and systems of the present disclosure. For example, the estimated gestational age (shown on the y-axis) may be determined using methods such as ultrasound imaging, a last menstrual period (LMP) date, or a combination thereof, and may range from 0 to about 42 weeks. The discovery cohorts includes subjects from who delivered at term and pre-term with blood collected between 1-10 weeks before delivery/birth.

FIG. 15C-15D show a distribution of participants in the Discovery 1 mixed race cohort and the Discovery 2 Caucasian cohort, respectively, based on blood sample collection gestation. FIGS. 15E-15F show a distribution of samples collection in the Discovery 1 mixed race cohort and the Discovery 2 Caucasian cohort, respectively, by weeks before birth.

Table 9 shows validation cohorts for imminent birth comprising subjects from whom different sample types were collected for use in different studies, including studies for the prediction of pre-term birth (e.g., as controls), prediction of delivery, prediction of due date, and prediction of actual gestational age of a fetus of each subject.

TABLE 9
Discovery and validation cohorts

| | | | | Vali- | Vali- | |
|---|---|---|---|---|---|---|
| | Discovery | Discovery | Discovery | dation | dation | Discovery |
| | 1 Mixed | 1 CAU | 1 AA | 1 AA | 2 Mixed | 2 CAU |
| N | 310 | 128 | 177 | 108 | 56 | 86 |

Differential expression analysis of the cohort data sets was performed as follows. All samples from the discovery cohort were binned in 1 to 10 weeks gestation at blood collection from birth as presented in FIG. 15E. A differential analysis for genes that are correlated to the time to delivery was performed, revealing that 9 genes show a significant correlation up to 10 weeks close to birth. A set of 9 genes (HTRA1, PAPPA2, ADCY6, PTPRB, TANGO2, IGFBP7, EFHD1, NFYB, ITGA5) that are predictive of birth 1 to 10 weeks before birth are listed in Table 10. The HTRA1 gene is particularly important. HTRA1 is a serine protease that cleaves fetal fibronectin, which may be present in vaginal secretion right before or at birth.

TABLE 10
Genes Predictive for Birth Within 1 to 3 Weeks

| Gene | Correlation | P-value |
|---|---|---|
| HTRA1 | −0.469584 | 0.000005 |
| PAPPA2 | −0.454334 | 0.000011 |
| ADCY6 | 0.453381 | 0.000012 |
| PTPRB | −0.450201 | 0.000014 |
| TANGO2 | 0.447341 | 0.000016 |
| IGFBP7 | −0.435855 | 0.000027 |
| EFHD1 | −0.425501 | 0.000044 |
| NFYB | −0.415233 | 0.00007 |
| ITGA5 | −0.415205 | 0.00007 |

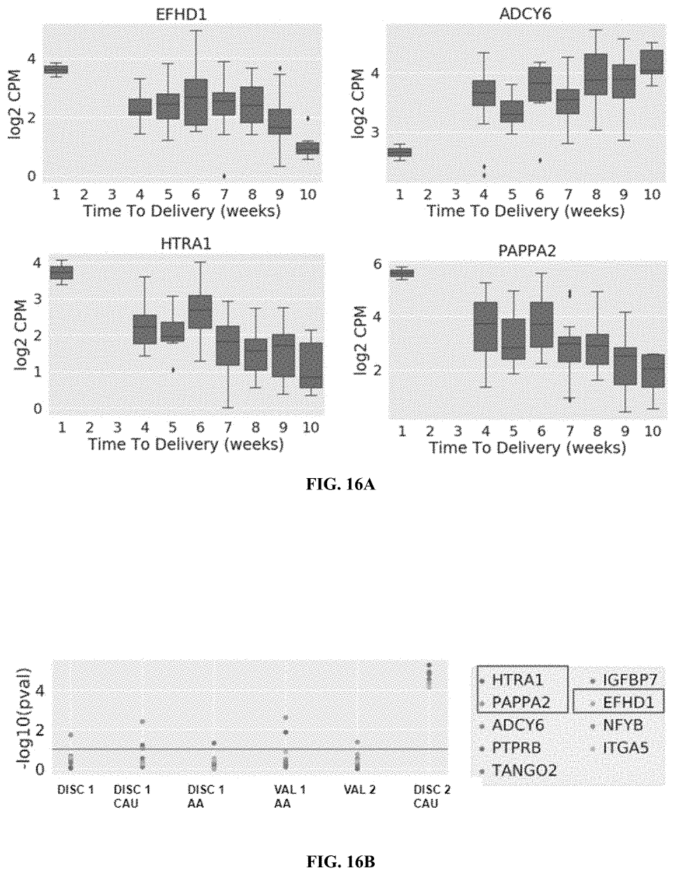

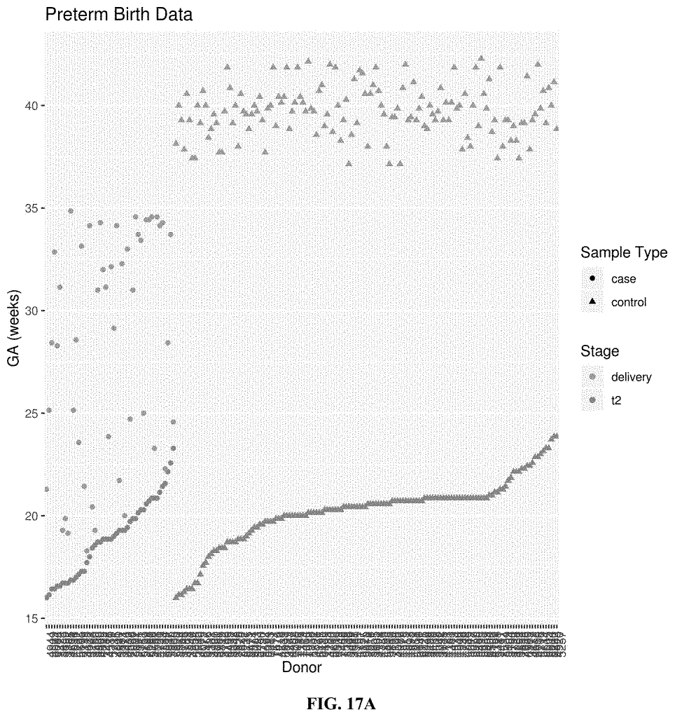

FIG. 16A shows expression trends and significant abundance level separation for a set of top 4 genes (EFHD1, ADCY6, HTR1, PAPPA2) between samples collected at 1 week before birth. FIG. 16B shows an example of genes showing significant correlation to being close to delivery. This figure demonstrates that correlation p-value significance of log₁₀(p-value) exceeds a threshold of 1 for 3 genes (HTRA1, PAPPA2, and EFHD1) in several discovery and validation cohorts.

### Example 8: Prediction of Pre-Term Birth (PTB)

Using systems and methods of the present disclosure, a prediction model was developed to detect or predict a risk of pre-term birth (PTB) of a pregnant subject. The prediction model development comprised obtaining a cohort of subjects and training the prediction model on a training dataset corresponding to the cohort of subjects.

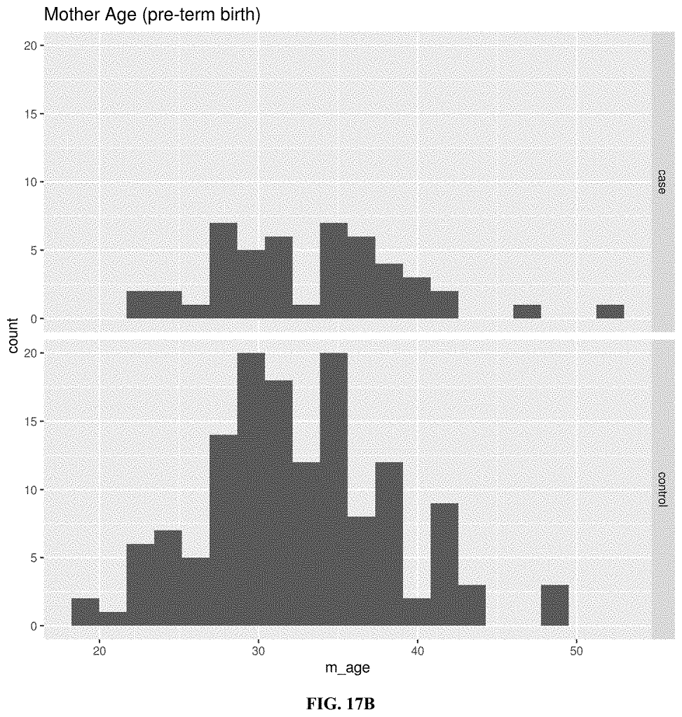

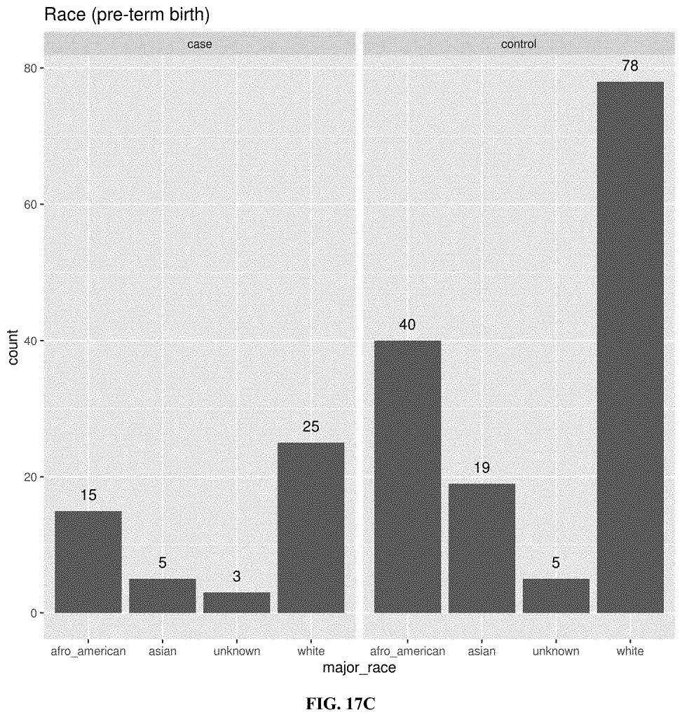

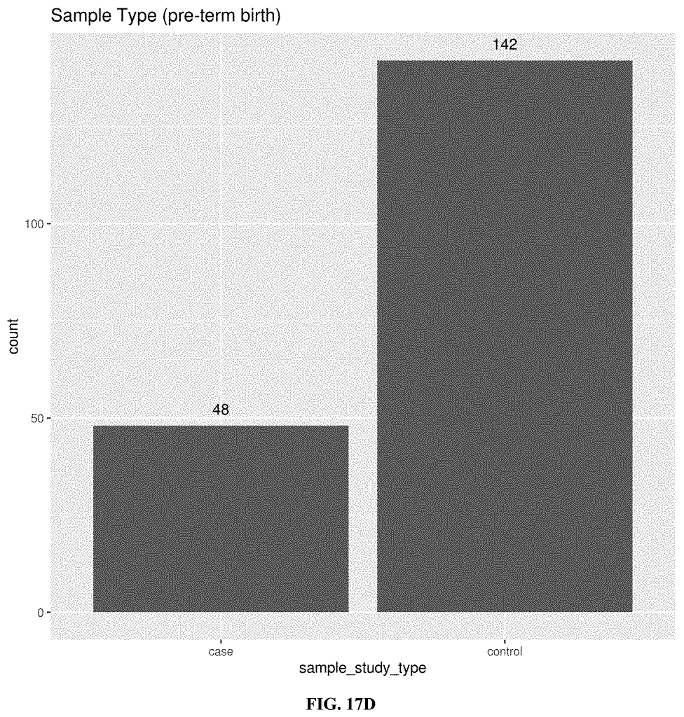

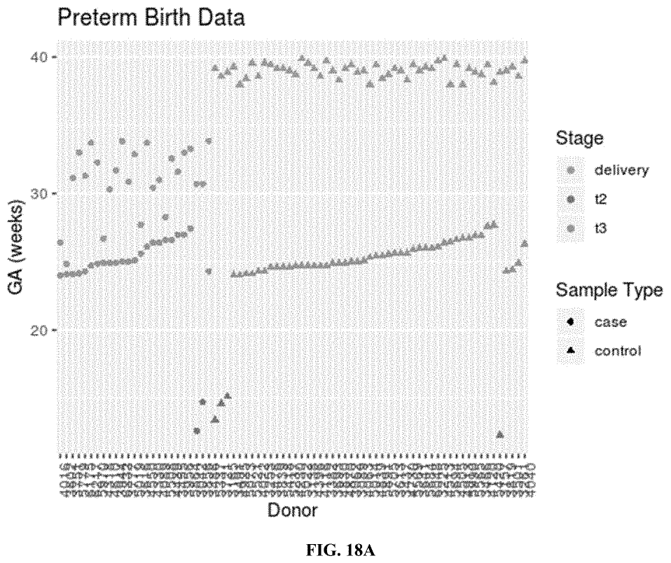

The cohort of subjects was obtained as follows. As shown in FIG. 17A, a first cohort of 192 subjects (e.g., pregnant women) was established (with patient identification numbers shown on the x-axis). From this cohort, one or more biological samples (e.g., 1 or 2) were collected and assayed at different time points corresponding to an estimated gestational age (shown on the y-axis, in increasing order of estimated gestational age at delivery) of a fetus of each subject, using methods and systems of the present disclosure. For example, the estimated gestational age (shown on the y-axis) may be determined using methods such as ultrasound imaging, a last menstrual period (LMP) date, or a combination thereof, and may range from 0 to about 42 weeks. The first cohort includes subjects from whom different sample types (preterm, high risk preterm, miscarriages, or stillbirth) were collected for use in different types of modeling with sample classifications to identify markers associated preterm, miscarriages, or stillbirth in different subtypes or classes.

FIG. 17B shows a distribution of participants in the first cohort based on each participant's age at the time of medical record abstraction. FIG. 17C shows a distribution of 192 participants in the first cohort based on each participant's race. FIG. 17D shows a distribution of 192 collected samples in the first cohort based on the study sample type of the collected samples.

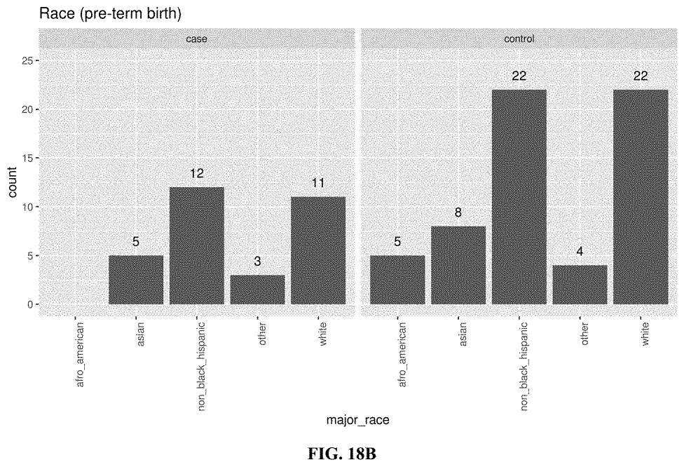

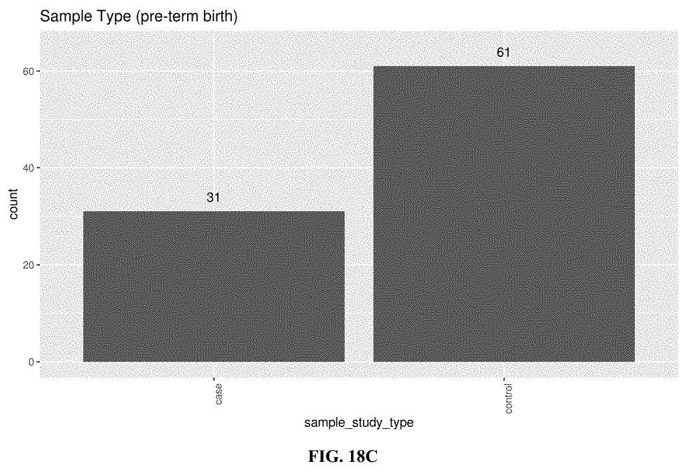

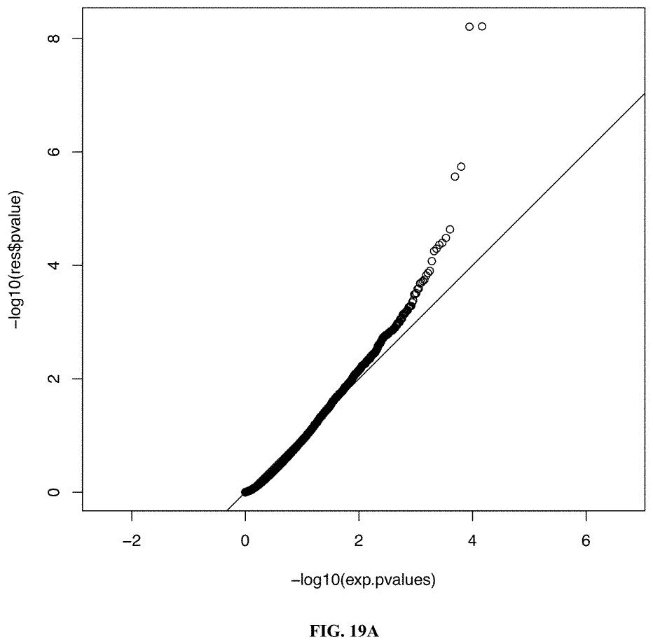

Further, as shown in FIG. 18A, a second cohort of 76 subjects (e.g., pregnant women) was established (with patient identification numbers shown on the x-axis). From this cohort, one or more biological samples (e.g., 1 or 2) were collected and assayed at different time points corresponding to an estimated gestational age (shown on the y-axis, in increasing order of estimated gestational age at delivery) of a fetus of each subject, using methods and systems of the present disclosure. For example, the estimated gestational age (shown on the y-axis) may be determined using methods such as ultrasound imaging, a last menstrual period (LMP) date, or a combination thereof, and may range from 0 to about 42 weeks.

FIG. 18B shows a distribution of 76 participants in the second cohort based on each participant's race. FIG. 18C shows a distribution of 76 collected samples (25 pre-term samples and 51 full-term controls) in the second cohort based on the study sample type of the collected samples. FIG. 18D shows a distribution of 76 collected samples (25 pre-term samples and 51 full-term controls) in the second cohort based on the study sample type of the collected samples.

Differential expression analysis of the first cohort data set was performed as follows. An analysis for differentially expressed genes between the pre-term case samples and control samples was performed, revealing a set of 100 differentially expressed genes across all cases and controls.

For example, Table 11 shows the differential gene expression between different subclasses for PTB cases. Samples were classified into a high-risk group if they were associated with having a previous history of at least one of following pregnancy complications: spontaneous PTB, PPROM, late miscarriage (e.g., after 14 weeks of gestational age), cervical surgery, and uterine anomaly. Samples were classified into a low-risk group if they were associated with a general antenatal population with none of the above risk factors. Miscarriage was characterized by having delivered before 24 weeks of gestational age.

TABLE 11
Pre-Term Birth Signal in Different Sub-Types of PTB

| | Cases/ | DE genes | DE genes | |
|---|---|---|---|---|
| | Controls | up | down | Top Genes |
| All PTB | 49/144 | 15 | 83 | Shared |
| High risk | 44/123 | 18 | 172 | Shared |
| Low risk | 5/14 | 0 | 1 | Different genes |
| Miscarriage | 14/41 | 0 | 0 | Different genes |
| or stillbirth | | | | |

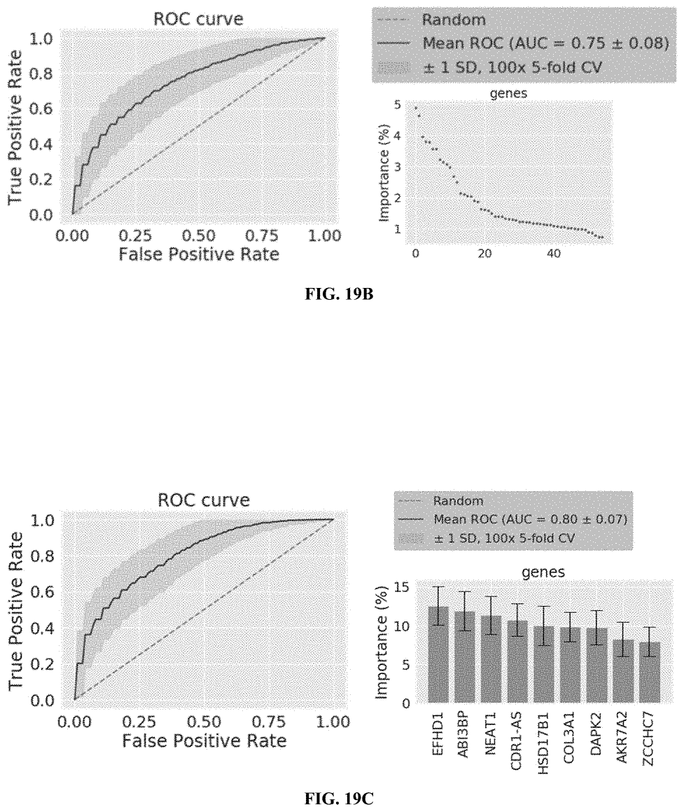

A signal in pre-term birth-associated genes in different sub-types of PTB was observed to be driven by a high-risk group as shown in FIG. 19A, which shows a quantile-quantile (QQ) plot of a graphical representation of the deviation of the observed P values from the null hypothesis for individual genes. Genes which are deviated from the middle line at the log₁₀(p-value) of 3.5 are considered to be truly differentially expressed in high-risk populations relative to healthy controls. A set of top genes that are predictive for high risk pre-term birth (PTB) are listed in Table 12.

FIG. 19B shows a receiver-operator characteristic (ROC) curve for the high pre-term birth prediction model, using all differentially expressed genes from Table 11 for a set of 167 samples obtained from a high-risk subclass cohort of Caucasian subjects. Of the 167 total samples, 44 had early PTB (e.g., delivery before 34 weeks of estimated gestational age). The mean area-under-the-curve (AUC) for the ROC curve was 0.75±0.08. FIG. 19C shows a receiver-operator characteristic (ROC) curve for a set of top 9 genes (EFHD1, ABI3BP, NEAT1, HSD17B1, CDR1-AS, GCM1, DAPK2, ZCCHC7, COL3A1, and AKR7A2). The mean area-under-the-curve (AUC) for the ROC curve was 0.80±0.07, with relative contributions from each gene.

TABLE 12
Top Set of Predictive Genes for High-Risk Pre-Term Birth (PTB)

| Gene | P-adj | log2 Fold Change |
|---|---|---|
| CDR1-AS | 0.000006232042908 | 1.531899181 |
| COL3A1 | 0.0001829599367 | 2.296099004 |
| DCN | 0.007756452652 | 1.959492728 |
| DAPK2 | 0.008577062504 | −0.6538136896 |
| ABI3BP | 0.01846895706 | 1.253946028 |
| NEAT1 | 0.02229732621 | −0.8955349534 |
| ANTXR1 | 0.02229732621 | 1.307627338 |
| PLEKHM1P1 | 0.02229732621 | −0.9490980614 |
| TNFRSF25 | 0.02563117996 | −2.074833817 |
| MEGF6 | 0.02563117996 | −1.616170492 |
| PGGHG | 0.02563117996 | −1.312523641 |
| TNFRSF10B | 0.02728425554 | −1.202142785 |
| LUM | 0.0273958536 | 2.615661527 |
| MMP2 | 0.0273958536 | 1.511005424 |
| MYO18B | 0.02810913316 | −1.11864242 |
| TMC8 | 0.03087184347 | −0.8337355677 |
| EME2 | 0.03087184347 | −1.563909654 |
| GCM1 | 0.03087184347 | −1.537115843 |
| COL14A1 | 0.03163361683 | 1.743013436 |
| ZCCHC7 | 0.0323639933 | 0.222285457 |
| EIF4A1 | 0.0323639933 | −1.02093915 |
| ABCC10 | 0.03655742169 | −1.21406946 |
| PABPC1L | 0.03944887005 | −1.272184265 |
| LILRA6 | 0.03981500296 | −1.225586629 |
| ADCY7 | 0.03981500296 | −0.911845995 |
| HSD17B1 | 0.03981500296 | −1.112912409 |
| SLC24A4 | 0.03981500296 | −1.36958566 |
| PIEZO1 | 0.03981500296 | −0.7881581173 |
| SLC27A3 | 0.03981500296 | −0.9788188364 |
| FBN2 | 0.03981500296 | −1.075292442 |
| SLC12A9 | 0.03981500296 | −0.9818661938 |
| SLC43A2 | 0.03981500296 | −0.9510233821 |
| ABCA7 | 0.03981500296 | −0.7356204689 |
| SPOCK2 | 0.03981500296 | −0.8143930692 |
| AL773572.7 | 0.03981500296 | −1.667040365 |
| SEC31B | 0.03981500296 | −1.197850588 |
| ARRDC5 | 0.03981500296 | −1.690147984 |
| APBB3 | 0.03981500296 | −1.393590176 |
| SLC11A1 | 0.03981500296 | −0.9838153699 |
| APOBR | 0.04450245034 | −0.7589482093 |
| GH2 | 0.04450245034 | −1.47585156 |
| TLR2 | 0.04636265694 | −0.8826852522 |
| GAA | 0.04636265694 | −0.987530859 |
| NTNG2 | 0.04656847046 | −1.541500092 |
| SNORD46 | 0.04656847046 | −1.96052151 |
| PBXIP1 | 0.04656847046 | −0.5065889974 |
| S1PR3 | 0.04690323503 | −1.664837438 |
| FRAT2 | 0.04845006461 | −0.7376686877 |
| FLG2 | 0.04845006461 | −1.678849501 |
| CLASRP | 0.04845006461 | −0.6278945866 |
| FCGRT | 0.04921060752 | −0.797948221 |
| PDE3B | 0.04951788766 | −0.6367484205 |
| TMC6 | 0.04951788766 | −0.718127351 |
| EFHD1 | 0.04951788766 | −1.17965089 |
| AKR7A2 | 0.04958579441 | 0.4800853396 |
| ITGAM | 0.05150923955 | −0.3518160003 |
| PLXNA3 | 0.05220665814 | −0.8351641135 |
| NUP210 | 0.05279441154 | −0.5578845296 |
| SSH3 | 0.05279441154 | −0.6053200011 |
| NPEPL1 | 0.05515096309 | −0.9625781876 |
| COL9A2 | 0.05544088408 | −0.9036988185 |
| SULF2 | 0.05931148621 | −0.8282550008 |
| ATG16L2 | 0.06093047358 | −0.8232810424 |
| LENG8 | 0.06137133329 | −0.5229381575 |
| DNHD1 | 0.06137133329 | −0.8242614989 |
| MYH3 | 0.06137133329 | −1.027874258 |
| SIGLEC14 | 0.06137133329 | −0.969520126 |
| ODF3B | 0.06137133329 | −0.9851026487 |
| CSH1 | 0.06167244945 | −0.8095712072 |
| TAP1 | 0.06167244945 | −0.5279898052 |
| TCIRG1 | 0.06167244945 | −0.8389438684 |
| TMTC2 | 0.06167244945 | −0.8691690267 |
| AOAH | 0.06167244945 | −0.6439585779 |
| TLR8 | 0.06663109333 | −0.8023150795 |
| DIRC2 | 0.06663109333 | −0.8674598547 |
| MPEG1 | 0.06663109333 | −0.6624359256 |
| RAB44 | 0.06663109333 | −0.8997466671 |
| NLRP1 | 0.06663109333 | −0.6868095141 |
| UVSSA | 0.06663109333 | −0.6160785003 |
| PLXNB2 | 0.06663109333 | −0.6271170344 |
| IGF2R | 0.06663109333 | −0.6918340652 |
| NOTCH1 | 0.06663109333 | −0.4765941786 |
| ARPC4- | | |
| TTLL3 | 0.06663109333 | −0.7045393297 |
| CD300C | 0.06663109333 | −1.144634751 |
| SH2B1 | 0.06663109333 | −0.578963839 |
| LGALS14 | 0.06663109333 | −1.125378735 |
| CCDC88B | 0.06663109333 | −0.6836681428 |
| GTPBP3 | 0.06663109333 | −0.7362739174 |
| ATP10A | 0.06663109333 | −0.7959520418 |
| SIGLEC7 | 0.06663109333 | −0.6692818639 |
| COLGALT1 | 0.06663109333 | −0.730199416 |
| SUN2 | 0.06663109333 | −0.6109180612 |
| ABCA2 | 0.06663109333 | −0.9002282272 |
| CSF3R | 0.06663109333 | −0.8347284824 |
| NSUN5P2 | 0.06678833246 | −1.567214574 |
| LRP1 | 0.06678911515 | −0.7509418684 |
| MRI1 | 0.06680407486 | −0.8427458222 |
| KLC4 | 0.0675554476 | −0.4761855735 |
| C1S | 0.06874852119 | 0.8897786067 |
| RPS24P8 | 0.07310321208 | −0.8139181709 |
| RSRP1 | 0.07328786935 | −0.5165840992 |
| TMEM173 | 0.07328786935 | −0.6198609879 |
| ZNF767P | 0.07328786935 | −1.328460916 |
| LILRB2 | 0.07328786935 | −0.7255314572 |
| MBOAT7 | 0.07328786935 | −0.6439778317 |
| EP400NL | 0.07505883827 | −0.5986535479 |
| SNORA74B | 0.07505883827 | −2.153171587 |
| COL1A1 | 0.07649313302 | 1.467807155 |
| NSRP1P1 | 0.07819752186 | −0.8798559714 |
| ATP10D | 0.07819752186 | −0.5973763959 |
| VGLL3 | 0.07819752186 | −0.8564161572 |
| POGLUT1 | 0.07819752186 | −0.7284583558 |
| SENP3 | 0.07819752186 | −0.4415204386 |
| RELT | 0.07819752186 | −0.9387042103 |
| MGAT1 | 0.07819752186 | −0.5057774794 |
| EPPK1 | 0.07836403686 | −0.7908834718 |
| SIRPB1 | 0.07915186374 | −0.9127490872 |
| ZNF90 | 0.07915186374 | 0.3357861199 |
| CAPN13 | 0.07915186374 | 1.39545777 |
| POLM | 0.07915186374 | −0.652546798 |
| SIRPB2 | 0.07915186374 | −1.001548716 |
| CAPN6 | 0.07977866418 | −1.027198094 |
| AC004951.6 | 0.07977866418 | 1.695803913 |
| COL5A1 | 0.07977866418 | 1.080964445 |
| CCNL1 | 0.07977866418 | −0.5394395627 |
| CCDC80 | 0.07977866418 | 0.7506926428 |
| LZTR1 | 0.07977866418 | −0.3694662723 |
| CORO7 | 0.0823144424 | −0.6671451408 |
| SGSM2 | 0.0823144424 | −0.5107151598 |
| REC8 | 0.0823144424 | −0.6811017805 |
| CSHL1 | 0.0823144424 | −1.128469072 |
| PLAC4 | 0.0823144424 | −0.9715559701 |
| KIFC2 | 0.0823144424 | −1.318471383 |
| TRABD2A | 0.08455470118 | −0.916025636 |
| C7orf43 | 0.08521222818 | −0.6290196123 |
| LTBR | 0.08576238338 | −0.6873265786 |
| NLRC5 | 0.08576238338 | −0.3309468614 |
| CD93 | 0.08716347419 | −0.7630469638 |
| TNFRSF1A | 0.08716347419 | −0.6552554162 |
| CDK5RAP3 | 0.08716347419 | −0.5267137109 |
| FGL2 | 0.08828798716 | −0.5520944536 |
| HIC2 | 0.08828798716 | −0.8628085035 |
| TRAF1 | 0.08828798716 | −0.7507113762 |
| DNAH1 | 0.08828798716 | −0.6269726561 |
| SERINC5 | 0.08828798716 | 0.4411719721 |
| ITGB2 | 0.08828798716 | −0.5961969581 |
| AGAP9 | 0.08828798716 | −0.7465933148 |
| MYO15B | 0.08871590633 | −0.5886292587 |
| ALG2 | 0.08871590633 | −0.5054504041 |
| LFNG | 0.08885322846 | −0.872300955 |
| SORL1 | 0.08929473343 | −0.6423125952 |
| SLC2A6 | 0.09076981423 | −1.013599518 |
| TRIM56 | 0.09076981423 | −0.3351847824 |
| GGA3 | 0.09076981423 | −0.1917226273 |
| ADAMTSL4 | 0.09076981423 | −0.8144474405 |
| AAK1 | 0.09076981423 | −0.2503087338 |
| PLEC | 0.09228195226 | −0.5019996265 |
| KLC1 | 0.09228195226 | −0.3215539114 |
| SETD1B | 0.09228195226 | −0.3296507553 |
| SLC38A10 | 0.09228195226 | −0.4899444244 |
| EXOC3 | 0.09228195226 | −0.1717569971 |
| CSH2 | 0.09228195226 | −0.6712648492 |
| P2RX7 | 0.09228195226 | −0.8696358362 |
| ZNF335 | 0.0925066107 | −0.4051906146 |
| TSPOAP1 | 0.0925066107 | −0.6263300552 |
| MROH1 | 0.0925066107 | −0.4067563819 |
| MAN2C1 | 0.0925066107 | −0.457260922 |
| SCPEP1 | 0.0925066107 | −0.58621504 |
| FRS3 | 0.09340243497 | −0.7845220185 |
| FCN1 | 0.094079047 | −0.6393500511 |
| CSRNP1 | 0.094079047 | −0.4135881931 |
| CPVL | 0.09479121535 | −0.6477578756 |
| PLAC9 | 0.09491876413 | 1.510583009 |
| TNFRSF1B | 0.09506645739 | −0.7048093579 |
| CCDC142 | 0.09569299562 | −0.9093263547 |
| PLCH2 | 0.09569299562 | −0.9376399083 |
| ITGA5 | 0.09632706616 | −0.5427180069 |
| ARHGAP33 | 0.09632706616 | −0.9479851887 |
| MT1E | 0.09715293572 | 0.6727425964 |
| OBSCN | 0.09794438812 | −0.5382292327 |
| TRPM2 | 0.09952076687 | −0.8305205972 |
| MMP17 | 0.09960934016 | −0.9364206448 |
| C3AR1 | 0.09960934016 | −0.5520165487 |
| VIPR1 | 0.09960934016 | −1.165669094 |
| SREBF1 | 0.09960934016 | −0.6029100137 |
| RREB1 | 0.09960934016 | −0.1587187676 |
| TMEM256- | | |
| PLSCR3 | 0.09960934016 | −1.22479337 |
| CREBZF | 0.09960934016 | −0.4118130094 |
| ADAM8 | 0.09999909729 | −0.8574616833 |
| HSPA7 | 0.09999909729 | −1.129374439 |

Differential expression analysis of the second cohort data set was performed as follows. Biomarker discovery was performed to identify early diagnostic markers of pre-term using cell-free RNA samples in the second cohort. In order to reduce the effect of gestational age, the sample set was reduced to 27 plasma samples from pregnant women who delivered pre-term and 53 plasma samples from matched controls that were collected at equivalent weeks of gestation (e.g., about 25 weeks of gestational age), as shown in Table 13.

TABLE 13
Demographics of Early PTB Samples in the Second Cohort

| | Samples | GA at collection (weeks) | BMI |
|---|---|---|---|
| Pre-term cases | 27 | 25.4 ± 1.0 | 29.5 ± 6.5 |
| controls | 53 | 25.4 ± 1.0 | 26.2 ± 8.0 |

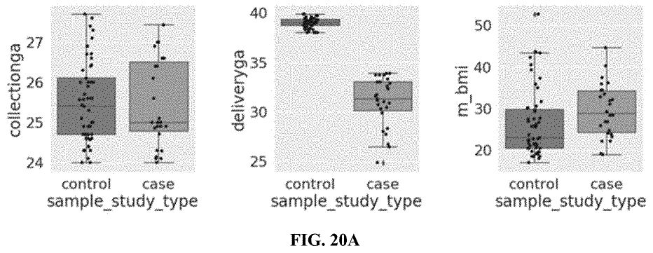

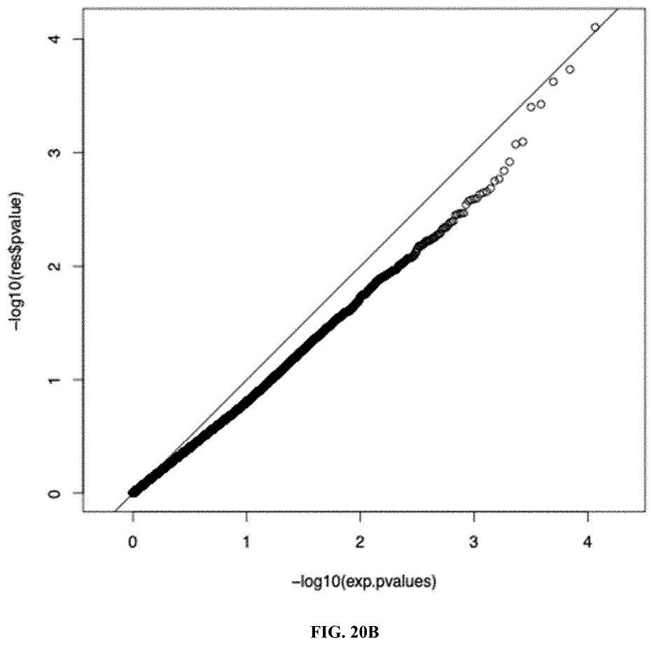

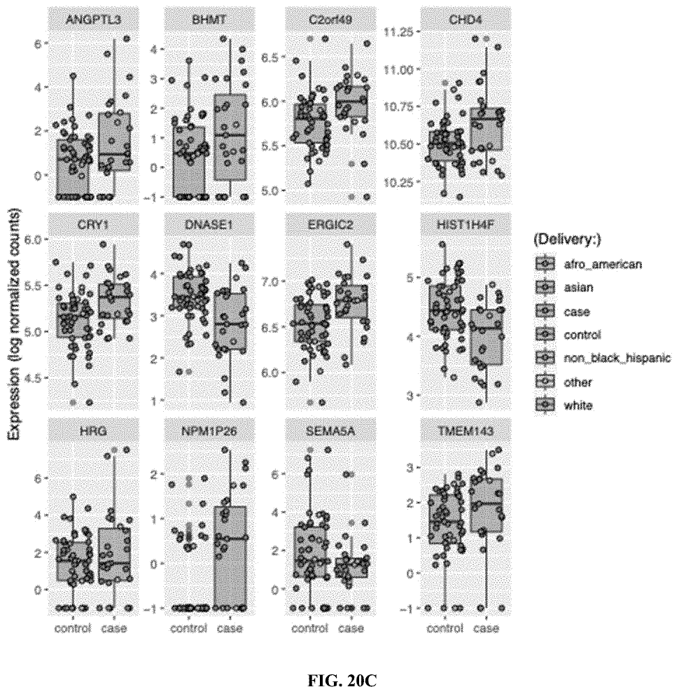

FIG. 20A shows a distribution of demographic statistics for this subset of early PTB samples and controls in the second cohort that were included in the analysis. An analysis for differentially expressed genes between the pre-term case samples and pre-term control samples was performed. A set of top 30 genes that are predictive for high risk pre-term birth (PTB) were determined, as shown in Table 14.

TABLE 14
Statistical Values for Top Differentially Expressed
Genes for Early PTB in the Second Cohort

| | Mean | Log2 Fold | |
|---|---|---|---|
| Gene | Expression | Change | P−value |
| HRG | 8.140452 | 1.920363 | 7.89E−05 |
| ANGPTL3 | 3.847834 | 1.83131 | 0.000185 |
| NPM1P26 | 0.671245 | 1.936622 | 0.000237 |
| HIST1H4F | 20.91216 | −0.47087 | 0.000377 |
| CRY | 36.99376 | 0.257658 | 0.000399 |
| BHMT | 2.291833 | 1.484639 | 0.000806 |
| C2orf49 | 57.97035 | 0.249506 | 0.000848 |
| OASL | 26.75105 | 0.719533 | 0.001211 |
| SELE | 1.296385 | 1.631514 | 0.001446 |
| CHD4 | 1515.132 | 0.15261 | 0.001708 |
| IFIT1 | 115.1264 | 0.672503 | 0.001787 |
| DHX38 | 418.0855 | 0.182905 | 0.00207 |
| DNASE1 | 10.21555 | −0.53365 | 0.002209 |
| CEACAM6 | 25.49209 | −0.69758 | 0.002253 |
| AGPAT4 | 6.973746 | −0.56801 | 0.002335 |
| SERPING1 | 172.2336 | −0.75404 | 0.002538 |
| PLCXD1 | 12.50904 | −0.52192 | 0.002565 |
| ARFGEF3 | 5.735036 | −0.73881 | 0.002608 |
| ERGIC2 | 99.542 | 0.222491 | 0.002671 |
| SH2D1A | 33.09903 | −0.48059 | 0.002872 |
| AEBP1 | 7.716002 | −0.87421 | 0.00341 |
| SIGLEC6 | 4.86553 | −0.90286 | 0.003431 |
| PIP5K1A | 53.89827 | −0.17974 | 0.003437 |
| IGHV3-48 | 1.871432 | 1.118533 | 0.003499 |
| TRBV4-2 | 0.981817 | −1.54074 | 0.003557 |
| PHC1P1 | 8.194502 | 0.412459 | 0.003999 |
| FAM76B | 128.4759 | 0.151824 | 0.004071 |
| PDE6H | 2.829983 | 0.905734 | 0.004152 |
| PDAP1 | 670.607 | 0.159327 | 0.004326 |

FIG. 20B shows a QQ plot for early PTB in the second cohort, which is a graphical representation of the deviation of the observed P values from the null hypothesis for individual genes. Genes which are deviated from the middle line at the log₁₀(p-value) of 3.5 are considered to be truly differentially expressed in between case and healthy controls.

FIG. 20C shows boxplots and significant abundance level separation for the top 12 differentially expressed genes (ANGPTL3, NPM1P26, HIST1H4F, CRY1, BHMT, C2orf49, OASL, SELE, CHD4, IIFIT1, DHX38, and DNASE1) for early PTB in the second cohort. The results indicate that differential expression was not driven by ethnic differences in maternal subjects.

### Example 9: Prediction of Preeclampsia (PE)

Using systems and methods of the present disclosure, a prediction model was developed to detect or predict a risk of preeclampsia (PE) of a pregnant subject. The prediction model development comprised obtaining a cohort of subjects and training the prediction model on a training dataset corresponding to the cohort of subjects.

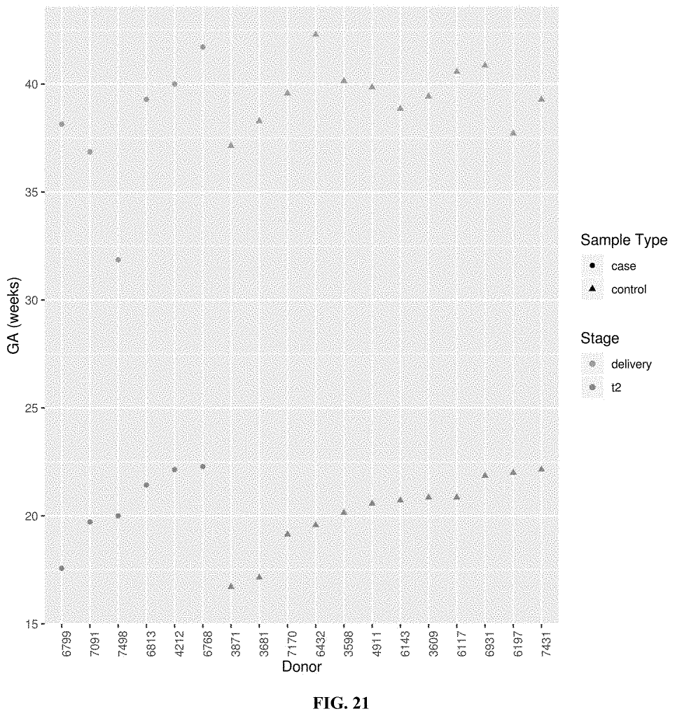

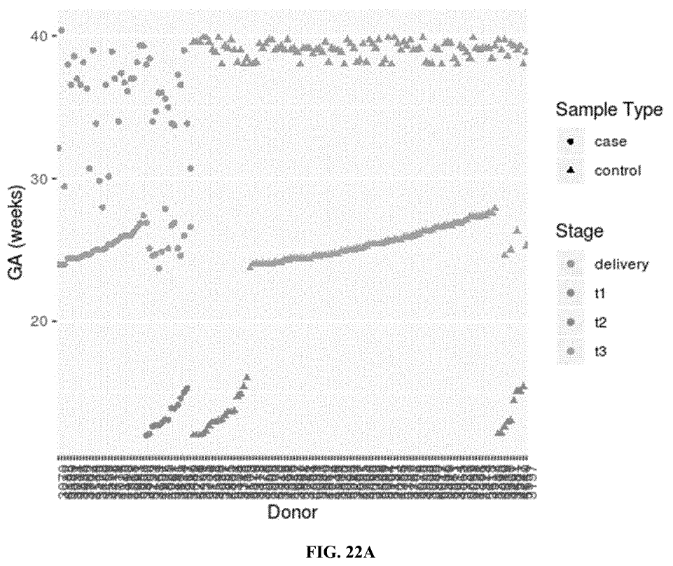

The cohort of subjects was obtained as follows. As shown in FIG. 21, a first cohort of 18 subjects (e.g., pregnant women) was established (with delivery on the x-axis). From this cohort, one or more biological samples were collected and assayed at different time points corresponding to an estimated gestational age (shown on the x-axis, in increasing order of estimated gestational age at delivery) of a fetus of each subject, using methods and systems of the present disclosure. For example, the estimated gestational age (shown on the x- and y-axis) may be determined using methods such as ultrasound imaging, a last menstrual period (LMP) date, or a combination thereof, and may range from 0 to approximately 42 weeks. The first cohort includes 6 cases of PE with 1 subject of early onset of PE resulting in delivery before 32 weeks of gestation, and 5 subjects with late onset of PE with delivery after 36 weeks of gestation.

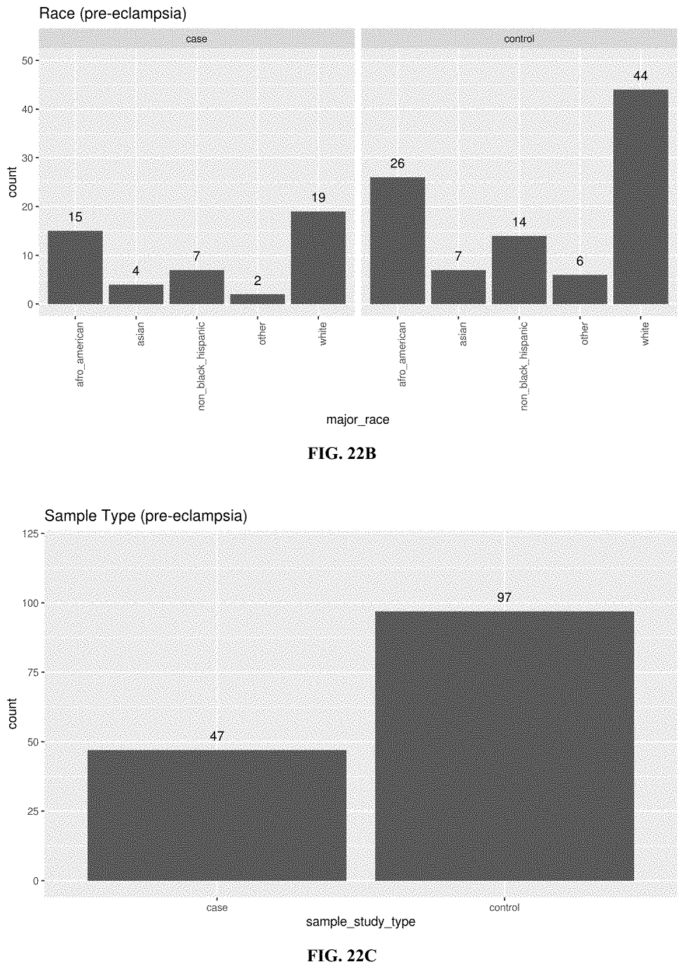

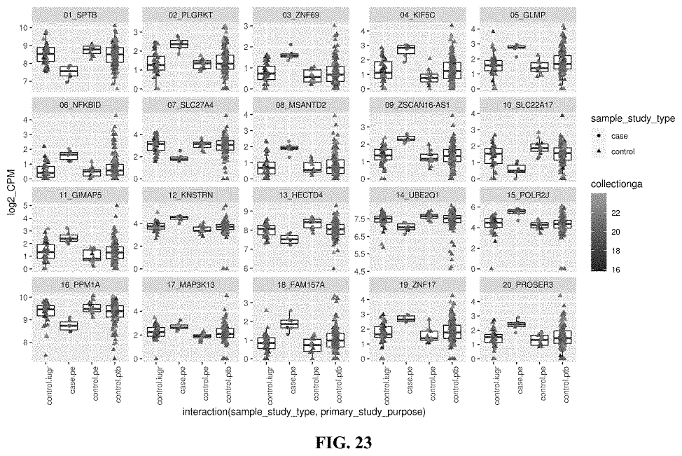

Further, as shown in FIG. 22A, a second cohort of 130 subjects (pregnant women) was established (with patient identification numbers shown on the x-axis). From this cohort, one or more biological samples (e.g., 1 or 2) were collected and assayed at different time points corresponding to an estimated gestational age (shown on the y-axis, in increasing order of estimated gestational age at delivery) of a fetus of each subject, using methods and systems of the present disclosure. For example, the estimated gestational age (shown on the y-axis) may be determined using methods such as ultrasound imaging, a last menstrual period (LMP) date, or a combination thereof, and may range from 0 to about 42 weeks. The first cohort includes subjects from whom different sample types were collected for use in different types of modeling with sample classifications to identify markers associated preterm in different subtypes or classes.

FIG. 22B shows a distribution of 130 participants in the second cohort based on each participant's race. FIG. 22C shows a distribution of 144 collected samples in the second cohort based on the study sample type of the collected samples.

Differential expression analysis of the first cohort data set was performed as follows. An analysis for de novo discovery for statistically significant genes between the preeclampsia case samples and healthy control samples was performed, revealing a set of 3,869 differentially expressed genes.

For example, Table 15 shows the top 20 differential expressed genes with top 4 genes (SPTB, PLGRKT, ZNF69, and KIF5C) satisfying a threshold of a Bonferroni correction of p-value less than 0.05 between cases and controls for preeclampsia.

TABLE 15
Top 20 Statistically Significant Differentially
Expressed Genes in Preeclampsia (PE)

| Gene | P-value | bh adjusted | bonferroni adjusted |
|---|---|---|---|
| SPTB | 7.21E−07 | 0.009338582 | 0.009338582 |
| PLGRKT | 1.61E−06 | 0.009585951 | 0.020811664 |
| ZNF69 | 2.73E−06 | 0.009585951 | 0.035325024 |
| KIF5C | 2.96E−06 | 0.009585951 | 0.038343805 |
| GLMP | 5.44E−06 | 0.01128075 | 0.070507842 |
| NFKBID | 5.47E−06 | 0.01128075 | 0.070885069 |
| SLC27A4 | 6.60E−06 | 0.01128075 | 0.085479797 |
| MSANTD2 | 6.96E−06 | 0.01128075 | 0.090246002 |
| ZSCAN16-AS1 | 8.26E−06 | 0.011898545 | 0.107086908 |
| SLC22A17 | 1.18E−05 | 0.015324382 | 0.153559972 |
| GIMAP5 | 1.38E−05 | 0.015324382 | 0.178203029 |
| KNSTRN | 1.47E−05 | 0.015324382 | 0.191059786 |
| HECTD4 | 1.54E−05 | 0.015324382 | 0.199216971 |
| UBE2Q1 | 2.04E−05 | 0.018495821 | 0.264604216 |
| POLR2J | 2.14E−05 | 0.018495821 | 0.277437317 |
| PPM1A | 2.40E−05 | 0.019438155 | 0.311010475 |
| MAP3K13 | 2.78E−05 | 0.02120929 | 0.360557924 |
| FAM157A | 3.57E−05 | 0.02405401 | 0.462147561 |
| ZNF17 | 3.67E−05 | 0.02405401 | 0.475265105 |
| PROSER3 | 3.88E−05 | 0.02405401 | 0.503185564 |

FIG. 23 shows a significant abundance level separation between cases and healthy controls for the top 20 differentially expressed genes for preeclampsia (PE) in the first cohort. An additional set of 192 healthy controls with blood collection at the same gestation and similar demographic profile added as the second healthy control group to show good differential expression separation for preeclampsia subjects.

Differential expression analysis of the second cohort data set was performed as follows. We performed biomarker discovery to identify early diagnostic markers of preeclampsia using cell-free RNA in the second cohort. In order to reduce the effect of gestational age, the sample set was reduced to 36 plasma samples from pregnant women who developed preeclampsia, and 74 plasma samples from matched controls that were collected at equivalent weeks of gestation (e.g., about 25 weeks of gestational age) and comparable maternal body mass index (BMI), as shown in Table 16.

TABLE 16
Demographics of PE Samples in the Second Cohort

| | Samples | GA at Collection (weeks) | BMI |
|---|---|---|---|
| Cases | 36 | 25.3 ± 1.0 | 29.8 ± 7.2 |
| Controls | 74 | 25.4 ± 1.1 | 28.5 ± 7.2 |

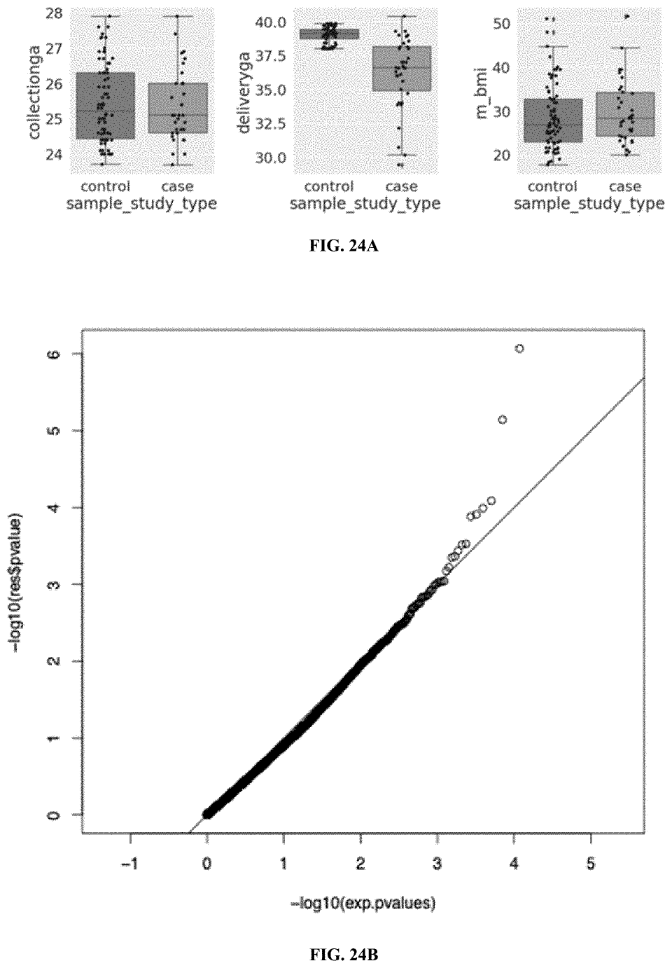

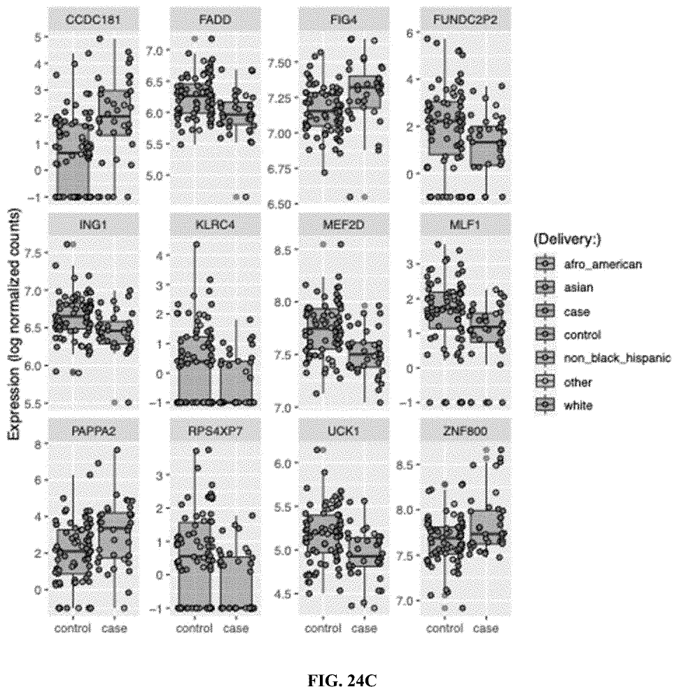

FIG. 24A shows a distribution of demographic statistics for the subset of PE samples and controls in the second cohort that were included in the analysis. Differential expression analysis was performed between cases and controls using a Wald test, thereby obtaining a set of differentially expressed genes between pregnancies that developed preeclampsia and matched controls.

Table 17 shows the top 19 differentially expressed genes for PE. Notably, among the top genes found, several genes were associated with placental development, such as PAPPA2. It was observed that PAPPA2 showed significant statistical significance after adjustment for multiple hypothesis correction, and also showed a significant deviation from the null hypothesis in a QQ plot for differentially expressed in PE (as shown in FIG. 24B).

Additionally, as shown in the boxplots of FIG. 24C, the differences in top 12 genes (AGAP9, ANKRD1, CIS, CCDC181, CIAPIN1, EPS8L1, FBLN1, FUNDC2P2, KISS1, MLF1, PAPPA2, and TFPI2) expression were not driven by maternal ethnic differences supporting its role as early predictors of preeclampsia. The top 19 genes from differential expression analysis of the second cohort are summarized in Table 17.

TABLE 17
Top 19 Differentially Expressed Genes Predictive
of Preeclampsia (PE) in the Second Cohort

| | Mean | | |
|---|---|---|---|
| Gene | expression | Log2 fold change | p-value |
| PAPPA2 | 10.91463 | 1.634397 | 8.49E−07 |
| MEF2D | 206.7518 | −0.23456 | 7.2E−06 |
| FUNDC2P2 | 5.743276 | −1.3228 | 8.15E−05 |
| CCDC181 | 3.281346 | 1.391803 | 0.000102 |
| FADD | 73.29945 | −0.26702 | 0.000123 |
| RPS4XP7 | 1.418757 | −1.51346 | 0.000131 |
| KLRC4 | 1.187923 | −1.67053 | 0.000297 |
| MLF1 | 2.769177 | −0.80739 | 0.000304 |
| ING1 | 97.81814 | −0.21556 | 0.000366 |
| ZNF800 | 215.7781 | 0.210542 | 0.000433 |
| FIG4 | 148.146 | 0.135923 | 0.000447 |
| UCK1 | 34.70849 | −0.23788 | 0.0006 |
| CD276 | 1.633719 | 1.027845 | 0.00067 |
| PCED1B | 108.4184 | −0.30617 | 0.000909 |
| TRIM8 | 236.5823 | −0.16905 | 0.000918 |
| TMEM129 | 5.657795 | −0.55383 | 0.000937 |
| RP13-383K5.4 | 1.808696 | −0.95442 | 0.000947 |
| CIC | 428.9098 | −0.18848 | 0.001008 |
| CLAPIN1 | 26.95064 | −0.26888 | 0.001031 |

### Example 10: Prediction of Preeclampsia (PE) for Subjects with Blood Collected after 18 Weeks of Gestation Age and Validation Between Two Cohorts

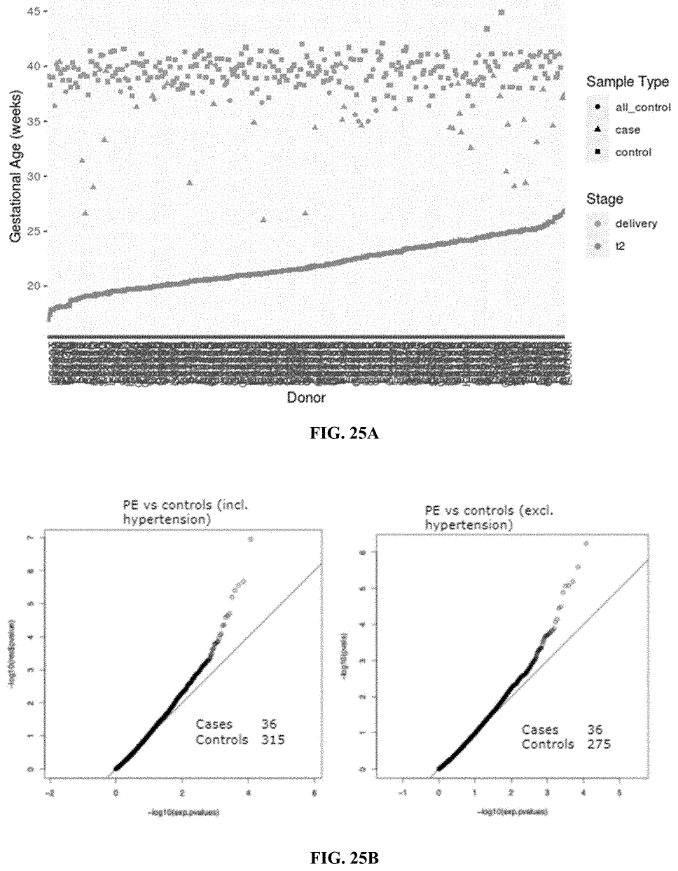

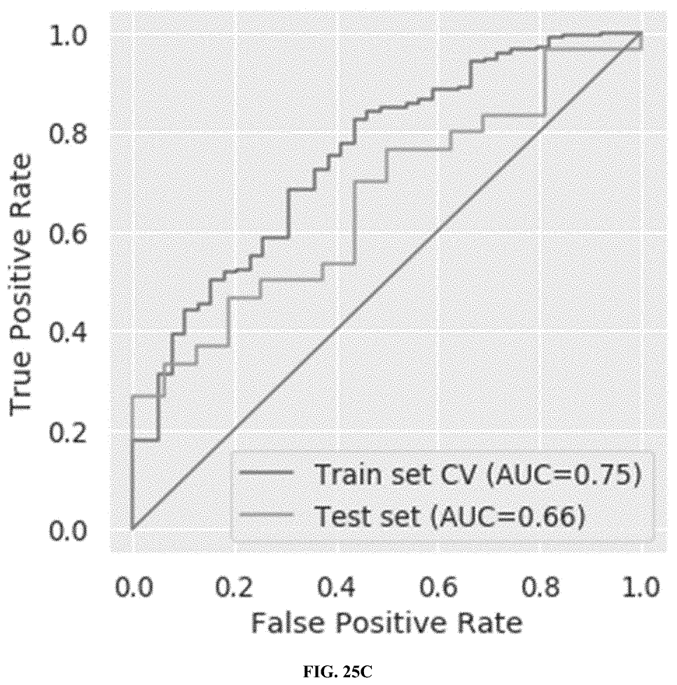

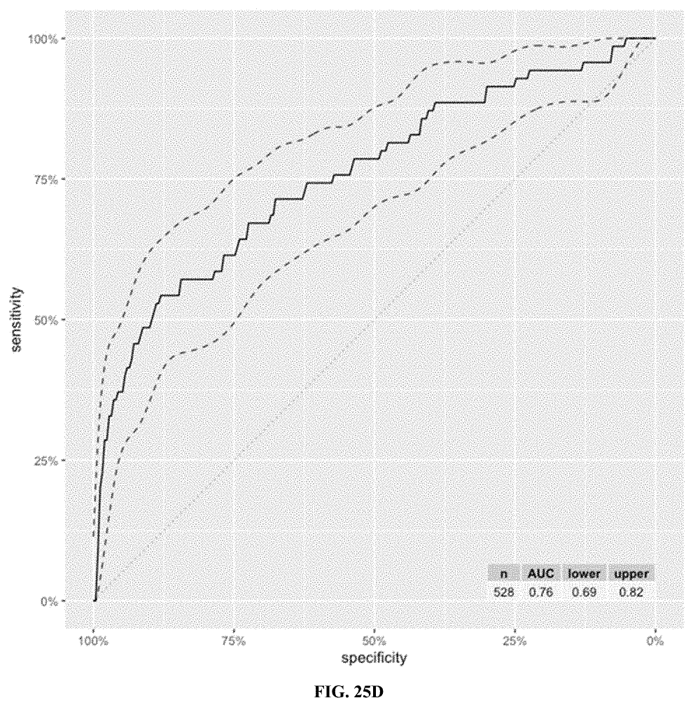

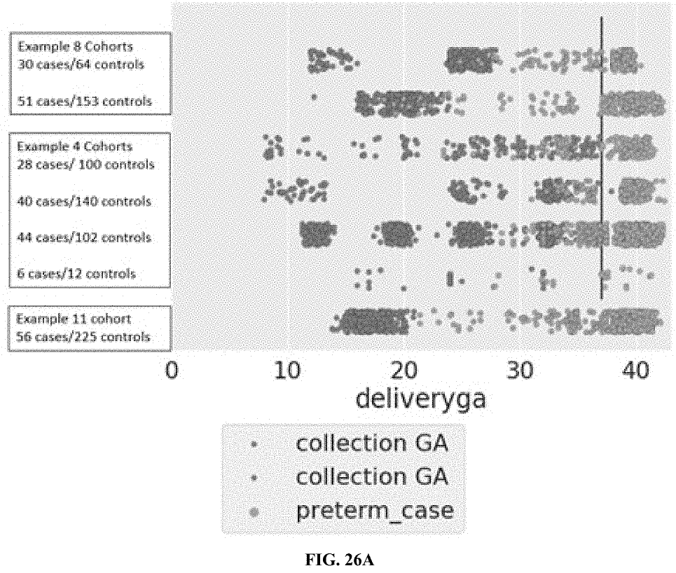

Further, as shown in FIG. 25A, a cohort of 351 subjects (pregnant women) was established (with patient identification numbers shown on the x-axis). From this cohort, one or more biological samples (e.g., 1 or 2) were collected and assayed at different time points corresponding to an estimated gestational age (shown on the y-axis, in increasing order of estimated gestational age at delivery) of a fetus of each subject, using methods and systems of the present disclosure. For example, the estimated gestational age (shown on the y-axis) may be determined using methods such as ultrasound imaging, a last menstrual period (LMP) date, or a combination thereof, and may range from 0 to about 42 weeks. The first cohort includes subjects from whom different sample types were collected for use in different types of modeling with sample classifications to identify markers associated preterm in different subtypes or classes.

Further, a cohort of 351 subjects included 315 control subjects with delivery after 37 weeks of gestational age. 275 control subjects were classified as healthy controls, 40 control subjects had a history of chronic hypertension without preeclampsia. 36 case subjects were diagnosed with preeclampsia and delivered before 37 weeks of gestational age. 24 case subjects were diagnosed with de novo preeclampsia, and 12 case subjects had preeclampsia with a history of chronic hypertension.

Differential expression analysis of the cohort data set was performed as follows. Biomarker discovery was performed to identify early diagnostic markers of preeclampsia using cell-free RNA in the second cohort. In order to estimate the effect of chronic hypertension, two separate differential expression analyses were performed to estimate the effect of chronic hypertension. A first analysis was performed on 36 preeclampsia cases and 275 healthy controls; further, a second analysis was performed, in which 40 control subjects with chronic hypertension were added, thereby totaling 315 control subjects.

Table 18 shows the top differentially expressed genes for PE in the cohort for both comparisons including chronic hypertension and excluding chronic hypertension. The top genes from both analyses overlap, which is indicative of a signal associated with preeclampsia, and not chronic hypertension.

The PAPPA2 gene was among one of the significantly expressed gene list for both comparisons. It was observed that PAPPA2 showed significant statistical significance after adjustment for multiple hypothesis correction, and also showed a significant deviation from the null hypothesis in a QQ plots for differentially expressed in PE (as shown in FIG. 25B). Notably, the PAPPA2 gene is among the top genes found also in Example 9. Table 17 indicates its significance and consistency in preeclampsia associated signal between two different cohorts. The top genes from both differential expression analyses of the cohort are summarized in Table 18.

TABLE 18
Top Differentially Expressed Genes Predictive
of Preeclampsia (PE) in two cohort analyses

| | | | |
|---|---|---|---|
| | Log2 fold | | P-value |
| Gene | change | P-value | (adjusted) |

Including hypertension samples:

| | | | |
|---|---|---|---|
| CDCP1 | 1.77396 | 1.13E−07 | 0.001979 |
| DNAH10 | 0.892914 | 2.17E−06 | 0.016422 |
| ANXA1 | 0.601279 | 2.8E−06 | 0.016422 |
| KLF5 | 1.003333 | 4.03E−06 | 0.017725 |
| PKP1 | 2.050461 | 6.39E−06 | 0.022462 |
| RHBDL2 | 2.548792 | 2.01E−05 | 0.057368 |
| CXCL6 | 1.518407 | 2.34E−05 | 0.057368 |
| PAPPA2 | 1.35799 | 2.61E−05 | 0.057368 |
| SLPI | 1.194633 | 4.39E−05 | 0.08179 |

Excluding hypertension samples:

| | | | |
|---|---|---|---|
| CDCP1 | 1.726904 | 5.82E−07 | 0.010243 |
| DNAH10 | 0.895177 | 2.54E−06 | 0.022396 |
| ANXA1 | 0.590151 | 6.53E−06 | 0.029986 |
| KLF5 | 0.984511 | 8.36E−06 | 0.029986 |
| PAPPA2 | 1.416309 | 8.52E−06 | 0.029986 |
| PKP1 | 1.986776 | 1.29E−05 | 0.037916 |
| SLPI | 1.20008 | 3.25E−05 | 0.078277 |
| RHBDL2 | 2.44919 | 3.56E−05 | 0.078277 |
| CXCL6 | 1.472772 | 7.1E−05 | 0.138954 |

Additional differential expression analysis was performed on combined preeclampsia data sets for cohorts from Example 9 and current cohort totaling 72 preeclampsia cases and 452 controls.

Table 19 shows the top 13 differentially expressed genes for PE for the combined set. Notably, it was observed that PAPPA2 showed on the top with significant statistical significance after adjustment for multiple hypothesis correction.

TABLE 19
Top 13 Differentially Expressed Genes Predictive of
Preeclampsia (PE) in a combined cohort analysis

| Gene | P-value | P-value (adjusted) |
|---|---|---|
| PAPPA2 | 1.14E−10 | 3.82E−06 |
| FABP1 | 9.07E−09 | 3.05E−04 |
| SNORD14A | 1.56E−07 | 5.26E−03 |
| AOX1 | 3.01E−07 | 1.01E−02 |
| SALL1 | 3.29E−07 | 1.11E−02 |
| HP | 3.88E−07 | 1.30E−02 |
| KIAA1211L | 5.15E−07 | 1.73E−02 |
| OLFM4 | 6.29E−07 | 2.11E−02 |
| CLDN7 | 9.66E−07 | 3.25E−02 |
| ANXA1 | 4.43E−06 | 1.49E−01 |
| DNAH10 | 1.68E−05 | 5.63E−01 |
| GPSM2 | 3.02E−05 | 1.00E+00 |
| PKP1 | 1.23E−04 | 1.00E+00 |

To validate the preeclampsia prediction modeling, the PE data set (36 cases and 137 controls) from Example 9 was used for gene selection and training, and the modeling was tested for predictability using the current cohort (36 cases and 315 controls).

FIG. 25C shows a receiver-operator characteristic (ROC) curve for the preeclampsia prediction model, using all differentially expressed genes from top 10 expressed genes discovered in the training cohort. The mean area-under-the-curve (AUC) for the ROC curve for the training set was 0.75 and 0.66 for the test set, indicating a strong signal correlation.

Cross-validation PE modeling was performed on a combined cohort data set of 528 subjects. FIG. 25D shows a receiver-operator characteristic (ROC) curve for the preeclampsia prediction model, using all differentially expressed genes from Table 19. The mean area-under-the-curve (AUC) for the ROC curve was 0.76.

### Example 11: Prediction of Pre-Term Birth (PTB) on Combined Multiple Cohorts

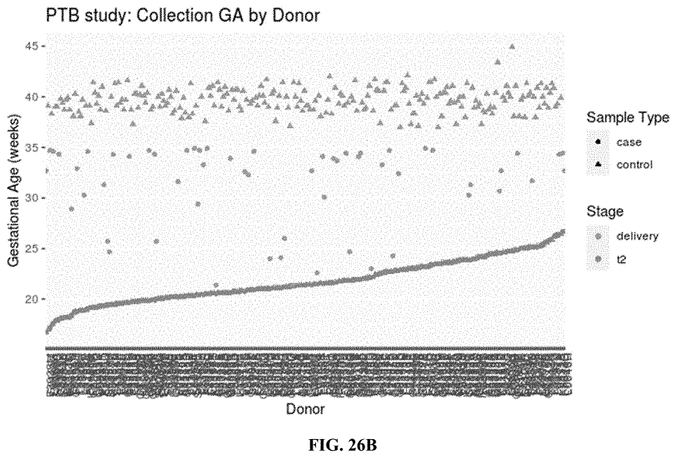

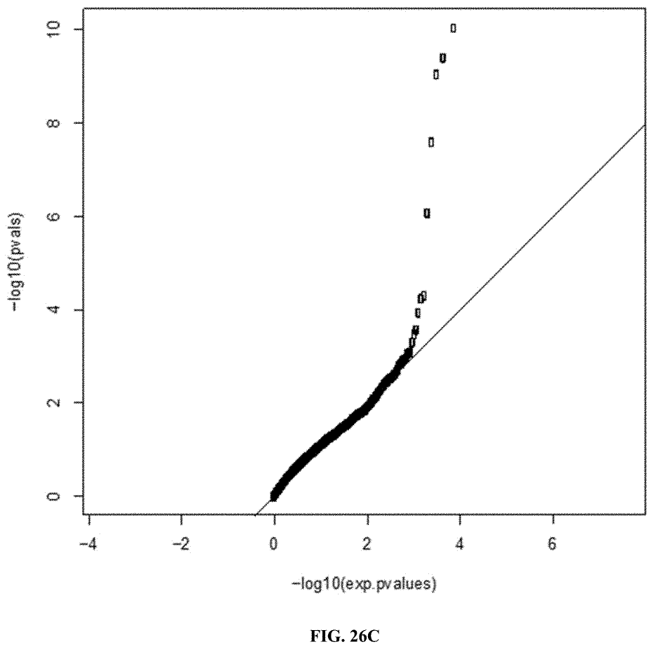

All PTB cohorts from Example 4 and Example 8 plus an additional cohort were combined in a single data set, as shown in FIG. 26A, totaling 255 case subjects with pre-term delivery before 38 weeks of gestation age and 796 healthy control subjects with delivery at gestational age after 38 weeks.

An additional cohort of subjects was obtained as follows. As shown in FIG. 26B, a cohort of 281 subjects (56 pre-term birth and 225 full-term controls) was established (with patient identification numbers shown on the x-axis). From this cohort, one or more biological samples (e.g., 1 or 2) were collected and assayed at different time points corresponding to an estimated gestational age (shown on the y-axis, in increasing order of estimated gestational age at delivery) of a fetus of each subject, using methods and systems of the present disclosure. For example, the estimated gestational age (shown on the y-axis) may be determined using methods such as ultrasound imaging, a last menstrual period (LMP) date, or a combination thereof, and may range from 0 to about 42 weeks.

In order to mitigate gestational age effects for blood collection, two separate differential expression analyses for combined cohorts were performed as follows. First, an analysis for differentially expressed genes between the pre-term birth case samples (delivered between 28 to 35 weeks) and control samples (delivered after 38 weeks) was performed for blood samples collected between 20 to 28 weeks of gestational age. In the second analysis, differentially expressed genes between the pre-term birth case samples (delivered between 28 to 35 weeks) and control samples (delivered after 38 weeks) were performed for blood samples collected between more narrow window of 23 to 28 weeks of gestational age.

Table 20 shows the top 9 differentially expressed genes for predicting pre-term births between 28 to 35 weeks with blood samples collected from subjects at between 20 to 28 weeks of gestational age, which showed significant statistical significance after adjustment for multiple hypothesis correction, and also showed a significant deviation from the null hypothesis in a QQ plot for differentially expressed in pre-term cases (as shown in FIG. 26C). Differential expression analysis was performed using EdgeR and accounting for ethnicity and cohort effects (113 PTB cases and 647 controls).

TABLE 20
Top set of genes that are predictive for preterm
births between 28-35 weeks with blood collected
between 20-28 weeks of gestational age

| Genes | logFC | Log2 fold change | P-value | FDR |
|---|---|---|---|---|
| APOB | −1.00993 | 2.099877 | 9.01E−11 | 1.02E−06 |
| FGA | −0.99345 | 1.545815 | 3.93E−10 | 2.23E−06 |
| FGB | −0.94881 | 1.60352 | 8.94E−10 | 3.38E−06 |
| HPD | −0.79382 | 1.627429 | 2.52E−08 | 7.15E−05 |
| ALB | −0.67556 | 5.147333 | 8.32E−07 | 0.001887 |
| CYP2E1 | −0.57371 | 1.757078 | 4.85E−05 | 0.091585 |
| FABP1 | −0.57173 | 2.092466 | 5.66E−05 | 0.091661 |
| OPA3 | 0.423862 | 1.482142 | 0.000113 | 0.160133 |
| TMEM56 | −0.38129 | 2.720486 | 0.000265 | 0.333199 |

Table 21 shows the top 11 differentially expressed genes for predicting pre-term births between 28 to 35 weeks with blood samples collected from subjects at between 23 to 28 weeks of gestational age, which showed significant statistical significance after adjustment for multiple hypothesis correction, and also showed a significant deviation from the null hypothesis in a QQ plot for differentially expressed in pre-term birth cases. Differential expression analysis was performed using EdgeR and accounting for ethnicity and cohort effects (73 PTB cases and 335 controls).

Only about half of the genes from Table 20 and Table 21 overlap, indicating a strong effect of gestational age at blood collection on the gene list that is predictive for pre-term birth.

TABLE 21
Top set of genes that are predictive for preterm birth between
28-35 weeks with blood collected between 23-28 week

| Genes | logFC | Log2 fold change | P-value | FDR |
|---|---|---|---|---|
| HRG | 1.3829 | 1.507414 | 2.45E−08 | 0.000283 |
| APOB | −0.9663 | 2.503944 | 2.93E−07 | 0.001692 |
| FGA | −0.98087 | 1.986942 | 1.11E−06 | 0.003309 |
| FGB | −0.98335 | 1.9955 | 1.15E−06 | 0.003309 |
| PAPPA2 | −0.89151 | 1.504208 | 3.73E−06 | 0.008605 |
| APOH | −0.98788 | 1.572287 | 1.02E−05 | 0.019636 |
| HPD | −0.78336 | 2.01557 | 2.4E−05 | 0.037305 |
| FGG | −0.9384 | 1.369466 | 2.58E−05 | 0.037305 |
| ALB | −0.71179 | 5.593431 | 7.75E−05 | 0.099401 |
| COL19A1 | −0.66394 | 1.852947 | 9.37E−05 | 0.108189 |

### Example 12: Prediction of GA on Combined Multiple Cohorts Using Training and Test Sets

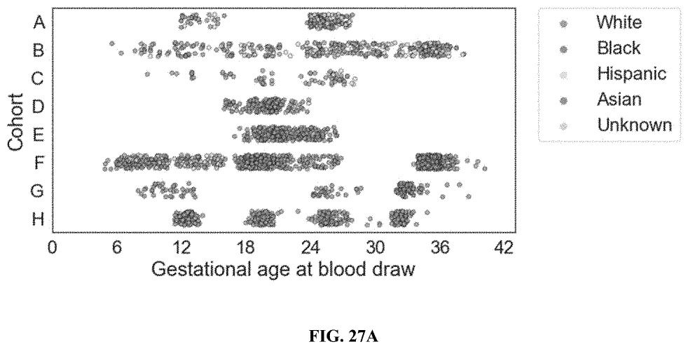

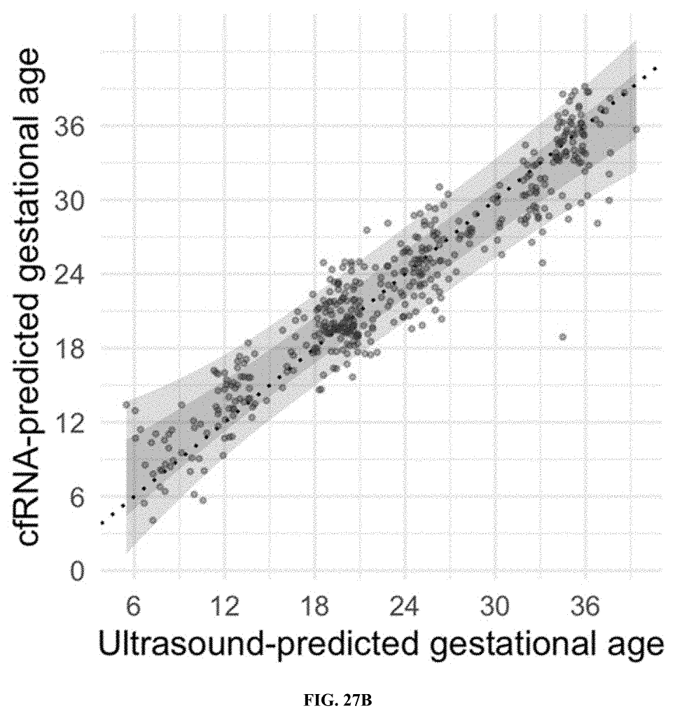

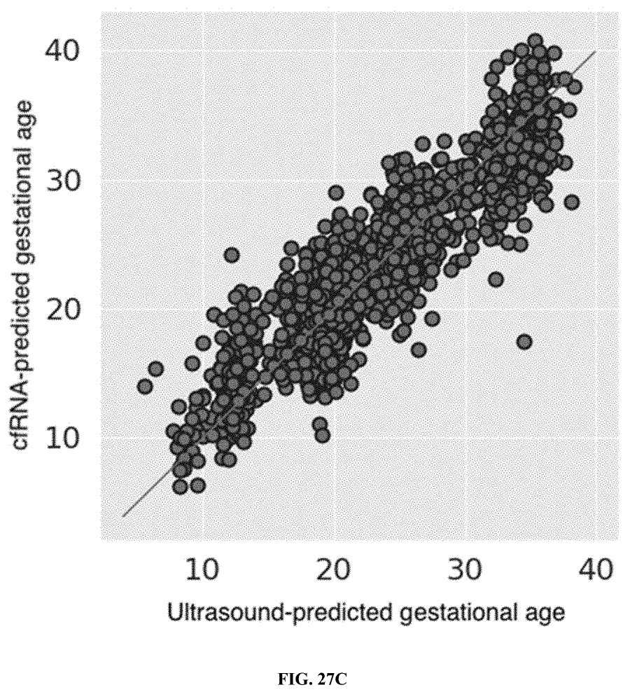

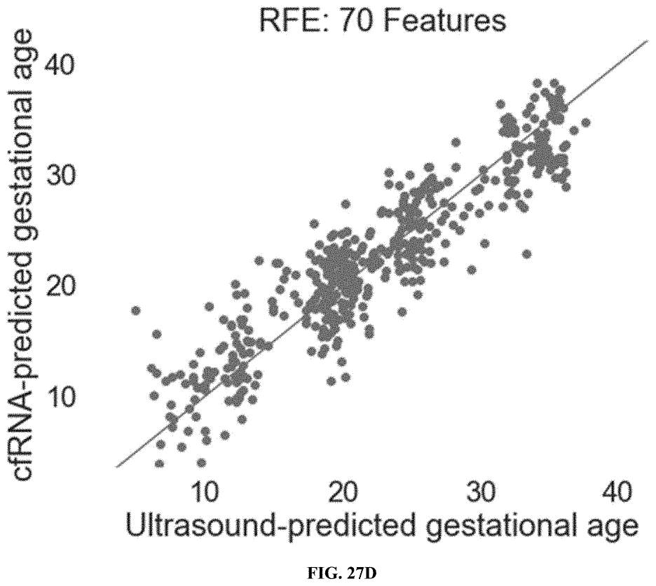

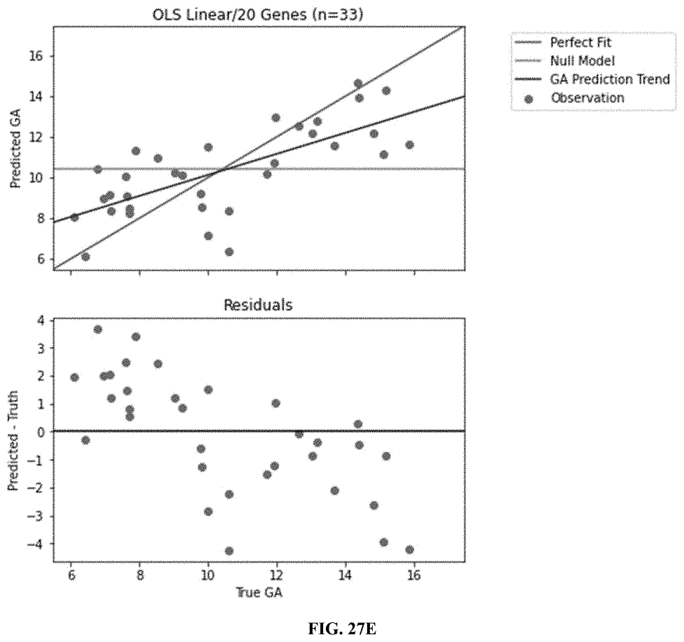

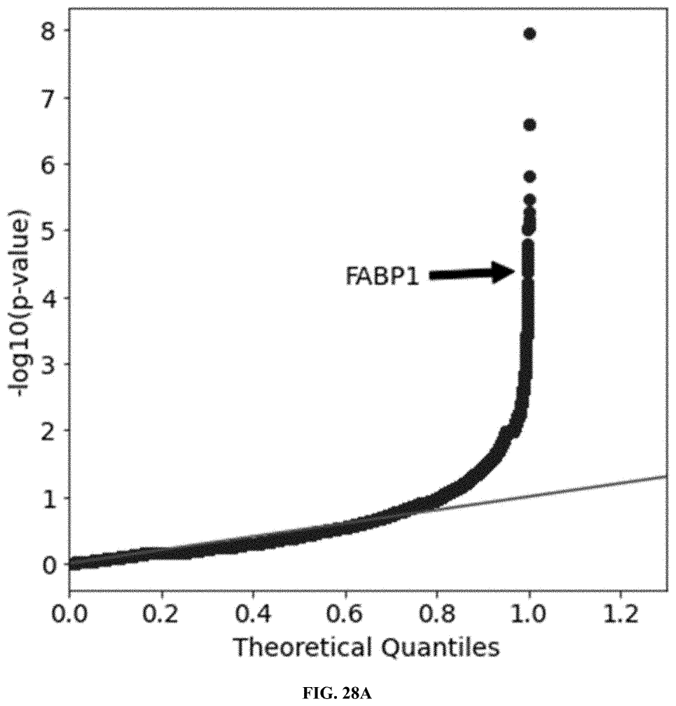

The gestational age cohort includes subjects from whom different sample types were collected for use in different studies, including studies for the prediction of actual gestational age of a fetus of each subject at the time of blood collection. All healthy pregnancy samples from retrospective cohorts presented in Examples 1-11 were combined in a single data set, as shown in FIG. 27A. By combining samples from 8 prospectively collected pregnancy cohorts, we amass a set of 2,428 plasma samples from 1,652 pregnancies across a diverse set of ethnicities and covering a broad range of gestational ages. Combined data demographic is represented in Table 22. The 8 different cohorts were treated as batches and a correction was applied prior to modeling of the data.

TABLE 22
Combined data set demographic

| | | | | | | | | Range of | | | | |
|---|---|---|---|---|---|---|---|---|---|---|---|---|
| | | | | | | | | Gesta- | Gesta- | | | |
| | | | | | | | | tional | tional | Gesta- | Pre- | Mother's |
| | | | | | % | | | Age at | Age at | tional | pregnancy | Age at |
| | | Passing | % | % | His- | % | % | Blood | Blood | Age at | Body Mass | Blood |
| | Cohort | Count | Asian | Black | panic | White | Unknown | Draw | Draw | Delivery | Index | Draw |
| 1 | A | 161 | 9.31 | 21.1 | 22.9 | 39.7 | 6.83 | 12-27.7 | 23.4 +/− 4.60 | 38.9 +/− 0.65 | 27.2 +/− 7.40 | 32.6 +/− 5.49 |
| 2 | B | 385 | 13.5 | 9.35 | 20 | 53.5 | 3.63 | 5.57-38.2 | 26.3 +/− 8.45 | 39.3 +/− 1.08 | 26.9 +/− 6.26 | 30.0 +/− 5.08 |
| 3 | C | 82 | 0.84 | 9.24 | 15.1 | 74.8 | 0 | 8.85-28.2 | 22.8 +/− 5.00 | 39.4 +/− 1.06 | 32.8 +/− 9.57 | 29.4 +/− 5.6 |
| 4 | D | 194 | 9.79 | 27.3 | 0 | 59.7 | 3.09 | 12.2-23.8 | 19.9 +/− 1.77 | 39.6 +/− 1.27 | 26.6 +/− 6.31 | 32.8 +/− 5.38 |
| 5 | E | 258 | 0 | 46.1 | 0 | 53.8 | 0 | 16.9-26.4 | 21.7 +/− 2.12 | 39.5 +/− 1.20 | 28.6 +/− 8.08 | 26.5 +/− 5.51 |
| 6 | F | 796 | 0.75 | 51.6 | 0 | 41.9 | 5.65 | 4.91-40.2 | 22.8 +/− 10.0 | 39.5 +/− 1.10 | 29.9 +/− 7.70 | 24.1 +/− 4.33 |
| 7 | G | 140 | 0 | 100 | 0 | 0 | 0 | 8-38.7 | 25.2 +/− 9.66 | 39.8 +/− 0.91 | 24.5 +/− 5.12 | — |
| 8 | H | 412 | 0 | 0 | 0 | 100 | 0 | 11.4-34.8 | 22.5 +/− 7.35 | 39.8 +/− 1.19 | 25.5 +/− 6.13 | 30.4 +/− 4.62 |

Three separate approaches were used to develop GA modeling based on combined cohorts.

In the first approach, the predicted gestational ages were generated using a predictive model for gestational age. The Lasso linear model predicts gestational age in the training set, with test set performance of a mean absolute error of 2.0 weeks, when using ultrasound estimated gestational age as ground truth. This model uses 494 genes listed in Table 23.

TABLE 23
Sets of 494 Genes Predictive for Gestational Age by Lasso linear model

| # | Gene | P-value | P-value adjusted | # | Gene | P-value | P-value adjusted |
|---|---|---|---|---|---|---|---|
| 1 | CAPN6 | 1.86E−303 | 1.21E−300 | 247 | C18orf54 | 1.31E−30 | 5.43E−28 |
| 2 | CSH1 | 1.86E−303 | 1.21E−300 | 248 | PLPP3 | 1.77E−30 | 7.33E−28 |
| 3 | CSHL1 | 1.86E−303 | 1.21E−300 | 249 | STAG3 | 2.10E−30 | 8.66E−28 |
| 4 | EXPH5 | 1.86E−303 | 1.21E−300 | 250 | CBR4 | 2.22E−30 | 9.12E−28 |
| 5 | HSD17B1 | 1.86E−303 | 1.21E−300 | 251 | GTSF1 | 4.17E−30 | 1.71E−27 |
| 6 | LGALS14 | 1.86E−303 | 1.21E−300 | 252 | ZSCAN21 | 1.06E−29 | 4.32E−27 |
| 7 | PAPPA | 1.86E−303 | 1.21E−300 | 253 | CRCP | 1.76E−29 | 7.16E−27 |
| 8 | SVEP1 | 1.86E−303 | 1.21E−300 | 254 | PROS2P | 2.25E−29 | 9.15E−27 |
| 9 | TACC2 | 1.86E−303 | 1.21E−300 | 255 | ALG11 | 2.46E−29 | 9.97E−27 |
| 10 | VGLL3 | 1.86E−303 | 1.21E−300 | 256 | PSG9 | 2.85E−29 | 1.15E−26 |
| 11 | HSD3B1 | 1.86E−303 | 1.21E−300 | 257 | ARL11 | 5.80E−29 | 2.34E−26 |
| 12 | NAPA | 1.26E−299 | 8.16E−297 | 258 | TRERF1 | 8.87E−29 | 3.57E−26 |
| 13 | CYP19A1 | 6.06E−289 | 3.93E−286 | 259 | SPATA6 | 1.25E−28 | 5.04E−26 |
| 14 | MYL12B | 6.60E−279 | 4.27E−276 | 260 | TNFSF8 | 1.75E−28 | 7.02E−26 |
| 15 | CSH2 | 2.72E−278 | 1.76E−275 | 261 | PCSK1 | 1.91E−28 | 7.62E−26 |
| 16 | PLAC4 | 5.84E−267 | 3.77E−264 | 262 | C12orf45 | 2.71E−28 | 1.08E−25 |
| 17 | BEX1 | 1.03E−259 | 6.64E−257 | 263 | ATF4P3 | 4.39E−28 | 1.75E−25 |
| 18 | OSTF1 | 1.62E−255 | 1.04E−252 | 264 | C15orf61 | 7.40E−28 | 2.94E−25 |
| 19 | CARD16 | 1.17E−246 | 7.52E−244 | 265 | CDCA4 | 8.76E−28 | 3.47E−25 |
| 20 | EFHD1 | 3.86E−242 | 2.47E−239 | 266 | ARHGAP42 | 9.61E−28 | 3.80E−25 |
| 21 | PHTF2 | 6.62E−239 | 4.24E−236 | 267 | IFT172 | 1.11E−27 | 4.38E−25 |
| 22 | TFAP2A | 2.13E−231 | 1.36E−228 | 268 | HCG4P5 | 1.19E−27 | 4.69E−25 |
| 23 | STAT1 | 4.67E−230 | 2.98E−227 | 269 | RPP25L | 2.95E−27 | 1.16E−24 |
| 24 | FNBP1L | 3.21E−228 | 2.05E−225 | 270 | SMAD1 | 3.82E−27 | 1.50E−24 |
| 25 | UBE2L6 | 1.39E−220 | 8.83E−218 | 271 | C11orf21 | 7.09E−27 | 2.77E−24 |
| 26 | NTAN1 | 9.12E−220 | 5.79E−217 | 272 | VASH1 | 1.09E−26 | 4.25E−24 |
| 27 | RBM3 | 6.17E−209 | 3.91E−206 | 273 | RNLS | 1.33E−26 | 5.17E−24 |
| 28 | ADAM12 | 7.37E−198 | 4.67E−195 | 274 | WDR25 | 1.39E−26 | 5.37E−24 |
| 29 | AP2S1 | 3.69E−196 | 2.33E−193 | 275 | LEMD3 | 2.21E−26 | 8.52E−24 |
| 30 | CDC37 | 1.39E−184 | 8.74E−182 | 276 | TMEM56-RWDD3 | 7.82E−26 | 3.01E−23 |
| 31 | NKIRAS2 | 1.36E−176 | 8.56E−174 | 277 | WIZ | 1.08E−25 | 4.17E−23 |
| 32 | CDC16 | 8.09E−175 | 5.09E−172 | 278 | TRIM62 | 1.09E−25 | 4.17E−23 |
| 33 | FRMD4B | 2.34E−173 | 1.47E−170 | 279 | UPRT | 1.29E−25 | 4.92E−23 |
| 34 | SKIL | 1.68E−171 | 1.05E−168 | 280 | TM2D2 | 1.59E−25 | 6.04E−23 |
| 35 | MMP8 | 1.57E−170 | 9.80E−168 | 281 | SPON2 | 1.91E−25 | 7.26E−23 |
| 36 | KRT8 | 2.82E−170 | 1.77E−167 | 282 | PTPRM | 2.17E−25 | 8.24E−23 |
| 37 | RAD23B | 2.76E−169 | 1.72E−166 | 283 | ADSSL1 | 1.62E−24 | 6.13E−22 |
| 38 | HIST1H2AI | 5.59E−164 | 3.48E−161 | 284 | PHLDA2 | 3.77E−24 | 1.42E−21 |
| 39 | ASNA1 | 1.07E−153 | 6.66E−151 | 285 | RRP1 | 3.81E−24 | 1.43E−21 |
| 40 | COMT | 2.70E−153 | 1.68E−150 | 286 | TMEM184B | 4.93E−24 | 1.85E−21 |
| 41 | CPT1A | 5.76E−153 | 3.57E−150 | 287 | METTL1 | 4.97E−24 | 1.86E−21 |
| 42 | COX17 | 2.71E−152 | 1.67E−149 | 288 | PFAS | 5.65E−24 | 2.11E−21 |
| 43 | GPC3 | 1.85E−150 | 1.14E−147 | 289 | MYO1B | 6.63E−24 | 2.47E−21 |
| 44 | GCNT1 | 2.61E−150 | 1.61E−147 | 290 | TMEM53 | 6.81E−24 | 2.53E−21 |
| 45 | REEP5 | 1.48E−149 | 9.10E−147 | 291 | DDX3Y | 8.21E−24 | 3.04E−21 |
| 46 | ZSWIM7 | 4.83E−144 | 2.97E−141 | 292 | ABL2 | 8.31E−24 | 3.07E−21 |
| 47 | RAP2A | 1.14E−143 | 7.00E−141 | 293 | PLAU | 1.25E−23 | 4.61E−21 |
| 48 | RAB6B | 2.30E−142 | 1.41E−139 | 294 | MON1A | 1.78E−23 | 6.54E−21 |
| 49 | KRT18 | 6.62E−138 | 4.05E−135 | 295 | DGAT2 | 2.59E−23 | 9.48E−21 |
| 50 | ACCSL | 3.97E−136 | 2.43E−133 | 296 | TMEM86B | 4.23E−23 | 1.54E−20 |
| 51 | ALDH2 | 1.44E−135 | 8.76E−133 | 297 | NR1D1 | 5.52E−23 | 2.01E−20 |
| 52 | FGA | 1.94E−135 | 1.18E−132 | 298 | F12 | 6.10E−23 | 2.21E−20 |
| 53 | MSR1 | 1.01E−134 | 6.12E−132 | 299 | FARP1 | 6.70E−23 | 2.43E−20 |
| 54 | CD36 | 1.91E−134 | 1.16E−131 | 300 | IFT81 | 9.06E−23 | 3.27E−20 |
| 55 | CD5L | 1.19E−133 | 7.20E−131 | 301 | KIAA1324 | 9.09E−23 | 3.27E−20 |
| 56 | SLC7A5 | 1.97E−131 | 1.19E−128 | 302 | NHLRC3 | 9.24E−23 | 3.32E−20 |
| 57 | NXF3 | 2.08E−129 | 1.26E−126 | 303 | PDSS1 | 1.09E−22 | 3.91E−20 |
| 58 | CAMP | 1.51E−128 | 9.08E−126 | 304 | CCDC107 | 1.39E−22 | 4.96E−20 |
| 59 | SERPINE1 | 1.29E−127 | 7.78E−125 | 305 | NETO1 | 1.64E−22 | 5.83E−20 |
| 60 | NREP | 6.93E−127 | 4.17E−124 | 306 | ASCL1 | 1.82E−22 | 6.48E−20 |
| 61 | KLF10 | 1.76E−126 | 1.05E−123 | 307 | GXYLT1 | 3.13E−22 | 1.11E−19 |
| 62 | TCN1 | 2.65E−126 | 1.59E−123 | 308 | PSG7 | 4.19E−22 | 1.48E−19 |
| 63 | FABP1 | 1.01E−120 | 6.06E−118 | 309 | ITPKC | 4.51E−22 | 1.59E−19 |
| 64 | CEACAM6 | 1.04E−119 | 6.19E−117 | 310 | BAG2 | 1.35E−21 | 4.72E−19 |
| 65 | GK | 1.52E−118 | 9.06E−116 | 311 | ERP27 | 1.56E−21 | 5.46E−19 |
| 66 | BCL2L15 | 1.56E−115 | 9.29E−113 | 312 | IPP | 1.81E−21 | 6.30E−19 |
| 67 | GNAI1 | 1.87E−115 | 1.11E−112 | 313 | GALNT7 | 4.39E−21 | 1.53E−18 |
| 68 | BEX4 | 1.24E−111 | 7.33E−109 | 314 | TXLNG | 8.89E−21 | 3.08E−18 |
| 69 | TEX9 | 4.76E−111 | 2.82E−108 | 315 | CYB5RL | 9.26E−21 | 3.20E−18 |
| 70 | PYGB | 9.74E−110 | 5.76E−107 | 316 | UBE3D | 1.01E−20 | 3.50E−18 |
| 71 | INHBA | 3.76E−109 | 2.22E−106 | 317 | CA3 | 1.40E−20 | 4.83E−18 |
| 72 | ARHGAP12 | 7.25E−109 | 4.27E−106 | 318 | WI2-1896O14.1 | 1.75E−20 | 6.01E−18 |
| 73 | PSMG2 | 1.11E−108 | 6.52E−106 | 319 | RRP9 | 2.10E−20 | 7.18E−18 |
| 74 | PZP | 1.67E−106 | 9.80E−104 | 320 | AC108488.4 | 2.25E−20 | 7.67E−18 |
| 75 | NUSAP1 | 1.67E−106 | 9.81E−104 | 321 | ZNF174 | 3.02E−20 | 1.03E−17 |
| 76 | EPSTI1 | 1.07E−105 | 6.27E−103 | 322 | IL16 | 4.41E−20 | 1.49E−17 |
| 77 | ELK3 | 1.47E−105 | 8.57E−103 | 323 | TXNDC15 | 4.41E−20 | 1.49E−17 |
| 78 | NPLOC4 | 3.62E−105 | 2.11E−102 | 324 | MCEE | 1.39E−19 | 4.68E−17 |
| 79 | ARL6IP1 | 5.19E−105 | 3.02E−102 | 325 | MSTO1 | 1.52E−19 | 5.10E−17 |
| 80 | TPPP3 | 2.26E−104 | 1.31E−101 | 326 | SCN9A | 2.27E−19 | 7.59E−17 |
| 81 | SLTM | 5.24E−104 | 3.04E−101 | 327 | YAP1 | 3.42E−19 | 1.14E−16 |
| 82 | TTK | 1.05E−101 | 6.07E−99 | 328 | AC012507.4 | 8.96E−19 | 2.98E−16 |
| 83 | SFT2D1 | 4.41E−100 | 2.55E−97 | 329 | AQP3 | 8.99E−19 | 2.99E−16 |
| 84 | CD209 | 4.85E−100 | 2.80E−97 | 330 | NEBL | 1.02E−18 | 3.38E−16 |
| 85 | DPM3 | 9.22E−100 | 5.31E−97 | 331 | ANGPT2 | 1.81E−18 | 5.98E−16 |
| 86 | CARHSP1 | 1.94E−99 | 1.12E−96 | 332 | DDX31 | 2.11E−18 | 6.95E−16 |
| 87 | KRT7 | 5.26E−99 | 3.02E−96 | 333 | E2F6 | 2.82E−18 | 9.24E−16 |
| 88 | KIF18B | 1.33E−97 | 7.64E−95 | 334 | YWHAZP3 | 3.74E−18 | 1.22E−15 |
| 89 | MCEMP1 | 1.50E−97 | 8.55E−95 | 335 | CYTOR | 5.21E−18 | 1.70E−15 |
| 90 | LATS2 | 9.93E−96 | 5.67E−93 | 336 | FBXO15 | 5.51E−18 | 1.79E−15 |
| 91 | AP5M1 | 1.30E−95 | 7.40E−93 | 337 | ZFP69 | 7.23E−18 | 2.34E−15 |
| 92 | SPCS3 | 4.66E−95 | 2.65E−92 | 338 | RCN2 | 7.47E−18 | 2.41E−15 |
| 93 | WDR7 | 8.65E−95 | 4.92E−92 | 339 | TMEM203 | 7.63E−18 | 2.46E−15 |
| 94 | CMBL | 1.17E−94 | 6.61E−92 | 340 | MEI1 | 7.71E−18 | 2.48E−15 |
| 95 | SCIN | 2.40E−93 | 1.36E−90 | 341 | PGAP2 | 7.77E−18 | 2.49E−15 |
| 96 | GFOD1 | 2.72E−93 | 1.54E−90 | 342 | MCCC1 | 1.04E−17 | 3.31E−15 |
| 97 | FAM32A | 3.19E−93 | 1.80E−90 | 343 | COX18 | 1.27E−17 | 4.03E−15 |
| 98 | DNAJC1 | 4.52E−93 | 2.54E−90 | 344 | LAMP5 | 1.75E−17 | 5.55E−15 |
| 99 | RIMKLB | 1.48E−92 | 8.34E−90 | 345 | FTH1P12 | 1.82E−17 | 5.76E−15 |
| 100 | GAS2L3 | 4.90E−92 | 2.75E−89 | 346 | MT1E | 2.79E−17 | 8.79E−15 |
| 101 | RUNDC3A | 9.20E−92 | 5.15E−89 | 347 | MEX3D | 4.57E−17 | 1.44E−14 |
| 102 | ASUN | 5.29E−91 | 2.95E−88 | 348 | TSGA10 | 4.69E−17 | 1.47E−14 |
| 103 | NQO2 | 6.74E−90 | 3.76E−87 | 349 | PDLIM1P1 | 5.57E−17 | 1.74E−14 |
| 104 | NFU1 | 1.54E−89 | 8.60E−87 | 350 | JADE3 | 7.26E−17 | 2.26E−14 |
| 105 | MTHFD1L | 2.59E−89 | 1.44E−86 | 351 | SPR | 1.60E−16 | 4.96E−14 |
| 106 | DPY19L1 | 2.69E−89 | 1.50E−86 | 352 | MYO18B | 1.77E−16 | 5.46E−14 |
| 107 | GCSAML | 1.01E−88 | 5.59E−86 | 353 | KISS1 | 2.49E−16 | 7.67E−14 |
| 108 | GLTP | 6.35E−88 | 3.51E−85 | 354 | METTL7A | 2.80E−16 | 8.60E−14 |
| 109 | CASP7 | 7.14E−88 | 3.94E−85 | 355 | CYB561D2 | 4.18E−16 | 1.28E−13 |
| 110 | CACUL1 | 3.87E−87 | 2.13E−84 | 356 | HLCS | 4.21E−16 | 1.29E−13 |
| 111 | ABCC1 | 4.99E−87 | 2.75E−84 | 357 | NAIF1 | 4.75E−16 | 1.44E−13 |
| 112 | FAM105A | 1.52E−86 | 8.33E−84 | 358 | EPHX2 | 5.90E−16 | 1.79E−13 |
| 113 | RAB3IL1 | 2.80E−86 | 1.54E−83 | 359 | COQ8B | 6.23E−16 | 1.88E−13 |
| 114 | PRKAR1B | 6.96E−86 | 3.80E−83 | 360 | MICA | 7.49E−16 | 2.25E−13 |
| 115 | TF | 7.30E−86 | 3.99E−83 | 361 | PPT2-EGFL8 | 8.88E−16 | 2.66E−13 |
| 116 | MORC4 | 1.74E−85 | 9.49E−83 | 362 | PNPLA1 | 1.09E−15 | 3.27E−13 |
| 117 | NIT2 | 3.38E−85 | 1.84E−82 | 363 | ALPK3 | 1.33E−15 | 3.96E−13 |
| 118 | TMEM91 | 5.90E−85 | 3.21E−82 | 364 | PTP4A3 | 2.34E−15 | 6.96E−13 |
| 119 | DIAPH3 | 5.82E−84 | 3.15E−81 | 365 | ZFP30 | 3.45E−15 | 1.02E−12 |
| 120 | KATNB1 | 1.60E−81 | 8.63E−79 | 366 | ZNF606 | 3.53E−15 | 1.04E−12 |
| 121 | ATP1B2 | 1.96E−80 | 1.06E−77 | 367 | ZNF229 | 4.74E−15 | 1.39E−12 |
| 122 | ZMIZ2 | 1.74E−79 | 9.38E−77 | 368 | MST1 | 6.33E−15 | 1.85E−12 |
| 123 | VSIG4 | 4.17E−79 | 2.24E−76 | 369 | RAB15 | 9.31E−15 | 2.72E−12 |
| 124 | GLB1 | 9.18E−79 | 4.93E−76 | 370 | TCL6 | 1.18E−14 | 3.44E−12 |
| 125 | SLC2A1 | 1.16E−78 | 6.22E−76 | 371 | TTLL1 | 1.36E−14 | 3.95E−12 |
| 126 | OSER1 | 4.09E−78 | 2.19E−75 | 372 | SKOR1 | 1.38E−14 | 3.98E−12 |
| 127 | AMIGO2 | 1.06E−77 | 5.65E−75 | 373 | KIAA0895L | 1.78E−14 | 5.14E−12 |
| 128 | NIPSNAP3B | 1.28E−77 | 6.80E−75 | 374 | CCDC58 | 2.61E−14 | 7.49E−12 |
| 129 | MAP2 | 2.19E−77 | 1.17E−74 | 375 | AMMECR1L | 3.17E−14 | 9.05E−12 |
| 130 | SMIM12 | 2.31E−76 | 1.23E−73 | 376 | C16orf96 | 3.31E−14 | 9.45E−12 |
| 131 | ACHE | 2.33E−76 | 1.24E−73 | 377 | IGF2 | 6.64E−14 | 1.89E−11 |
| 132 | DIAPH1 | 4.29E−75 | 2.27E−72 | 378 | CXorf40A | 1.01E−13 | 2.85E−11 |
| 133 | LYRM9 | 3.34E−73 | 1.76E−70 | 379 | ARSG | 1.07E−13 | 3.01E−11 |
| 134 | DYNLT3 | 8.40E−73 | 4.43E−70 | 380 | TMEM116 | 1.27E−13 | 3.56E−11 |
| 135 | KCNH2 | 2.81E−72 | 1.48E−69 | 381 | SPRY3 | 2.68E−13 | 7.50E−11 |
| 136 | GINS2 | 3.39E−72 | 1.78E−69 | 382 | BTN2A2 | 3.09E−13 | 8.64E−11 |
| 137 | MOSPD3 | 5.36E−72 | 2.81E−69 | 383 | FAM114A1 | 3.17E−13 | 8.80E−11 |
| 138 | PHF5A | 3.89E−70 | 2.03E−67 | 384 | C4orf48 | 3.65E−13 | 1.01E−10 |
| 139 | SLC16A7 | 1.58E−68 | 8.23E−66 | 385 | HACD1 | 4.11E−13 | 1.13E−10 |
| 140 | STX18 | 1.82E−68 | 9.49E−66 | 386 | DNAJB5 | 4.15E−13 | 1.14E−10 |
| 141 | ZMAT5 | 1.90E−68 | 9.86E−66 | 387 | WASH6P | 5.29E−13 | 1.45E−10 |
| 142 | APOL4 | 5.51E−68 | 2.86E−65 | 388 | GCSH | 9.75E−13 | 2.66E−10 |
| 143 | SLC7A11 | 1.17E−67 | 6.04E−65 | 389 | C12orf73 | 1.61E−12 | 4.37E−10 |
| 144 | CPNE4 | 6.51E−67 | 3.37E−64 | 390 | ABTB2 | 1.99E−12 | 5.40E−10 |
| 145 | NOP14 | 9.23E−67 | 4.76E−64 | 391 | KHK | 3.02E−12 | 8.14E−10 |
| 146 | PLPP1 | 1.67E−65 | 8.60E−63 | 392 | ZNF565 | 5.08E−12 | 1.37E−09 |
| 147 | FABP3 | 2.37E−65 | 1.22E−62 | 393 | DMD | 5.21E−12 | 1.40E−09 |
| 148 | BACE1 | 3.23E−65 | 1.66E−62 | 394 | LINC00853 | 7.39E−12 | 1.97E−09 |
| 149 | ITIH2 | 1.83E−63 | 9.36E−61 | 395 | CALML4 | 8.94E−12 | 2.38E−09 |
| 150 | HEXA | 7.34E−62 | 3.75E−59 | 396 | AC113189.5 | 9.23E−12 | 2.44E−09 |
| 151 | KIF16B | 1.03E−61 | 5.24E−59 | 397 | PDGFD | 9.52E−12 | 2.51E−09 |
| 152 | PTGER2 | 1.74E−61 | 8.87E−59 | 398 | RBPMS | 1.08E−11 | 2.84E−09 |
| 153 | HENMT1 | 1.81E−61 | 9.22E−59 | 399 | RERG | 2.78E−11 | 7.28E−09 |
| 154 | FAM149B1 | 4.19E−61 | 2.12E−58 | 400 | FAM84B | 2.83E−11 | 7.39E−09 |
| 155 | TMEM204 | 4.19E−60 | 2.12E−57 | 401 | GGTA1P | 2.84E−11 | 7.39E−09 |
| 156 | MOB3C | 2.79E−59 | 1.41E−56 | 402 | ZSCAN12 | 3.51E−11 | 9.10E−09 |
| 157 | ZBTB16 | 5.67E−59 | 2.86E−56 | 403 | FAT4 | 3.79E−11 | 9.78E−09 |
| 158 | MED16 | 1.81E−58 | 9.12E−56 | 404 | GOLGA8R | 8.50E−11 | 2.19E−08 |
| 159 | DDX58 | 2.08E−58 | 1.04E−55 | 405 | SHROOM2 | 8.51E−11 | 2.19E−08 |
| 160 | TESK1 | 2.95E−57 | 1.48E−54 | 406 | ZNF670 | 1.19E−10 | 3.04E−08 |
| 161 | OLR1 | 1.91E−56 | 9.53E−54 | 407 | ST7-AS1 | 1.24E−10 | 3.15E−08 |
| 162 | RBM14 | 2.65E−56 | 1.32E−53 | 408 | MXRA7 | 1.78E−10 | 4.50E−08 |
| 163 | TTC28 | 3.22E−56 | 1.60E−53 | 409 | ARHGAP22 | 1.81E−10 | 4.55E−08 |
| 164 | CEBPZOS | 6.36E−55 | 3.16E−52 | 410 | PHKA1 | 1.84E−10 | 4.61E−08 |
| 165 | IFIT1 | 7.00E−55 | 3.47E−52 | 411 | PLCE1 | 2.72E−10 | 6.81E−08 |
| 166 | PLBD2 | 7.06E−55 | 3.49E−52 | 412 | OAZ3 | 2.88E−10 | 7.17E−08 |
| 167 | FANCB | 8.81E−55 | 4.35E−52 | 413 | SMO | 3.71E−10 | 9.21E−08 |
| 168 | BCL2 | 1.12E−54 | 5.53E−52 | 414 | DOLK | 4.62E−10 | 1.14E−07 |
| 169 | UBXN11 | 9.85E−54 | 4.85E−51 | 415 | AMOT | 4.82E−10 | 1.19E−07 |
| 170 | SYPL1 | 1.22E−53 | 6.01E−51 | 416 | SLX4IP | 5.03E−10 | 1.23E−07 |
| 171 | CCDC15 | 1.51E−53 | 7.39E−51 | 417 | KLRC1 | 5.15E−10 | 1.26E−07 |
| 172 | IL15 | 3.13E−53 | 1.53E−50 | 418 | WDR90 | 5.21E−10 | 1.27E−07 |
| 173 | TMEM14A | 3.79E−53 | 1.85E−50 | 419 | ATP5L2 | 5.89E−10 | 1.42E−07 |
| 174 | METTL21EP | 1.89E−52 | 9.21E−50 | 420 | FBXL13 | 6.84E−10 | 1.65E−07 |
| 175 | DSEL | 5.57E−52 | 2.70E−49 | 421 | SIGLEC12 | 7.08E−10 | 1.70E−07 |
| 176 | STYXL1 | 4.94E−51 | 2.40E−48 | 422 | KCND3 | 9.17E−10 | 2.19E−07 |
| 177 | TMC1 | 1.10E−50 | 5.32E−48 | 423 | ABCB8 | 9.84E−10 | 2.34E−07 |
| 178 | SEC14L2 | 6.34E−50 | 3.06E−47 | 424 | AARS2 | 1.18E−09 | 2.79E−07 |
| 179 | IL1RAP | 3.85E−49 | 1.86E−46 | 425 | ARHGAP20 | 1.19E−09 | 2.81E−07 |
| 180 | CAPN11 | 3.96E−49 | 1.91E−46 | 426 | PRR4 | 1.23E−09 | 2.90E−07 |
| 181 | SEC22C | 4.44E−49 | 2.13E−46 | 427 | FBXO36 | 1.34E−09 | 3.15E−07 |
| 182 | PHF19 | 1.30E−48 | 6.24E−46 | 428 | GYPB | 1.50E−09 | 3.49E−07 |
| 183 | HSPBAP1 | 5.04E−48 | 2.41E−45 | 429 | RPP14 | 1.78E−09 | 4.14E−07 |
| 184 | EXOC6B | 2.62E−47 | 1.25E−44 | 430 | NUDT7 | 2.20E−09 | 5.09E−07 |
| 185 | KIF24 | 3.38E−47 | 1.61E−44 | 431 | NSUN3 | 3.12E−09 | 7.18E−07 |
| 186 | GLYATL1 | 1.01E−46 | 4.78E−44 | 432 | LRIG3 | 3.88E−09 | 8.89E−07 |
| 187 | ALDOC | 1.82E−46 | 8.61E−44 | 433 | TCEANC2 | 4.18E−09 | 9.54E−07 |
| 188 | PCBD1 | 2.04E−46 | 9.65E−44 | 434 | NME3 | 4.37E−09 | 9.92E−07 |
| 189 | UBBP4 | 4.64E−46 | 2.19E−43 | 435 | NEURL1 | 5.97E−09 | 1.35E−06 |
| 190 | MYO19 | 1.19E−45 | 5.62E−43 | 436 | MYL12AP1 | 1.32E−08 | 2.96E−06 |
| 191 | NUS1 | 3.27E−45 | 1.54E−42 | 437 | GRTP1 | 1.39E−08 | 3.12E−06 |
| 192 | CAV2 | 5.05E−45 | 2.37E−42 | 438 | PLS3 | 1.84E−08 | 4.11E−06 |
| 193 | HELLS | 8.27E−45 | 3.87E−42 | 439 | ZNF569 | 2.25E−08 | 5.00E−06 |
| 194 | PIGW | 9.54E−45 | 4.46E−42 | 440 | ZXDA | 2.49E−08 | 5.51E−06 |
| 195 | PSG3 | 5.19E−44 | 2.42E−41 | 441 | ENO2 | 2.93E−08 | 6.45E−06 |
| 196 | ABHD12 | 1.85E−43 | 8.60E−41 | 442 | CA4 | 3.57E−08 | 7.83E−06 |
| 197 | EFCAB2 | 2.09E−43 | 9.71E−41 | 443 | FAM161B | 4.46E−08 | 9.71E−06 |
| 198 | DUSP4 | 2.25E−43 | 1.04E−40 | 444 | SNX21 | 9.08E−08 | 1.97E−05 |
| 199 | FASN | 3.03E−43 | 1.40E−40 | 445 | SYTL2 | 1.03E−07 | 2.24E−05 |
| 200 | KDELC2 | 4.74E−43 | 2.19E−40 | 446 | PLCXD1 | 1.07E−07 | 2.29E−05 |
| 201 | ZMYM1 | 7.98E−43 | 3.67E−40 | 447 | TM9SF1 | 1.10E−07 | 2.36E−05 |
| 202 | PHKG2 | 2.23E−42 | 1.02E−39 | 448 | C17orf105 | 1.18E−07 | 2.51E−05 |
| 203 | VSTM1 | 2.36E−42 | 1.08E−39 | 449 | EIF1P3 | 1.91E−07 | 4.05E−05 |
| 204 | FCF1 | 4.12E−42 | 1.88E−39 | 450 | IL 1RAPL1 | 2.44E−07 | 5.14E−05 |
| 205 | NIPA1 | 4.57E−42 | 2.09E−39 | 451 | CASKIN2 | 2.72E−07 | 5.71E−05 |
| 206 | PPP2R3B | 8.37E−42 | 3.81E−39 | 452 | CYP2S1 | 3.13E−07 | 6.55E−05 |
| 207 | SEC14L5 | 1.63E−41 | 7.39E−39 | 453 | SNHG20 | 3.15E−07 | 6.55E−05 |
| 208 | BMT2 | 1.65E−41 | 7.47E−39 | 454 | SLC26A6 | 6.18E−07 | 0.000128 |
| 209 | SMIM20 | 2.01E−41 | 9.07E−39 | 455 | RPL23AP38 | 6.35E−07 | 0.000131 |
| 210 | MMP9 | 2.50E−41 | 1.13E−38 | 456 | CAMK4 | 7.60E−07 | 0.000156 |
| 211 | QPCT | 2.54E−41 | 1.14E−38 | 457 | KCNN4 | 8.94E−07 | 0.000182 |
| 212 | HTR2A | 3.15E−41 | 1.41E−38 | 458 | GCAT | 9.12E−07 | 0.000185 |
| 213 | CXCL16 | 6.34E−41 | 2.84E−38 | 459 | KIF7 | 1.87E−06 | 0.000378 |
| 214 | C19orf33 | 2.47E−40 | 1.11E−37 | 460 | NR4A2 | 3.86E−06 | 0.000776 |
| 215 | SPNS3 | 2.52E−40 | 1.13E−37 | 461 | FAM221A | 4.13E−06 | 0.000826 |
| 216 | C17orf53 | 6.25E−40 | 2.78E−37 | 462 | EEF1A1P11 | 4.53E−06 | 0.000902 |
| 217 | ZNHIT3 | 1.07E−39 | 4.75E−37 | 463 | FBXO40 | 4.58E−06 | 0.000906 |
| 218 | GLDC | 1.39E−39 | 6.17E−37 | 464 | GSTM1 | 5.41E−06 | 0.001066 |
| 219 | LURAP1L | 1.23E−38 | 5.45E−36 | 465 | SH3RF3 | 5.88E−06 | 0.001153 |
| 220 | RND3 | 3.19E−38 | 1.41E−35 | 466 | CD28 | 6.82E−06 | 0.001330 |
| 221 | ZNF554 | 3.35E−38 | 1.47E−35 | 467 | TRAV12-3 | 7.33E−06 | 0.001422 |
| 222 | WRAP73 | 4.75E−38 | 2.09E−35 | 468 | NHEJ1 | 7.47E−06 | 0.001441 |
| 223 | AP1G1 | 5.05E−38 | 2.21E−35 | 469 | ZNF19 | 8.37E−06 | 0.001606 |
| 224 | NDFIP2 | 6.04E−38 | 2.64E−35 | 470 | CCDC40 | 1.18E−05 | 0.002254 |
| 225 | PTENP1 | 1.10E−37 | 4.79E−35 | 471 | CH507-42P11.1 | 1.52E−05 | 0.002883 |
| 226 | SUSD6 | 1.20E−37 | 5.22E−35 | 472 | RPL34P27 | 1.56E−05 | 0.002946 |
| 227 | FAM212B | 1.96E−37 | 8.50E−35 | 473 | C9orf172 | 2.52E−05 | 0.004735 |
| 228 | DZIP1L | 4.10E−37 | 1.78E−34 | 474 | PPP1R9A | 2.87E−05 | 0.005360 |
| 229 | GABRE | 1.08E−36 | 4.68E−34 | 475 | CEP126 | 3.38E−05 | 0.006289 |
| 230 | RARRES1 | 6.15E−36 | 2.65E−33 | 476 | IL13RA2 | 3.83E−05 | 0.007083 |
| 231 | HSPA1B | 1.21E−35 | 5.18E−33 | 477 | FKBP14 | 3.91E−05 | 0.007186 |
| 232 | TCTA | 1.54E−35 | 6.59E−33 | 478 | FBXL6 | 4.62E−05 | 0.008460 |
| 233 | CD68 | 4.23E−35 | 1.81E−32 | 479 | PTPRH | 4.86E−05 | 0.008851 |
| 234 | POLR3B | 5.08E−35 | 2.17E−32 | 480 | GDPGP1 | 5.74E−05 | 0.010390 |
| 235 | ZNF79 | 3.84E−34 | 1.63E−31 | 481 | CFAP43 | 7.05E−05 | 0.012690 |
| 236 | B4GALT2 | 4.89E−34 | 2.08E−31 | 482 | CCDC73 | 7.35E−05 | 0.013158 |
| 237 | MYLIP | 1.28E−33 | 5.44E−31 | 483 | SBF2-AS1 | 7.62E−05 | 0.013571 |
| 238 | CAPN3 | 1.92E−33 | 8.11E−31 | 484 | CDH5 | 7.88E−05 | 0.013943 |
| 239 | FBXO28 | 2.20E−32 | 9.29E−30 | 485 | CCDC102A | 8.87E−05 | 0.015618 |
| 240 | ZNF226 | 2.82E−32 | 1.19E−29 | 486 | TMCO6 | 0.000109 | 0.019146 |
| 241 | ATP2B2 | 4.97E−32 | 2.09E−29 | 487 | TMEM217 | 0.000138 | 0.024093 |
| 242 | TAPBPL | 2.02E−31 | 8.45E−29 | 488 | NKD1 | 0.000140 | 0.024259 |
| 243 | CHMP6 | 2.50E−31 | 1.04E−28 | 489 | RP5-837I24.1 | 0.000169 | 0.028995 |
| 244 | ELOVL6 | 3.68E−31 | 1.54E−28 | 490 | RPL13AP6 | 0.000181 | 0.030876 |
| 245 | B4GALT7 | 3.68E−31 | 1.54E−28 | 491 | TJP3 | 0.000188 | 0.031989 |
| 246 | MRPL55 | 9.27E−31 | 3.85E−28 | 492 | CHCHD2P6 | 0.000190 | 0.032131 |
| 247 | C18orf54 | 1.31E−30 | 5.43E−28 | 493 | OLIG1 | 0.000247 | 0.041456 |
| 248 | PLPP3 | 1.77E−30 | 7.33E−28 | 494 | RN7SL5P | 0.000251 | 0.041953 |

FIG. 27B is a plot showing the relationship between a predicted gestational age (in weeks) and the measured gestational age (in weeks) for the subjects in the gestational age cohort in held-out test data. The error across the predicted range from 6 to 36 weeks is constant and does not show any correlation with GA. This is in contrast to ultrasound-based dating, which has a gradual increase in error as pregnancy progresses. Overall, the error of the model is equivalent to that of second trimester ultrasound and superior to third trimester. ANOVA analysis indicates most of the signal in the model is driven by RNA transcripts, and BMI, maternal age and race or ethnicity accounting for less than 0.5% of the signal. The gestational biomarkers model (e.g., prediction of gestational age based on a set of gestational age-associated biomarker genes) is independent of race or ethnicity.

In the second approach, whole transcriptome data from all healthy pregnancies was divided into a training set (1482 samples) and a held-out test set (495 samples), making sure to stratify by gestational age so all ranges are represented equally in training and held-out test sets.

Whole transcriptome data from the training set was subjected to a Lasso model. Table 24 shows the top 57 transcriptomic features for predicting predicted gestational ages in a training set generated using a Lasso method after restricting the space search to genes with average counts per million above 1 cpm. The model uses 54 genes and 3 additional transcriptomic features that are selected using Lasso to predict gestational age in test set performance of a mean absolute error of 2.33 weeks, when using ultrasound estimated gestational age as ground truth.

TABLE 24
Sets of 57 Transcriptomic Features Predictive
for Gestational Age by Lasso Method

| | | | | | BH- |
|---|---|---|---|---|---|
| | Transcriptomic | Feature | | | corrected |
| # | features | type | Correlation | P-value | P-value |
| 1 | CAPN6 | gene | 0.584328 | 2.04E−136 | 1.17E−134 |
| 2 | LGALS14 | gene | 0.556407 | 3.24E−121 | 9.23E−120 |
| 3 | SVEP1 | gene | 0.54131 | 1.40E−113 | 2.58E−112 |
| 4 | CSHL1 | gene | 0.541084 | 1.81E−113 | 2.58E−112 |
| 5 | EXPH5 | gene | 0.533408 | 9.75E−110 | 1.11E−108 |
| 6 | PAPPA | gene | 0.508472 | 2.97E−98 | 2.82E−97 |
| 7 | VGLL3 | gene | 0.489895 | 2.68E−90 | 2.19E−89 |
| 8 | BEX1 | gene | 0.489431 | 4.18E−90 | 2.98E−89 |
| 9 | TACC2 | gene | 0.450982 | 3.85E−75 | 2.44E−74 |
| 10 | STAT1 | gene | 0.419325 | 3.50E−64 | 1.99E−63 |
| 11 | PLAC4 | gene | 0.369908 | 2.87E−49 | 1.49E−48 |
| 12 | UBE2L6 | gene | 0.363607 | 1.52E−47 | 7.21E−47 |
| 13 | % ERCC | QC | −0.356695 | 1.07E−45 | 4.67E−45 |
| | | metrics | | | |
| 14 | CPNE2 | gene | 0.339643 | 2.46E−41 | 1.00E−40 |
| 15 | NXF3 | gene | 0.337411 | 8.77E−41 | 3.33E−40 |
| 16 | PAPPA2 | gene | 0.315658 | 1.21E−35 | 4.31E−35 |
| 17 | CSH1 | gene | 0.313818 | 3.15E−35 | 1.06E−34 |
| 18 | SLC7A5 | gene | 0.290907 | 2.71E−30 | 8.57E−30 |
| 19 | LTF | gene | 0.279006 | 6.65E−28 | 2.00E−27 |
| 20 | TMSB10P1 | gene | 0.273393 | 8.13E−27 | 2.32E−26 |
| 21 | SEC14L2 | gene | 0.271602 | 1.79E−26 | 4.85E−26 |
| 22 | SKIL | gene | 0.258285 | 5.16E−24 | 1.34E−23 |
| 23 | FABP1 | gene | 0.254356 | 2.58E−23 | 6.40E−23 |
| 24 | MEF2A | gene | 0.253145 | 4.22E−23 | 1.00E−22 |
| 25 | SLC7A11 | gene | 0.23882 | 1.15E−20 | 2.62E−20 |
| 26 | Unique_reads | QC | 0.229539 | 3.59E−19 | 7.88E−19 |
| | | metrics | | | |
| 27 | ANXA11 | gene | 0.186124 | 5.11E−13 | 1.08E−12 |
| 28 | IFIT1 | gene | 0.169894 | 4.62E−11 | 9.40E−11 |
| 29 | MYL12B | gene | 0.168367 | 6.90E−11 | 1.36E−10 |
| 30 | ANGPT2 | gene | −0.168225 | 7.17E−11 | 1.36E−10 |
| 31 | MCEMP1 | gene | 0.157461 | 1.10E−09 | 2.02E−09 |
| 32 | IGF2 | gene | −0.154093 | 2.48E−09 | 4.42E−09 |
| 33 | RNLS | gene | 0.153744 | 2.70E−09 | 4.66E−09 |
| 34 | MYCNOS | gene | 0.149773 | 6.89E−09 | 1.15E−08 |
| 35 | PSG3 | gene | 0.131688 | 3.63E−07 | 5.91E−07 |
| 36 | CXCR4 | gene | 0.124867 | 1.42E−06 | 2.25E−06 |
| 37 | JCHAIN | gene | −0.117279 | 5.99E−06 | 9.23E−06 |
| 38 | KLK1 | gene | −0.108699 | 2.75E−05 | 4.12E−05 |
| 39 | PLS3 | gene | −0.098127 | 1.55E−04 | 2.23E−04 |
| 40 | TNFAIP6 | gene | 0.098058 | 1.56E−04 | 2.23E−04 |
| 41 | DDX58 | gene | 0.089527 | 5.60E−04 | 7.78E−04 |
| 42 | IGHA1 | gene | −0.085325 | 1.01E−03 | 1.37E−03 |
| 43 | CH507-9B2.5 | gene | −0.082546 | 1.47E−03 | 1.95E−03 |
| 44 | RGPD2 | gene | −0.079216 | 2.27E−03 | 2.95E−03 |
| 45 | OIT3 | gene | −0.068552 | 8.29E−03 | 1.05E−02 |
| 46 | NR4A1 | gene | −0.065645 | 1.15E−02 | 1.42E−02 |
| 47 | CACUL1 | gene | −0.064953 | 1.24E−02 | 1.50E−02 |
| 48 | KISS1 | gene | 0.060214 | 2.04E−02 | 2.43E−02 |
| 49 | RASIP1 | gene | −0.060011 | 2.09E−02 | 2.43E−02 |
| 50 | CGA | gene | −0.059406 | 2.22E−02 | 2.53E−02 |
| 51 | CCDC15 | gene | 0.047547 | 6.73E−02 | 7.52E−02 |
| 52 | % | QC | −0.039872 | 1.25E−01 | 1.37E−01 |
| | mithocondrial | metrics | | | |
| | RNA | | | | |
| 53 | SH2D1B | gene | −0.030152 | 2.46E−01 | 2.65E−01 |
| 54 | PARGP1 | gene | 0.021481 | 4.09E−01 | 4.31E−01 |
| 55 | MYLIP | gene | 0.020002 | 4.42E−01 | 4.58E−01 |
| 56 | C18orf8 | gene | −0.018013 | 4.88E−01 | 4.97E−01 |
| 57 | PPM1H | gene | 0.016917 | 5.15E−01 | 5.15E−01 |

In the third approach, genes predictive of gestational age were identified by recursive feature elimination (RFE). A combined dataset of healthy individuals from 5 cohorts (cohorts with less than 100 samples were excluded, e.g. B, C, and F) was randomly split into 80% training (2390 samples) and 20% testing sets (478 samples) making sure to stratify by gestational age so all ranges are represented equally in training and held-out testing sets. Outliers identified by lab QC metrics were removed prior to modeling. Expression levels were converted to log 2 CPM levels. A linear model fit to gene features by ordinary least squares predicted gestational age at blood draw. Features were selected by performing feature ranking with RFE, which recursively reduces the feature set by pruning features with the least importance based on the estimated coefficients in the linear model. Prior to recursive feature elimination, gene features were filtered for transcripts whose expression levels had a minimum strength of relationship to gestational age. Spearman rank correlation coefficients were computed for the pairwise relationships of raw gene counts with gestational age at blood draw to assess the strength of each gene in predicting gestational age in the linear model. Based on the threshold set for the minimum Spearman rank correlation, e.g. 0.3, 0.4, 0.5, or 0.6, the whole transcriptome is down-selected to a pool of genes analyzed by RFE. A 5-fold cross validation tuned the hyperparameter with respect to the number of genes to target by RFE. The final linear model was trained on the training set by RFE set to the best number of genes identified by cross validation. Models were evaluated based on root mean squared error, mean absolute error (MAE), median absolute error performance between the estimated and observed gestational age on the testing dataset.

Table 25 shows the top 70 genes model identified for predicting predicted gestational ages in a training set generated using the RFE method with Spearman threshold of 0.4. This 70 gene linear model identified by RFE predicted gestational age in the testing set with a mean absolute error performance of 2.5 weeks, when using ultrasound estimated gestational age as ground truth.

TABLE 25
70 Genes from the Linear Model fit by
RFE Predictive for Gestational Age

| # | Gene | P-value |
|---|---|---|
| 1 | ALS2CR12 | 1.58E−05 |
| 2 | ANGPT2 | 2.18E−26 |
| 3 | APOBEC3G | 0.01150902 |
| 4 | BCAP29 | 0.00052699 |
| 5 | BLOC1S3 | 0.00011045 |
| 6 | C1orf115 | 1.31E−08 |
| 7 | CAPN6 | 1.14E−18 |
| 8 | CAPNS1 | 0.03519931 |
| 9 | CARMIL2 | 2.18E−05 |
| 10 | CBWD5 | 2.38E−05 |
| 11 | CEP152 | 0.00166964 |
| 12 | CGA | 4.40E−73 |
| 13 | CMC1 | 0.03732266 |
| 14 | CSH1 | 1.14E−17 |
| 15 | CSH2 | 0.00019274 |
| 16 | CXCR4 | 2.28E−08 |
| 17 | CYP19A1 | 9.74E−05 |
| 18 | DDX58 | 7.24E−15 |
| 19 | DYNLT3 | 1.87E−09 |
| 20 | EXPH5 | 5.48E−07 |
| 21 | FGG | 7.86E−16 |
| 22 | GCLC | 0.00401303 |
| 23 | GP9 | 2.05E−06 |
| 24 | GPR65 | 0.00102721 |
| 25 | HIST1H3G | 8.21E−09 |
| 26 | HMGB3 | 0.00977082 |
| 27 | HSPB1 | 0.0021566 |
| 28 | KISS1 | 3.52E−07 |
| 29 | KRT8 | 0.00010513 |
| 30 | KRTCAP2 | 9.90E−05 |
| 31 | LAP3 | 0.0004834 |
| 32 | LEMD3 | 3.36E−05 |
| 33 | LIMS1 | 5.85E−17 |
| 34 | LRSAM1 | 0.00082994 |
| 35 | MCM6 | 6.27E−05 |
| 36 | MCM9 | 8.71E−05 |
| 37 | MEIS1 | 0.00455709 |
| 38 | METTL7A | 0.0001903 |
| 39 | MICB | 0.00049999 |
| 40 | MIGA1 | 0.00308384 |
| 41 | MPLKIP | 0.00023848 |
| 42 | MS4A3 | 8.93E−10 |
| 43 | PAPPA | 6.57E−10 |
| 44 | PITHD1 | 2.54E−13 |
| 45 | PLAC4 | 5.82E−08 |
| 46 | PNKD | 0.00632914 |
| 47 | PRDX2 | 9.14E−08 |
| 48 | PSG3 | 6.65E−05 |
| 49 | PTGER2 | 0.00031855 |
| 50 | RGP1 | 0.02456697 |
| 51 | RN7SL1 | 0.00022625 |
| 52 | RNLS | 2.66E−05 |
| 53 | RRAGD | 4.00E−06 |
| 54 | RTTN | 0.00220346 |
| 55 | SIMC1 | 0.01018069 |
| 56 | SLC7A11 | 9.86E−06 |
| 57 | STAG3L3 | 9.77E−05 |
| 58 | STAT1 | 3.25E−27 |
| 59 | STOM | 9.27E−12 |
| 60 | SVEP1 | 7.84E−09 |
| 61 | TACC2 | 1.56E−05 |
| 62 | TAF3 | 0.00247011 |
| 63 | TBC1D22B | 0.00336354 |
| 64 | TCTA | 0.00020092 |
| 65 | TFEC | 0.01982375 |
| 66 | TPTEP1 | 2.08E−07 |
| 67 | TRERF1 | 0.00075604 |
| 68 | VGLL3 | 1.17E−08 |
| 69 | ZNF189 | 0.00149201 |
| 70 | ZNF79 | 0.00061504 |

FIG. 27D is a plot showing the concordance between a predicted gestational age (in weeks) and the measured gestational age (in weeks) for the subjects in the gestational age cohort in the held-out testing data for RFE gestation age modeling.

In the other approach, a linear regression model was developed to predict gestational age as a function of transcript expression levels in more narrow gestation age. A single cohort whole transcriptome dataset was collected focusing on the first trimester between 6-16 weeks. A single cohort whole transcriptome dataset was collected focusing on the first trimester. The data was split into 80% training data (164 samples) and 20% held-out testing data (33 samples), making sure to stratify by gestational age so all ranges are represented equally in training and held-out test sets. The training dataset was used in a 5-fold cross validation to select gene features and perform modeling with linear regression fit by ordinary least squares. Feature selection was performed by hierarchical clustering. First, the whole transcriptome was filtered based on a minimal magnitude of the Pearson correlation coefficient threshold to gestational age, e.g. |R|≥0.2 would reduce the genes to 3.7% of the whole transcriptome to 547 genes for clustering. The filtered genes are then clustered based on gene-to-gene similarity across the observations as calculated by pairwise Pearson correlation coefficients. A cutoff was then identified to trim the hierarchical clustering to reduce the features to a target number of clusters. A representative gene feature is the selected or computed for each cluster. Cluster representatives can be selected based on identifying a single gene with the largest Pearson correlation coefficient magnitude to gestational age or could be an aggregate measurement representing the mean or median of all genes within the cluster. In each round of cross validation, the identified features are then used to train a linear regression on the training folds and the model evaluated on the fold not used for training. The final features were identified based on the minimal RMSE performance between the observed and predicted gestational from the linear model.

Table 26 shows the 20 predictive genes for gestational age in a linear model as identified by hierarchical clustering. The linear model to predict gestational age in the first trimester (6 to 16 weeks) had a test set performance of a RMSE of 2.1 weeks, when using ultrasound estimated gestational age as ground truth.

TABLE 26
Set of 20 Genes Predictive for Gestational Age
identified by hierarchical clustering in samples
collected between 6-16 weeks of gestation.

| # | Gene | Pearson Correlation Coefficient |
|---|---|---|
| 1 | ARL6IP1 | 0.290774 |
| 2 | HMGB3 | 0.327823 |
| 3 | NLRC3 | −0.345206 |
| 4 | TRAF5 | −0.29844 |
| 5 | CD44 | −0.274007 |
| 6 | CSH1 | 0.713144 |
| 7 | CCDC157 | −0.301364 |
| 8 | ANLN | 0.328642 |
| 9 | RCHY1 | 0.256837 |
| 10 | PRRC2C | −0.270451 |
| 11 | CYFIP1 | 0.284176 |
| 12 | SERPINB1 | 0.294268 |
| 13 | GPR18 | −0.267355 |
| 14 | TRIM58 | 0.279979 |
| 15 | NCOA4 | 0.298769 |
| 16 | C1QA | 0.346268 |
| 17 | AMMECR1L | −0.261443 |
| 18 | GPC3 | 0.339435 |
| 19 | EOGT | −0.226626 |
| 20 | CTSB | 0.249796 |

FIG. 27E is a plot showing the concordance between a predicted gestational age (in weeks) and the measured gestational age (in weeks) for the subjects in the gestational age cohort in held-out test data in first trimester modeling.

### Example 13: Prediction of Preeclampsia (PE) Using Genes Selected by Medium-to-High Level Expression Genes

Further, whole transcriptome data from two cohorts described in Examples 9 and 10 were combined and analyzed by the abundant gene search method. The combined cohort of 541 samples contains 469 control samples with gestational age at blood draw of at least 17 weeks and delivery as low as 21 weeks of gestational age. Additionally, this combined cohort contains 72 case samples diagnosed with preeclampsia with gestational age at blood draw of at least 18 weeks and deliveries as early as 26 weeks of gestational age.

Logistic regression was performed to model the probability of preeclampsia in a pregnant individual from transcript expression data. Selection methods were applied to identify genes predictive of preeclampsia that are expressed at medium-to-high abundance. Genes were filtered based on a minimal median fold change of raw counts per gene between individuals with and without preeclampsia prior to modeling. One embodiment includes filtering for genes that have a median fold change in expression between case and control of <=0.5 and >1.5 to include abundant genes that are both upregulated and downregulated in preeclampsia. Additionally, genes are filtered to have a minimum number of reads across a set percentage of the training data. One embodiment filters genes with at least 5 reads in more than 50% of the training samples. These two filters are applied to reduce the transcriptome to an initial gene pool of abundant genes that are then ranked as features for the logistic model through recursive feature elimination (RFE). Prior to modeling, raw gene counts are converted to standardized log 2 CPM levels.

Nested resampling is performed to estimate the performance of abundant gene sets identified by RFE without data leakage between training and testing required to tune the best number of features to target by RFE. The outer resampling loop is used to test performance of logistic models trained on identified gene features by RFE whereas the inner resampling loop is used to tune the target number of features needed for RFE. The combined dataset of from 2 cohorts was randomly split one hundred times into 80% training (432 samples) and 20% held-out testing (109 samples) to comprise the outer resampling loop, making sure to stratify by case and control, gestational age, and cohort to ensure each are represented equally in both the training and held-out testing sets.

For each training and testing outer split, the training data was further split into 80% training (345 samples) and 20% held-out testing (87 samples) sets to comprise the inner resampling loop. This inner resampling split was randomly performed one hundred times to estimate the robustness of the gene features identified in a given training/testing split.

To identify the abundant gene features for a given inner training/testing dataset split, cross validation (CV) was performed on the inner resampling loop to identify the best number of features prior to training a logistic model on the outer training dataset. A 4-fold cross validation (CV) is performed on each inner training dataset to identify the best number of features for training a logistic model by RFE by maximizing the AUC performance on a test set. In each CV round, the target number of genes is optimized by performing RFE from 1 to a maximum number of features. In one embodiment, the maximum number of features was set to 20 to reduce overfitting given the size of the training dataset. A mean AUC is computed across the 4 CV test folds for each of the number of RFE features used, and the best number of features is selected based on the maximum mean AUC across the 4 CV folds. Then the full inner training set is used to train a logistic regression model by RFE with the best number of features to identify the abundant genes, and the AUC performance of the model is calculated on paired inner testing dataset. The frequency of abundant genes was computed across the one hundred random inner splits, and these data were filtered to generate the final gene features used to train a final logistic model on the outer training dataset. Performance of features sets were then compared by evaluating the trained logistic models on the held-out outer testing dataset. Cutoffs to identify gene features include selection based on most frequently observed across the inner loops, e.g. selecting the top two most frequently identified genes, or based on those abundant genes that showed significant differential expression between preeclampsia cases versus controls as computed by the Mann-Whitney rank test with p-values corrected for multiple tests via the Holm step-down method using Bonferroni adjustments.

Table 27 shows the 132 genes identified in the abundant gene search across the one hundred inner resampling training and test splits.

TABLE 27
132 genes identified in the abundant gene search across the
one hundred inner resampling training and test splits.

| # | Gene | P-value_mw | P-value_adjusted_holm |
|---|---|---|---|
| 1 | FABP1 | 6.23E−07 | 8.23E−05 |
| 2 | CDCA2 | 3.14E−06 | 0.00041104 |
| 3 | HMGB3 | 0.00010898 | 0.01416703 |
| 4 | ELANE | 0.00012196 | 0.01573288 |
| 5 | CDC20 | 0.00015193 | 0.01944651 |
| 6 | SHCBP1 | 0.00020189 | 0.02563957 |
| 7 | OLFM4 | 0.00027466 | 0.03460665 |
| 8 | S100A9 | 0.00034386 | 0.04298208 |
| 9 | S100A12 | 0.00039749 | 0.04928901 |
| 10 | STK33 | 0.00045608 | 0.05609825 |
| 11 | PLS1 | 0.00046166 | 0.056323 |
| 12 | APOB | 0.00048905 | 0.05917536 |
| 13 | PCNA | 0.00121359 | 0.14563076 |
| 14 | S100A16 | 0.0014132 | 0.16817071 |
| 15 | DEFA3 | 0.00142513 | 0.16817071 |
| 16 | PLEKHA6 | 0.00201857 | 0.23617235 |
| 17 | CDR1-AS | 0.00216043 | 0.25060948 |
| 18 | KIF20A | 0.00229895 | 0.26437936 |
| 19 | CLC | 0.00244557 | 0.27879471 |
| 20 | PEG10 | 0.00256623 | 0.28998356 |
| 21 | CEACAM6 | 0.00294602 | 0.32995372 |
| 22 | HIST1H3G | 0.00297726 | 0.3304754 |
| 23 | KIF18B | 0.00308089 | 0.3388975 |
| 24 | ABCA13 | 0.00325526 | 0.35482292 |
| 25 | PRDM5 | 0.00344753 | 0.37233343 |
| 26 | KRT23 | 0.004504 | 0.48192809 |
| 27 | PLAC4 | 0.00461967 | 0.48968489 |
| 28 | CEACAM8 | 0.00465489 | 0.48968489 |
| 29 | HIST1H2BM | 0.00482249 | 0.50153917 |
| 30 | TRMT10A | 0.00485911 | 0.50153917 |
| 31 | CAMP | 0.00543939 | 0.55481806 |
| 32 | TCN1 | 0.0058169 | 0.58750665 |
| 33 | SULT1B1 | 0.00594789 | 0.59478851 |
| 34 | RETN | 0.00617211 | 0.61103934 |
| 35 | HIST1H4H | 0.00679116 | 0.66553325 |
| 36 | MGST1 | 0.00759263 | 0.73648489 |
| 37 | BPI | 0.00790964 | 0.75932584 |
| 38 | MYO1B | 0.00833748 | 0.79206037 |
| 39 | RNASE2 | 0.00903946 | 0.84970968 |
| 40 | PLK1 | 0.00908236 | 0.84970968 |
| 41 | FOXM1 | 0.00927762 | 0.85354118 |
| 42 | HIST1H2AH | 0.00988609 | 0.89963399 |
| 43 | ENSG00000188206 | 0.01021538 | 0.91938418 |
| 44 | MMP8 | 0.01100497 | 0.97944234 |
| 45 | NLRP2 | 0.01147255 | 1 |
| 46 | CTSG | 0.0121512 | 1 |
| 47 | ANXA3 | 0.01243247 | 1 |
| 48 | AKR1C3 | 0.01349336 | 1 |
| 49 | KLRG1 | 0.01352394 | 1 |
| 50 | TEK | 0.01389568 | 1 |
| 51 | AC078883.3 | 0.01389568 | 1 |
| 52 | SELENOP | 0.01408491 | 1 |
| 53 | TRPM6 | 0.01443775 | 1 |
| 54 | ARG1 | 0.01450273 | 1 |
| 55 | CEACAM1 | 0.01460069 | 1 |
| 56 | ROBO1 | 0.01473221 | 1 |
| 57 | AZU1 | 0.01493144 | 1 |
| 58 | CLIC5 | 0.01496488 | 1 |
| 59 | CHMP4C | 0.01499838 | 1 |
| 60 | FCGR1A | 0.01705805 | 1 |
| 61 | ALPK3 | 0.01724672 | 1 |
| 62 | LTF | 0.01857887 | 1 |
| 63 | U2AF1 | 0.01861938 | 1 |
| 64 | ALDH1L2 | 0.01886405 | 1 |
| 65 | MPO | 0.02240514 | 1 |
| 66 | PRTN3 | 0.02352466 | 1 |
| 67 | BCL6B | 0.02397577 | 1 |
| 68 | SMAD5 | 0.02428066 | 1 |
| 69 | JAKMIP1 | 0.02751905 | 1 |
| 70 | TNNT1 | 0.03006317 | 1 |
| 71 | CDH6 | 0.03347483 | 1 |
| 72 | PHGDH | 0.03381315 | 1 |
| 73 | DSP | 0.03540731 | 1 |
| 74 | HIST1H2AL | 0.03583358 | 1 |
| 75 | AFMID | 0.03691843 | 1 |
| 76 | PGLYRP1 | 0.03736014 | 1 |
| 77 | ASL | 0.04310444 | 1 |
| 78 | MUC3A | 0.0442874 | 1 |
| 79 | ME1 | 0.04514905 | 1 |
| 80 | SNAPC2 | 0.04576058 | 1 |
| 81 | LAMP5 | 0.0471846 | 1 |
| 82 | PHACTR1 | 0.0480934 | 1 |
| 83 | MYOM2 | 0.04836889 | 1 |
| 84 | PRR16 | 0.05207253 | 1 |
| 85 | HACD3 | 0.05590646 | 1 |
| 86 | JUN | 0.05877114 | 1 |
| 87 | CEBPE | 0.06063659 | 1 |
| 88 | MS4A3 | 0.06097083 | 1 |
| 89 | METTL17 | 0.07353507 | 1 |
| 90 | KCNN3 | 0.07471534 | 1 |
| 91 | TCL1A | 0.07604486 | 1 |
| 92 | MRAS | 0.07739361 | 1 |
| 93 | FMO2 | 0.07931455 | 1 |
| 94 | STEAP1B | 0.07945323 | 1 |
| 95 | SERPINB10 | 0.08042952 | 1 |
| 96 | MT-TI | 0.08241133 | 1 |
| 97 | TMEM176B | 0.0884438 | 1 |
| 98 | FPR3 | 0.08859527 | 1 |
| 99 | MT-TT | 0.11415812 | 1 |
| 100 | MT-TG | 0.12956794 | 1 |
| 101 | CTSW | 0.14995411 | 1 |
| 102 | RSAD1 | 0.15133406 | 1 |
| 103 | RELN | 0.17681601 | 1 |
| 104 | SLC43A2 | 0.17995066 | 1 |
| 105 | CHI3L1 | 0.18661349 | 1 |
| 106 | BTBD11 | 0.18932905 | 1 |
| 107 | SULT1A1 | 0.20048273 | 1 |
| 108 | ALPL | 0.24393954 | 1 |
| 109 | RPL23AP7 | 0.25526013 | 1 |
| 110 | DDAH1 | 0.26624377 | 1 |
| 111 | MT-TC | 0.27540426 | 1 |
| 112 | RIPK3 | 0.28223297 | 1 |
| 113 | RPL23AP82 | 0.28623848 | 1 |
| 114 | VSIG4 | 0.33770179 | 1 |
| 115 | DDX11L10 | 0.35259587 | 1 |
| 116 | FFAR2 | 0.42464406 | 1 |
| 117 | BTLA | 0.43505175 | 1 |
| 118 | FOSB | 0.46417303 | 1 |
| 119 | FCGBP | 0.46714367 | 1 |
| 120 | GSTM1 | 0.48114512 | 1 |
| 121 | TLE1P1 | 0.50050691 | 1 |
| 122 | GSTA1 | 0.50205287 | 1 |
| 123 | SORBS2 | 0.50722428 | 1 |
| 124 | SERTAD3 | 0.514511 | 1 |
| 125 | MMP25 | 0.52290481 | 1 |
| 126 | RPL23AP97 | 0.55662534 | 1 |
| 127 | OVOS2 | 0.55771295 | 1 |
| 128 | TRHDE | 0.61336971 | 1 |
| 129 | RAP1GAP | 0.61450747 | 1 |
| 130 | HLA-DQA2 | 0.69692228 | 1 |
| 131 | CTD-3088G3.8 | 0.81560517 | 1 |
| 132 | EMCN | 0.92709603 | 1 |

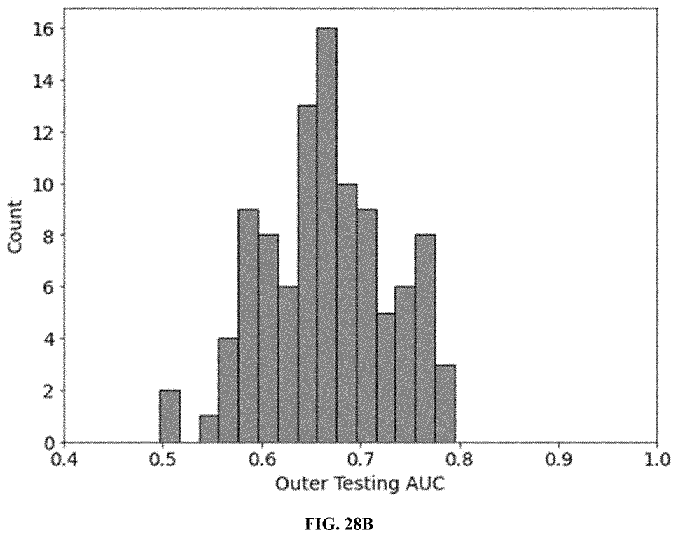

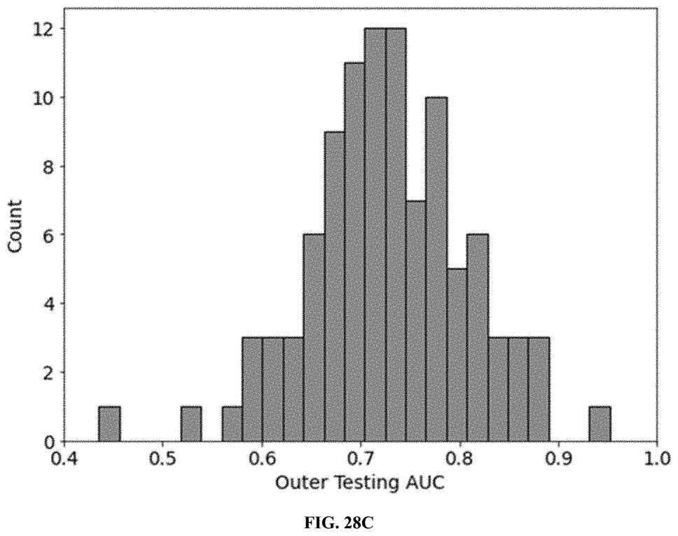

FABP1 was among the top significantly expressed genes for both Examples 9 and 10 and this analysis. It was observed that FABP1 showed significant statistical significance after adjustment for multiple hypothesis correction, and also showed a significant deviation from the null hypothesis in a QQ plots for differentially expressed in PE (as shown in FIG. 28A).

To evaluate the preeclampsia prediction modeling, the multiples splits of PE data into 80% training and 20% held-out testing (87 samples) were used to build predictive linear modeling with estimation of AUC on testing sets. Single FABP1 gene modeling in one hundreds splits produced the area-under-the-curve (AUC) for the ROC curve values with mean at 0.67 (FIG. 28B).

Combining best gene PAPPA2 from Examples 9 and 10 with the nine abundant genes include FABP1, CDCA2, HMGB3, ELANE, CDC20, SHCBP1, OLFM4, S100A9, S100A12 with significant differential expression (adjusted p-value<0.05) from Table 27 provide significant increase in predictive modeling with the mean AUC across the outer testing sets is 0.73 (FIG. 28C)

### Example 14: Detection and Monitoring Fetal Organ Development in Mother Plasma Across Pregnancy Progression Using Gene Sets

Using systems and methods of the present disclosure, a method of detection and measurement of the fetal organ transcriptional RNA signals in mother plasma were developed to monitor various fetal developmental stages during pregnancy.

The transcriptome data obtained from cohorts A, B, G and H as described in Example 12 (FIG. 27A) were split into a training set (cohort H) and a held-out test set (cohorts A, B, and G). The training set contains four longitudinal blood samples per subject collected at approximate gestational ages of 12, 20, 25 and 32 weeks.

Cell-type specific gene sets represented in Table 28 were derived from a publicly available database of gene ontologies (gsea-msigdb.org) and used to identify the fetal organ development signal in plasma of pregnant subjects.

TABLE 28
Cell-type specific gene set collections (C8)
used in the gene set enrichment analysis

| | Number of | | |
|---|---|---|---|
| Focus organ | cell types | Adult or fetal | PMID |
| Liver | 31 | adult | 31292543 |
| Developing heart | 25 | Fetal 5-25 w | 31292543 |
| Olfactory | 26 | adult | 32066986 |
| Embryonic cortex | 31 | fetal 22-23 w | 29867213 |
| Esophagus | 4 | fetal 25 w | 29802404 |
| Large intestine | 9 | fetal 24 w | 29802404 |
| Large intestine | 7 | adult | 29802404 |
| Small intestine | 7 | fetal 24 w | 29802404 |
| Stomach | 5 | fetal 24 w | 29802404 |
| Bone marrow | 29 | adult | 30243574 |
| Fetal retina | 11 | fetal 5-25 w | 31269016 |
| Kidney | 30 | adult | 31249312 |
| Kidney | 11 | fetal 12-19 w | 30166318 |
| Midbrain | 26 | fetal and progenitor | 27716510 |
| Pancreas | 9 | adult | 27693023 |
| Cord blood | 10 | adult and progenitor | 29545397 |
| Prefrontal cortex | 31 | fetal 8-26 w | 29539641 |

Samples collected from early and late pregnancy (12 and 32 weeks, respectively) were compared across 302 cell-type specific gene sets (Table 28). 80 of those gene sets were identified as significantly enriched, including 31 upregulated and 4 downregulated fetal cell types (Table 29). Discovered gene sets associated with cell participating in fetal organ development of heart, large and small intestine, retina, prefrontal cortex, midbrain, kidney, and esophagus. To further evaluate changes in activity of significantly enriched fetal organ gene sets in the course of pregnancy, normalized transcriptome fraction for each of the sets was calculated for every cfRNA sample and the fraction was modeled as a linear function of the recorded gestational age. As a result, 19 out of those 31 significantly enriched fetal gene sets were found to have significant temporal upward trends along the pregnancy timeline, and 3 out 4—significant downward trend.

TABLE 30
Fetal organ gene sets significantly enriched in the comparison between samples collected at 32 and 12 weeks
of gestation age; P-value was adjusted using Benjamini-Hochberg correction; NES (normalized enrichment score)

| | P-value | | |
|---|---|---|---|
| Gene set | adjusted | NES3 | Trend |
| CUI_DEVELOPING_HEART_C6_EPICARDIAL_CELL | 1.46E−03 | 1.67 | upward |
| CUI_DEVELOPING_HEART_C8_MACROPHAGE | 4.17E−06 | 1.75 | upward |
| FAN EMBRYONIC CTX BIG GROUPS CAJAL RETZIUS | 1.11E−03 | 1.49 | upward |
| FAN_EMBRYONIC_CTX_BIG_GROUPS_MICROGLIA | 1.37E−09 | 1.9 | upward |
| FAN_EMBRYONIC_CTX_MICROGLIA_1 | 1.37E−09 | 2.43 | upward |
| FAN_EMBRYONIC_CTX_MICROGLIA_3 | 7.12E−03 | 1.78 | upward |
| FAN_EMBRYONIC_CTX_NSC_2 | 1.37E−09 | 2.3 | upward |
| GAO_LARGE_INTESTINE_24W_C11_PANETH_LIKE_CELL | 1.46E−03 | 1.51 | upward |
| GAO_SMALL_INTESTINE_24W_C3_ENTEROCYTE_PROGENITOR_SUBTYPE_1 | 3.90E−04 | 1.93 | upward |
| GAO_SMALL_INTESTINE_24W_C4_ENTEROCYTE_PROGENITOR_SUBTYPE_2 | 3.33E−06 | 2.06 | upward |
| HU_FETAL_RETINA_BLOOD | 2.91E−08 | 1.89 | upward |
| HU_FETAL_RETINA_MICROGLIA | 8.18E−09 | 1.8 | upward |
| HU_FETAL_RETINA_RGC | 1.23E−04 | 1.57 | upward |
| HU_FETAL_RETINA_RPC | 6.55E−03 | 1.63 | upward |
| HU_FETAL_RETINA_RPE | 8.32E−03 | 1.48 | upward |
| MANNO MIDBRAIN NEUROTYPES HMGL | 2.37E−05 | 1.53 | upward |
| MANNO_MIDBRAIN_NEUROTYPES_HNPROG | 3.93E−04 | 1.73 | upward |
| MANNO_MIDBRAIN_NEUROTYPES_HPROGBP | 1.37E−09 | 2 | upward |
| MANNO MIDBRAIN NEUROTYPES HPROGFPL | 1.37E−09 | 2.03 | upward |
| MANNO MIDBRAIN NEUROTYPES HPROGFPM | 3.02E−08 | 1.86 | upward |
| MANNO_MIDBRAIN_NEUROTYPES_HPROGM | 4.56E−06 | 1.79 | upward |
| MENON_FETAL_KIDNEY_5_PROXIMAL_TUBULE_CELLS | 2.36E−03 | 1.69 | upward |
| MENON_FETAL_KIDNEY_7_LOOPOF_HENLE_CELLS_DISTAL | 4.13E−05 | 1.71 | upward |
| MENON_FETAL_KIDNEY_8_CONNECTING_TUBULE_CELLS | 9.01E−03 | 1.49 | upward |
| ZHONG_PFC_C1_MICROGLIA | 1.37E−09 | 2.02 | upward |
| ZHONG_PFC_C1_OPC | 1.37E−09 | 2.31 | upward |
| ZHONG_PFC_C2_UNKNOWN_NPC | 1.37E−09 | 2.31 | upward |
| ZHONG PFC C3 UNKNOWN INP | 4.25E−04 | 1.96 | upward |
| ZHONG_PFC_C8_ORG_PROLIFERATING | 3.96E−07 | 2.15 | upward |
| ZHONG_PFC_MAJOR_TYPES_MICROGLIA | 4.24E−08 | 1.75 | upward |
| ZHONG_PFC_MAJOR_TYPES_NPCS | 1.37E−09 | 2.17 | upward |
| ZHONG_PFC_C4_UNKNOWN_INP | 5.28E−03 | −1.82 | downward |
| FAN_EMBRYONIC_CTX_BRAIN_B_CELL | 5.32E−03 | −1.6 | downward |
| GAO_ESOPHAGUS_25W_C4_FGFR1HIGH_EPITHELIAL_CELLS | 5.81E−03 | −1.42 | downward |
| MENON_FETAL_KIDNEY_2_NEPHRON_PROGENITOR_CELLS | 7.23E−03 | −0.91 | downward |

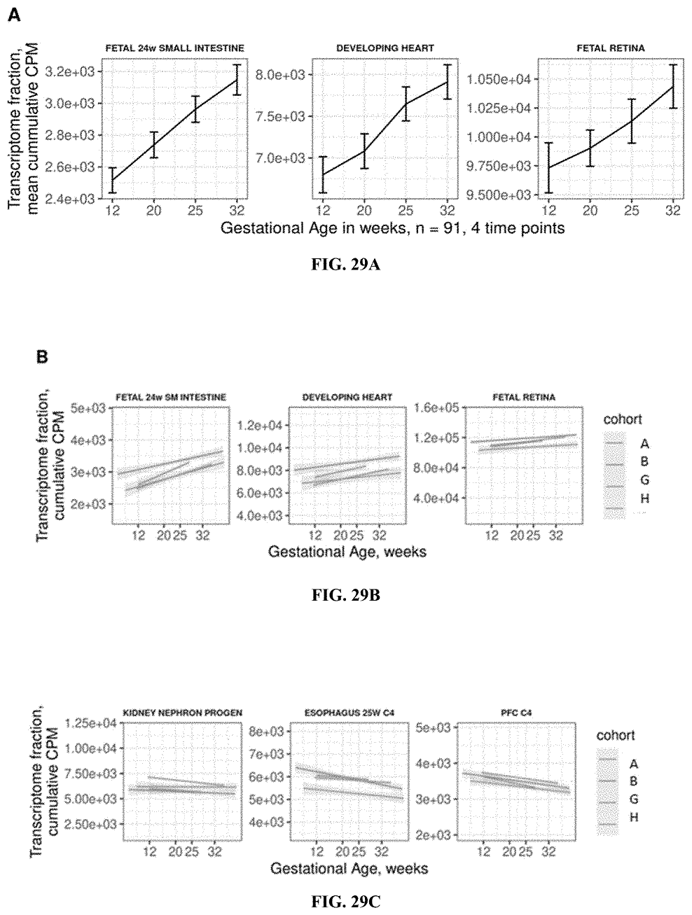

Top three fetal organ gene sets with the most significant upward trends (based on the p-value of the collection age coefficient at a confidence level of 0.05) are depicted in FIG. 29A. Those sets are “24-week small intestine enterocyte progenitor cell”, “fetal retina microglia”, and “developing heart C6 epicardial cell”.

To verify if the fetal cell-type signature trends can be generalized from training cohort to held out test cohorts (A, B, and G). The selected fetal cell-type signatures were models as a linear function of gestational age in held-out cohorts. FIG. 29B shows indistinguishable trends for each the signatures gene sets in trained and tested cohorts.

In addition, 3 fetal organ gene sets were independently identified as having significant downward trajectories in the transcriptome fraction space (3 of those were also significantly enriched in samples collected at 12 weeks of gestation age compared to sample from 32 weeks). It indicates that these analyses, gene set enrichment in the individual gene space and analysis of linear trends in the transcriptome fraction space) are not equivalent in tracking fetal fractions. FIG. 29C shows the verification modeling of the top three downward trending gene sets with gestation age (kidney nephron progenitor cells, esophagus C4 epithelial cells, and prefrontal cortex brain C4 cells in held out test cohorts A, B, and G.

### Example 15: Human cfRNA Profiling from Liquid Biopsies Provide a Molecular Window into Maternal-Fetal Health

A liquid biopsy of the maternal circulation offers a non-invasive window into the biological progression of the maternal-fetal dyad [Koh et al]. We show that cell-free RNA (cfRNA) signatures from such liquid biopsy provide accurate information on gestational age, on monitoring the progression of fetal organ development and offer an early warning of potential risk of developing preeclampsia.

Results center on a comprehensive transcriptome data set from eight independent prospectively collected cohorts comprising 1,724 racially and ethnically diverse pregnancies, and retrospective analysis of 2,536 banked blood plasma samples. This data set includes samples from 72 patients with preeclampsia matched to 469 non-cases obtained from two independent cohorts. Liquid biopsies were collected 14.5 weeks (SD 4.5 weeks) prior to delivery.

We show that cfRNA signatures can accurately date gestation with a mean absolute error of 15 days across the entire pregnancy. Importantly, the molecular signatures are independent of clinical factors, such as BMI, maternal age, and race or ethnicity, which cumulatively account for less than 1% of model variance, the model is overwhelmingly driven by transcripts (p<2e-16). Additionally, using longitudinal samples at 4 gestational time points, we show an increase in fetal signals from heart, kidney and small intestine as gestation progresses; an observation confirmed in three other cohorts with longitudinal data (p<1e-5). Further, we have identified a cfRNA signature with biologically relevant gene features (p<1e-12) to enable early detection of preeclampsia with a sensitivity of 75% and a positive predictive value of 30% given our study incidence rate of 13%.

A cfRNA profile can be analyzed to provide a non-invasive method to assess maternal-fetal health as well as assess the risk for perinatal pathologies like preeclampsia. This approach overcomes biases from the risk assumptions based on clinical factors, including race. Thus, the test is broadly applicable and provides new opportunities to identify at-risk pregnancies allowing for more precision based therapeutic approaches and improved maternal-fetal health outcomes.

Contemporary obstetrics has a long and successful history of minimally invasive screening for fetal aneuploidy (Rose et al 2020). As a result, aneuploidy screening may be a common aspect of prenatal care despite its low incidence (estimated <1%, Nussbaum et al 2016) compared to the more frequent rates of early delivery due either to preterm labor or preeclampsia which occur over ten-fold more frequently (5-18% of deliveries globally, Blencowe et al, 2102). These obstetric complications are the leading cause of maternal and neonatal morbidity and mortality worldwide (WHO). An early detection cfRNA test, aimed at these more frequent complications, may represent a long overdue advance to obstetric practice with implications for maternal and child health globally.

Beyond this potential for developing a more effective stratification of prenatal risk, cfRNA analyses may also provide a deeper understanding of molecular intricacies and biologic systematics, particularly those that vary longitudinally with the progression of pregnancy. The dynamic and complex nature of pregnancy necessitates assessment of a tissue-specific molecular analyte, such as RNA, to adequately capture the molecular messaging from maternal, placental and fetal cells. Such an examination may enable avenues of diagnostic and therapeutic intervention that are presently not available.

In this work, we demonstrate that cfRNA signatures may meet these multiple objectives by both providing accurate information on gestational age progression, time dependent process of fetal organ development and identification of individual's risk for adverse pregnancy outcomes such as preeclampsia.

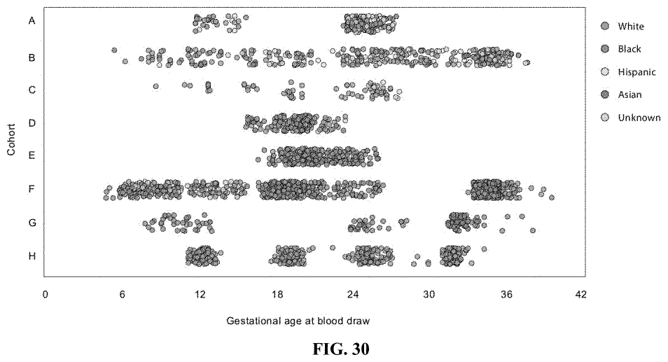

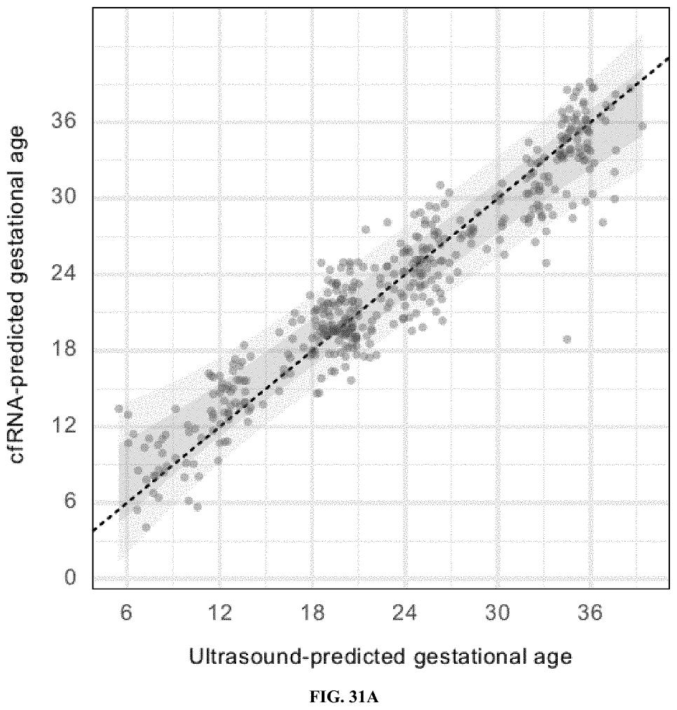

The study design is described as follows. Other studies may use cfRNA to monitor pregnancy and detect or diagnose adverse pregnancy outcomes such as preeclampsia (Koh et al 2014, Ngo et al 2018, Munchel et al 2020, Del Vecchio et al 2020, Moufarrej et al 2021). A common limitation of these and other studies has been the use of relatively small sample sizes with low ethnic & racial diversity, with incomplete validation, has hindered use in the clinical setting. In this study, generalizability has been improved by applying the techniques to a larger and more diverse sample set. Combination of samples from eight prospectively collected pregnancy cohorts provided n=2,536 plasma samples from n=1,652 pregnancies across a diverse set of ethnicities and covering a broad range of gestational ages (FIG. 30). The broad demography of our data (Table 31) enabled us to test if initial findings could be applied widely. All study procedures involving human subjects were reviewed and approved by the appropriate local institutional review board. All samples were collected under controlled conditions and only included samples with a time from collection to spin down and freezer storage less than 8 hrs. All plasma samples were processed following main laboratory protocol with minor variations (supplementary methods) and a standardized bioinformatic pipeline to measure gene counts and multiple sample quality metrics for each cfRNA sample. The eight different cohorts were treated as batches and a correction was applied prior to modeling of the data. A more detailed description of each cohort and the correction method is available in the supplementary information.

TABLE 31
Summary of samples collected from different cohorts

| | | | | Pre- | |
|---|---|---|---|---|---|
| | | Gestational | | pregnancy | Mother's |
| | | Age at | Gestational | Body | Age at |
| | | Blood | Age at | Mass | Blood |
| cohort | count | Draw | Delivery | Index | Draw |
| A | 161 | 23.4 +/− 4.60 | 38.9 +/− 0.65 | NA | NA |
| B | 385 | 26.3 +/− 8.45 | 39.3 +/− 1.08 | NA | NA |
| C | 70 | 22.5 +/− 5.00 | 39.3 +/− 1.08 | 33.5 +/− 9.27 | 29.8 +/− 5.16 |
| D | 194 | 19.9 +/− 1.77 | 39.6 +/− 1.27 | 26.6 +/− 6.31 | 32.8 +/− 5.38 |
| E | 282 | 21.8 +/− 2.16 | 39.5 +/− 1.22 | 28.6 +/− 7.94 | 26.4 +/− 5.52 |
| F | 594 | 27.1 +/− 7.78 | 39.5 +/− 1.11 | NA | NA |
| G | 140 | 25.2 +/− 9.66 | 39.9 +/− 0.91 | 24.5 +/− 5.12 | NA |
| H | 412 | 22.5 +/− 7.35 | 39.8 +/− 1.19 | 25.5 +/− 6.13 | NA |

| | | | | | Pre- | |
|---|---|---|---|---|---|---|
| | | | Gestational | | pregnancy | Mother's |
| | | | Age at | Gestational | Body | Age at |
| | Sample | | Blood | Age at | Mass | Blood |
| Cohort | Type | Count | Draw | Delivery | Index | Draw |
| A | case | 46 | 22.6 +/− 5.17 | 36.2 +/− 2.42 | NA | NA |
| A | control | 88 | 22.8 +/− 5.00 | 39.0 +/− 0.57 | 27.5 +/− 7.19 | NA |
| E | case | 39 | 22.5 +/− 2.53 | 34.6 +/− 3.97 | 29.8 +/− 7.31 | 26.2 +/− 5.86 |
| E | control | 271 | 21.8 +/− 2.09 | 39.5 +/− 1.34 | 28.5 +/− 8.06 | 26.7 +/− 5.56 |

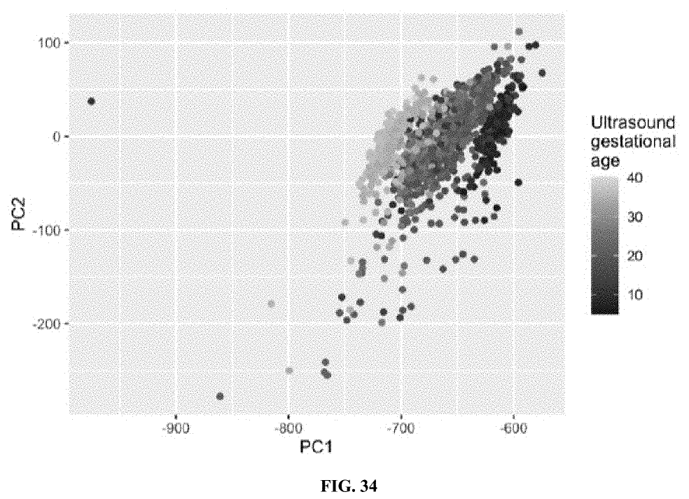

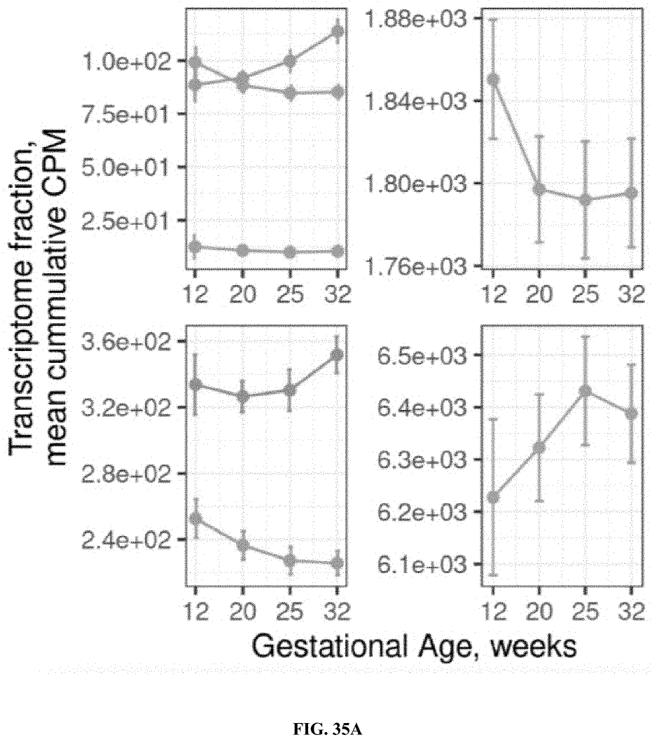

It was observed that molecular signature of gestational age is independent of clinical factors. While gestational age may be predicted using multiple samples over a pregnancy (Ngo et al 2018), we aimed to test performance using a single blood sample to predict gestational age. The potential to create a predictive model for gestational age given the transcription counts for a sample, can be seen in a principal components analyses (FIG. 34). In FIG. 34, the first principal component separates the samples by the gestational age at sample collection, indicating that gestational age is one of main driver of transcriptomic variability across the dataset. Before beginning to develop a machine-learning model to capture this signal, we divided our data from all full-term pregnancies without preeclampsia into a training set (n=1,924 samples) and a held-out test set (n=480 samples), making sure to stratify by gestational age so all age bands were represented equally in both sets.

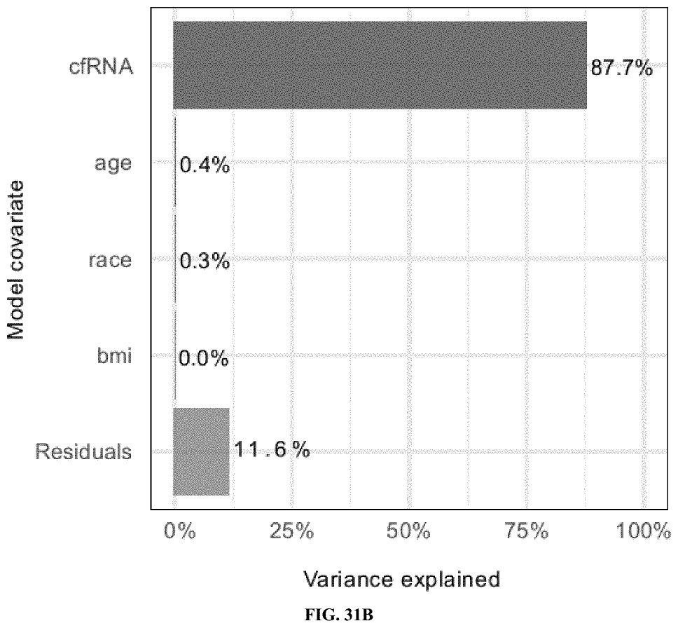

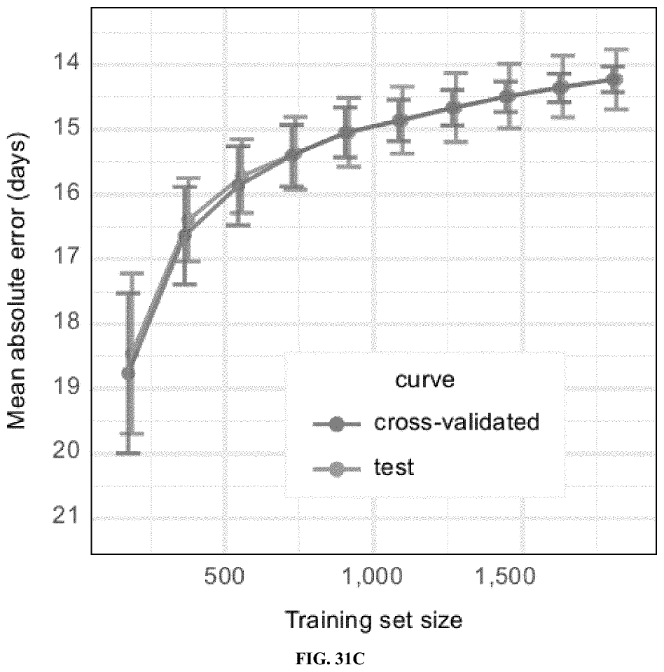

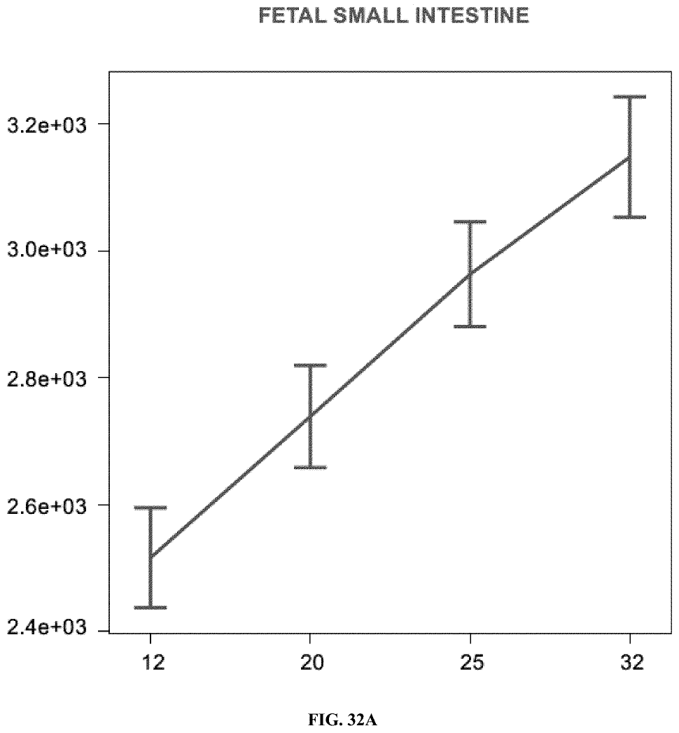

Prior to modeling the counts for each gene were first normalized to account for variation due to sequencing depth and then transformed so that the mean of each gene is the same across cohorts (see Supplementary text for details). We limited our feature space to genes with a median expression greater than zero across all samples (14,628 genes). A Lasso linear model was fitted to predict gestational age in the training set, with test set performance of a mean absolute error of 15 days (SD 1 day) (FIG. 31A), when using first trimester fetal ultrasound biometry as the gold standard measurement. Of note, we model against ultrasound as the true gestational age, thus the known error of 5-7 days when measured in first trimester (Hadlock et al, 1987) in ultrasound estimated gestational age is a limitation to assess the true performance of our model. The model uses 699 of the available gene features, although this includes a long tail of features with low contribution. Using the top-50 most informative features, it was possible to train a linear model to achieve a mean absolute error of 2.3 weeks.

To assess whether adding further samples to our data set would increase model learning, modeling was repeated with progressively smaller subsets of the data to construct a learning curve (FIG. 31C). The continued reduction in error as we reached our complete training set of n=1,924 samples, indicated that model learning was not exhausted and additional samples would increase our performance. Notably, as seen in FIG. 31C, the similar performance in cross-validation and on the independent held-out test data indicated that the model was not overfit. To determine how far the model could be extrapolated, a final model was built using all data, this gave a mean absolute error of 13 days across the entire data set, improvements beyond adding more samples could come from samples with known conception date, e.g. from in vitro fertilized pregnancies. Compared to prior published results (Ngo et al 2018), this model outperforms the accuracy across all trimesters. In our data set, the error in cfRNA gestational dating was consistent across the predicted range from 6 to 36 weeks (FIG. 31A). This result is in contrast to ultrasound-based dating, which has a gradual increase in error as pregnancy progresses, increasing to over 20 days in the third trimester (Skupski et al 2017). Overall, the error of our model is equivalent to that of second trimester ultrasound and superior to third trimester ultrasound (Skupski et al 2017).

Next, we explored if the inclusion of clinical factors improved the performance of the model. By analysis of variance (ANOVA), we showed that the model was driven almost entirely by information from the cfRNA transcripts with body mass index, maternal age and race/ethnicity accounting for less than 1% of total variance (FIG. 31B). A liquid biopsy test based on molecular signatures, therefore, worked independently of clinical factors and could help reduce biases introduced from risk assumptions based on clinical and demographic factors.

These data indicate that a simple blood test that can be shipped to a central lab has broad applicability and may be used as the primary assessment of gestational age in low resources settings, where timely access to trained ultrasonographers may be limited, and the high proportion of small for gestational age pregnancies further degrades accuracy of the translation of fetal ultrasound biometry to gestational age estimates. There may also be an adjunct value for suboptimally dated pregnancies where a confirmatory ultrasound was not able to be obtained before third trimester.

Further, we observed molecular signature for fetal organ development. We explored whether transcripts found in maternal circulation during pregnancy encode information regarding fetal organ development. As individual transcripts from the fetus are relatively rare in the maternal plasma, we investigated fetal organ signal by analyzing gene sets and by targeting gene sets discovered in human embryonic cells for this analysis. We used longitudinal samples from the cohort H (Gybel-Brask et al 2014), where pregnant individuals were sampled up to four times during pregnancy. A total of 91 women had data available for all four collections, which were carried out at gestational weeks 12, 20, 25, and 32 (within a given std dev).

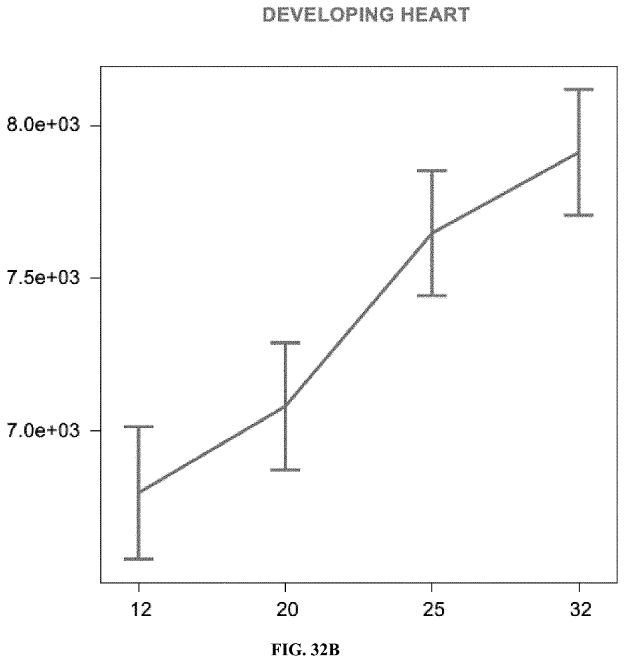

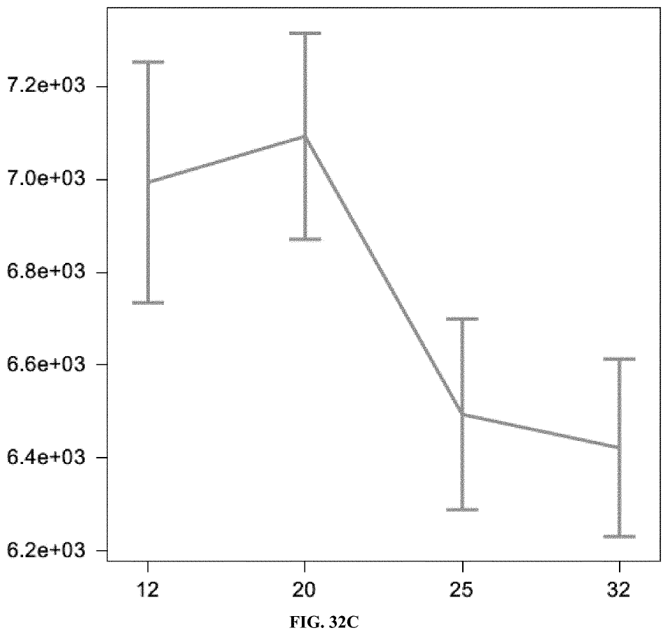

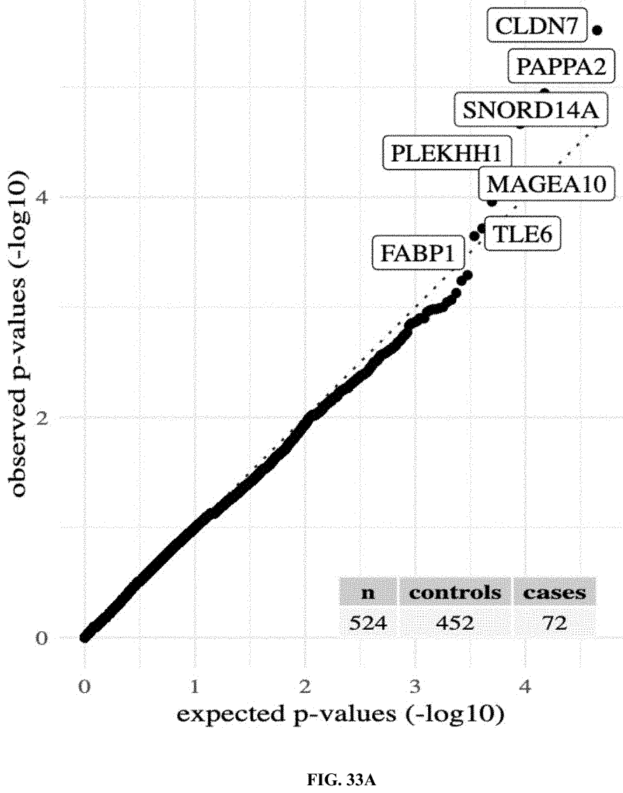

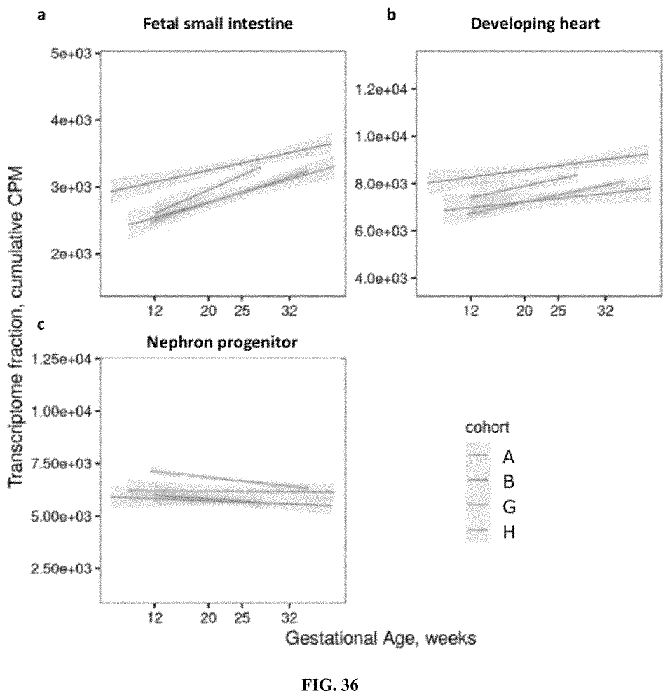

Based on a pairwise comparison between samples from early and late pregnancy (collections at 12 and 32 weeks), we identified 80 cell-type specific gene sets that were significantly enriched (Table 32). Of these, 33 sets were characteristic of embryonic cell types of which 19 showed significant temporal upward trends along the pregnancy timeline. Of all the analyzed gene sets, including fetal and adult, the “24-week small intestine enterocyte progenitor cell” type (Gao et al 2018) showed the most significant trend (FIG. 32A) For the small intestine gene set we evaluated how many of the samples monotonically increased over the four time points and identified 36 study participants that followed this strict criterion (p<2e-16). Another example of increasing signal with gestational age was observed from “developing heart C6 epicardial cell” (FIG. 32B, Cui et al 2019). Of the remaining gene sets thirteen displayed downward trajectories, examples of a gene sets that decrease in expression were kidney nephron progenitor cells (FIG. 32C, Menon et al 2018), which aligns with the decreasing nephrogenic zone width as a function of gestational age (Ryan et al 2018). Additionally, for these gene sets, we confirmed the directional change in expression in three other cohorts: A, B and G, where at least 2 longitudinal samples were processed (FIG. 36).

TABLE 32
Cell-type specific gene set collections (C8)
used in the gene set enrichment analysis

| Primary | | Number of | | |
|---|---|---|---|---|
| author | Focus organ | cell types | Adult or fetal | PMID |
| Aizarani | Liver | 31 | adult | 31292543 |
| Cui | Developing heart | 25 | Fetal 5-25 w | 31292543 |
| Durante | Olfactory | 26 | adult | 32066986 |
| Fan | Embryonic cortex | 31 | fetal 22-23 w | 29867213 |
| Gao | Esophagus | 4 | fetal 25 w | 29802404 |
| Gao | Large intestine | 9 | fetal 24 w | 29802404 |
| Gao | Large intestine | 7 | adult | 29802404 |
| Gao | Small intestine | 7 | fetal 24 w | 29802404 |
| Gao | Stomach | 5 | fetal 24 w | 29802404 |
| Hay | Bone marrow | 29 | adult | 30243574 |
| Hu | Fetal retina | 11 | fetal 5-25 w | 31269016 |
| Lake | Kidney | 30 | adult | 31249312 |
| Menon | Kidney | 11 | fetal 12-19 w | 30166318 |
| Manno | Midbrain | 26 | fetal and | 27716510 |
| | | | progenitor | |
| Muraro | Pancreas | 9 | adult | 27693023 |
| Zheng | Cord blood | 10 | adult and | 29545397 |
| | | | progenitor | |
| Zhong | Prefrontal cortex | 31 | fetal 8-26 w | 29539641 |

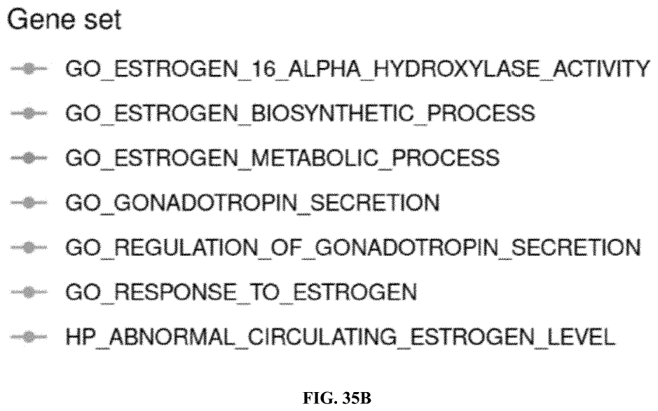

Using a gene ontology (GO) collection of gene sets, we identified seven pregnancy related sets that were significantly enriched in the comparison between early and late pregnancy samples (FIGS. 35A-35B). Three gene sets in the gonadotropin and estrogen pathways exhibited significant changes consistent with their known physiology (Tal et al 2015).

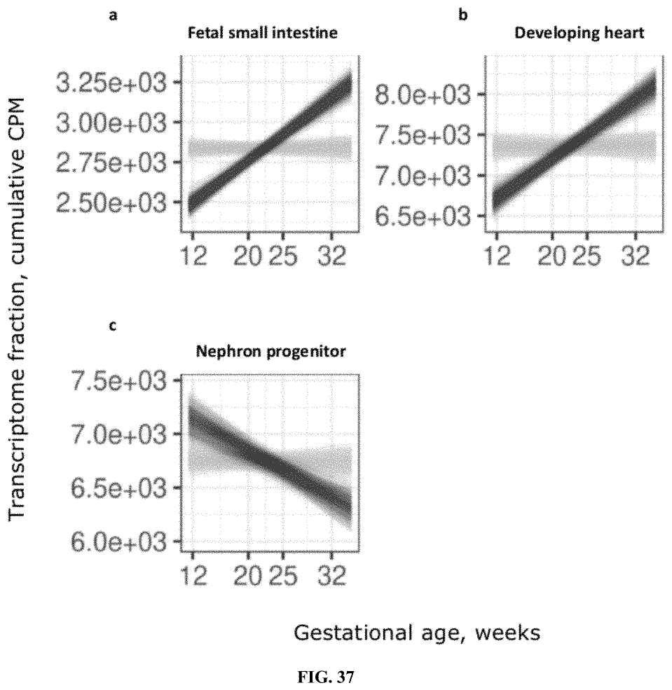

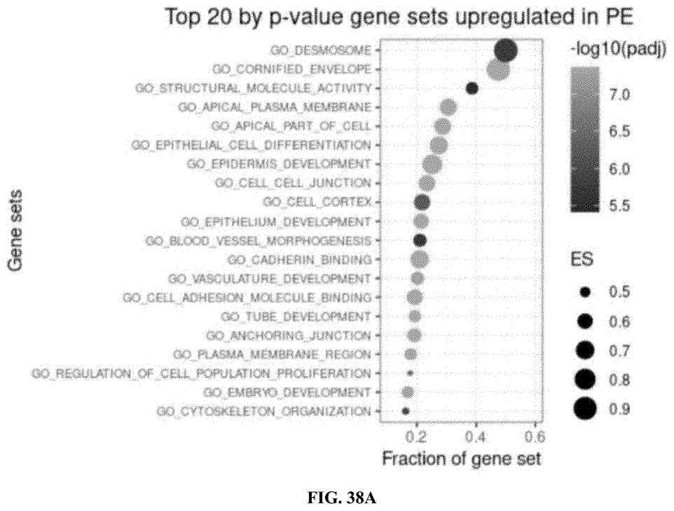

We next compared the observed collection time labels to a set of randomly permuted collection time labels. This comparison certified that all selected gene sets were, in fact, associated with the longitudinal progression of pregnancy (FIG. 37). Furthermore, we repeated the gene set analyses after removing all 699 genes used in the gestational age model and rediscovered the same 80 gene sets were differentially expressed. As changes in gene sets, up or down, were only significant in the context of gestational age, with or without the gestational age model genes, we showed the first window into fetal development from a maternal liquid biopsy sample.

Preeclampsia is a leading cause of maternal morbidity and mortality. A diagnosis of preeclampsia confers a lifetime increased risk for cardiovascular disease for the mother (Haug et al, 2018). Yet, despite the signification health implications of this diagnosis for a woman's pregnancy and her lifetime, there remains challenges to developing reliable methods to identify women at risk early in pregnancy.

We evaluated the predictability of preeclampsia from molecular signatures measured in blood draws taken during the second trimester (16-27 weeks), on average 14.5 weeks (SD 4.5 weeks) before delivery. A case-control study with 72 cases of preeclampsia and 469 matched non-cases selected from two independent cohorts (cohorts A and E) was performed. Cohort E included 34 controls with chronic hypertension and 19 with gestational hypertension, both cohorts included preterm birth samples in the non-case population. Preeclampsia was defined by criteria consistent with those of the 2013 Task Force on Hypertension in Pregnancy (ACOG 2013), and each case was adjudicated by two board certified physicians. Blood samples were collected at gestational weeks 16-27, before the onset of signs or symptoms of preeclampsia. As before, a cohort correction was applied prior to modeling.

We used Spearman correlation tests to identify transcriptional signatures that can differentially separate the preeclampsia cases and controls presented in Table 33.

TABLE 33
Set of 38 Differentially Expressed Transcriptional
Features Predictive of Preeclampsia (PE)

| Transcriptional feature | P-value | P-value adj |
|---|---|---|
| CLDN7 | 4.20E−10 | 1.40E−05 |
| PAPPA2 | 3.94E−09 | 1.32E−04 |
| SNORD14A | 1.17E−08 | 3.91E−04 |
| PLEKHH1 | 3.76E−08 | 0.0012570947 |
| MAGEA10 | 1.86E−07 | 0.006203178738 |
| IGKV2OR22-4 | 3.76E−07 | 0.01257256125 |
| CH17-335B8.4 | 3.76E−07 | 0.01257503174 |
| TLE6 | 4.82E−07 | 0.01610065186 |
| FABP1 | 6.32E−07 | 0.02112300951 |
| AC015977.5 | 9.57E−07 | 0.03196867232 |
| GJC1 | 2.53E−06 | 0.08459648949 |
| PTPRQ | 3.10E−06 | 0.1035580684 |
| GJD4 | 4.79E−06 | 0.1599066029 |
| TEAD3 | 6.09E−06 | 0.2033532195 |
| RNA5SP71 | 6.64E−06 | 0.2217167558 |
| SALL1 | 7.90E−06 | 0.2638484427 |
| GPSM2 | 8.20E−06 | 0.2737536288 |
| SLC27A2 | 8.52E−06 | 0.2845032434 |
| CRH | 8.53E−06 | 0.2847182052 |
| TRIM29 | 8.84E−06 | 0.2953097559 |
| GTSF1L | 9.41E−06 | 0.3143403365 |
| DEFB132 | 1.18E−05 | 0.3929372843 |
| OR7E158P | 1.18E−05 | 0.3929372843 |
| RNU6-708P | 1.18E−05 | 0.3929372843 |
| SAA2-SAA4 | 1.18E−05 | 0.3929372843 |
| HP | 1.29E−05 | 0.4322689364 |
| ITGB6 | 1.34E−05 | 0.4480987694 |
| KIAA1211L | 1.39E−05 | 0.4638821437 |
| OR4S1 | 1.41E−05 | 0.4721774325 |
| NOC2LP1 | 1.45E−05 | 0.4849266379 |
| HRH4 | 1.53E−05 | 0.5103650892 |
| CFAP57 | 1.95E−05 | 0.649835203 |
| THEM6 | 2.11E−05 | 0.7046812124 |
| S100A14 | 2.18E−05 | 0.7271782584 |
| DPCR1 | 2.39E−05 | 0.7967427421 |
| GPC1 | 2.58E−05 | 0.8613470703 |
| MYOM3 | 2.69E−05 | 0.8978677978 |
| BHMT2 | 2.79E−05 | 0.9319628309 |

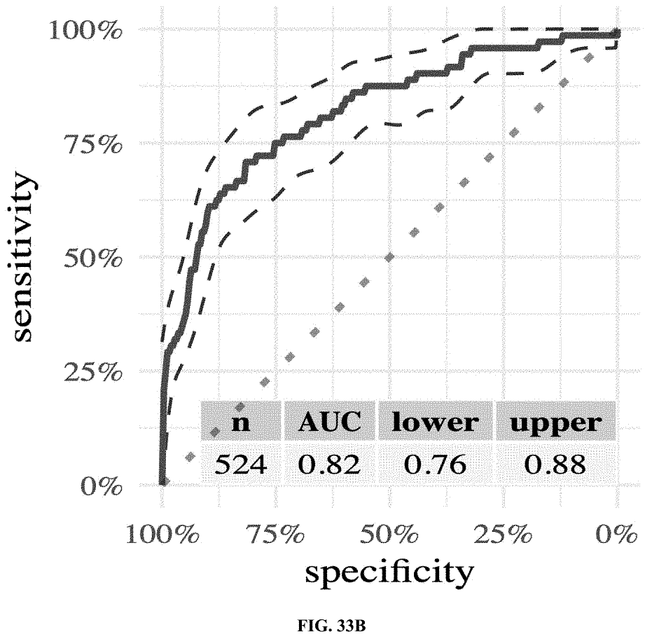

During in each round of cross-validation we kept features with adjusted p-value below 0.05 and consistently identified seven genes: CLDN7, PAPPA2, SNORD14A, PLEKHH1, MAGEA10, TLE6 and FABP1 (FIG. 33A). Each of the seven genes selected for modeling may have a function relevant to preeclampsia or fetal development. PAPPA2, or pregnancy associated plasma protein 2, is expressed primarily in placenta (Uhlén et al 2015) and specifically in trophoblast cells. It may be linked to the development of preeclampsia (Kramer et al 2016, Chen et al 2019), and associated with inhibition of trophoblast migration, invasion and tube formation. PAPPA2 is a protease that cleaves insulin growth factor binding protein 5 (IGFBP5) and impacts the pathway of insulin growth factor 2 in which higher levels lead to increased fetal growth (White et al 2018). Claudin 7 (CLDN7) a protein involved in tight cell junction formation, may be implicated in blastocyst implantation; in a healthy pregnancies CLDN7 is reduced in response to estrogen at time of implantation (Poon et al 2013). Fatty acid Binding Protein 1 (FABP1) may be detected and purified from human cytotrophoblasts and may be highly expressed in fetal liver, it is critical for fatty acid uptake and transport (Wang et al 2020) and is upregulated 3-fold when cytotrophoblasts differentiate to syncytiotrophoblasts around the time of implantation (Cunningham and McDermott 2009).

Based on these identified gene features, a logistic regression model, in a leave-one-out cross validation setup, was used to estimate the likelihood of preeclampsia. At a sensitivity of 75%, our model achieves a positive predictive value of 32.3% (SD 3%) given a 13.7% occurrence in our study; AUC for the model is 0.82 (FIG. 33B). Similar to the gestational age model, adding in clinical factors (BMI, maternal age, and race/ethnicity) has no significant effect and account for less than 1% of variance based on ANOVA analyses.

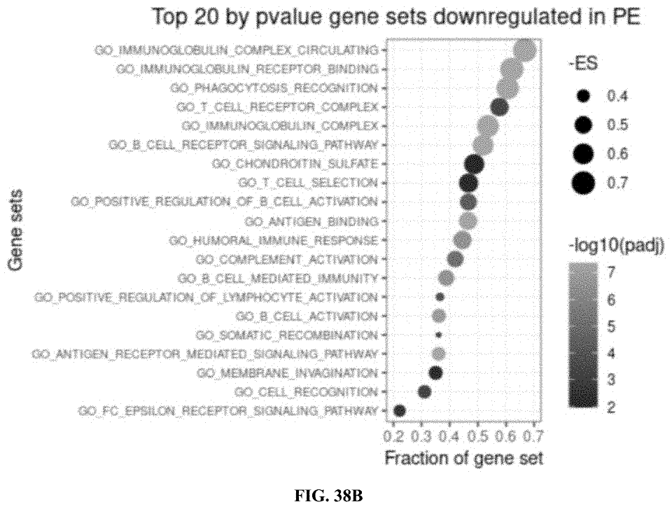

To further understand the molecular signature changes and how they might reflect the pathophysiology driving preeclampsia, a differential gene set analysis was performed. The top upregulated gene sets are dominated by structural cell functions including desmosome, blood vessel morphogenesis and vasculature development (FIG. 38A), while the vast majority of downregulated gene sets were related to immune pathways (FIG. 38B). Both aligned well with what is known about preeclampsia pathophysiology (Redman & Sargent, 2005).

The control group contained both normotensive women (n=416) and women with chronic hypertension (n=34) and gestational hypertension (n=19). Comparison of the chronic or gestational hypertensive groups to the normotensive group, showed no overlap with genes significant for preeclampsia (no gene achieved an adjusted p-value below 0.05). While others have published studies designed to determine the effect of hypertension per se on gene expression (e.g. Zeller et al 2017), here we demonstrate that the signal for preeclampsia, is independent of any signal associated with chronic or gestational hypertension. As preeclampsia and spontaneous preterm birth are theorized by some to have overlapping molecular pathways (REF), we also excluded samples with delivery prior to gestational week 37 (n=89) from the non-case group. Removal of preterm delivery samples had no impact on our model performance (supplementary methods), indicating that our signature can separate preeclampsia from spontaneous preterm delivery. We report a stand-alone molecular predictor that has the potential to be a reliable, early detection of preeclampsia, that is based entirely on transcripts and is independent of clinical factors such as body mass index, maternal age and race/ethnicity.

The transcriptome data set presented here shows that comprehensive molecular profiling from liquid biopsies can provide a robust window into maternal-fetal health. We have shown that transcript signatures from a single liquid biopsy can: (i) accurately estimate gestational age at performance levels comparable to ultrasound, making it a viable option for rural and low-resource settings, as well as to confirm gestational age beyond the first trimester where ultrasound accuracy is limited (Skupski et al 2017), (ii) provide non-invasive monitoring of fetal organ development including the fetal heart, small intestine and kidney, and (iii) has the potential to reliably identify risk of preeclampsia prior to onset of disease using novel transcript signatures, whose biological significance adds further rigor to our findings.

These findings expand on other studies from tens of pregnancies (Koh et al 2014, Ngo et al 2018) by moving to over a thousand pregnancies. This scale allows us to non-invasively assess molecular foundation of pregnancy health, with the ability to develop signatures from specific fetal organs that may give an early warning of birth defects such as congenital heart disease. We further improved the accuracy of gestational age assessment to be equivalent to ultrasound. The generalizability of these results is afforded by the large and racially diverse cohorts utilized in this work.

We establish specific transcript signatures that inform the early identification of the risk of preeclampsia. However, we do not replicate the differential gene expression for preeclampsia seen in Moufarraj et al (2021) (collected before week 16) in the samples used for preeclampsia modeling (collected week 16-27). Nor did we replicate the final genes selected in Munchel et al (2020)(collected at time of diagnosis, typically after week 34). Comparison of differential gene expression across studies may be confounded by varying trimesters of sample collection.

The data presented here are strengthened by the study size and the use of geographically distinct cohorts. This ensures diversity in our sample composition and generalizability of our conclusions. However, due to small differences in collection protocols for the different cohorts required cohort correction, prospective studies may combine diversity and size with a consistent framework for collecting samples, for clinical validation and utility studies.

The presented results demonstrate improved methods to overcome current limitations in our ability to assess maternal-fetal health during a pregnancy. Importantly, a liquid biopsy approach overcomes biases introduced by risk assumption based only clinical factors, including race and BMI. As such, molecular tests, based on cfRNA, are broadly applicable and provide new opportunities to identify at-risk pregnancies allowing for more precision based therapeutic approaches and improved maternal-fetal health outcomes. A cfRNA platform enables early detection of multiple clinically relevant endpoints (e.g. gestational age and preeclampsia) from a single sample without the need of local specialized point-of-care testing facilities.

In addition to a more effective approach to risk stratification for adverse pregnancy outcomes, liquid biopsies of the maternal-fetal-placental transcriptome also present a vehicle by which understanding of the biological underpinnings of maternal-fetal health and disease can be improved and provide novel insight into interactions across maternal-fetal dyad. This holds the promise of more effective, precision therapeutic interventions that can then target molecular subtypes of preeclampsia and preterm birth.

The impact from the use of non-invasive assessment of molecular signatures can be appreciated from its role in advancing breast cancer diagnosis (Alimirzale et al, 2019). We now have the opportunity to similarly advance the field of maternal and child health by identifying those at risk for adverse outcomes such as preeclampsia, preterm birth and gestational diabetes in this decade. Given the 60 million women who experience some form of pregnancy complication each year, a molecular, precision diagnostic and precision medicine approach has the potential to transform many lives.

In this work, we have demonstrated the potential of obtaining transcript signatures obtained in pregnancy allow us insight into three novel aspects of pregnancy: The estimation of gestational age, the monitoring of fetal organ development, and the assessment of risk for preeclampsia later in gestation. These insights were all obtained via a single liquid biopsy obtained on average 14.5 weeks before delivery.

Cohort Descriptions

Cohort A (BWH)

LIFECODES is a prospective pregnancy biorespository that has been recruiting pregnant women in the greater Boston, MA area since 2006. Women 18 yrs. and older and plan to deliver at Brigham and Women's Hospital are eligible. Higher order pregnancies (triplets or greater) are excluded. To date N=5,569 pregnant women have been enrolled and followed, providing longitudinal samples and data, through delivery. Racial and ethnic makeup of LIFECODES follows the general US trend with 55% being Caucasian, 14.8% African American, 7.3% Asian, 18.4% Hispanic, and 4.5% Mixed/Other. The medical record for each subject in LIFECODES is independently reviewed by two certified Maternal Fetal Medicine physicians. Complications and outcomes for each subject are coded using a structured coding tool. The codes from each reviewer are then compared with disagreement in either pregnancy outcome or complication and is decided by a review committee. Ref PMID 25797229

Cohort B (GAPPS)

The Global Alliance to Prevent Prematurity and Stillbirth (GAPPS) (www.gapps.org) has developed a continually recruiting cohort of pregnant women and their babies designed to combat the deficit of pregnancy-related specimens and accompanying data available for research. Participants for this study were enrolled at all gestational ages from obstetric and antepartum clinic sites in Washington State under the Advarra IRB (FWA00023875) protocol number Pro00036408. Written informed consent was obtained from all participants and parental permission and assent were obtained for participating minors aged at least 15 years. A repository of biospecimens collected longitudinally at each trimester of pregnancy and the postpartum period are linked to comprehensive patient data across the gestation. Biospecimens were collected from ten maternal body sites (vaginal, cervical, buccal and rectal mucosa, blood, urine, chest, dominant palm, antecubital fossa and nares), five types of birth products (amniotic fluid, cord blood, placental membranes, placental tissue and umbilical cord) and seven infant body sites (right palm, buccal and rectal mucosa, meconium/stool, chest, nares and respiratory secretions if intubated). All blood is processed and stored at −80C within two hours of collection. The data repository was developed with the goal of supporting prematurity and stillbirth research and to better understand associated risk factors.

Pregnant women were provided literature describing the repository project and invited to participate in the study. Women who were incapable of understanding the informed consent or assent forms or were incarcerated were excluded from the study. Comprehensive demographic, health history and dietary assessment surveys were administered, and relevant clinical data (for example, gestational age, height, weight, blood pressure, vaginal pH, diagnosis) were recorded. Relevant clinical information was obtained from neonates at birth and discharge and six weeks postpartum.

At subsequent prenatal visits, labor and delivery, and at discharge, characterizing surveys were administered, relevant clinical data were recorded and samples were collected. Vaginal and rectal samples were not collected at labor and delivery or at discharge. Women with any of the following conditions were excluded from sampling at a given visit: (1) Incapable of self-sampling due to mental, emotional or physical limitations; (2) More than minimal vaginal bleeding as judged by the clinician; (3) Ruptured membranes before 37 weeks; (4) Active herpes lesions in the vulvovaginal region; and (5) Experiencing active labor.

Cohort C (IO)

Informed consent for sample and data collection was obtained at the University of Iowa by the Maternal Fetal Tissue Bank (IRB #200910784). Blood samples were collected in ACD-A tubes (Becton Dickinson). Plasma was aliquoted, snap frozen, and stored at −80C. All freezers are alarmed with temperature monitors. Time of sample collection and processing are recorded within the research information system managed by the UI Bioshare service (Labmatrix, Biofortis). All samples are coded and are annotated with clinical information. (PMID: 24965987)

Cohort D (KCL)

INSIGHT: Biomarkers to predict premature birth is an ongoing observational cohort study designed to study women at high risk of spontaneous preterm birth (sPTB) compared to low-risk controls. Plasma samples (taken between 16-23⁺⁶ weeks of gestation) provided for the current analyses were obtained from women with singleton pregnancies participants recruited from four tertiary antenatal clinics in the UK. High-risk pregnancies are defined by at least one of; prior sPTB or late miscarriage (between 16 to 37 weeks of gestation), previous destructive cervical surgery or incidental finding of a cervical length <25 mm on transvaginal ultrasound scan. Women with no risk factors for sPTB and otherwise well at the time of recruitment are recruited as low-risk controls from either routine antenatal or ultrasonography clinics at these centres. Exclusion criteria for both the high and low risk groups were multiple pregnancy, known major congenital fetal abnormality, rupture of membranes or current vaginal bleeding. Approval from London City and East Research Ethics Committee was granted (13/LO/1393). Informed written consent was obtained from all participants.

Reference: PMID: 32694552, Cervicovaginal natural antimicrobial expression in pregnancy and association with spontaneous preterm birth (Hezelgrave et al., 2020) is incorporated by reference herein in its entirety.

Reference: Hezelgrave N L, Seed P T, Chin-Smith E C, Ridout A E, Shennan A H, Tribe R M. Cervicovaginal natural antimicrobial expression in pregnancy and association with spontaneous preterm birth. Sci Rep. 2020 Jul. 21; 10(1):12018. doi: 10.1038/s41598-020-68329-z is incorporated by reference herein in its entirety.

Cohort E (MSU)

The Pregnancy Outcomes and Community Health (POUCH) Study cohort includes 3,019 pregnant women enrolled at 16-27 weeks' gestation (1998-2004) from 52 clinics in five Michigan communities. Eligibility included singleton pregnancy and no known congenital anomaly, maternal age ≥15, maternal serum alpha-fetoprotein (MSAFP) screening, no pre-pregnancy diabetes mellitus, and English speaking. At enrollment study nurses interviewed participants and collected biologic samples (blood, urine, hair, vaginal fluid). An additional at-home data collection protocol included ambulatory blood pressure monitoring and three consecutive days of saliva and urine collection for measuring stress hormones. To conserve resources, a sub-cohort of 1,371 participants were studied in greater depth, i.e., medical records abstracted, biological samples analyzed, and placentas examined.¹ The sub-cohort is 42% primiparous, 57% 20-30 years of age, 42% African American and 49% non-Hispanic white, and 57% were insured through Medicaid.

Holzman C, Senagore P K, Wang J. Mononuclear leukocyte infiltrate in the extra-placental membranes and preterm delivery. Am J Epidemiol 2013; 177(10):1053-64. PMCID: PMC3649632 is incorporated by reference herein in its entirety.

Cohort F (PITT)

Samples were provided from biobanks collected in association with NIH P01 HD HD030367. These samples were part of 3 successive renewals of the PPG and collected between 2001 and 2012. In all cases samples were collected longitudinally across pregnancy from low risk pregnant women cared for at Magee-Womens Hospital Pittsburgh Pennsylvania. Exclusion criteria were pre-existing hypertension, diabetes, multiple gestation or renal disease. Charts were abstracted and reviewed by a jury of 5 clinicians. The population was approximately 50% African American, 50% Caucasian with very few other race/ethnicities included.

Powers R W, Roberts J M, Plymire D A, Pucci D, Datwyler S A, Laird D M, Sogin D C, Jeyabalan A, Hubel C A, Gandley R E. Low Placental Growth Factor Across Pregnancy Identifies a Subset of Women With Preterm Preeclampsia Type 1 Versus Type 2 Preeclampsia? Hypertension. 2012; 60:239-46 is incorporated by reference herein in its entirety.

Cohort G (PM)

The Pemba Pregnancy and Discovery Cohort (PPNDC) study is being undertaken in Pemba Island, Zanzibar, Tanzania. This ongoing study is follow-up continuation with methods similar to the AMANHI bio-repository study which involved 3 sites (Pakistan, Bangladesh and Pemba), methods already published (ref: DOI: 10.7189/jogh. 07.021202 is incorporated by reference herein in its entirety).

Demography: The population is a mix of Arab and original Waswahili inhabitants of the island. A significant portion of the population also identifies as Shirazi people.

Study Goal: The main purpose of the study is to identify important biomarkers as predictors of important pregnancy-related outcomes and to extend bio-bank in Pemba (started with AMANHI) for future research as new methods and technologies become available.

Study Participants: Women of Reproductive Age (18-49 years), resident of the island who intended to stay in the study areas for the entire duration of follow-up and consented for collection of epidemiological data as well as biological samples are being enrolled in the study

Method: Trained women fieldworkers (FWs), performed home visits every 2-3 months to all women of reproductive age in the study area to enquire about pregnancy. If a woman reported two or more consecutive missed period or suspected a pregnancy, FWs conducted a urine pregnancy test to confirm it. Pregnant women who provided consent underwent a screening ultrasound to date the pregnancy. All women in their early pregnancies with ultrasound confirmed gestational age between 8 and 19 weeks were consented for participation in the study. Women were randomized for antenatal maternal sample collection at either 24-28 weeks or 32-36 weeks gestation. The fathers of the babies also consented for their saliva sample collection.

A trained study worker conducted four home visits to all women in the cohort; at baseline (immediately after enrolment), at 24-28 weeks, 32-36 weeks and after 37 completed weeks of pregnancy to collect self-reported morbidity data from these women. Blood pressure and protein urea was measured by the study staff during these visits.

Bio-specimens (blood and urine) were collected from the pregnant women at the time of enrollment (between 8 and 19 weeks) and once during the antenatal period (24-28 or 32-26 weeks of gestation.

Reference: AMANHI (Alliance for Maternal and Newborn Health Improvement) Bio-banking Study group); Understanding biological mechanisms underlying adverse birth outcomes in developing (PMID: 29163938) is incorporated by reference herein in its entirety.

Cohort H (RS)

This prospectively collected cohort from Roskilde hospital in Denmark, sampled participants 4 times during pregnancy at weeks 12, 20, 25 and 32. All Danish-speaking women over the age of 18 were eligible for inclusion. At each visit a blood sample was collected and we performed a detailed ultrasound examination. At end of collection in 2010 the cohort included 1,214 participants.

Reference: Gybel-Brask, D., Hegdall, E., Johansen, J., Christensen, I. J. & Skibsted, L. Serum YKL-40 and uterine artery Doppler—a prospective cohort study, with focus on preeclampsia and small-for-gestational-age. Acta Obstet Gynecol Scand 93, 817-824 (2014) is incorporated by reference herein in its entirety.

Methods

cfRNA Isolation

Plasma samples received on dry ice from our collaborators were stored at −80° C. until further processing. Total circulating nucleic acid was extracted from plasma ranging in volume from ˜215 ul to 1 ml, using a column-based commercially available extraction kit, following the manufacturer's instructions (Plasma/Serum Circulating and Exosomal RNA purification kit, Norgen, cat 42800). We added in spike-in control RNA during extraction to monitor the yield.

Following extraction cfDNA was digested using Baseline-ZERO DNase (Epicentre) and the remaining cfRNA purified using RNA Clean and Concentrator-5 kit (Zymo, cat R1016) or RNeasy MinElute Cleanup Kit (Qiagen, cat 74204).

RT-qPCR Assay

We developed a RT-qPCR based method to assess the relative amount of cfRNA extracted from each sample. We measured and compared the threshold Cycles (Ct) values from each RNA extraction using a 3 color multiplex qPCR assay using TaqPath™ 1-Step Multiplex Master Mix kit (Catalog A28526) and Quant Studio 5 system. We measured the Ct values for an endogenous housekeeping gene (ACTB; Thermofisher Scientific, cat 4351368) and a spike-in control RNA as well as an assay to monitor presence of DNA contamination (IDT).

cfRNA Library Preparation

cfRNA libraries were prepared using the SMARTer Stranded Total RNAseq-Pico Input Mammalian kit (Takara, Cat 634418). following the manufacturer's instructions except we did not use ribo depletion. Library quality was assessed by RT-qPCR following the method described for assessing RNA extraction and Fragment analyzer analysis 5300 (Agilent Technologies).

Enrichment and Sequencing

Libraries were normalized before pooling for target capture. We used SureSelect Target Enrichment kit (Agilent Technologies, cat 5190-8645) and followed the manufacturer's instructions for hybrid capture. Samples were quantitated and 50 base-pair, paired-end sequencing was performed on a Novaseq S2. Between 98 and 144 samples were pooled and sequenced per sequencing run.

Analysis for Outliers

qPCR of ACTB and a spike-in control RNA as well as MultiQC sequencing metrics were monitored to eliminate sample outliers before performing gene expression analyses. Individual samples more than 3 standard deviations from the mean were removed as outliers. A set of samples were removed following this filtering.

Feature Normalization

For each gene, its relationship to total counts per sample is measured and corrected for using linear model residuals (e.g., gene ACTB). We also thought to correct the genes such that each cohort has the same mean value for each gene. However, the cohorts come from different parts of the gestational age spectrum. Therefore, only cohort effects orthogonal to the gestational age effect are corrected (e.g., gene CAPN6). Each cohort has its own color. The benefit of this correction becomes clearer if we zoom in to the second trimester. In this range, the CAPN6 counts from the bright green-colored cohort were unusually high and in the corrected version, this effect has been removed.

Mathematical Details

The steps for the above correction are as follows.

For each gene, model its counts as a function of total counts, cohort and gestational age. This gets a linear model gene=β₀+β₁totcounts+β₂cohort+β₃GA.

Once this model is fit, we can correct for the effect of these variables by taking the model residuals as the corrected values.

However, we don't want to correct for the gestational age effect (we want that to remain in the data because it's a variable of interest). To avoid doing so, set the coefficient 3 to zero before calculating fitted values and residuals.

Gestational Age Model without Cohort Correction

In this approach, we selected all samples from healthy pregnancies and split the dataset into a training set (1482 samples, 75% of data) and a test set (495 samples, 25% of data), in which samples were stratified by cohort. Samples that did not pass QC filtering based on basic sequencing metrics had been previously excluded from analysis (70 samples, 3.5% of total). We trained a Lasso model to predict the gestational age at collection for each sample using the mean absolute error as optimization metric and 10-fold cross-validation in the training set. We used all genes with mean log 2(CPM+1)>1 (12894 genes) plus a set of sequencing metrics as features for training. Modeling was performed in log 2(CPM+1) space and all data was centered and scaled prior to modeling using the training set statistics. This led to a model with mean absolute error of 15.9 days in the with-hold test set using 455 transcriptomic features. We then selected the top 55 features of this model and retrained the Lasso using the same approach described above achieving a mean absolute error of 16.3 days in the withhold test set.

Gene Set Enrichment Analysis (GSEA)

GSEA<PMIDs: 12808457, 16199517> was done with fast gsea algorithm <doi: doi.org/10.1101/060012> using Bioconductor fgsea package <DOI: 10.18129/B9.bioc.fgsea>. Gene sets were compiled from the Molecular Signatures Database (MSigDB)<21546393, 16199517> using CRAN msigdbr v7.2 API. We focused on two collections of gene sets: Gene Ontology (GO) sub-collection of the ontology gene sets, C5:GO, and the cell type signature gene sets, C8 (Table 32). Genes were ranked based on their log-fold change and associated Wald-test p-value obtained from the analysis of differential expression using Bioconductor's DESeq2, DOI: 10.18129/B9.bioc.DESeq2, <25516281> as a −log₁₀(p-value)*shrunkenLFC. GSEA was carried out on 364 samples from the Roskilde cohort collected from 91 women with healthy pregnancies over 4 time intervals during pregnancy, 11-14 weeks, 17-xxx w, xxx-xxx w, and xxx-xxx w. Log-fold changes and corresponding p-values were obtained from pairwise comparisons between collections 1 and 2, 1 and 3, and 1 and 4. Significantly enriched gene sets (Benjamini-Hochberg adjusted p-value<0.01), whose number varied predictably with the distance between the comparators (e.g., Table 33), were used in downstream analyses, including analysis of plasma transcriptome partitioning and set-specific longitudinal trends.

Evaluating Changes in Plasma Transcriptome Partitioning

Plasma transcriptome can be phenomenologically viewed as being partitioned between characteristic sets of genes. We assessed this partitioning in each RNAseq sample by converting raw gene counts to counts per million (CPM) and summing these CPMs over all genes in each of the sets. The resulting cumulative CPM score, which is a relative measure of abundance of each gene set in the overall transcriptome, was used to directly compare gene sets across collection time points. Cumulative CPM scores for all gene sets significantly enriched between collections 1 and 4 were calculated for every RNAseq sample. The scores for each sample were regressed onto the recorded gestational age (in weeks) using a linear model. Gene sets with an adjusted p-value for the gestational age coefficient <0.01 were considered to be having a significant (positive or negative) trend in their relative abundance. The association of these trends with the time component in the data was further verified by scrambling the temporal structure and re-examining the trends along the original time variable. For each mother we also evaluated the monotonicity of the cumulative CPM score function along the collection times. Since there are 24 possible permutations of order of the 4 collection times and only one of those permutations allows for a monotonic upward trend (and one—for downward), we were able to analytically assess the significance of observed number monotonic trends among 91 mothers using a Chi-squared test.

### REFERENCES

ACOG. Committee Opinion No. 688: Management of Suboptimally Dated Pregnancies. Obstetrics & Gynecology 129, e29-e32 (2017) is incorporated by reference herein in its entirety.
ACOG. Hypertension in pregnancy. Report of the American College of Obstetricians and Gynecologists' Task Force on Hypertension in Pregnancy. in 122, 1122-1131 (2013) is incorporated by reference herein in its entirety.
Alimirzaie, S., Bagherzadeh, M. & Akbari, M. R. Liquid biopsy in breast cancer: A comprehensive review. Clin Genet 95, 643-660 (2019) is incorporated by reference herein in its entirety.
Blencowe, H. et al. National, regional, and worldwide estimates of preterm birth rates in the year 2010 with time trends since 1990 for selected countries: a systematic analysis and implications. Lancet 379, 2162-2172 (2012) is incorporated by reference herein in its entirety.
Chen, X. et al. The potential role of pregnancy-associated plasma protein-A2 in angiogenesis and development of preeclampsia. Hypertension Research 1-11 (2019). doi:10.1038/s41440-019-0224-8 is incorporated by reference herein in its entirety.
Cui, Y. et al. Single-Cell Transcriptome Analysis Maps the Developmental Track of the Human Heart. CellReports 26, 1934-1950.e5 (2019) is incorporated by reference herein in its entirety.
Cunningham, P. & McDermott, L. Long chain PUFA transport in human term placenta. J Nutr 139, 636-639 (2009) is incorporated by reference herein in its entirety.
Feingold, K. R., Anawalt, B., Boyce, A. & Chrousos, G. Endocrinology of Pregnancy—Endotext. (2000) is incorporated by reference herein in its entirety.
Gao, S. et al. Tracing the temporal-spatial transcriptome landscapes of the human fetal digestive tract using single-cell RNA-sequencing. Nat Cell Biol 20, 721-734 (2018) is incorporated by reference herein in its entirety.
Gybel-Brask, D., Høgdall, E., Johansen, J., Christensen, I. J. & Skibsted, L. Serum YKL-40 and uterine artery Doppler—a prospective cohort study, with focus on preeclampsia and small-for-gestational-age. Acta Obstet Gynecol Scand 93, 817-824 (2014) is incorporated by reference herein in its entirety.
Hadlock, F. P. et al. Estimating fetal age using multiple parameters: a prospective evaluation in a racially mixed population. American Journal of Obstetrics & Gynecology MFM 156, 955-957 (1987) is incorporated by reference herein in its entirety.
Haug, E. B. et al. Life Course Trajectories of Cardiovascular Risk Factors in Women With and Without Hypertensive Disorders in First Pregnancy: The HUNT Study in Norway. J Am Heart Assoc 7, e009250 (2018) is incorporated by reference herein in its entirety.
Koh, W. et al. Noninvasive in vivo monitoring of tissue-specific global gene expression in humans. Proc. Natl. Acad. Sci. U.S.A. 111, 7361-7366 (2014) is incorporated by reference herein in its entirety.
Kramer, A. W., Lamale-Smith, L. M. & Winn, V. D. Differential expression of human placental PAPP-A2 over gestation and in preeclampsia. Placenta 37, 19-25 (2016) is incorporated by reference herein in its entirety.
Marinić, M. & Lynch, V. J. Relaxed constraint and functional divergence of the progesterone receptor (PGR) in the human stem-lineage. PLoS Genet 16, e1008666 (2020) is incorporated by reference herein in its entirety.
McLean, M. et al. A placental clock controlling the length of human pregnancy. Nature Medicine 1, 460-463 (1995) is incorporated by reference herein in its entirety.
Moufarrej, M. N. et al. Early prediction of preeclampsia in pregnancy with circulating, cell-free RNA. medRxiv 2021.03.11.21253393 (2021). doi:10.1101/2021.03.11.21253393 is incorporated by reference herein in its entirety.
Munchel, S. et al. Circulating transcripts in maternal blood reflect a molecular signature of early-onset preeclampsia. Sci Transl Med 12, eaaz0131 (2020) is incorporated by reference herein in its entirety.
Myatt, L. & Roberts, J. M. Preeclampsia: Syndrome or Disease? Curr Hypertens Rep 17, 83-8 (2015) is incorporated by reference herein in its entirety.
Ngo, T. T. M. et al. Noninvasive blood tests for fetal development predict gestational age and preterm delivery. Science 360, 1133-1136 (2018) is incorporated by reference herein in its entirety.
Nussbaum et al. Principles of clinical cytogenetics and genome analysis. In: Thompson & Thompson genetics in medicine. (Elsevier, 2016) is incorporated by reference herein in its entirety.
Paik Soonmyung, S. S. T. G. K. C. B. J. C. M. B. F. L. W. M. G. W. D. P. T. H. W. F. E. R. W. D. L. B. J. W. N. A Multigene Assay to Predict Recurrence of Tamoxifen-Treated, Node-Negative Breast Cancer. 1-10 (2004) is incorporated by reference herein in its entirety.
Pennington, K. A., Schlitt, J. M., Jackson, D. L., Schulz, L. C. & Schust, D. J. Preeclampsia: multiple approaches for a multifactorial disease. Dis Model Mech 5, 9-18 (2012) is incorporated by reference herein in its entirety.
Perschbacher, K. J. et al. Reduced mRNA Expression of RGS2 (Regulator of G Protein Signaling-2) in the Placenta Is Associated With Human Preeclampsia and Sufficient to Cause Features of the Disorder in Mice. Hypertension 75, 569-579 (2020) is incorporated by reference herein in its entirety.
Poon, C. E., Madawala, R. J., Day, M. L. & Murphy, C. R. Claudin 7 is reduced in uterine epithelial cells during early pregnancy in the rat. Histochem Cell Biol 139, 583-593 (2013).
Redman, C. W. & Sargent, I. L. Latest advances in understanding preeclampsia. Science 308, 1592-1594 (2005) is incorporated by reference herein in its entirety.
Ryan, D. et al. Development of the Human Fetal Kidney from Mid to Late Gestation in Male and Female Infants. EBioMedicine 27, 275-283 (2018) is incorporated by reference herein in its entirety.
Savitz, D. A. et al. Comparison of pregnancy dating by last menstrual period, ultrasound scanning, and their combination. YMOB 187, 1660-1666 (2002) is incorporated by reference herein in its entirety.
Skupski, D. W. et al. Estimating Gestational Age From Ultrasound Fetal Biometrics. Obstetrics & Gynecology 130, 433-441 (2017) is incorporated by reference herein in its entirety.
Uhlén, M. et al. Proteomics. Tissue-based map of the human proteome. Science 347, 1260419 (2015) is incorporated by reference herein in its entirety.
Del Vecchio, G. et al. Cell-free DNA Methylation and Transcriptomic Signature Prediction of Pregnancies with Adverse Outcomes. Epigenetics 00, 1-20 (2020) is incorporated by reference herein in its entirety.
Wang, G., Bonkovsky, H. L., de Lemos, A. & Burczynski, F. J. Recent insights into the biological functions of liver fatty acid binding protein 1. Journal Lipid Research 56, 2238-2247 (2020) is incorporated by reference herein in its entirety.
White, V. et al. IGF2 stimulates fetal growth in a sex- and organ-dependent manner. Pediatric Research 83, 183-189 (2017) is incorporated by reference herein in its entirety.
Wildman, D. E. Review: Toward an integrated evolutionary understanding of the mammalian placenta. Placenta 32 Suppl 2, S142-5 (2011) is incorporated by reference herein in its entirety.
Yuqiong Hu, X. W. B. H. Y. M. Y. C. L. Y. J. Y. J. D. Y. W. W. W. L. W. J. Q. F. T. Dissecting the transcriptome landscape of the human fetal neural retina and retinal pigment epithelium by single-cell RNA-seq analysis. 1-26 (2019). doi:10.1371/journal.pbio.3000365 is incorporated by reference herein in its entirety.
Yuqiong Hu, X. W. B. H. Y. M. Y. C. L. Y. J. Y. J. D. Y. W. W. W. L. W. J. Q. F. T. Dissecting the transcriptome landscape of the human fetal neural retina and retinal pigment epithelium by single-cell RNA-seq analysis. 1-26 (2019). doi:10.1371/journal.pbio.3000365 is incorporated by reference herein in its entirety.
Zeller, T. et al. Transcriptome-Wide Analysis Identifies Novel Associations With Blood Pressure. Hypertension 70, 743-750 (2017) is incorporated by reference herein in its entirety.

### Example 16: Prediction of Very Early Pre-Term Birth (ePTB) on Combined Multiple Cohorts

All PTB cohorts from Example 4 and Example 8 were combined in a single data set, as shown in FIG. 26A, totaling 58 case subjects with very early preterm delivery and 487 full-term deliveries. Very early Pre-term Birth (ePTB) was defined as deliveries occurring after 16 weeks of gestation and before 32 weeks of gestation (including cases of late miscarriages).

As shown in FIG. 26B, a cohort of 545 subjects (58 very early pre-term and 487 full-term controls) was established (with patient identification numbers shown on the x-axis). From this cohort, one or more biological samples (e.g., 1 or 2) were collected and assayed at different time points corresponding to an estimated gestational age (shown on the y-axis, in increasing order of estimated gestational age at delivery) of a fetus of each subject, using methods and systems of the present disclosure. For example, the estimated gestational age (shown on the y-axis) may be determined using methods such as ultrasound imaging, a last menstrual period (LMP) date, or a combination thereof, and may range from 0 to about 42 weeks

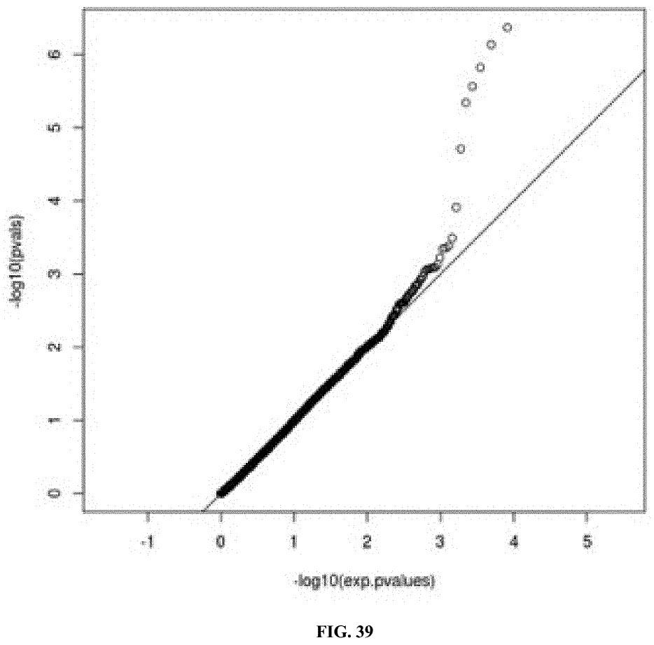

In order to mitigate the gestational age effect for blood collection in this analysis, only samples collected between 16 and 27 weeks of gestational age were included. Table 34 shows the top 30 differentially expressed genes for predicting very early preterm birth between 16 to 32 weeks with blood collected between 16 to 27 weeks, with significant statistical significance after adjustment for multiple hypothesis correction; the results summarized in this table also showed a significant deviation from the null hypothesis in a QQ plot for differential expression in very early pre-term cases (as shown in FIG. 39). Differential expression analysis was performed using EdgeR, and accounting for ethnicity and cohort effects (58 ePTB cases and 487 controls).

TABLE 34
Top set of genes that are predictive for ePTB between
16 and 32 weeks of gestational age with blood samples
collected between 16 and 27 weeks of gestational age

| Gene | logFC | log(CPM) | P-Value | FDR |
|---|---|---|---|---|
| COL3A1 | −1.554608 | 2.721233 | 4.30E−07 | 0.004491 |
| COL1A2 | −1.476499 | 2.139572 | 7.32E−07 | 0.004491 |
| COL1A1 | −1.60053 | 2.71966 | 1.51E−06 | 0.006179 |
| EPB41L4A | −0.580864 | 2.971978 | 2.75E−06 | 0.008421 |
| CDR1-AS | −0.983948 | 3.04125 | 4.57E−06 | 0.011204 |
| MMP2 | −1.182085 | 1.154661 | 1.94E−05 | 0.039687 |
| ATP5F1 | −0.130342 | 6.243824 | 1.23E−04 | 0.214913 |
| CDCA7L | −0.294654 | 5.140473 | 3.23E−04 | 0.495809 |
| CLSPN | −0.241616 | 4.865637 | 4.15E−04 | 0.504392 |
| RRM2 | −0.408065 | 4.269675 | 4.44E−04 | 0.504392 |
| ZCCHC7 | −0.144083 | 6.964859 | 4.52E−04 | 0.504392 |
| PDHA1 | −0.177542 | 5.60246 | 5.97E−04 | 0.574045 |
| TK1 | −0.528352 | 1.51427 | 7.36E−04 | 0.574045 |
| CCNA2 | −0.381202 | 2.852578 | 8.17E−04 | 0.574045 |
| TIPRL | −0.151145 | 5.006339 | 8.29E−04 | 0.574045 |
| TYMS | −0.330468 | 4.326804 | 8.35E−04 | 0.574045 |
| SNRPD3 | −0.14252 | 6.572218 | 8.62E−04 | 0.574045 |
| PSMD14 | −0.166879 | 4.365445 | 8.62E−04 | 0.574045 |
| CCDC80 | −0.773546 | 3.143176 | 8.89E−04 | 0.574045 |
| TUBB2A | −0.782378 | 3.745655 | 9.52E−04 | 0.583731 |
| C1S | −0.715219 | 0.853868 | 1.08E−03 | 0.633619 |
| CEP68 | 0.248055 | 4.095732 | 1.18E−03 | 0.636236 |
| TIMELESS | −0.261195 | 3.754269 | 1.19E−03 | 0.636236 |
| PER3 | 0.281305 | 4.239084 | 1.35E−03 | 0.668346 |
| RTEL1P1 | 1.337333 | 1.13544 | 1.38E−03 | 0.668346 |
| DCN | −1.031659 | 1.625258 | 1.46E−03 | 0.668346 |
| CD96 | −0.447194 | 5.016654 | 1.47E−03 | 0.668346 |
| LRRC23 | −0.288526 | 2.094129 | 1.63E−03 | 0.708272 |
| TRIM23 | 0.223815 | 5.477493 | 1.73E−03 | 0.708272 |
| TOP2A | −0.225064 | 5.946619 | 1.73E−03 | 0.708272 |

### Example 17: Prediction of Gestational Diabetes Mellitus (GDM) on Combined Multiple Cohorts

Using systems and methods of the present disclosure, a prediction model was developed to detect or predict a risk of gestational diabetes mellitus (GDM) of a pregnant subject. The prediction model development comprised obtaining a cohort of subjects and training the prediction model on a training dataset corresponding to the cohort of subjects represented in Table 35.

Further, whole transcriptome data from four cohorts were analyzed by the abundant gene search method. The three (K, M, P) cohorts contain combined 49 GDM samples and 430 control samples with gestational age at blood draw having a median of 21 weeks. Additionally, the R cohort comprised blood samples collected from 11 participants diagnosed with gestational diabetes and 119 healthy participants with multiple blood draws at gestational age of about 13, 20, 26, and 32 weeks.

TABLE 35
GDM cases & controls by cohort

| Cohort | Cases | Controls |
|---|---|---|
| K | 18 | 164 |
| M | 12 | 187 |
| P | 19 | 79 |
| R, Draw 1 (about13 weeks) | 9 | 105 |
| R, Draw 2 (about 20 weeks) | 8 | 109 |
| R, Draw 3 (about 26 weeks) | 11 | 119 |
| R. Draw 4 (about 32 weeks) | 9 | 116 |

Genes Predictive of GDM Determined by Differential Expression Analysis

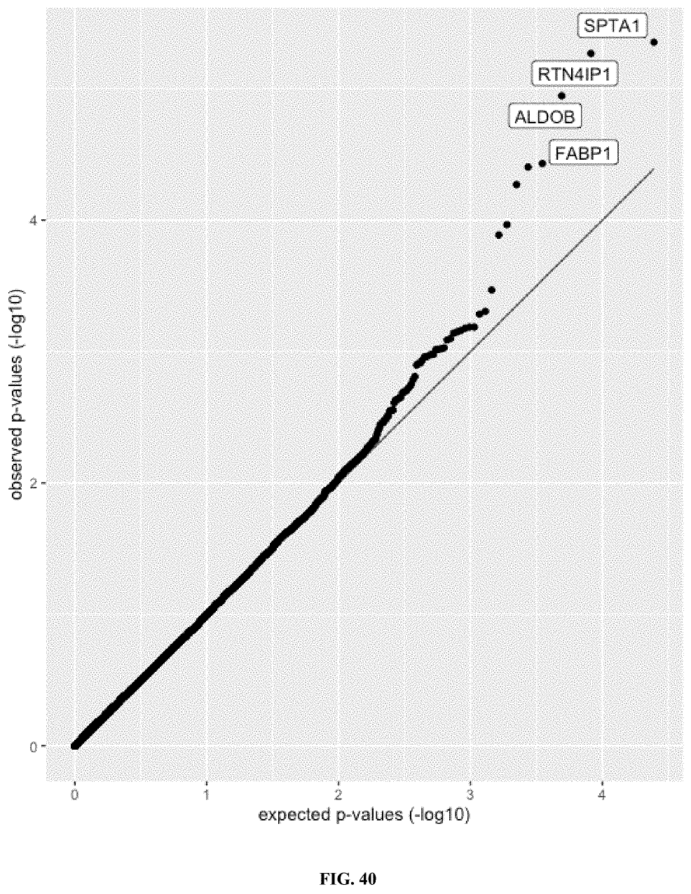

Differential expression analysis was performed with DESeq on gene expression data from a training dataset comprising three combined cohorts (P, M, and K). The training set comprised 49 GDM cases and 430 healthy controls. The top 4 differentially expressed genes were identified by QQ plot, as shown in FIG. 40. Log 2 RPM expression levels of the top 4 genes from the training set were used as features to train a logistic model (L2 penalty), where individual models were developed for each gene. The test set comprised an independent cohort (R) with multiple blood draws from a group of maternal subjects. The trained models were evaluated on draws 3 & 4 in the test cohort to yield AUC metrics at about 26 and 32 weeks of gestational age, respectively, as shown in Table 36.

TABLE 36
Performance of models developed for each of the top 4 genes identified
by differential expression evaluated on an independent test
cohort (R) at about 26 and 32 weeks gestational age

| | | | Test AUC | Test AUC |
|---|---|---|---|---|
| | | | RS Draw 3, | RS Draw 4, |
| | Log2 fold | | about | about |
| Gene | change | P-value | 26 weeks | 32 weeks |
| SPTA1 | 0.564 | 0.0000248 | 0.58 | 0.51 |
| RTN4IP1 | −0.324 | 0.0000564 | 0.55 | 0.48 |
| ALDOB | 0.945 | 0.0000716 | 0.62 | 0.77 |
| FABP1 | 0.732 | 0.0001020 | 0.52 | 0.75 |

Genes Predictive of GDM Discovered by a Leave-One-Cohort-Out Analysis

Robust feature discovery was performed on a training dataset by identifying genes that are consistently predictive of GDM from cohort to cohort. For a group of cohorts that comprise a training dataset, each cohort is held out as an independent test set, while the remaining cohorts are reserved for training. Gene expression values are expressed as standardized Log 2 RPM and combined from three cohorts (K, M, and P) with a total of 49 GDM cases and 430 controls with a median gestational age of 21 weeks, as shown in Table 35. In each round, two cohorts were used to train, while the remaining cohort was reserved for testing. Features were selected by filtering for genes with Mann Whitney p-values<0.05 when comparing GDM cases versus controls. Genes were then further filtered for those whose absolute GDM effect size had a mean value >0.5 and a coefficient of variation <0.5 across the training cohorts. Genes were then further filtered based on whether the trained logistic model (L2 penalty) for the gene had a mean AUC>0.6 when each training cohort was reserved for testing to further improve feature robustness across each cohort. The top 5 performing genes were then combined, and gene filtering was repeated as described above. Further, a leave-one-out analysis was performed across the full training set (3 cohorts combined), and a final AUC>0.6 threshold was applied. Seven genes were identified from the leave-one-cohort analysis across the training dataset, as shown in Table 37.

TABLE 37
Top 8 GDM genes identified by a leave-one-cohort-
out analysis within the training dataset

| # | Gene Name |
|---|---|
| 1 | TMEM101 |
| 2 | FCHO2 |
| 3 | PPP1R15A |
| 4 | NOMO3 |
| 5 | ANKRD54 |
| 6 | MT-TH |
| 7 | OARD1 |
| 8 | UBE2Q2 |

A logistic model (L2 penalty) based on the 8 genes was trained on the full 3-cohort training set and evaluated on an independent cohort RS (Table 35). Evaluation of the model on the independent test showed an AUC of 0.55 when predicting at about 20 weeks gestational age (Draw 2) and 0.57 at about 26 weeks gestational age (Draw 3).

Genes Predictive of GDM Discovered by Effect Size

A leave-one-out cross validation was performed on a small training set from one cohort with samples at about 13 weeks gestational age (R, Draw 1). The training set comprised 9 GDM cases and 105 controls. Gene collections that are upregulated and downregulated in GDM were selected from the training data as follows. Gene expression values were transformed into Log 2 counts. A gene collection was identified by finding the optimal gene set where the sum of counts maximized the GDM effect size. A grid search over the effect size threshold was performed to tune the hyperparameter used to select the highest effect genes based on the maximal GDM effect of the resultant summed collection. A gene collection was generated for both upregulated (n=7) and downregulated (n=2) GDM effects (Table 38). These two gene collections were then used as features in a logistic model (L2 penalty) trained on samples from R Draw 1 at about 13 weeks gestation and tested on sample collected at a later gestational age of about 20 weeks from the same cohort (R Draw 2 with 8 cases and 109 controls). Performance on the test set was observed with an AUC of 0.60.

TABLE 38
Genes comprising the upregulated and downregulated gene collections
identified from the first trimester (~13 weeks gestation)

| # | Gene Name | GDM Effect Size Collection |
|---|---|---|
| 1 | C1QTNF6 | Upregulated |
| 2 | AZIN2 | Upregulated |
| 3 | NEAT1 | Upregulated |
| 4 | PHYHD1 | Upregulated |
| 5 | PINK1-AS | Upregulated |
| 6 | NPIPA5 | Upregulated |
| 7 | PGS1 | Upregulated |
| 8 | ADIRF | Downregulated |
| 9 | PALMD | Downregulated |

PCA Components Predictive of GDM

Features were identified from a training set comprised of Log 2 RPM gene expression data from three cohorts (P, M, and K, ˜21 weeks gestation). Seventy percent of the training data was split into a training set (36 cases and 299 controls), while the remaining 30% was used as a test set (13 cases and 131 controls) for feature engineering. Candidate genes were selected for an upregulated effect size in GDM greater than an effect size threshold. Principal component analysis (PCA) was performed and trained on standardized Log 2 RPM counts from controls in the training set. The full training and test sets were then PCA transformed. A logistic model (L1 penalty) was trained on the PCA components calculated from the training data and then applied to principal components similarly calculated from the test dataset. The hyperparameters for the effect size threshold and the PCA variance threshold were optimized by a grid search based on optimizing the AUC on the test set. The effect size threshold was set to 0.6, yielding 15 high effect genes shown in Table 39, and the PCA variance threshold was set to 0.6, yielding 3 principal components after transforming the 15 high effect genes.

TABLE 39
15 high effect genes comprising the principal
component features in the GDM model

| # | Gene Name |
|---|---|
| 1 | SRP14 |
| 2 | ATP6V1G1 |
| 3 | METTL9 |
| 4 | OARD1 |
| 5 | HNRNPA2B1 |
| 6 | PPP1CB |
| 7 | FUNDC2 |
| 8 | BDH2 |
| 9 | C18orf32 |
| 10 | COPS3 |
| 11 | ALDOB |
| 12 | SMDT1 |
| 13 | VKORC1 |
| 14 | UBE2J1 |
| 15 | RHOA |

The final principal component transformation based on the 15 high effect genes was retrained on the full training dataset (P, M, and K) with 49 GDM cases and 430 controls, and then used as features in a logistic model trained on the full training dataset. The model was evaluated on an independent cohort (R), and performance was observed with an AUC of 0.59 for Draw 2 (8 cases and 109 controls at about 20 weeks) and an AUC of 0.60 for Draw 3 (11 cases and 119 controls at about 26 weeks).

### Example 18: Clinical Intervention Care Pathway to Improve Early Pre-Term Birth (ePTB) Outcomes Based on Prediction Test Administer in Second Trimester

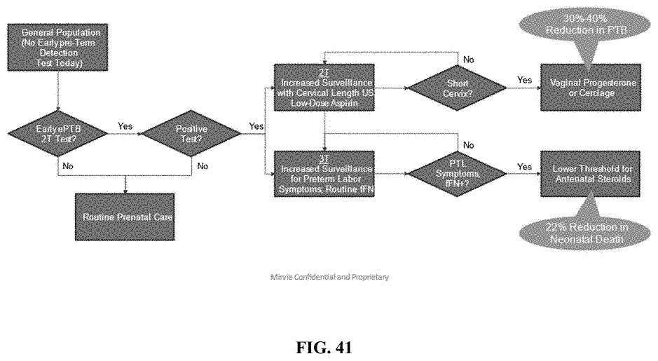

Using systems and methods of the present disclosure, a clinical intervention care plan algorithm was developed to improve early pre-term birth outcomes following results of predictive tests administered in the second trimester, as shown in FIG. 41.

Currently, there is no early pre-term test available for an asymptomatic general population without prior preterm history, and a majority of pregnancies are followed to routine prenatal care pathway. An ePTB prediction test is applied at early stage of pregnancy (13 to 26 weeks of gestational age), pregnant subjects who test positive are provided with two arm approaches. For a first arm, pregnant subjects who test positive at a second trimester are referred for increased surveillance with cervical length ultrasound and low dose aspirin treatment regimen. The pregnant subjects with short cervix then proceed for possible treatment with vaginal progesterone or surgical cerclage. In the first arm of the treatment, about 30-40% of spontaneous ePTB can be reduced or delayed.

On a second arm, pregnant subjects who test positive at a third trimester are referred for increased surveillance for preterm labor symptoms and routine fetal fibronectin testing (fFN) in cervical secretions. The pregnant subjects with active labor presentation and positive fFN test have a lower threshold for providing antennal steroid treatment to improve neonatal outcomes. In the second arm of the treatment, about 22% of neonatal death can be reduced.

### REFERENCES

Senarath, Sachintha; Ades, Alex; FRANZCOG; Nanayakkara, Pavitra; MRANZCOG, Cervical Cerclage: A Review and Rethinking of Current Practice, Obstetrical & Gynecological Survey: December 2020-Volume 75-Issue 12-p 757-765 is incorporated by reference in its entirety.
Child T, Leonard S A, Evans J S, Lass A. Systematic review of the clinical efficacy of vaginal progesterone for luteal phase support in assisted reproductive technology cycles. Reprod Biomed Online. 2018 June; 36(6):630-645. doi: 10.1016/j.rbmo.2018.02.001. Epub 2018 Feb. 22. PMID: 29550390 is incorporated by reference in its entirety.
McGoldrick E, Stewart F, Parker R, Dalziel S R. Antenatal corticosteroids for accelerating fetal lung maturation for women at risk of preterm birth. Cochrane Database of Systematic Reviews 2020, Issue 12. Art. No.: CD004454. DOI: 10.1002/14651858.CD004454.pub4. Accessed 20 Jul. 2021 is incorporated by reference in its entirety.

### Example 19: Clinical Intervention Care Pathway to Improve Preeclampsia (PE) Outcomes Based on Prediction Test Administer in Second Trimester

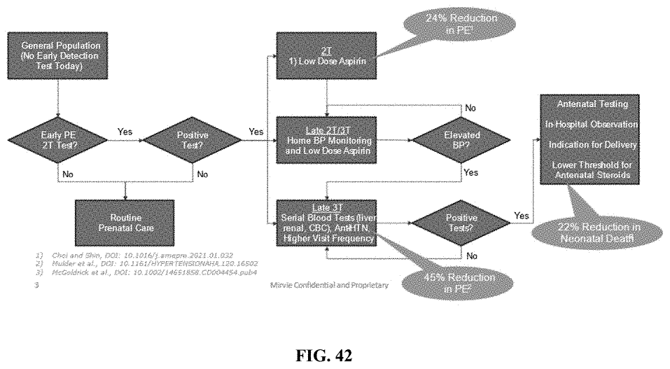

Using systems and methods of the present disclosure, a clinical intervention care plan algorithm was developed to improve preeclampsia outcomes following results of predictive tests administered in the second trimester, as shown in FIG. 42.

Currently, there is no preeclampsia test available for an asymptomatic general population without prior history of hypertension or prior preeclampsia, and a majority of pregnancies are followed to routine prenatal care pathway. If a PE prediction test is performed for subjects at an early stage of pregnancy (13 to 20 weeks of gestational age), pregnant subjects who test positive are provided three arm approaches. For a first arm, pregnant subjects who test positive at an early second trimester (13 to 16 weeks of gestation) are treated with low dose aspirin regime, which can result in a 24% reduction of early onset of preeclampsia.

In a second arm, pregnant subjects who test positive at a second or third trimester are referred for increased surveillance for home blood pressure monitoring and low dose aspirin treatment. In a third arm, pregnant subjects with elevated blood pregnancies proceed with serial blood tests for liver or renal dysfunction and treatment with anti-hypertension medications (e.g., hydralazine, labetalol and oral nifedipine), which can reduce incident of PE by 45%. By recommending the preeclampsia subjects with positive blood test for liver and renal dysfunctions for a combination of antenatal observation, indication for delivery, and possible lower threshold for antenatal steroid treatment, this can result in estimated 22% reduction in neonatal death.

### REFERENCES

Yeo Jin Choi, Sooyoung Shin, Aspirin Prophylaxis During Pregnancy: A Systematic Review and Meta-Analysis; Am J Prev Med, 2021 Jul; 61(1):e31-e45 is incorporated by reference in its entirety.
Eva G. Mulder, Chahinda Ghossein-Doha, Ella Cauffman, Veronica A. Lopes van Balen, Veronique M. M. M. Schiffer, Robert-Jan Alers, Jolien Oben, Luc Smits, Sander M. J. van Kuijk, Marc E. A. Spaanderman; Preventing Recurrent Preeclampsia by Tailored Treatment of Nonphysiologic Hemodynamic Adjustments to Pregnancy, Hypertension. 2021; 77:2045-2053 is incorporated by reference in its entirety.
McGoldrick E, Stewart F, Parker R, Dalziel S R. Antenatal corticosteroids for accelerating fetal lung maturation for women at risk of preterm birth. Cochrane Database Syst Rev. 2020 Dec. 25; 12(12):CD004454. doi: 10.1002/14651858.CD004454.pub4. PMID: 33368142; PMCID: PMC8094626 is incorporated by reference in its entirety.

### Example 20: Clinical Intervention Care Pathway to Improve Gestational Diabetes Mellitus (GDM) Outcomes Based on Prediction Test Administer in Second Trimester

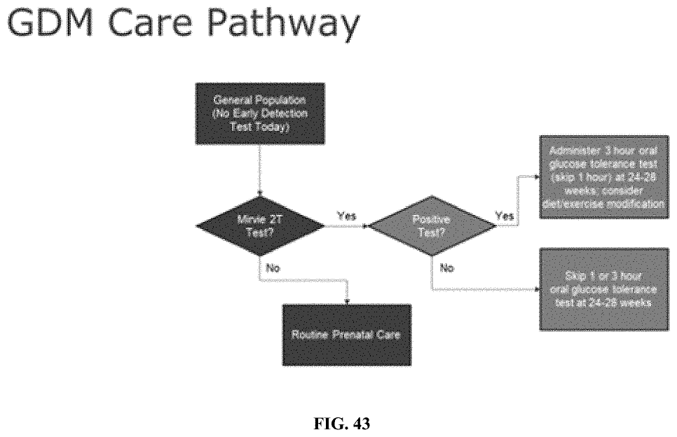

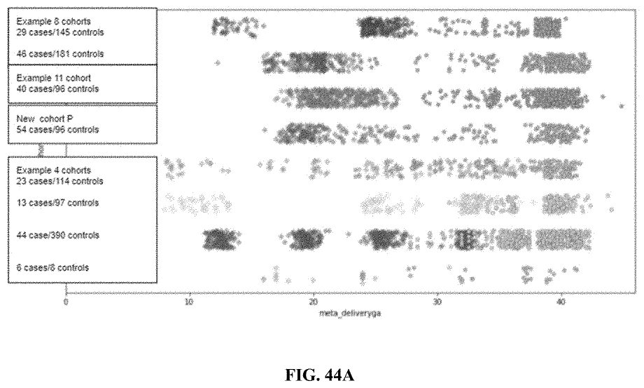

Using systems and methods of the present disclosure, a clinical intervention care plan algorithm was developed to improve GDM outcomes following results of predictive tests administered in the second trimester, as shown in FIG. 43.

Currently, there is no gestational diabetes mellitus test available for an asymptomatic general population in early second trimester and a majority of pregnancies are followed to routine prenatal care pathway with diagnostic oral glucose tolerance test at 24-28 weeks of gestational age. If a gestational diabetes prediction test is performed for subjects at an early stage of pregnancy (13 to 20 weeks of gestational age), pregnant subjects who test positive are provided two arm approaches. For a first arm, pregnant subjects who test negative at an early second trimester (13 to 16 weeks of gestation) are not recommended to take an oral glucose tolerance test at 24-28 weeks of gestational age.

In a second arm, pregnant subjects who test positive at a second trimester are recommended to skip a 1-hour glucose tolerance test and to proceed with taking a 3-hour glucose tolerance test for improved accuracy of diagnosis.

### Example 21: Prediction of Pre-Term Birth (PTB) on Combined Multiple Cohorts

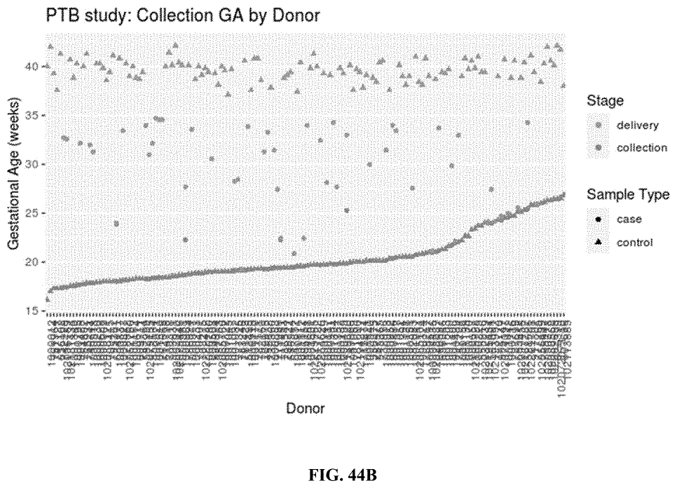

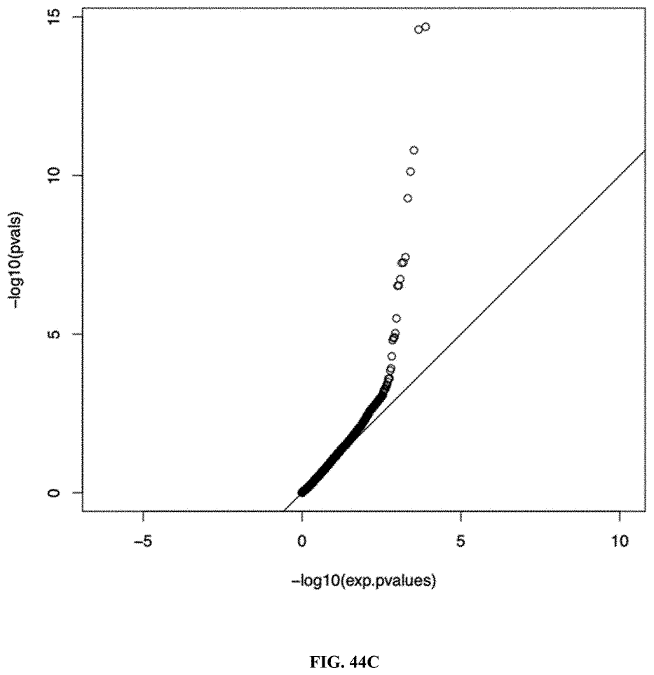

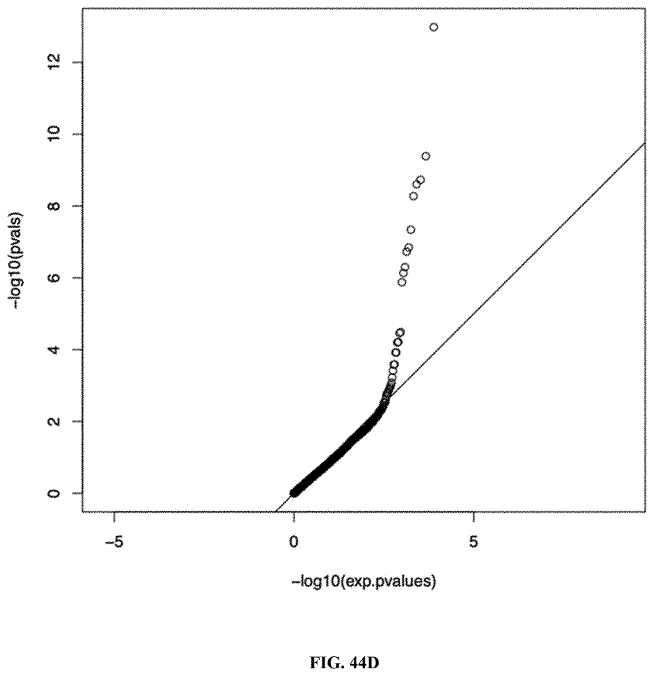

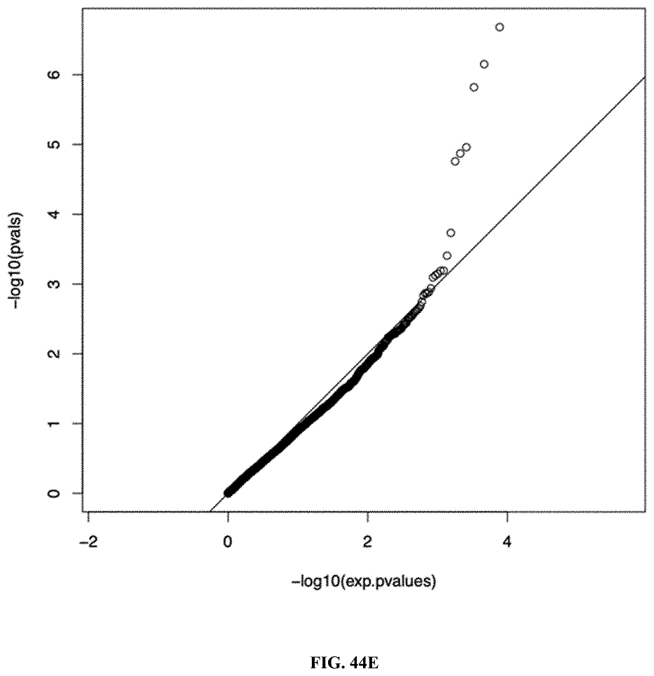

All PTB cohorts from Examples 4, 8, and 11, plus an additional cohort (P), were combined in a single data set, as shown in FIG. 44A, totaling 255 samples from subjects with preterm delivery before 35 weeks of gestation age and 1269 samples from healthy control subjects with delivery gestation age after 37 weeks.

An additional cohort (P) of subjects was obtained as follows. As shown in FIG. 44B, a cohort of 150 subjects (54 pre-term and 96 full-term controls) was established (with patient identification numbers shown on the x-axis). From this cohort, one or more biological samples (e.g., 1 or 2) were collected and assayed at different time points corresponding to an estimated gestational age (shown on the y-axis, in increasing order of estimated gestational age at delivery) of a fetus of each subject, using methods and systems of the present disclosure. For example, the estimated gestational age (shown on the y-axis) may be determined using methods such as ultrasound imaging, a last menstrual period (LMP) date, or a combination thereof, and may range from 0 to about 42 weeks.

In order to mitigate gestational age effects for blood collection, three separate differential expression analyses for combined cohorts were performed as follows. First, an analysis for differentially expressed genes between the pre-term birth case samples (delivered before 35 weeks) and control samples (delivered at or after 37 weeks) was performed for blood samples collected between 17-28 weeks of gestational age (190 cases and 859 controls). In the second analysis, differentially expressed genes between the pre-term birth case samples (delivered earlier than 35 weeks) and control samples (delivered after or at 37 weeks) were performed for blood samples collected between a narrow window of 23-26 weeks of gestational age (60 cases and 271 controls). In a third analysis, differentially expressed genes between the pre-term birth case samples (delivered earlier than 35 weeks) and control samples (delivered after or at 37 weeks) were performed for blood samples collected between at an earlier window between 17-23 weeks of gestational age (111 cases and 505 controls).

First differential expression analysis of predicting preterm birth earlier than 35 weeks of gestational age, with blood samples collected between 17-28 weeks of gestational age, was performed using EdgeR and accounting for ethnicity, and cohort effects and gestational age at collection (190 PTB cases and 859 controls). Table 40 shows a set of top 19 genes with p-value<0.1 after adjustment from multiple hypothesis correction (FDR value), and also showed a significant deviation from the null hypothesis in a QQ plot for differentially expressed in pre-term birth cases (as shown in FIG. 44C). Table 41 shows an additional set of genes with p-value<0.1 for predicting preterm birth earlier than 35 weeks of gestation, with blood samples collected between 17-28 weeks of gestational age. Genes are ordered according to their statistical significance (P-values).

TABLE 40
Top 19 genes with p-value < 0.1 after adjustment from multiple
hypothesis correction (FDR value), that are predictive for
preterm birth earlier than 35 weeks of gestation with blood
samples collected between 17-28 weeks of gestational age

| # | Gene | logFC | P-Value | FDR |
|---|---|---|---|---|
| 1 | FGA | −1.04779 | 2.04E−15 | 1.46E−11 |
| 2 | HRG | −1.14768 | 2.49E−15 | 1.46E−11 |
| 3 | FGB | −0.84237 | 1.60E−11 | 6.21E−08 |
| 4 | APOB | −0.78279 | 7.49E−11 | 2.19E−07 |
| 5 | APOH | −0.82927 | 5.19E−10 | 1.21E−06 |
| 6 | COL3A1 | −0.98584 | 3.76E−08 | 7.31E−05 |
| 7 | ALB | −0.57285 | 5.51E−08 | 8.32E−05 |
| 8 | HPD | −0.59372 | 5.70E−08 | 8.32E−05 |
| 9 | COL1A1 | −1.00293 | 1.84E−07 | 0.00023915 |
| 10 | FABP1 | −0.56313 | 2.94E−07 | 0.0003184 |
| 11 | CFH | −0.42425 | 3.00E−07 | 0.0003184 |
| 12 | COL1A2 | −0.81295 | 3.19E−06 | 0.00309871 |
| 13 | CYP2E1 | −0.47476 | 9.33E−06 | 0.00837437 |
| 14 | MUC3A | −0.5149 | 1.25E−05 | 0.01042708 |
| 15 | CDR1- | −0.537 | 1.34E−05 | 0.01043626 |
| | AS | | | |
| 16 | ALDOB | −0.48986 | 1.56E−05 | 0.01136251 |
| 17 | ADH1B | −0.46998 | 5.00E−05 | 0.03435136 |
| 18 | HP | −0.42634 | 0.0001198 | 0.07769152 |
| 19 | DCN | −0.66171 | 0.00014101 | 0.08662964 |

TABLE 41
Additional set of genes with p-value < 0.1 for predicting
preterm birth earlier than 35 weeks of gestation with blood
samples collected between 17-28 weeks of gestational age

| # | Gene | logFC | P-Value | FDR |
|---|---|---|---|---|
| 1 | INHBA | −0.37162 | 0.00024695 | 0.13632815 |
| 2 | MYH11 | −0.26583 | 0.00025577 | 0.13632815 |
| 3 | CCDC80 | −0.47289 | 0.00025694 | 0.13632815 |
| 4 | PLXNA3 | 0.43233 | 0.00032064 | 0.16273233 |
| 5 | HIST1H2AI | −0.17725 | 0.00039821 | 0.18855433 |
| 6 | AHNAK2 | −0.3859 | 0.00040383 | 0.18855433 |
| 7 | CCNA2 | −0.22972 | 0.00046407 | 0.2083505 |
| 8 | PRG4 | −0.43682 | 0.00053207 | 0.21732697 |
| 9 | 1-Mar | 0.347134 | 0.00053818 | 0.21732697 |
| 10 | CCR2 | 0.383962 | 0.00053992 | 0.21732697 |
| 11 | EZH1 | 0.090991 | 0.00056513 | 0.21989261 |
| 12 | MALAT1 | 0.384296 | 0.00063344 | 0.23852244 |
| 13 | KLF5 | −0.28811 | 0.00067648 | 0.24676558 |
| 14 | PLSCR1 | −0.13343 | 0.00084663 | 0.29328991 |
| 15 | UNK | 0.096595 | 0.00085524 | 0.29328991 |
| 16 | PAPPA2 | −0.40533 | 0.00090333 | 0.29328991 |
| 17 | PER3 | 0.171607 | 0.00090616 | 0.29328991 |
| 18 | CAMKK1 | 0.227011 | 0.00092964 | 0.29328991 |
| 19 | TMEM43 | 0.263695 | 0.00095742 | 0.29377879 |
| 20 | NBPF10 | 0.175322 | 0.00098153 | 0.29377879 |
| 21 | NELL2 | 0.356349 | 0.00109303 | 0.3034526 |
| 22 | ARG1 | −0.2776 | 0.00112046 | 0.3034526 |
| 23 | TEX30 | −0.19148 | 0.00112999 | 0.3034526 |
| 24 | TCN1 | −0.36384 | 0.00116198 | 0.3034526 |
| 25 | TK1 | −0.29507 | 0.0011672 | 0.3034526 |
| 26 | TMEM56 | −0.27078 | 0.00118023 | 0.3034526 |
| 27 | CLCN6 | 0.380015 | 0.00119582 | 0.3034526 |
| 28 | RNASE3 | −0.36576 | 0.00129822 | 0.31937455 |
| 29 | IL2RB | 0.220493 | 0.00134056 | 0.31937455 |
| 30 | DIRC2 | 0.317528 | 0.00139892 | 0.31937455 |
| 31 | PTGR1 | −0.19462 | 0.00140719 | 0.31937455 |
| 32 | ABCA13 | −0.30061 | 0.00142353 | 0.31937455 |
| 33 | PDE3B | 0.264993 | 0.00143959 | 0.31937455 |
| 34 | HSPA1B | 0.28971 | 0.00145009 | 0.31937455 |
| 35 | SH3BP5 | −0.13924 | 0.00149536 | 0.3232475 |
| 36 | SLC2A5 | −0.30138 | 0.0015704 | 0.33197687 |
| 37 | GPX3 | −0.24256 | 0.00161509 | 0.33197687 |
| 38 | PABPC1L | 0.456285 | 0.00162106 | 0.33197687 |
| 39 | ITGB7 | 0.287416 | 0.00167524 | 0.33715669 |
| 40 | MMP8 | −0.34981 | 0.00173049 | 0.33889101 |
| 41 | FERMT2 | −0.17972 | 0.0017688 | 0.33889101 |
| 42 | ATP10D | 0.248288 | 0.00179581 | 0.33889101 |
| 43 | PLK1 | −0.22723 | 0.00179999 | 0.33889101 |
| 44 | TYMS | −0.17849 | 0.00186307 | 0.34062912 |
| 45 | RRM2 | −0.21162 | 0.00186758 | 0.34062912 |
| 46 | ZBTB25 | 0.14581 | 0.00192423 | 0.34483979 |
| 47 | CD7 | 0.210869 | 0.00194975 | 0.34483979 |
| 48 | MTHFS | −0.11498 | 0.00205892 | 0.34711434 |
| 49 | IGFBP2 | −0.40481 | 0.002075 | 0.34711434 |
| 50 | PDK4 | −0.20835 | 0.00208199 | 0.34711434 |
| 51 | TTC14 | 0.287065 | 0.0020842 | 0.34711434 |
| 52 | CCNE2 | −0.17035 | 0.00213535 | 0.34711434 |
| 53 | EMB | −0.09234 | 0.00214103 | 0.34711434 |
| 54 | BEX1 | −0.26041 | 0.00217897 | 0.34842594 |
| 55 | TNNI2 | 0.242586 | 0.00225168 | 0.35053589 |
| 56 | DHX34 | 0.305572 | 0.00225222 | 0.35053589 |
| 57 | RETN | −0.3173 | 0.00232144 | 0.35239745 |
| 58 | CRISP3 | −0.36534 | 0.00234073 | 0.35239745 |
| 59 | CHPF2 | 0.296714 | 0.00235475 | 0.35239745 |
| 60 | CDH6 | 0.446673 | 0.00244603 | 0.3527879 |
| 61 | PGGHG | 0.451204 | 0.00247897 | 0.3527879 |
| 62 | SAYSD1 | −0.15461 | 0.0024981 | 0.3527879 |
| 63 | CANT1 | 0.189086 | 0.00250317 | 0.3527879 |
| 64 | TRIM8 | 0.088478 | 0.00250847 | 0.3527879 |
| 65 | ARHGEF18 | 0.184928 | 0.0025668 | 0.35669386 |
| 66 | GALNT7 | 0.171836 | 0.00266696 | 0.36327936 |
| 67 | LTF | −0.29442 | 0.00267643 | 0.36327936 |
| 68 | CEACAM8 | −0.29635 | 0.00272645 | 0.36581387 |
| 69 | PKP4 | −0.09544 | 0.00276342 | 0.36656121 |
| 70 | LENG8 | 0.264807 | 0.00283865 | 0.36910855 |
| 71 | ARL1 | −0.08755 | 0.00284586 | 0.36910855 |
| 72 | AZI2 | −0.07627 | 0.00296502 | 0.3803368 |
| 73 | SLC15A4 | 0.139099 | 0.00302285 | 0.38354039 |
| 74 | CCDC141 | 0.352908 | 0.00329923 | 0.40507236 |
| 75 | ANKRD36 | 0.143622 | 0.00330275 | 0.40507236 |
| 76 | APOC1 | −0.24152 | 0.00337521 | 0.40507236 |
| 77 | ZNF692 | 0.314622 | 0.0034314 | 0.40507236 |
| 78 | IL7R | 0.153439 | 0.00343657 | 0.40507236 |
| 79 | FN1 | −0.22938 | 0.0034427 | 0.40507236 |
| 80 | CKAP2L | −0.1414 | 0.00346852 | 0.40507236 |
| 81 | THBD | 0.31222 | 0.00355915 | 0.40507236 |
| 82 | OBSCN | 0.257153 | 0.00357239 | 0.40507236 |
| 83 | SELENOP | −0.2075 | 0.00358074 | 0.40507236 |
| 84 | PSMA3 | −0.07338 | 0.00358329 | 0.40507236 |
| 85 | PKD1 | 0.287392 | 0.00362194 | 0.40507236 |
| 86 | OLFM4 | −0.33973 | 0.00364367 | 0.40507236 |
| 87 | MANSC1 | −0.19999 | 0.00372481 | 0.40804253 |
| 88 | ACTA2 | −0.20389 | 0.0037403 | 0.40804253 |
| 89 | TMEM39A | 0.187568 | 0.00389507 | 0.42099242 |
| 90 | PLCH2 | 0.372379 | 0.00398863 | 0.42714967 |
| 91 | APBB3 | 0.429175 | 0.00413909 | 0.43923276 |
| 92 | ITGA9 | −0.22658 | 0.0041947 | 0.44112422 |
| 93 | EXOG | 0.166132 | 0.00429892 | 0.44263471 |
| 94 | HIST1H2AL | −0.15415 | 0.00431358 | 0.44263471 |
| 95 | CAMP | −0.29659 | 0.00432283 | 0.44263471 |
| 96 | MIB2 | 0.168881 | 0.00454601 | 0.4614398 |
| 97 | CCDC144B | 0.264578 | 0.00466679 | 0.46961576 |
| 98 | C1R | −0.35317 | 0.00470707 | 0.4696207 |
| 99 | SNX19 | −0.17109 | 0.00481307 | 0.47612692 |
| 100 | MEGF6 | 0.4601 | 0.00485623 | 0.47635988 |
| 101 | MNT | 0.09461 | 0.00492665 | 0.47700017 |
| 102 | RNF169 | 0.065814 | 0.00506902 | 0.47700017 |
| 103 | EPHB6 | 0.307981 | 0.00511012 | 0.47700017 |
| 104 | ITGA5 | 0.228836 | 0.0051295 | 0.47700017 |
| 105 | KIAA1143 | −0.07632 | 0.00513876 | 0.47700017 |
| 106 | RPS6KA5 | 0.107865 | 0.00519912 | 0.47700017 |
| 107 | C7orf31 | 0.095471 | 0.00523239 | 0.47700017 |
| 108 | VPS29 | −0.0608 | 0.00528375 | 0.47700017 |
| 109 | NUP210 | 0.223982 | 0.00530044 | 0.47700017 |
| 110 | ABCA7 | 0.306445 | 0.00534237 | 0.47700017 |
| 111 | KDM4B | 0.106133 | 0.00535228 | 0.47700017 |
| 112 | GALT | 0.229845 | 0.00535763 | 0.47700017 |
| 113 | NBPF26 | 0.170399 | 0.00543232 | 0.47700017 |
| 114 | HSPA1A | 0.178078 | 0.00543485 | 0.47700017 |
| 115 | FOXM1 | −0.18776 | 0.00569004 | 0.49567006 |
| 116 | TTN | 0.361796 | 0.00578995 | 0.50063788 |
| 117 | LUC7L3 | 0.076295 | 0.00588639 | 0.50106547 |
| 118 | SPOCK2 | 0.271026 | 0.00590797 | 0.50106547 |
| 119 | TESC | −0.11835 | 0.00594812 | 0.50106547 |
| 120 | NMRAL1 | 0.10644 | 0.0059666 | 0.50106547 |
| 121 | SERPINB10 | −0.27926 | 0.00603985 | 0.50359371 |
| 122 | S100A12 | −0.18638 | 0.00622577 | 0.51103623 |
| 123 | ATAD3B | 0.318935 | 0.00623391 | 0.51103623 |
| 124 | HELLS | −0.09181 | 0.00627331 | 0.51103623 |
| 125 | HIST1H3F | −0.14879 | 0.00630422 | 0.51103623 |
| 126 | NBPF8 | 0.167509 | 0.00652976 | 0.52466391 |
| 127 | FLT1 | −0.11643 | 0.00656771 | 0.52466391 |
| 128 | GINS2 | −0.26903 | 0.00660718 | 0.52466391 |
| 129 | COX20 | −0.08568 | 0.00680829 | 0.53399289 |
| 130 | SMIM20 | −0.12782 | 0.00681615 | 0.53399289 |
| 131 | PSMD14 | −0.07958 | 0.00689023 | 0.5361977 |
| 132 | CEACAM6 | −0.25445 | 0.00697169 | 0.53894431 |
| 133 | RPH3AL | −0.21896 | 0.0071488 | 0.54783785 |
| 134 | TRABD2A | 0.301776 | 0.0071806 | 0.54783785 |
| 135 | C3 | −0.18217 | 0.00732683 | 0.55510284 |
| 136 | PBXIP1 | 0.199065 | 0.00741578 | 0.55510284 |
| 137 | SULF2 | 0.258541 | 0.00741849 | 0.55510284 |
| 138 | NOTCH1 | 0.267867 | 0.00751332 | 0.55861766 |
| 139 | SMIM24 | −0.19888 | 0.00761332 | 0.56247034 |
| 140 | ERCC6L | −0.20093 | 0.00781274 | 0.56427079 |
| 141 | UNKL | 0.223599 | 0.00788269 | 0.56427079 |
| 142 | NBPF11 | 0.1189 | 0.00789503 | 0.56427079 |
| 143 | KRT8 | 0.193337 | 0.00795669 | 0.56427079 |
| 144 | MAST3 | 0.089153 | 0.00796759 | 0.56427079 |
| 145 | KCNH2 | −0.25824 | 0.00798896 | 0.56427079 |
| 146 | AC024560.3 | 0.202427 | 0.00803 | 0.56427079 |
| 147 | POLR2A | 0.050504 | 0.00808068 | 0.56427079 |
| 148 | DEFA3 | −0.32174 | 0.00814568 | 0.56427079 |
| 149 | SGSM3 | 0.101151 | 0.00829395 | 0.56427079 |
| 150 | LMTK2 | 0.161143 | 0.00832376 | 0.56427079 |
| 151 | SLC12A6 | 0.139805 | 0.00834325 | 0.56427079 |
| 152 | TOP2A | −0.10845 | 0.0083509 | 0.56427079 |
| 153 | MPO | −0.20111 | 0.00836113 | 0.56427079 |
| 154 | UVSSA | 0.2368 | 0.00836279 | 0.56427079 |
| 155 | ZNF865 | 0.175801 | 0.0084319 | 0.56550092 |
| 156 | TACC2 | 0.266062 | 0.00856314 | 0.56550092 |
| 157 | TMEM2 | 0.172006 | 0.00860142 | 0.56550092 |
| 158 | IDI1 | −0.07782 | 0.00860486 | 0.56550092 |
| 159 | HSPA7 | 0.400728 | 0.00877046 | 0.56550092 |
| 160 | HSPG2 | −0.1904 | 0.00877754 | 0.56550092 |
| 161 | RCN3 | 0.464299 | 0.00880775 | 0.56550092 |
| 162 | CAPN15 | 0.168296 | 0.00881938 | 0.56550092 |
| 163 | CAMLG | −0.06238 | 0.00887155 | 0.56550092 |
| 164 | DDX39B | 0.295788 | 0.00891392 | 0.56550092 |
| 165 | TOX4 | 0.047401 | 0.00892093 | 0.56550092 |
| 166 | NLRP1 | 0.236209 | 0.00899511 | 0.56550092 |
| 167 | VTI1A | 0.090232 | 0.00907805 | 0.56550092 |
| 168 | STIM2 | 0.112881 | 0.00911269 | 0.56550092 |
| 169 | AFF2 | −0.14313 | 0.00917015 | 0.56550092 |
| 170 | CYSTM1 | −0.1873 | 0.00920811 | 0.56550092 |
| 171 | ABCA2 | 0.32242 | 0.00920901 | 0.56550092 |
| 172 | TARBP2 | 0.189071 | 0.00925303 | 0.56550092 |
| 173 | EIF4A1 | 0.26069 | 0.00945454 | 0.57464107 |
| 174 | FCHO1 | 0.127726 | 0.00951062 | 0.57464107 |
| 175 | TMC6 | 0.223573 | 0.00956686 | 0.57464107 |
| 176 | CLEC4E | −0.18421 | 0.0095995 | 0.57464107 |
| 177 | THAP12 | −0.05666 | 0.0097045 | 0.57525432 |
| 178 | NFU1 | −0.07127 | 0.00973334 | 0.57525432 |
| 179 | KIAA0141 | 0.132062 | 0.0098395 | 0.57525432 |
| 180 | MS4A14 | 0.284113 | 0.00987025 | 0.57525432 |
| 181 | SLC25A30 | 0.135501 | 0.00988115 | 0.57525432 |
| 182 | FCGR2C | 0.369137 | 0.0099791 | 0.57525432 |
| 183 | ATP10A | 0.24706 | 0.01001119 | 0.57525432 |
| 184 | NINJ1 | 0.109417 | 0.01004847 | 0.57525432 |
| 185 | SEC31B | 0.370585 | 0.01005328 | 0.57525432 |
| 186 | FAM107A | −0.19884 | 0.01019154 | 0.57594247 |
| 187 | AGER | 0.330009 | 0.0102037 | 0.57594247 |
| 188 | IKBKB | 0.074524 | 0.01024932 | 0.57594247 |
| 189 | RPL3P4 | 0.290315 | 0.01026266 | 0.57594247 |
| 190 | DNMT3A | 0.092337 | 0.0104197 | 0.58195786 |
| 191 | ANKRD11 | 0.122861 | 0.01048561 | 0.58220313 |
| 192 | LILRA4 | 0.180795 | 0.01052385 | 0.58220313 |
| 193 | CPEB3 | 0.132065 | 0.01069118 | 0.58867045 |
| 194 | STRIP1 | 0.127331 | 0.01076033 | 0.58969665 |
| 195 | CLASRP | 0.216493 | 0.01096388 | 0.59804356 |
| 196 | CHMP4BP1 | 0.214505 | 0.0110522 | 0.59821642 |
| 197 | IFI6 | −0.258 | 0.0111135 | 0.59821642 |
| 198 | GAA | 0.270265 | 0.01112828 | 0.59821642 |
| 199 | HIKESHI | −0.09654 | 0.01117204 | 0.59821642 |
| 200 | ZNF276 | 0.149414 | 0.01129951 | 0.60227919 |
| 201 | ARIH1 | 0.077238 | 0.01140323 | 0.6034841 |
| 202 | NBPF9 | 0.147874 | 0.01149254 | 0.6034841 |
| 203 | GYG1 | −0.09593 | 0.01159812 | 0.6034841 |
| 204 | KCNC3 | 0.279616 | 0.01160066 | 0.6034841 |
| 205 | CEP68 | 0.118344 | 0.01160072 | 0.6034841 |
| 206 | AKAP17A | 0.179066 | 0.01166187 | 0.6034841 |
| 207 | RNF111 | 0.043219 | 0.01168401 | 0.6034841 |
| 208 | CCNL2 | 0.207683 | 0.0118058 | 0.6070888 |
| 209 | EP400NL | 0.218649 | 0.01187441 | 0.60793866 |
| 210 | FCRL5 | 0.305718 | 0.01196743 | 0.60908546 |
| 211 | IGF2R | 0.268732 | 0.01203031 | 0.60908546 |
| 212 | SMCR8 | 0.062574 | 0.01221539 | 0.60908546 |
| 213 | KLHL35 | 0.365873 | 0.012227 | 0.60908546 |
| 214 | VGLL3 | 0.286155 | 0.01225075 | 0.60908546 |
| 215 | PLPPR2 | 0.248368 | 0.01232664 | 0.60908546 |
| 216 | HBG1 | 0.488888 | 0.01237353 | 0.60908546 |
| 217 | CEACAM1 | −0.2294 | 0.01242269 | 0.60908546 |
| 218 | SELPLG | 0.172377 | 0.0124516 | 0.60908546 |
| 219 | TMEM106A | 0.235544 | 0.01247414 | 0.60908546 |
| 220 | SPAG5 | −0.13343 | 0.01250929 | 0.60908546 |
| 221 | IL6R | 0.235819 | 0.01253686 | 0.60908546 |
| 222 | RELT | 0.320346 | 0.0126367 | 0.60908546 |
| 223 | CAPN10 | 0.241909 | 0.01267804 | 0.60908546 |
| 224 | UBR2 | 0.05001 | 0.0126795 | 0.60908546 |
| 225 | BPI | −0.23487 | 0.01306896 | 0.61980568 |
| 226 | CPNE3 | −0.08843 | 0.01312473 | 0.61980568 |
| 227 | ITPRIP | 0.333223 | 0.01319897 | 0.61980568 |
| 228 | SUSD6 | 0.143109 | 0.01330757 | 0.61980568 |
| 229 | MYH3 | 0.319441 | 0.01337869 | 0.61980568 |
| 230 | NPIPB11 | 0.225074 | 0.01338374 | 0.61980568 |
| 231 | HIST1H2AH | −0.16579 | 0.01339516 | 0.61980568 |
| 232 | ARAP1 | 0.113937 | 0.01340864 | 0.61980568 |
| 233 | TNFRSF1B | 0.236397 | 0.01341026 | 0.61980568 |
| 234 | COQ7 | −0.10226 | 0.01343364 | 0.61980568 |
| 235 | NCKIPSD | −0.16181 | 0.01355632 | 0.62228365 |
| 236 | SORBS1 | −0.12546 | 0.01366928 | 0.62228365 |
| 237 | SLC11A2 | 0.131949 | 0.01367015 | 0.62228365 |
| 238 | ANXA1 | −0.12078 | 0.01370058 | 0.62228365 |
| 239 | DDX31 | 0.149845 | 0.01376824 | 0.62293282 |
| 240 | TSPYL2 | 0.152066 | 0.01392207 | 0.62746062 |
| 241 | MIA3 | 0.112725 | 0.01401485 | 0.62921269 |
| 242 | SRCAP | 0.087386 | 0.01421777 | 0.63587761 |
| 243 | TMUB2 | 0.179351 | 0.01427441 | 0.635974 |
| 244 | RICTOR | 0.047912 | 0.01443204 | 0.63701257 |
| 245 | B3GNT2 | −0.14535 | 0.0144994 | 0.63701257 |
| 246 | CLSPN | −0.09817 | 0.01450526 | 0.63701257 |
| 247 | RPRD2 | 0.046718 | 0.01451601 | 0.63701257 |
| 248 | KIFC1 | −0.18671 | 0.01460628 | 0.63717368 |
| 249 | ATG2A | 0.173904 | 0.01467416 | 0.63717368 |
| 250 | RAD51B | 0.182219 | 0.01477235 | 0.63717368 |
| 251 | KIF20A | −0.181 | 0.01482021 | 0.63717368 |
| 252 | MT2A | −0.1039 | 0.01487899 | 0.63717368 |
| 253 | LFNG | 0.284885 | 0.01494183 | 0.63717368 |
| 254 | TPD52L1 | −0.22667 | 0.01497767 | 0.63717368 |
| 255 | ADGRES | 0.179919 | 0.01500528 | 0.63717368 |
| 256 | EXO1 | −0.14261 | 0.01505712 | 0.63717368 |
| 257 | KLHL12 | 0.072157 | 0.01511598 | 0.63717368 |
| 258 | ZNF641 | 0.11215 | 0.01514451 | 0.63717368 |
| 259 | DCUN1D1 | 0.09413 | 0.01522795 | 0.63717368 |
| 260 | ATP2B1 | 0.125617 | 0.01522929 | 0.63717368 |
| 261 | ZCRB1 | −0.07944 | 0.01553718 | 0.63898806 |
| 262 | MKI67 | −0.11168 | 0.01563439 | 0.63898806 |
| 263 | NOTCH2 | 0.225099 | 0.01567665 | 0.63898806 |
| 264 | ELL2P1 | −0.28705 | 0.0156776 | 0.63898806 |
| 265 | TRAPPC12 | 0.078491 | 0.01568194 | 0.63898806 |
| 266 | ITPR3 | 0.184525 | 0.01570768 | 0.63898806 |
| 267 | PDPR | 0.159366 | 0.01572536 | 0.63898806 |
| 268 | C17orf80 | −0.0737 | 0.01574463 | 0.63898806 |
| 269 | KLC1 | 0.116093 | 0.01581611 | 0.63898806 |
| 270 | SUN2 | 0.2067 | 0.01585866 | 0.63898806 |
| 271 | ZNF587 | 0.148131 | 0.01590788 | 0.63898806 |
| 272 | SIGLEC7 | 0.193033 | 0.01592954 | 0.63898806 |
| 273 | SPC24 | −0.14702 | 0.01599473 | 0.63940564 |
| 274 | HIST1H3D | −0.10572 | 0.01613502 | 0.64281254 |
| 275 | PSMA3-AS1 | 0.156466 | 0.01629385 | 0.64451294 |
| 276 | IL1R1 | −0.15503 | 0.01635679 | 0.64451294 |
| 277 | GIGYF1 | 0.173191 | 0.01640429 | 0.64451294 |
| 278 | SLC43A2 | 0.271739 | 0.01642484 | 0.64451294 |
| 279 | IFIT1 | −0.20819 | 0.01645377 | 0.64451294 |
| 280 | EEF1E1 | −0.09811 | 0.01652464 | 0.64512425 |
| 281 | CAMK2G | 0.077266 | 0.01663281 | 0.64718269 |
| 282 | CPD | 0.150082 | 0.01669924 | 0.64760864 |
| 283 | NEK2 | −0.19375 | 0.01678854 | 0.6489159 |
| 284 | TUBGCP6 | 0.22681 | 0.01698933 | 0.65450974 |
| 285 | PIK3IP1 | 0.22368 | 0.0171141 | 0.65595108 |
| 286 | ARPC4- | 0.195999 | 0.01719787 | 0.65595108 |
| | TTLL3 | | | |
| 287 | HMCN1 | −0.22912 | 0.0171991 | 0.65595108 |
| 288 | DLK1 | 0.406847 | 0.01725152 | 0.65595108 |
| 289 | ISG15 | −0.19497 | 0.01732315 | 0.65653607 |
| 290 | CBX7 | 0.114646 | 0.01739648 | 0.65718171 |
| 291 | HCFC1R1 | −0.09912 | 0.0175175 | 0.65961868 |
| 292 | NEAT1 | 0.273427 | 0.01776116 | 0.6615242 |
| 293 | OTUD7B | −0.07552 | 0.01777955 | 0.6615242 |
| 294 | PLEKHM1P1 | 0.266675 | 0.01778405 | 0.6615242 |
| 295 | ZNF880 | −0.11044 | 0.01787496 | 0.6615242 |
| 296 | CD19 | 0.254783 | 0.01790047 | 0.6615242 |
| 297 | HIST1H2BL | −0.12878 | 0.01790813 | 0.6615242 |
| 298 | AUH | 0.099883 | 0.01821664 | 0.67079755 |
| 299 | DEF8 | 0.134343 | 0.01833732 | 0.67311793 |
| 300 | SLC19A1 | 0.300927 | 0.01844905 | 0.67481727 |
| 301 | SZT2 | 0.152443 | 0.01868453 | 0.67481727 |
| 302 | P2RY8 | 0.261269 | 0.01870759 | 0.67481727 |
| 303 | ADNP2 | 0.08817 | 0.01870974 | 0.67481727 |
| 304 | QSOX2 | 0.200001 | 0.01872196 | 0.67481727 |
| 305 | MYBL2 | −0.12281 | 0.01873047 | 0.67481727 |
| 306 | PCNX1 | 0.128145 | 0.01881993 | 0.67489532 |
| 307 | MCM4 | −0.0977 | 0.01901543 | 0.67489532 |
| 308 | PLA2G6 | 0.270264 | 0.01907223 | 0.67489532 |
| 309 | MAPK8IP3 | 0.168985 | 0.01914121 | 0.67489532 |
| 310 | ZNF628 | 0.201732 | 0.01915175 | 0.67489532 |
| 311 | LPCAT1 | 0.169393 | 0.01933296 | 0.67489532 |
| 312 | NCSTN | 0.142595 | 0.01937521 | 0.67489532 |
| 313 | FNBP4 | 0.080692 | 0.01938271 | 0.67489532 |
| 314 | NBN | −0.04407 | 0.01946149 | 0.67489532 |
| 315 | KMT2A | 0.046935 | 0.01964344 | 0.67489532 |
| 316 | DGKA | 0.12424 | 0.01965792 | 0.67489532 |
| 317 | RILPL1 | 0.110835 | 0.0197448 | 0.67489532 |
| 318 | TBL1X | 0.09656 | 0.01980309 | 0.67489532 |
| 319 | CNPY3 | 0.075107 | 0.01983667 | 0.67489532 |
| 320 | SLC12A9 | 0.299377 | 0.01992008 | 0.67489532 |
| 321 | BUB1B | −0.09969 | 0.0199485 | 0.67489532 |
| 322 | SLC25A17 | −0.11684 | 0.01999033 | 0.67489532 |
| 323 | PANX2 | 0.284076 | 0.02004928 | 0.67489532 |
| 324 | HEATR5A | −0.09643 | 0.02005246 | 0.67489532 |
| 325 | MYLIP | 0.104019 | 0.02006079 | 0.67489532 |
| 326 | RBMS3 | −0.19762 | 0.02006373 | 0.67489532 |
| 327 | ADAM28 | 0.183931 | 0.02013975 | 0.67489532 |
| 328 | UBR5 | 0.038568 | 0.02034022 | 0.67489532 |
| 329 | USP18 | −0.19703 | 0.02041136 | 0.67489532 |
| 330 | FAM161B | 0.182304 | 0.02043321 | 0.67489532 |
| 331 | CCDC84 | 0.26184 | 0.02043381 | 0.67489532 |
| 332 | PLCXD1 | 0.198888 | 0.02051062 | 0.67489532 |
| 333 | CLSTN3 | 0.237424 | 0.02051223 | 0.67489532 |
| 334 | C15orf39 | 0.105977 | 0.02052644 | 0.67489532 |
| 335 | GABBR1 | 0.284971 | 0.02052952 | 0.67489532 |
| 336 | PLCB2 | 0.17458 | 0.02053626 | 0.67489532 |
| 337 | ATG16L2 | 0.296619 | 0.0206175 | 0.67489532 |
| 338 | PRKCZ | 0.163892 | 0.02064059 | 0.67489532 |
| 339 | WBSCR22 | 0.085443 | 0.02076199 | 0.67696851 |
| 340 | TMCO6 | 0.173505 | 0.02091538 | 0.67883629 |
| 341 | PGLYRP1 | −0.22309 | 0.02093558 | 0.67883629 |
| 342 | TCIRG1 | 0.295107 | 0.02124424 | 0.68693636 |
| 343 | EGLN2 | 0.161778 | 0.02138346 | 0.689528 |
| 344 | MRPS36 | −0.07868 | 0.02158738 | 0.69271736 |
| 345 | SLC43A1 | −0.1344 | 0.02175011 | 0.69271736 |
| 346 | IFIT2 | −0.14909 | 0.02182304 | 0.69271736 |
| 347 | H2AFX | −0.1496 | 0.02184128 | 0.69271736 |
| 348 | TNFRSF8 | 0.174519 | 0.0218725 | 0.69271736 |
| 349 | NRROS | 0.12798 | 0.02193378 | 0.69271736 |
| 350 | EEPD1 | 0.225546 | 0.02195508 | 0.69271736 |
| 351 | EIF2AK3 | 0.147126 | 0.02205429 | 0.69271736 |
| 352 | POR | 0.219464 | 0.02205949 | 0.69271736 |
| 353 | PHF5A | −0.07449 | 0.0221504 | 0.69271736 |
| 354 | NQO1 | −0.20608 | 0.02220612 | 0.69271736 |
| 355 | PAN2 | 0.184904 | 0.02224324 | 0.69271736 |
| 356 | CD99P1 | −0.13373 | 0.02227539 | 0.69271736 |
| 357 | SLC45A4 | 0.118013 | 0.02236131 | 0.69271736 |
| 358 | LILRA6 | 0.307306 | 0.02240705 | 0.69271736 |
| 359 | SETD1B | 0.123318 | 0.0224899 | 0.69271736 |
| 360 | ZNF746 | 0.141649 | 0.02254211 | 0.69271736 |
| 361 | TDP2 | −0.05474 | 0.02255055 | 0.69271736 |
| 362 | CARS2 | 0.108206 | 0.02262887 | 0.6932987 |
| 363 | TMC8 | 0.212077 | 0.02273431 | 0.6934895 |
| 364 | ABHD11 | 0.115085 | 0.02291834 | 0.6934895 |
| 365 | UBE4A | 0.112898 | 0.02293195 | 0.6934895 |
| 366 | SREBF1 | 0.22463 | 0.02298465 | 0.6934895 |
| 367 | BBC3 | 0.136315 | 0.02300575 | 0.6934895 |
| 368 | IFIT3 | −0.17453 | 0.0230222 | 0.6934895 |
| 369 | DIDO1 | 0.101033 | 0.02306184 | 0.6934895 |
| 370 | BCAS4 | 0.156649 | 0.02311038 | 0.6934895 |
| 371 | FGD3 | 0.093298 | 0.0236161 | 0.70211107 |
| 372 | IGFBP7 | −0.15367 | 0.02372217 | 0.70211107 |
| 373 | MED12 | 0.053554 | 0.02378065 | 0.70211107 |
| 374 | NLRC4 | −0.11586 | 0.02380693 | 0.70211107 |
| 375 | SLC16A3 | 0.228567 | 0.02388297 | 0.70211107 |
| 376 | KXD1 | 0.051909 | 0.02391767 | 0.70211107 |
| 377 | FAM103A1 | −0.09355 | 0.02403275 | 0.70211107 |
| 378 | CDK5RAP3 | 0.165733 | 0.02404738 | 0.70211107 |
| 379 | IL17RA | 0.184535 | 0.02412421 | 0.70211107 |
| 380 | SLAMF1 | 0.217307 | 0.02413338 | 0.70211107 |

Second differential expression analysis of predicting preterm birth earlier than 35 weeks of gestational age, with blood samples collected between 23-26 weeks of gestational age, was performed using EdgeR and accounting for ethnicity, and cohort effects and gestational age at collection (60 PTB cases and 271 controls). Table 42 shows a set of top 17 genes with p-value<0.1 after adjustment from multiple hypothesis correction (FDR value), and also showed a significant deviation from the null hypothesis in a QQ plot for differentially expressed in pre-term birth cases (as shown in FIG. 44D). Table 43 shows an additional set of genes with p-value<0.1 for predicting preterm birth earlier than 35 weeks of gestation with blood samples collected between 23-26 weeks of gestational age. Genes are ordered according to their statistical significance (P-values).

TABLE 42
Top 17 genes with p-value < 0.1 after adjustment from multiple
hypothesis correction (FDR value), that are predictive for
preterm birth earlier than 35 weeks of gestation with blood
samples collected between 23-26 weeks of gestational age

| # | Gene | logFC | P-Value | FDR |
|---|---|---|---|---|
| 1 | HRG | −2.0501607 | 1.04E−13 | 1.21E−09 |
| 2 | APOH | −1.5623334 | 4.11E−10 | 2.38E−06 |
| 3 | HPD | −1.2263966 | 1.87E−09 | 7.21E−06 |
| 4 | FGA | −1.4396986 | 2.49E−09 | 7.21E−06 |
| 5 | FGB | −1.3687247 | 5.31E−09 | 1.23E−05 |
| 6 | ALB | −1.1326035 | 4.58E−08 | 8.85E−05 |
| 7 | FGG | −1.3587488 | 1.43E−07 | 0.000236 |
| 8 | APOB | −1.2053038 | 1.87E−07 | 0.000271 |
| 9 | FABP1 | −1.0001499 | 5.02E−07 | 0.000647 |
| 10 | ADH1B | −1.0046253 | 7.37E−07 | 0.000855 |
| 11 | CYP2E1 | −0.9826505 | 1.33E−06 | 0.001402 |
| 12 | PDK4 | −0.5034507 | 3.24E−05 | 0.030923 |
| 13 | SH3PXD2A | −0.2910378 | 3.47E−05 | 0.030923 |
| 14 | MUC3A | −0.8112918 | 6.09E−05 | 0.04865 |
| 15 | PCGF2 | −0.8084937 | 6.29E−05 | 0.04865 |
| 16 | LZTS2 | −0.3533705 | 0.00011954 | 0.08215 |
| 17 | APOC1 | −0.5631767 | 0.00012038 | 0.08215 |

TABLE 43
Additional set of genes with p-value < 0.1 for predicting
preterm birth earlier than 35 weeks of gestation with blood
samples collected between 23-26 weeks of gestational age

| # | Gene | logFC | P-Value | FDR |
|---|---|---|---|---|
| 1 | DLGAP4 | −0.1826629 | 0.00025723 | 0.15917 |
| 2 | PTGS2 | 0.84128363 | 0.00026069 | 0.15917 |
| 3 | PAPPA2 | −0.7793313 | 0.00038856 | 0.225385 |
| 4 | EMILIN1 | −0.4481043 | 0.00059221 | 0.327151 |
| 5 | KIAA1143 | −0.1572862 | 0.00082778 | 0.436505 |
| 6 | CLEC4E | −0.4112452 | 0.00097681 | 0.492696 |
| 7 | MBNL3 | 0.22423002 | 0.00111498 | 0.538953 |
| 8 | NUP98 | 0.09665667 | 0.00123335 | 0.572325 |
| 9 | C19orf43 | −0.0918831 | 0.00129597 | 0.578253 |
| 10 | RPH3AL | −0.4402562 | 0.00142451 | 0.612065 |
| 11 | FAM9C | −0.7142533 | 0.00159475 | 0.649768 |
| 12 | FKBP5 | −0.2820347 | 0.00167331 | 0.649768 |
| 13 | CFH | −0.4469532 | 0.00168029 | 0.649768 |
| 14 | YOD1 | 0.33247661 | 0.00192385 | 0.719956 |
| 15 | DPH3 | −0.1658585 | 0.00241433 | 0.875271 |
| 16 | FO538757.1 | −0.4227779 | 0.00289461 | 0.975219 |
| 17 | TXNDC5 | −0.3194514 | 0.00290269 | 0.975219 |
| 18 | ZNF483 | −0.3604009 | 0.00297885 | 0.975219 |
| 19 | SH2D1A | 0.31281166 | 0.00302628 | 0.975219 |
| 20 | PKP4 | −0.167658 | 0.00341057 | 0.999823 |
| 21 | KCTD2 | −0.2160454 | 0.00382209 | 0.999823 |
| 22 | CTD- | 0.88326474 | 0.00399624 | 0.999823 |
| | 3088G3.8 | | | |
| 23 | TM4SF1 | 0.40428082 | 0.00426688 | 0.999823 |
| 24 | UBE2B | 0.16850547 | 0.00435697 | 0.999823 |
| 25 | C3 | −0.3254057 | 0.00473421 | 0.999823 |
| 26 | KIAA0430 | 0.14144464 | 0.00478614 | 0.999823 |
| 27 | GPX3 | −0.3665209 | 0.00480981 | 0.999823 |
| 28 | ZBTB16 | −0.242741 | 0.00496256 | 0.999823 |
| 29 | UBR2 | 0.09842027 | 0.00508955 | 0.999823 |
| 30 | ARMC2 | 0.22755852 | 0.00517468 | 0.999823 |
| 31 | AIFM3 | 0.48184268 | 0.00521153 | 0.999823 |
| 32 | SOCS2 | −0.2791332 | 0.00547838 | 0.999823 |
| 33 | OPA1 | 0.16331524 | 0.0057958 | 0.999823 |
| 34 | PIP5K1B | 0.20202821 | 0.00581586 | 0.999823 |
| 35 | ERICH6 | −0.3921927 | 0.00593558 | 0.999823 |
| 36 | SESN1 | −0.1998035 | 0.00652404 | 0.999823 |
| 37 | ZNF462 | −0.1864143 | 0.00671098 | 0.999823 |
| 38 | IFI27L1 | −0.452319 | 0.00677637 | 0.999823 |
| 39 | REC8 | 0.4129679 | 0.00717734 | 0.999823 |
| 40 | ENG | −0.2243093 | 0.00726122 | 0.999823 |
| 41 | SLC18B1 | 0.39411126 | 0.00735385 | 0.999823 |
| 42 | MALAT1 | 0.5093659 | 0.00756213 | 0.999823 |
| 43 | TCP11L2 | 0.32943455 | 0.0076547 | 0.999823 |
| 44 | FECH | 0.33308949 | 0.00780277 | 0.999823 |
| 45 | ZNF518B | −0.1696499 | 0.00789717 | 0.999823 |
| 46 | CGNL1 | −0.3124707 | 0.00796199 | 0.999823 |
| 47 | MANSC1 | −0.3228849 | 0.00804338 | 0.999823 |
| 48 | ABCG2 | 0.38123408 | 0.00809224 | 0.999823 |
| 49 | CMKLR1 | −0.3742352 | 0.00819591 | 0.999823 |
| 50 | HIST1H2BB | −0.2704749 | 0.00846588 | 0.999823 |
| 51 | DHX34 | 0.39787335 | 0.00862585 | 0.999823 |
| 52 | MTHFS | −0.1745955 | 0.00871068 | 0.999823 |
| 53 | CNTROB | −0.1665571 | 0.00886627 | 0.999823 |
| 54 | ZBTB4 | −0.1300612 | 0.00887294 | 0.999823 |
| 55 | IGHA1 | −0.3745478 | 0.00991255 | 0.999823 |
| 56 | ATN1 | −0.1616119 | 0.00997235 | 0.999823 |
| 57 | TNFRSF8 | 0.34514822 | 0.01023486 | 0.999823 |
| 58 | SF3B6 | −0.1206185 | 0.01026664 | 0.999823 |
| 59 | ERCC6L | −0.3636561 | 0.01036967 | 0.999823 |
| 60 | ZNF282 | −0.1812759 | 0.01062498 | 0.999823 |
| 61 | VPS53 | 0.11170753 | 0.0106913 | 0.999823 |
| 62 | ZNF768 | −0.1353357 | 0.01077038 | 0.999823 |
| 63 | RNF145 | −0.1914913 | 0.01079595 | 0.999823 |
| 64 | CCDC134 | 0.25411934 | 0.01083317 | 0.999823 |
| 65 | MICALCL | 0.3554645 | 0.01092668 | 0.999823 |
| 66 | SH3BP5 | −0.171843 | 0.01098901 | 0.999823 |
| 67 | ACACB | −0.2045808 | 0.01119203 | 0.999823 |
| 68 | ETFB | −0.1510851 | 0.01121339 | 0.999823 |
| 69 | TRIM23 | 0.18470962 | 0.01121431 | 0.999823 |
| 70 | TDP2 | −0.1055306 | 0.01160123 | 0.999823 |
| 71 | RBFA | −0.1873702 | 0.01162321 | 0.999823 |
| 72 | ACD | −0.1391661 | 0.01181329 | 0.999823 |
| 73 | ITPRIP | 0.51076938 | 0.0119837 | 0.999823 |
| 74 | ZNF582 | −0.3109977 | 0.01200289 | 0.999823 |
| 75 | NAXD | 0.20887993 | 0.01206603 | 0.999823 |
| 76 | ULK2 | 0.13622427 | 0.01230707 | 0.999823 |
| 77 | B3GNT2 | −0.280015 | 0.01240541 | 0.999823 |
| 78 | ZNF354A | −0.2219853 | 0.01256182 | 0.999823 |
| 79 | AMOT | −0.2021322 | 0.01290087 | 0.999823 |
| 80 | RNF169 | 0.10073219 | 0.01297084 | 0.999823 |
| 81 | STAG3 | −0.4021953 | 0.01315327 | 0.999823 |
| 82 | NCR1 | 0.34775107 | 0.01385312 | 0.999823 |
| 83 | FAM46C | 0.23767656 | 0.01404483 | 0.999823 |
| 84 | BIRC2 | 0.14715869 | 0.01425473 | 0.999823 |
| 85 | COL3A1 | −0.7793199 | 0.01472776 | 0.999823 |
| 86 | NSRP1 | −0.1201089 | 0.01473527 | 0.999823 |
| 87 | FASLG | 0.39523963 | 0.01478741 | 0.999823 |
| 88 | ZMYND15 | 0.34817106 | 0.01480891 | 0.999823 |
| 89 | NCKIPSD | −0.2858192 | 0.01483803 | 0.999823 |
| 90 | MMP25 | 0.61695067 | 0.01504564 | 0.999823 |
| 91 | RNF14 | 0.17065401 | 0.01507707 | 0.999823 |
| 92 | TAF6L | 0.33757278 | 0.01508158 | 0.999823 |
| 93 | GHR | −0.4175955 | 0.01518602 | 0.999823 |
| 94 | PIAS4 | −0.1382704 | 0.01536949 | 0.999823 |
| 95 | CELF1 | 0.10670906 | 0.01545935 | 0.999823 |
| 96 | FOXO3B | 0.28663588 | 0.01577862 | 0.999823 |
| 97 | ZNF880 | −0.1974472 | 0.01578517 | 0.999823 |
| 98 | SOX6 | 0.3209163 | 0.01579766 | 0.999823 |
| 99 | PRG4 | −0.5432311 | 0.0159479 | 0.999823 |
| 100 | UCK1 | −0.1613335 | 0.01620986 | 0.999823 |
| 101 | C7orf31 | 0.14545571 | 0.01648371 | 0.999823 |
| 102 | PLA2G7 | 0.31700117 | 0.01648608 | 0.999823 |
| 103 | OTUD7B | −0.129247 | 0.01659747 | 0.999823 |
| 104 | DYM | 0.11498399 | 0.01661968 | 0.999823 |
| 105 | LMTK2 | 0.22610005 | 0.01689268 | 0.999823 |
| 106 | DMPK | −0.3229673 | 0.01693248 | 0.999823 |
| 107 | FAM107A | −0.3305965 | 0.01696118 | 0.999823 |
| 108 | FGD5 | −0.2571516 | 0.01704237 | 0.999823 |
| 109 | INHBA | −0.417118 | 0.01716363 | 0.999823 |
| 110 | MOSPD3 | −0.2189547 | 0.01723402 | 0.999823 |
| 111 | CAMLG | −0.0990098 | 0.01729544 | 0.999823 |
| 112 | APOBEC3C | −0.1071202 | 0.01738431 | 0.999823 |
| 113 | CHMP4BP1 | 0.33535436 | 0.01759232 | 0.999823 |
| 114 | KLHL9 | 0.12519507 | 0.01767043 | 0.999823 |
| 115 | NOTCH1 | 0.37680237 | 0.01779583 | 0.999823 |
| 116 | ADGRE5 | 0.28079719 | 0.01796911 | 0.999823 |
| 117 | PLEKHM3 | 0.1673145 | 0.01808403 | 0.999823 |
| 118 | ITGAX | 0.47545536 | 0.01830889 | 0.999823 |
| 119 | NEUROD2 | −0.3566226 | 0.01847832 | 0.999823 |
| 120 | FRY | 0.15403656 | 0.01856121 | 0.999823 |
| 121 | MAGI2 | −0.4263608 | 0.0187085 | 0.999823 |
| 122 | PTDSS2 | −0.3127907 | 0.01872473 | 0.999823 |
| 123 | SORBS1 | −0.2354539 | 0.01902384 | 0.999823 |
| 124 | ARFGAP3 | 0.08070118 | 0.01908572 | 0.999823 |
| 125 | SLC9A8 | 0.27458933 | 0.01951124 | 0.999823 |
| 126 | FLT1 | −0.1862232 | 0.01956642 | 0.999823 |
| 127 | FAM206A | −0.1844597 | 0.01976687 | 0.999823 |
| 128 | SNX8 | −0.1606373 | 0.01992467 | 0.999823 |
| 129 | EGR2 | 0.40055113 | 0.02001137 | 0.999823 |
| 130 | CRIP2 | −0.2769295 | 0.02007045 | 0.999823 |
| 131 | FBXO18 | −0.0995458 | 0.02013104 | 0.999823 |
| 132 | THBD | 0.40966091 | 0.02015288 | 0.999823 |
| 133 | SACS | 0.13073475 | 0.02017999 | 0.999823 |
| 134 | LPIN2 | 0.1659817 | 0.02018442 | 0.999823 |
| 135 | ATG16L2 | 0.47066975 | 0.0203194 | 0.999823 |
| 136 | DAP3 | 0.08230965 | 0.0206098 | 0.999823 |
| 137 | NBPF26 | 0.21725083 | 0.02068397 | 0.999823 |
| 138 | SKI | −0.1495791 | 0.02079017 | 0.999823 |
| 139 | ZNF628 | 0.33399888 | 0.02092355 | 0.999823 |
| 140 | LILRA6 | 0.50709887 | 0.02103163 | 0.999823 |
| 141 | AKAP10 | 0.11183522 | 0.02103648 | 0.999823 |
| 142 | EED | 0.14941401 | 0.02104887 | 0.999823 |
| 143 | IGLV2-14 | −0.4599037 | 0.02118479 | 0.999823 |
| 144 | CUL4A | 0.19550185 | 0.02120272 | 0.999823 |
| 145 | SESN3 | 0.21352389 | 0.02122431 | 0.999823 |
| 146 | GGH | −0.286244 | 0.02123904 | 0.999823 |
| 147 | RBMS3 | −0.3370053 | 0.02131978 | 0.999823 |
| 148 | EPG5 | 0.12765985 | 0.02167255 | 0.999823 |
| 149 | ROMO1 | −0.1350013 | 0.02170047 | 0.999823 |
| 150 | PSMA2 | −0.1500424 | 0.02176662 | 0.999823 |
| 151 | JCHAIN | −0.2717374 | 0.0218627 | 0.999823 |
| 152 | TCF4 | −0.1022857 | 0.02194006 | 0.999823 |
| 153 | ANPEP | 0.40564921 | 0.02206361 | 0.999823 |
| 154 | GNL1 | −0.0997968 | 0.02226215 | 0.999823 |
| 155 | IFITM2 | −0.1759504 | 0.0225286 | 0.999823 |
| 156 | C19orf47 | 0.21854524 | 0.02262179 | 0.999823 |
| 157 | NUS1 | 0.14799733 | 0.02271065 | 0.999823 |
| 158 | RCN3 | 0.68134501 | 0.02306315 | 0.999823 |
| 159 | THAP12 | −0.0859371 | 0.02311962 | 0.999823 |
| 160 | MICU3 | 0.28981943 | 0.02338403 | 0.999823 |
| 161 | PLTP | −0.2540581 | 0.0234384 | 0.999823 |
| 162 | SOX12 | −0.225235 | 0.02344202 | 0.999823 |
| 163 | NFKBID | 0.49807675 | 0.0236816 | 0.999823 |
| 164 | SPAG1 | −0.2060284 | 0.02381805 | 0.999823 |
| 165 | GCLC | 0.25921593 | 0.02387105 | 0.999823 |
| 166 | SMPD1 | −0.3658053 | 0.02409033 | 0.999823 |
| 167 | CYP19A1 | 0.31658844 | 0.02416579 | 0.999823 |
| 168 | IGF2R | 0.37123383 | 0.02422257 | 0.999823 |
| 169 | SRGAP2C | −0.2674164 | 0.02428598 | 0.999823 |
| 170 | NBPF10 | 0.21328924 | 0.02445397 | 0.999823 |
| 171 | ZNF706 | −0.1029408 | 0.02454303 | 0.999823 |
| 172 | SLC11A1 | 0.47849014 | 0.0246525 | 0.999823 |
| 173 | NEAT1 | 0.44914561 | 0.02469506 | 0.999823 |
| 174 | RP3- | −0.2996412 | 0.02479862 | 0.999823 |
| | 370M22.8 | | | |
| 175 | MPRIP | −0.1062469 | 0.02481405 | 0.999823 |
| 176 | CYP4F3 | 0.48971249 | 0.02494545 | 0.999823 |
| 177 | SF3A2 | −0.1064816 | 0.02501017 | 0.999823 |
| 178 | HP | −0.4687396 | 0.02506622 | 0.999823 |
| 179 | IGFBP7 | −0.2605503 | 0.02517671 | 0.999823 |
| 180 | RAB11FIP3 | −0.181872 | 0.02531611 | 0.999823 |
| 181 | ALDOB | −0.4368653 | 0.025317 | 0.999823 |
| 182 | BCL7A | −0.2317492 | 0.02552236 | 0.999823 |
| 183 | SOCS4 | −0.1297161 | 0.02559725 | 0.999823 |
| 184 | ANAPC15 | −0.1113047 | 0.02562734 | 0.999823 |
| 185 | PRICKLE1 | −0.1549395 | 0.02592533 | 0.999823 |
| 186 | CEP55 | −0.2088249 | 0.02594296 | 0.999823 |
| 187 | BCKDHA | 0.27552704 | 0.02596038 | 0.999823 |
| 188 | PLCXD1 | 0.30232113 | 0.02636879 | 0.999823 |
| 189 | USP53 | −0.2299264 | 0.02639874 | 0.999823 |
| 190 | FAM103A1 | −0.1655768 | 0.02640089 | 0.999823 |
| 191 | ARHGEF10 | −0.2302561 | 0.02654062 | 0.999823 |
| 192 | ASS1 | −0.3371256 | 0.0266732 | 0.999823 |
| 193 | CAMKMT | 0.18688262 | 0.02713489 | 0.999823 |
| 194 | PRR13 | −0.118958 | 0.02756679 | 0.999823 |
| 195 | PTGIR | −0.2526015 | 0.02759952 | 0.999823 |
| 196 | ADPGK | 0.22144726 | 0.02760505 | 0.999823 |
| 197 | TSEN2 | 0.17037095 | 0.02765733 | 0.999823 |
| 198 | ADAM8 | 0.52818264 | 0.02769841 | 0.999823 |
| 199 | MARK3 | 0.10173154 | 0.02771626 | 0.999823 |
| 200 | TVP23C | −0.2478444 | 0.02772386 | 0.999823 |
| 201 | TMEM232 | 0.3877995 | 0.027959 | 0.999823 |
| 202 | ATG2A | 0.24751798 | 0.02811799 | 0.999823 |
| 203 | ADHFE1 | 0.28113267 | 0.02824963 | 0.999823 |
| 204 | CCDC6 | −0.0907515 | 0.02831569 | 0.999823 |
| 205 | CCR2 | 0.40104756 | 0.02845943 | 0.999823 |
| 206 | HIST1H3F | −0.2252338 | 0.02846834 | 0.999823 |
| 207 | TIMP3 | −0.3519568 | 0.0285298 | 0.999823 |
| 208 | DIRC2 | 0.35441835 | 0.02860835 | 0.999823 |
| 209 | TCEB3 | −0.0868661 | 0.02863146 | 0.999823 |
| 210 | ZNF175 | −0.23782 | 0.02873465 | 0.999823 |
| 211 | DCUN1D1 | 0.14426954 | 0.02884704 | 0.999823 |
| 212 | PITPNM3 | −0.3213807 | 0.02888684 | 0.999823 |
| 213 | FOSB | 0.6135836 | 0.02896411 | 0.999823 |
| 214 | AQR | 0.06441042 | 0.02897575 | 0.999823 |
| 215 | GINS2 | −0.3871113 | 0.02900555 | 0.999823 |
| 216 | COPB1 | 0.06632984 | 0.02901851 | 0.999823 |
| 217 | IFIT1B | 0.32407614 | 0.02902811 | 0.999823 |
| 218 | CHMP6 | −0.2003379 | 0.02908907 | 0.999823 |
| 219 | NES | −0.2500724 | 0.02911141 | 0.999823 |
| 220 | CLSPN | −0.1648583 | 0.02920979 | 0.999823 |
| 221 | ZNF688 | −0.1424407 | 0.02923402 | 0.999823 |
| 222 | FAM69B | −0.3101323 | 0.02924848 | 0.999823 |
| 223 | APOE | −0.3243643 | 0.02940223 | 0.999823 |
| 224 | IGHG2 | −0.3336143 | 0.02945943 | 0.999823 |
| 225 | SLC25A32 | 0.13035519 | 0.02956385 | 0.999823 |
| 226 | APBB3 | 0.53377928 | 0.02960979 | 0.999823 |
| 227 | ARG1 | −0.3553876 | 0.02985572 | 0.999823 |
| 228 | SLC43A2 | 0.3769808 | 0.02989364 | 0.999823 |
| 229 | FABP4 | −0.2559567 | 0.02991405 | 0.999823 |
| 230 | HABP4 | 0.24172857 | 0.03005608 | 0.999823 |
| 231 | C2CD3 | 0.10120882 | 0.03017285 | 0.999823 |
| 232 | ORAI2 | −0.1762831 | 0.03018521 | 0.999823 |
| 233 | PER3 | 0.21521013 | 0.03029788 | 0.999823 |
| 234 | AC093673.5 | −0.2891258 | 0.03051499 | 0.999823 |
| 235 | KIF20A | −0.2844225 | 0.03053083 | 0.999823 |
| 236 | TBCK | 0.16579385 | 0.03066786 | 0.999823 |
| 237 | MT2A | −0.1566396 | 0.03087897 | 0.999823 |
| 238 | ALG8 | 0.20954186 | 0.03090105 | 0.999823 |
| 239 | LIN52 | 0.26231885 | 0.03095795 | 0.999823 |
| 240 | EPN2 | −0.3096568 | 0.03100399 | 0.999823 |
| 241 | ARIH1 | 0.09621805 | 0.0310866 | 0.999823 |
| 242 | ALDH1A1 | 0.22786487 | 0.0312975 | 0.999823 |
| 243 | ZNF703 | 0.27576921 | 0.03137979 | 0.999823 |
| 244 | ACPP | 0.29430814 | 0.03144763 | 0.999823 |
| 245 | TMEM234 | 0.28955944 | 0.03163473 | 0.999823 |
| 246 | RORA | 0.18907074 | 0.03167226 | 0.999823 |
| 247 | PSMA7 | −0.0670017 | 0.03173471 | 0.999823 |
| 248 | ING2 | −0.1277887 | 0.03182283 | 0.999823 |
| 249 | DUS3L | −0.2256817 | 0.03187092 | 0.999823 |
| 250 | SFMBT2 | 0.11771092 | 0.03207741 | 0.999823 |
| 251 | DDI2 | 0.10736217 | 0.03228297 | 0.999823 |
| 252 | AATK | 0.38287082 | 0.03238781 | 0.999823 |
| 253 | EOMES | 0.25204548 | 0.03245533 | 0.999823 |
| 254 | UNKL | 0.28483329 | 0.03253455 | 0.999823 |
| 255 | RACGAP1 | −0.1425339 | 0.03254637 | 0.999823 |
| 256 | MICALL2 | −0.2695713 | 0.03298099 | 0.999823 |
| 257 | CHTF8 | −0.0944541 | 0.03303854 | 0.999823 |
| 258 | EML2 | 0.12500876 | 0.03315582 | 0.999823 |
| 259 | VTI1A | 0.11874312 | 0.03326678 | 0.999823 |
| 260 | CKLF | −0.1923901 | 0.03339663 | 0.999823 |
| 261 | VWF | −0.3119939 | 0.03341445 | 0.999823 |
| 262 | AHNAK2 | −0.3975013 | 0.03341731 | 0.999823 |
| 263 | BET1L | −0.1441156 | 0.03349439 | 0.999823 |
| 264 | ENOX2 | 0.11686247 | 0.03380531 | 0.999823 |
| 265 | ZNF280C | 0.14656363 | 0.03385665 | 0.999823 |
| 266 | DNAJB4 | 0.15647994 | 0.03396513 | 0.999823 |
| 267 | FAM96B | −0.0996577 | 0.03432174 | 0.999823 |
| 268 | PRX | −0.2526297 | 0.0344957 | 0.999823 |
| 269 | RNF5 | −0.1396363 | 0.03478149 | 0.999823 |
| 270 | FAM212A | −0.1897578 | 0.03483004 | 0.999823 |
| 271 | DOCK10 | 0.10839726 | 0.0350643 | 0.999823 |
| 272 | PFN2 | −0.3192937 | 0.03507091 | 0.999823 |
| 273 | TGFBR3 | 0.25019499 | 0.03509169 | 0.999823 |
| 274 | C7orf50 | −0.1730759 | 0.03510597 | 0.999823 |
| 275 | OXSR1 | 0.10426307 | 0.03514952 | 0.999823 |
| 276 | PLSCR1 | −0.1539301 | 0.0352033 | 0.999823 |
| 277 | CDKN3 | −0.1793994 | 0.03526916 | 0.999823 |
| 278 | PTPRG | −0.2728392 | 0.03529744 | 0.999823 |
| 279 | SLC24A1 | −0.1781733 | 0.03535686 | 0.999823 |
| 280 | TFEC | 0.13865261 | 0.03540698 | 0.999823 |
| 281 | LFNG | 0.41498618 | 0.03546648 | 0.999823 |
| 282 | FOLR3 | −0.4824429 | 0.0356224 | 0.999823 |
| 283 | TCIRG1 | 0.42460234 | 0.03566012 | 0.999823 |
| 284 | ZNF248 | −0.1482991 | 0.03607008 | 0.999823 |
| 285 | SYTL2 | 0.22099325 | 0.03625104 | 0.999823 |
| 286 | GABARAP | −0.0681237 | 0.03665675 | 0.999823 |
| 287 | LYL1 | −0.1235543 | 0.03691445 | 0.999823 |
| 288 | ABHD8 | 0.27374966 | 0.03696402 | 0.999823 |
| 289 | ATL2 | 0.10911832 | 0.03696907 | 0.999823 |
| 290 | VAC14 | 0.12159626 | 0.03727137 | 0.999823 |
| 291 | MCM7 | −0.133427 | 0.03753042 | 0.999823 |
| 292 | WLS | 0.31920592 | 0.03777635 | 0.999823 |
| 293 | GMFG | −0.0762437 | 0.03777639 | 0.999823 |
| 294 | MIPEP | 0.19756689 | 0.0378531 | 0.999823 |
| 295 | MYBL1 | 0.13609471 | 0.03788196 | 0.999823 |
| 296 | CENPP | −0.1775462 | 0.03806583 | 0.999823 |
| 297 | C15orf52 | −0.2739874 | 0.03807024 | 0.999823 |
| 298 | PLK1 | −0.2821968 | 0.03807628 | 0.999823 |
| 299 | KIAA1324 | 0.38983772 | 0.03836171 | 0.999823 |
| 300 | TNNI2 | 0.28261991 | 0.03837332 | 0.999823 |
| 301 | ZNF629 | −0.2118135 | 0.03841179 | 0.999823 |
| 302 | ARHGEF10L | 0.28102719 | 0.03850904 | 0.999823 |
| 303 | SUSD6 | 0.19967273 | 0.0388163 | 0.999823 |
| 304 | MYL4 | −0.3963638 | 0.03884241 | 0.999823 |
| 305 | SMIM12 | −0.1271663 | 0.03896514 | 0.999823 |
| 306 | SREBF1 | 0.32605041 | 0.03909875 | 0.999823 |
| 307 | SVIL-AS1 | −0.2266914 | 0.03923228 | 0.999823 |
| 308 | ZFP91 | −0.1216083 | 0.03933035 | 0.999823 |
| 309 | SH3RF1 | 0.15044488 | 0.03937422 | 0.999823 |
| 310 | ATXN10 | 0.10995568 | 0.03956122 | 0.999823 |
| 311 | CSF3R | 0.40657663 | 0.03957007 | 0.999823 |
| 312 | ZNF362 | 0.09743055 | 0.03961429 | 0.999823 |
| 313 | NFU1 | −0.100997 | 0.03985893 | 0.999823 |
| 314 | PLXNB3 | −0.3310656 | 0.04054132 | 0.999823 |
| 315 | ARL2 | −0.161297 | 0.04070359 | 0.999823 |
| 316 | IGFBP2 | −0.5246938 | 0.04072204 | 0.999823 |
| 317 | APEX2 | −0.1420479 | 0.04090007 | 0.999823 |
| 318 | TMF1 | −0.0636947 | 0.04102724 | 0.999823 |
| 319 | SLC15A4 | 0.16273554 | 0.04117683 | 0.999823 |
| 320 | ANKRD33B | −0.2529753 | 0.04118417 | 0.999823 |
| 321 | ALG5 | 0.22362176 | 0.04129761 | 0.999823 |
| 322 | IGKV4-1 | −0.2543051 | 0.04167867 | 0.999823 |
| 323 | SNPH | −0.3155746 | 0.04194896 | 0.999823 |
| 324 | DNAJC24 | −0.1508193 | 0.04197652 | 0.999823 |
| 325 | TACC3 | −0.1476047 | 0.04202318 | 0.999823 |
| 326 | GK5 | 0.16735486 | 0.04214779 | 0.999823 |
| 327 | ALKBH5 | −0.0874234 | 0.04218493 | 0.999823 |
| 328 | CLEC7A | 0.21728275 | 0.04220416 | 0.999823 |
| 329 | KANK1 | −0.2255087 | 0.0422137 | 0.999823 |
| 330 | RNF8 | −0.1465837 | 0.04278441 | 0.999823 |
| 331 | COA5 | −0.0930276 | 0.04296264 | 0.999823 |
| 332 | TSPYL4 | −0.1347864 | 0.04312105 | 0.999823 |
| 333 | PID1 | 0.23786205 | 0.04317041 | 0.999823 |
| 334 | FAM32A | −0.1070765 | 0.04322635 | 0.999823 |
| 335 | YWHAZP4 | 0.22146435 | 0.04349002 | 0.999823 |
| 336 | SDHAP1 | 0.32501671 | 0.04367187 | 0.999823 |
| 337 | ADAP1 | 0.29057012 | 0.04368926 | 0.999823 |
| 338 | KIF26B | −0.3342392 | 0.04382832 | 0.999823 |
| 339 | RRN3P1 | 0.2103656 | 0.04410024 | 0.999823 |
| 340 | SIGIRR | 0.21434437 | 0.04419149 | 0.999823 |
| 341 | FAM127B | −0.1588417 | 0.0442788 | 0.999823 |
| 342 | COX8A | −0.1234086 | 0.04430464 | 0.999823 |
| 343 | BRI3BP | 0.26908104 | 0.04451084 | 0.999823 |
| 344 | GOLGA2 | −0.1421676 | 0.04455463 | 0.999823 |
| 345 | LNX2 | 0.13956437 | 0.04463541 | 0.999823 |
| 346 | RELT | 0.42035408 | 0.04485223 | 0.999823 |
| 347 | AMPD2 | 0.16253961 | 0.04491238 | 0.999823 |
| 348 | COL1A1 | −0.6942388 | 0.04500516 | 0.999823 |
| 349 | PRDM4 | −0.1005633 | 0.04520397 | 0.999823 |
| 350 | MAZ | −0.1086896 | 0.04529317 | 0.999823 |
| 351 | ERCC1 | −0.1098209 | 0.04537037 | 0.999823 |
| 352 | MXI1 | 0.23509908 | 0.04549618 | 0.999823 |
| 353 | THOC1 | 0.09635068 | 0.04565955 | 0.999823 |
| 354 | AK1 | −0.211156 | 0.04577507 | 0.999823 |
| 355 | ADGRF5 | −0.2657715 | 0.04607249 | 0.999823 |
| 356 | HELLS | −0.1233562 | 0.04608852 | 0.999823 |
| 357 | H2AFV | −0.1114127 | 0.04633008 | 0.999823 |
| 358 | SAMD14 | −0.2708931 | 0.04634534 | 0.999823 |
| 359 | RAB13 | −0.1397459 | 0.0466095 | 0.999823 |
| 360 | ITLN1 | 0.32354922 | 0.04674951 | 0.999823 |
| 361 | TTC39C | 0.09049556 | 0.04675678 | 0.999823 |
| 362 | IL2RB | 0.23545479 | 0.04691262 | 0.999823 |
| 363 | TMEM43 | 0.25763206 | 0.04733173 | 0.999823 |
| 364 | LDLRAD4 | −0.1447728 | 0.04766856 | 0.999823 |
| 365 | ZNF333 | 0.20134639 | 0.04775679 | 0.999823 |
| 366 | PLPP3 | −0.2300937 | 0.04776469 | 0.999823 |
| 367 | CRY1 | −0.1198904 | 0.04788717 | 0.999823 |
| 368 | TTC30B | −0.2580155 | 0.04798778 | 0.999823 |
| 369 | MEIS2 | −0.3392974 | 0.04815618 | 0.999823 |
| 370 | RBM17 | −0.0958349 | 0.04818096 | 0.999823 |
| 371 | MLEC | −0.2367412 | 0.04843225 | 0.999823 |
| 372 | UBE2R2 | −0.0875255 | 0.04870795 | 0.999823 |
| 373 | LTN1 | 0.07955132 | 0.04882314 | 0.999823 |
| 374 | KIAA1211 | −0.2514489 | 0.04887108 | 0.999823 |
| 375 | FGD6 | 0.14050951 | 0.04888819 | 0.999823 |
| 376 | FOXO3 | 0.21676256 | 0.04899547 | 0.999823 |
| 377 | CISD2 | 0.17691071 | 0.04913734 | 0.999823 |
| 378 | PAFAH2 | 0.22118013 | 0.04915197 | 0.999823 |
| 379 | LMBRD2 | 0.18522972 | 0.0492318 | 0.999823 |
| 380 | ZNF720 | −0.0931394 | 0.04930151 | 0.999823 |
| 381 | CHN2 | 0.18167055 | 0.04944251 | 0.999823 |
| 382 | RTEL1P1 | 0.65717329 | 0.04949181 | 0.999823 |
| 383 | DGAT2 | 0.41471623 | 0.04958542 | 0.999823 |
| 384 | CHMP3 | −0.1236621 | 0.04981575 | 0.999823 |
| 385 | CEP295NL | 0.64735357 | 0.04994012 | 0.999823 |

Third differential expression analysis of predicting preterm birth earlier than 35 weeks of gestational age, with blood samples collected between 17-23 weeks of gestational age, was performed using EdgeR and accounting for ethnicity, and cohort effects and gestational age at collection (111 PTB cases and 505 controls). Table 44 shows a set of top 6 genes with p-value<0.1 after adjustment from multiple hypothesis correction (FDR value), and also showed a significant deviation from the null hypothesis in a QQ plot for differentially expressed in pre-term birth cases (as shown in FIG. 44E). Table 45 shows an additional set of genes with p-value<0.1 for predicting preterm birth earlier than 35 weeks of gestation with blood samples collected between 17-23 weeks of gestational age. Genes are ordered according to their statistical significance (P-values).

TABLE 44
Top 6 genes with p-value < 0.1 after adjustment from multiple
hypothesis correction (FDR value), that are predictive for
preterm birth earlier than 35 weeks of gestation with blood
samples collected between 17-23 weeks of gestational age

| # | Gene | logFC | P-Value | FDR |
|---|---|---|---|---|
| 1 | FGA | −0.8922522 | 2.07E−07 | 0.002408 |
| 2 | COL3A1 | −1.1822498 | 7.06E−07 | 0.004095 |
| 3 | COL1A1 | −1.2205151 | 1.51E−06 | 0.005844 |
| 4 | COL1A2 | −1.0088068 | 1.09E−05 | 0.031216 |
| 5 | CDR1- | −0.7115165 | 1.35E−05 | 0.031216 |
| | AS | | | |
| 6 | HSPA1B | 0.57245175 | 1.74E−05 | 0.03368 |

TABLE 45
Additional set of genes with p-value < 0.1 for predicting
preterm birth earlier than 35 weeks of gestation with blood
samples collected between 17-23 weeks of gestational age

| # | Gene | logFC | P-Value | FDR |
|---|---|---|---|---|
| 1 | APOB | −0.5826059 | 0.00018491 | 0.306558 |
| 2 | NUP62CL | 0.36283704 | 0.00039242 | 0.569258 |
| 3 | CFH | −0.3925453 | 0.00064396 | 0.718794 |
| 4 | EZH1 | 0.10917121 | 0.00064612 | 0.718794 |
| 5 | FGB | −0.5417924 | 0.00071031 | 0.718794 |
| 6 | CPNE3 | −0.1598343 | 0.00075069 | 0.718794 |
| 7 | HIST1H2AI | −0.2214732 | 0.0008052 | 0.718794 |
| 8 | ABCA13 | −0.4106282 | 0.00115275 | 0.925144 |
| 9 | PLXNA3 | 0.53018951 | 0.00130431 | 0.925144 |
| 10 | KLF5 | −0.3693255 | 0.00135386 | 0.925144 |
| 11 | DCN | −0.7354785 | 0.00135523 | 0.925144 |
| 12 | ZBTB25 | 0.21316372 | 0.00146636 | 0.945397 |
| 13 | BEX1 | −0.3482247 | 0.00180193 | 0.999753 |
| 14 | PTGR1 | −0.2413271 | 0.00205964 | 0.999753 |
| 15 | CCDC80 | −0.5093286 | 0.00221921 | 0.999753 |
| 16 | FABP1 | −0.4395804 | 0.00232075 | 0.999753 |
| 17 | NABP2 | −0.2123718 | 0.00240932 | 0.999753 |
| 18 | MMP8 | −0.4528477 | 0.00248249 | 0.999753 |
| 19 | TMEM56 | −0.3358729 | 0.00262098 | 0.999753 |
| 20 | UNK | 0.10740632 | 0.00278715 | 0.999753 |
| 21 | CEACAM8 | −0.3912624 | 0.00290442 | 0.999753 |
| 22 | TK1 | −0.3710566 | 0.0029977 | 0.999753 |
| 23 | OLFM4 | −0.4569144 | 0.00307192 | 0.999753 |
| 24 | RETN | −0.4096121 | 0.00313118 | 0.999753 |
| 25 | POSTN | −0.4541202 | 0.0033519 | 0.999753 |
| 26 | POLR2A | 0.07393081 | 0.00360939 | 0.999753 |
| 27 | AMT | 0.23843514 | 0.00368187 | 0.999753 |
| 28 | ERLEC1 | 0.12130672 | 0.00377886 | 0.999753 |
| 29 | ALB | −0.3771048 | 0.00382494 | 0.999753 |
| 30 | GALNT7 | 0.22055918 | 0.00397611 | 0.999753 |
| 31 | TCN1 | −0.4369808 | 0.00418378 | 0.999753 |
| 32 | SEMA3C | −0.3609237 | 0.00437721 | 0.999753 |
| 33 | TYMS | −0.2121301 | 0.00439571 | 0.999753 |
| 34 | SERPINB10 | −0.3835561 | 0.00446509 | 0.999753 |
| 35 | KXD1 | 0.08832161 | 0.0046164 | 0.999753 |
| 36 | CRISP3 | −0.4517656 | 0.00464372 | 0.999753 |
| 37 | DLK1 | 0.61460928 | 0.00470334 | 0.999753 |
| 38 | APOH | −0.4805561 | 0.00477496 | 0.999753 |
| 39 | LTF | −0.3761597 | 0.00483032 | 0.999753 |
| 40 | IRAK2 | 0.19067454 | 0.0050855 | 0.999753 |
| 41 | CAMP | 0.3878126 | 0.00516332 | 0.999753 |
| 42 | CNPY3 | 0.11633546 | 0.00517313 | 0.999753 |
| 43 | VPS37B | 0.15814742 | 0.00518814 | 0.999753 |
| 44 | SAYSD1 | −0.1950745 | 0.00519864 | 0.999753 |
| 45 | AC005795.1 | 0.20057776 | 0.00526874 | 0.999753 |
| 46 | PSMD14 | −0.1158157 | 0.00538832 | 0.999753 |
| 47 | CST7 | −0.5217516 | 0.00539692 | 0.999753 |
| 48 | CAMKK1 | 0.26063751 | 0.00549614 | 0.999753 |
| 49 | VPS29 | −0.0830259 | 0.00560881 | 0.999753 |
| 50 | ARL1 | −0.1206514 | 0.00564317 | 0.999753 |
| 51 | PIAS4 | 0.11228955 | 0.00579437 | 0.999753 |
| 52 | ARPC4-TTLL3 | 0.2947005 | 0.00579671 | 0.999753 |
| 53 | CEACAM6 | −0.3567903 | 0.00583167 | 0.999753 |
| 54 | CCDC18-AS1 | 0.28958197 | 0.00632943 | 0.999753 |
| 55 | SF3A1 | 0.0783621 | 0.00639703 | 0.999753 |
| 56 | SLC2A5 | −0.3531257 | 0.00649409 | 0.999753 |
| 57 | IDI1 | −0.1187531 | 0.00657305 | 0.999753 |
| 58 | HSPA1A | 0.25560927 | 0.00674572 | 0.999753 |
| 59 | AHNAK2 | 0.391944 | 0.00690585 | 0.999753 |
| 60 | TPT1P4 | 0.23092184 | 0.00696854 | 0.999753 |
| 61 | ANXA1 | −0.1853844 | 0.00745635 | 0.999753 |
| 62 | TACC3 | 0.12955759 | 0.00747907 | 0.999753 |
| 63 | HBG1 | 0.6911507 | 0.00751888 | 0.999753 |
| 64 | NEK3 | −0.1559149 | 0.00776413 | 0.999753 |
| 65 | 1-Mar | 0.35690649 | 0.00795965 | 0.999753 |
| 66 | TMEM14C | −0.1709381 | 0.0079713 | 0.999753 |
| 67 | CCNA2 | −0.2263652 | 0.00801614 | 0.999753 |
| 68 | MTX2 | −0.1547208 | 0.0081661 | 0.999753 |
| 69 | IRS2 | 0.20766438 | 0.00820013 | 0.999753 |
| 70 | COQ7 | −0.1466541 | 0.00833708 | 0.999753 |
| 71 | S100B | −0.3287938 | 0.00861007 | 0.999753 |
| 72 | TSC22D4 | 0.11843984 | 0.00864383 | 0.999753 |
| 73 | OBSCN | 0.32640506 | 0.00888143 | 0.999753 |
| 74 | TPPP3 | −0.2379465 | 0.00899679 | 0.999753 |
| 75 | HIST1H4I | −0.1672515 | 0.00903644 | 0.999753 |
| 76 | PLD1 | −0.1847271 | 0.00992616 | 0.999753 |
| 77 | PER3 | 0.17292321 | 0.01018427 | 0.999753 |
| 78 | CTB-50L17.10 | 0.10225093 | 0.01026921 | 0.999753 |
| 79 | TEX30 | −0.2110864 | 0.01047769 | 0.999753 |
| 80 | AFF2 | −0.19233 | 0.01048049 | 0.999753 |
| 81 | INHBA | −0.3622862 | 0.01049335 | 0.999753 |
| 82 | RNF111 | 0.05623506 | 0.01080035 | 0.999753 |
| 83 | PABPC1L | 0.49410783 | 0.01080075 | 0.999753 |
| 84 | GPBP1L1 | 0.05507902 | 0.01090532 | 0.999753 |
| 85 | BPI | −0.3221364 | 0.01104231 | 0.999753 |
| 86 | SLC3A2 | 0.18156536 | 0.0112006 | 0.999753 |
| 87 | MYH11 | −0.254936 | 0.01126761 | 0.999753 |
| 88 | ALDH1A2 | −0.2305017 | 0.0113409 | 0.999753 |
| 89 | TTN | 0.46246546 | 0.01139138 | 0.999753 |
| 90 | ABHD16A | 0.20970139 | 0.01140776 | 0.999753 |
| 91 | GS1-44D20.1 | 0.17063532 | 0.0114796 | 0.999753 |
| 92 | NR1D2 | 0.10785231 | 0.0115101 | 0.999753 |
| 93 | RNASE3 | −0.3866944 | 0.01159032 | 0.999753 |
| 94 | TRAPPC12 | 0.1120295 | 0.01183535 | 0.999753 |
| 95 | RAD51B | 0.2566469 | 0.01191832 | 0.999753 |
| 96 | POLR2K | −0.1549786 | 0.01203891 | 0.999753 |
| 97 | CDH6 | 0.47160832 | 0.01203921 | 0.999753 |
| 98 | ANKRD36 | 0.15136038 | 0.01212896 | 0.999753 |
| 99 | ZNF550 | 0.30399132 | 0.01222071 | 0.999753 |
| 100 | SNX19 | −0.1850206 | 0.0123524 | 0.999753 |
| 101 | PSMA3 | −0.0935928 | 0.01294008 | 0.999753 |
| 102 | SF3A2 | 0.0822754 | 0.01294752 | 0.999753 |
| 103 | PDE3B | 0.30101247 | 0.01297583 | 0.999753 |
| 104 | NELL2 | 0.3861488 | 0.01304957 | 0.999753 |
| 105 | KATNA1 | −0.0912704 | 0.01308488 | 0.999753 |
| 106 | WASH6P | 0.45059223 | 0.01322944 | 0.999753 |
| 107 | ITGA9 | −0.2609704 | 0.0134086 | 0.999753 |
| 108 | LGALS1 | −0.1618404 | 0.01363949 | 0.999753 |
| 109 | GALT | 0.29467619 | 0.01376172 | 0.999753 |
| 110 | TRIM8 | 0.09716423 | 0.01403662 | 0.999753 |
| 111 | NICN1 | −0.2172396 | 0.01419089 | 0.999753 |
| 112 | FERMT2 | −0.1951171 | 0.01422377 | 0.999753 |
| 113 | PDIA4 | 0.09664602 | 0.01450684 | 0.999753 |
| 114 | EPB42 | −0.2430774 | 0.01452652 | 0.999753 |
| 115 | RIPK2 | −0.110475 | 0.01457411 | 0.999753 |
| 116 | PELI2 | 0.14817975 | 0.01479923 | 0.999753 |
| 117 | KLHL35 | 0.46532872 | 0.01484529 | 0.999753 |
| 118 | SLC15A4 | 0.14721116 | 0.01489834 | 0.999753 |
| 119 | TGFB2 | 0.28472572 | 0.01507659 | 0.999753 |
| 120 | RUNDC3A | −0.2992381 | 0.01523721 | 0.999753 |
| 121 | SGSM3 | 0.12690997 | 0.01548659 | 0.999753 |
| 122 | LTA4H | −0.1483382 | 0.01558966 | 0.999753 |
| 123 | CANT1 | 0.20605193 | 0.01570725 | 0.999753 |
| 124 | PPP1R35 | 0.18021209 | 0.01616723 | 0.999753 |
| 125 | MPO | −0.2474597 | 0.01617706 | 0.999753 |
| 126 | FOXJ2 | 0.11503104 | 0.01621339 | 0.999753 |
| 127 | SELENBP1 | −0.2532564 | 0.01622888 | 0.999753 |
| 128 | CCDC173 | 0.37753916 | 0.01632994 | 0.999753 |
| 129 | CTDSP2 | 0.07518886 | 0.01636667 | 0.999753 |
| 130 | NUDT9 | −0.1365469 | 0.01656297 | 0.999753 |
| 131 | ATP10D | 0.26481636 | 0.01656597 | 0.999753 |
| 132 | AZI2 | −0.086938 | 0.01659226 | 0.999753 |
| 133 | FUCA2 | 0.14782949 | 0.01669051 | 0.999753 |
| 134 | PRRC2C | 0.05896815 | 0.01677844 | 0.999753 |
| 135 | DEFA4 | −0.3046262 | 0.01684177 | 0.999753 |
| 136 | ZNF257 | 0.18123619 | 0.01690074 | 0.999753 |
| 137 | H3F3B | 0.0730957 | 0.01711348 | 0.999753 |
| 138 | FGGY | −0.1220351 | 0.01712126 | 0.999753 |
| 139 | TTC38 | −0.1944937 | 0.01714651 | 0.999753 |
| 140 | PGM2 | −0.0807912 | 0.01752113 | 0.999753 |
| 141 | SH3BP5 | −0.1490668 | 0.0175562 | 0.999753 |
| 142 | FAM133B | 0.12698846 | 0.01767701 | 0.999753 |
| 143 | ARHGEF18 | 0.20558778 | 0.01790049 | 0.999753 |
| 144 | SREK1 | 0.07846238 | 0.017972 | 0.999753 |
| 145 | C7orf31 | 0.10246202 | 0.01799207 | 0.999753 |
| 146 | CTD-2017F17.2 | 0.46727872 | 0.0183904 | 0.999753 |
| 147 | STIM2 | 0.12847968 | 0.01859262 | 0.999753 |
| 148 | EP400NL | 0.28376719 | 0.01862442 | 0.999753 |
| 149 | NUDCD2 | −0.165063 | 0.01909539 | 0.999753 |
| 150 | ZBTB16 | 0.13331658 | 0.01913721 | 0.999753 |
| 151 | GRPEL2 | −0.1877752 | 0.01927475 | 0.999753 |
| 152 | NLRC4 | −0.1701506 | 0.0195017 | 0.999753 |
| 153 | HIST1H3I | −0.1866323 | 0.01966998 | 0.999753 |
| 154 | IL2RB | 0.22901014 | 0.01978275 | 0.999753 |
| 155 | IL7R | 0.17493298 | 0.02021919 | 0.999753 |
| 156 | TMEM43 | 0.25352755 | 0.02060582 | 0.999753 |
| 157 | NBPF11 | 0.1485556 | 0.02075834 | 0.999753 |
| 158 | ANKRD36B | 0.1927486 | 0.02126847 | 0.999753 |
| 159 | HIKESHI | −0.1211526 | 0.02130131 | 0.999753 |
| 160 | ADSS | −0.0950366 | 0.02138402 | 0.999753 |
| 161 | CCDC141 | 0.3919521 | 0.02152967 | 0.999753 |
| 162 | PKD1 | 0.30833702 | 0.02177052 | 0.999753 |
| 163 | CCR2 | 0.34638257 | 0.02194942 | 0.999753 |
| 164 | MS4A3 | −0.2869229 | 0.02244994 | 0.999753 |
| 165 | MUT | −0.1097854 | 0.02273149 | 0.999753 |
| 166 | IGF1R | 0.1945484 | 0.02282841 | 0.999753 |
| 167 | CASS4 | 0.12014184 | 0.02291597 | 0.999753 |
| 168 | DLD | −0.0865122 | 0.02300047 | 0.999753 |
| 169 | NFXL1 | −0.1051861 | 0.02334338 | 0.999753 |
| 170 | QSOX2 | 0.26727564 | 0.0235745 | 0.999753 |
| 171 | MSNP1 | 0.15424572 | 0.02358748 | 0.999753 |
| 172 | GPAT4 | 0.14540808 | 0.02361456 | 0.999753 |
| 173 | GSKIP | −0.1367002 | 0.02403918 | 0.999753 |
| 174 | RHOU | −0.149483 | 0.02406404 | 0.999753 |
| 175 | TKFC | 0.12691977 | 0.02437814 | 0.999753 |
| 176 | ATP10A | 0.30508566 | 0.02446292 | 0.999753 |
| 177 | PTP4A3 | 0.1449434 | 0.02472307 | 0.999753 |
| 178 | MEI1 | −0.2446254 | 0.02495366 | 0.999753 |
| 179 | IL7 | 0.18042937 | 0.02506084 | 0.999753 |
| 180 | HIST1H3D | −0.1312724 | 0.02506997 | 0.999753 |
| 181 | SMIM20 | −0.1498791 | 0.02509728 | 0.999753 |
| 182 | AK5 | 0.2135572 | 0.02522872 | 0.999753 |
| 183 | ARG1 | −0.2523013 | 0.02529551 | 0.999753 |
| 184 | MLLT11 | 0.2563372 | 0.02546545 | 0.999753 |
| 185 | CTD-2319112.10 | 0.21588609 | 0.02551335 | 0.999753 |
| 186 | EEF1E1 | −0.1263748 | 0.02554448 | 0.999753 |
| 187 | CKAP2L | −0.1360314 | 0.0255639 | 0.999753 |
| 188 | SLC4A4 | −0.2360361 | 0.02587196 | 0.999753 |
| 189 | NMRAL1 | 0.12247516 | 0.02597727 | 0.999753 |
| 190 | PRG4 | −0.3738295 | 0.02605235 | 0.999753 |
| 191 | SELPLG | 0.21964904 | 0.02605785 | 0.999753 |
| 192 | MALAT1 | 0.33881384 | 0.02614156 | 0.999753 |
| 193 | EIF4HP1 | 0.25442345 | 0.02616057 | 0.999753 |
| 194 | COX5A | −0.0822105 | 0.02621488 | 0.999753 |
| 195 | SPOCK2 | 0.31101424 | 0.02634448 | 0.999753 |
| 196 | RILPL1 | 0.12949377 | 0.02640549 | 0.999753 |
| 197 | CHD2 | 0.05277056 | 0.02651847 | 0.999753 |
| 198 | TCTN3 | 0.23335682 | 0.02665692 | 0.999753 |
| 199 | STYXL1 | 0.10093585 | 0.02710051 | 0.999753 |
| 200 | TM2D3 | 0.11782763 | 0.02742488 | 0.999753 |
| 201 | HIST1H2AH | −0.1930756 | 0.0277185 | 0.999753 |
| 202 | C1orf123 | −0.1423279 | 0.0277822 | 0.999753 |
| 203 | B3GNT5 | −0.2444396 | 0.02804637 | 0.999753 |
| 204 | TPD52L1 | −0.2825496 | 0.0282404 | 0.999753 |
| 205 | MIER3 | −0.1124144 | 0.02851633 | 0.999753 |
| 206 | TMEM35B | 0.20806256 | 0.02864175 | 0.999753 |
| 207 | TSPYL2 | 0.1697368 | 0.02864491 | 0.999753 |
| 208 | ADA | −0.1589866 | 0.02866328 | 0.999753 |
| 209 | ARID1B | 0.0528842 | 0.02870548 | 0.999753 |
| 210 | FN1 | −0.2404726 | 0.02905857 | 0.999753 |
| 211 | SELENOP | −0.2151347 | 0.0291476 | 0.999753 |
| 212 | RBM6 | 0.07373482 | 0.02920453 | 0.999753 |
| 213 | CEP68 | 0.14191808 | 0.02945737 | 0.999753 |
| 214 | MTCL1 | 0.18237028 | 0.02957545 | 0.999753 |
| 215 | ALAS2 | −0.2291027 | 0.02974141 | 0.999753 |
| 216 | EXOG | 0.1727914 | 0.02989632 | 0.999753 |
| 217 | GLTSCR1 | 0.19245341 | 0.02998657 | 0.999753 |
| 218 | PGLYRP1 | −0.2830829 | 0.02998786 | 0.999753 |
| 219 | SMIM5 | 0.20149599 | 0.0300126 | 0.999753 |
| 220 | CDC6 | −0.1658365 | 0.0300815 | 0.999753 |
| 221 | CAV2 | 0.21059274 | 0.03018762 | 0.999753 |
| 222 | NBPF9 | 0.17983382 | 0.0302083 | 0.999753 |
| 223 | PTGIR | 0.17136031 | 0.0304244 | 0.999753 |
| 224 | SNRPG | −0.1371207 | 0.03044173 | 0.999753 |
| 225 | WBP1L | 0.12104254 | 0.03044713 | 0.999753 |
| 226 | TOR1AIP2 | 0.08360512 | 0.03048316 | 0.999753 |
| 227 | EMB | −0.0897702 | 0.0305139 | 0.999753 |
| 228 | AVPR1A | 0.21704274 | 0.03059684 | 0.999753 |
| 229 | P4HA2 | 0.37243812 | 0.03060348 | 0.999753 |
| 230 | GYG1 | −0.1125703 | 0.03083176 | 0.999753 |
| 231 | C3 | −0.1993848 | 0.03100619 | 0.999753 |
| 232 | DOC2B | −0.2537712 | 0.03104329 | 0.999753 |
| 233 | HEATR5A | −0.1057825 | 0.03105816 | 0.999753 |
| 234 | G2E3 | −0.0844544 | 0.03111066 | 0.999753 |
| 235 | PCNT | 0.06710106 | 0.03115947 | 0.999753 |
| 236 | CYP2E1 | −0.2906311 | 0.03118366 | 0.999753 |
| 237 | ZDHHC5 | 0.09675558 | 0.03122839 | 0.999753 |
| 238 | KDM4B | 0.11555829 | 0.03124625 | 0.999753 |
| 239 | TIPRL | −0.0841239 | 0.03126632 | 0.999753 |
| 240 | PIWIL4 | −0.1441967 | 0.03128178 | 0.999753 |
| 241 | TOX4 | 0.05298922 | 0.03128257 | 0.999753 |
| 242 | CYB5D2 | 0.17434026 | 0.03151201 | 0.999753 |
| 243 | MCTS1 | −0.1283583 | 0.03162187 | 0.999753 |
| 244 | ARPC1A | −0.0762396 | 0.03166386 | 0.999753 |
| 245 | GAB1 | 0.10675688 | 0.03177612 | 0.999753 |
| 246 | KIAA1328 | 0.08801699 | 0.03179623 | 0.999753 |
| 247 | CBX7 | 0.14747089 | 0.03216422 | 0.999753 |
| 248 | MYBL2 | −0.1459055 | 0.03222052 | 0.999753 |
| 249 | COX20 | −0.0940038 | 0.03228853 | 0.999753 |
| 250 | S100A12 | −0.2026783 | 0.0324576 | 0.999753 |
| 251 | DCUN1D1 | 0.10810631 | 0.03255478 | 0.999753 |
| 252 | CEP97 | −0.1203253 | 0.03257225 | 0.999753 |
| 253 | CCR7 | 0.27413875 | 0.03272345 | 0.999753 |
| 254 | IGFBP2 | −0.3549402 | 0.03305778 | 0.999753 |
| 255 | PROSER2 | 0.18257741 | 0.03312428 | 0.999753 |
| 256 | POLE4 | −0.1296828 | 0.03313182 | 0.999753 |
| 257 | CIC | 0.10838803 | 0.03321301 | 0.999753 |
| 258 | ING1 | 0.08081968 | 0.03322562 | 0.999753 |
| 259 | PPIL1 | −0.1927958 | 0.03327341 | 0.999753 |
| 260 | C3orf14 | −0.2563693 | 0.03333526 | 0.999753 |
| 261 | SF3B5 | −0.116132 | 0.03338042 | 0.999753 |
| 262 | ISCU | 0.08400156 | 0.03338527 | 0.999753 |
| 263 | IGHG2 | 0.26195808 | 0.03380502 | 0.999753 |
| 264 | CHPF2 | 0.28256794 | 0.03383726 | 0.999753 |
| 265 | E2F8 | −0.2465367 | 0.03388536 | 0.999753 |
| 266 | Metazoa_SRP_ENSG00000278771 | −0.2058012 | 0.033919 | 0.999753 |
| 267 | MIB2 | 0.17694897 | 0.03404959 | 0.999753 |
| 268 | CCNK | 0.0529718 | 0.03421768 | 0.999753 |
| 269 | ZNF292 | 0.06953068 | 0.03431769 | 0.999753 |
| 270 | PPP1R15A | 0.13124538 | 0.0343715 | 0.999753 |
| 271 | ATP7B | 0.21466598 | 0.03451874 | 0.999753 |
| 272 | ANKS6 | 0.24689062 | 0.03469057 | 0.999753 |
| 273 | PCP2 | 0.22564137 | 0.03478878 | 0.999753 |
| 274 | RRM2 | −0.1881119 | 0.03494304 | 0.999753 |
| 275 | CPEB3 | 0.15049772 | 0.03504406 | 0.999753 |
| 276 | FOXM1 | −0.1910254 | 0.03513846 | 0.999753 |
| 277 | HIST1H2AL | −0.1450165 | 0.03532496 | 0.999753 |
| 278 | NEFH | −0.1914372 | 0.035411 | 0.999753 |
| 279 | MAST3 | 0.10031607 | 0.03547816 | 0.999753 |
| 280 | ZFAT | 0.12262196 | 0.03593907 | 0.999753 |
| 281 | CUL3 | −0.0453055 | 0.03610051 | 0.999753 |
| 282 | BBC3 | 0.17360764 | 0.03631048 | 0.999753 |
| 283 | TAOK2 | 0.10209633 | 0.03647822 | 0.999753 |
| 284 | BICD1 | 0.11544926 | 0.03677942 | 0.999753 |
| 285 | AC006116.22 | 0.2292784 | 0.03678963 | 0.999753 |
| 286 | ING4 | 0.09297105 | 0.03695455 | 0.999753 |
| 287 | MT-TP | −0.2835665 | 0.03697 | 0.999753 |
| 288 | DNAJB1 | 0.1476015 | 0.03700129 | 0.999753 |
| 289 | ADAP2 | −0.1722998 | 0.03712279 | 0.999753 |
| 290 | PREP | −0.1098884 | 0.0379176 | 0.999753 |
| 291 | FAM49B | −0.0952589 | 0.0379976 | 0.999753 |
| 292 | PLK1 | −0.2051848 | 0.03801488 | 0.999753 |
| 293 | SYNJ2 | 0.13699949 | 0.03801954 | 0.999753 |
| 294 | INO80C | −0.1330365 | 0.03804286 | 0.999753 |
| 295 | HBE1 | 0.42870509 | 0.03830571 | 0.999753 |
| 296 | USP11 | 0.06798314 | 0.03840566 | 0.999753 |
| 297 | MCM6 | 0.15356415 | 0.03843693 | 0.999753 |
| 298 | MRPL36 | −0.134445 | 0.03855475 | 0.999753 |
| 299 | BBOF1 | 0.13716434 | 0.0385769 | 0.999753 |
| 300 | TTC14 | 0.26365258 | 0.03869701 | 0.999753 |
| 301 | ZNF746 | 0.18539114 | 0.0388262 | 0.999753 |
| 302 | SMCR8 | 0.07266396 | 0.03890485 | 0.999753 |
| 303 | DGKA | 0.16075717 | 0.03895777 | 0.999753 |
| 304 | C3orf58 | 0.13596494 | 0.03904565 | 0.999753 |
| 305 | CD7 | 0.20770221 | 0.03920229 | 0.999753 |
| 306 | EPPK1 | 0.3359978 | 0.03929967 | 0.999753 |
| 307 | ATAD3B | 0.33834265 | 0.03931759 | 0.999753 |
| 308 | APBB1 | 0.19196402 | 0.03941002 | 0.999753 |
| 309 | UBR5 | 0.03721083 | 0.03951333 | 0.999753 |
| 310 | SLC14A1 | −0.2118413 | 0.03955782 | 0.999753 |
| 311 | GOLGA8R | 0.20030818 | 0.03963813 | 0.999753 |
| 312 | S100A4 | −0.1270935 | 0.03978126 | 0.999753 |
| 313 | NAT1 | −0.1691511 | 0.04054604 | 0.999753 |
| 314 | CASP5 | −0.1777435 | 0.04055036 | 0.999753 |
| 315 | DDX31 | 0.17809076 | 0.04063238 | 0.999753 |
| 316 | LUC7L3 | 0.07402997 | 0.04065676 | 0.999753 |
| 317 | PSMA3-AS1 | 0.18324627 | 0.04089756 | 0.999753 |
| 318 | MUC3A | −0.3375097 | 0.04093926 | 0.999753 |
| 319 | PRR5L | −0.0957441 | 0.04096973 | 0.999753 |
| 320 | SETD4 | 0.18086207 | 0.04126734 | 0.999753 |
| 321 | PRPSAP1 | −0.1033051 | 0.04149971 | 0.999753 |
| 322 | MRPL51 | −0.0994934 | 0.04151102 | 0.999753 |
| 323 | LENG8 | 0.24702492 | 0.04167004 | 0.999753 |
| 324 | TMEM55B | 0.12862126 | 0.04179192 | 0.999753 |
| 325 | UBXN4 | 0.07134072 | 0.04180286 | 0.999753 |
| 326 | PABPN1 | 0.07244813 | 0.04195609 | 0.999753 |
| 327 | TRAFD1 | 0.06658772 | 0.04213277 | 0.999753 |
| 328 | SNTB2 | −0.1100601 | 0.04233428 | 0.999753 |
| 329 | MRPL48 | −0.1195106 | 0.04241753 | 0.999753 |
| 330 | SPATA5 | 0.09150062 | 0.04246213 | 0.999753 |
| 331 | H2AFX | −0.1776987 | 0.04275797 | 0.999753 |
| 332 | IGFBP4 | −0.2246328 | 0.04288488 | 0.999753 |
| 333 | GFI1 | −0.2316195 | 0.04296089 | 0.999753 |
| 334 | HBS1L | −0.0546702 | 0.04320669 | 0.999753 |
| 335 | TMUB2 | 0.19402025 | 0.04323319 | 0.999753 |
| 336 | QRSL1 | −0.1400253 | 0.04327588 | 0.999753 |
| 337 | MKI67 | −0.1150793 | 0.04343116 | 0.999753 |
| 338 | SMIM24 | −0.2066749 | 0.04344628 | 0.999753 |
| 339 | FAM78A | 0.09176017 | 0.04368267 | 0.999753 |
| 340 | AHR | −0.0810842 | 0.0439174 | 0.999753 |
| 341 | PLXNA2 | 0.17677215 | 0.04405629 | 0.999753 |
| 342 | ANKMY1 | 0.12999115 | 0.0440723 | 0.999753 |
| 343 | MEGF6 | 0.44577879 | 0.0443392 | 0.999753 |
| 344 | NBPF10 | 0.14614391 | 0.04464845 | 0.999753 |
| 345 | TMEM206 | 0.1606816 | 0.04479684 | 0.999753 |
| 346 | CD24 | −0.2078109 | 0.04489029 | 0.999753 |
| 347 | RPAP3 | 0.08627224 | 0.0450221 | 0.999753 |
| 348 | KLHL12 | 0.07504398 | 0.04508842 | 0.999753 |
| 349 | FAM208A | −0.0419344 | 0.04534657 | 0.999753 |
| 350 | FAM26E | 0.18269354 | 0.04536151 | 0.999753 |
| 351 | C10orf11 | −0.153169 | 0.04553543 | 0.999753 |
| 352 | COPS5 | −0.0541677 | 0.04564979 | 0.999753 |
| 353 | SNX29 | 0.08506495 | 0.04565399 | 0.999753 |
| 354 | SLC7A6 | 0.21035707 | 0.04576956 | 0.999753 |
| 355 | CD19 | 0.29589004 | 0.04584316 | 0.999753 |
| 356 | CNNM4 | 0.22034199 | 0.04589658 | 0.999753 |
| 357 | NIF3L1 | −0.1567129 | 0.04591594 | 0.999753 |
| 358 | PBX2 | 0.09040127 | 0.04600611 | 0.999753 |
| 359 | MAPK1IP1L | 0.08569724 | 0.04627337 | 0.999753 |
| 360 | EFCAB5 | 0.17026595 | 0.0462916 | 0.999753 |
| 361 | MISP3 | 0.19341489 | 0.04640056 | 0.999753 |
| 362 | PAICS | −0.1323756 | 0.0466355 | 0.999753 |
| 363 | NBN | −0.0542005 | 0.04667697 | 0.999753 |
| 364 | PIK3IP1 | 0.26921035 | 0.046751 | 0.999753 |
| 365 | TMEM106B | 0.0814957 | 0.04676457 | 0.999753 |
| 366 | ANP32B | 0.07359856 | 0.04691678 | 0.999753 |
| 367 | NBEAL1 | 0.0661075 | 0.04723681 | 0.999753 |
| 368 | FPGT | −0.1115372 | 0.04771241 | 0.999753 |
| 369 | MYLIP | 0.12467534 | 0.04805567 | 0.999753 |
| 370 | SDHA | 0.09790987 | 0.04806401 | 0.999753 |
| 371 | STX11 | 0.09670973 | 0.04819952 | 0.999753 |
| 372 | MT-TM | −0.2647748 | 0.04824865 | 0.999753 |
| 373 | ZNF865 | 0.18795028 | 0.04828377 | 0.999753 |
| 374 | FAN1 | 0.12049483 | 0.04840424 | 0.999753 |
| 375 | CYSLTR1 | −0.1743521 | 0.04873218 | 0.999753 |
| 376 | CACNB4 | −0.2114985 | 0.04891416 | 0.999753 |
| 377 | HPD | −0.2728785 | 0.04892793 | 0.999753 |
| 378 | ZNF630 | −0.1900738 | 0.04907291 | 0.999753 |
| 379 | RPA3 | −0.1355575 | 0.04911536 | 0.999753 |
| 380 | ADRA2A | 0.24629972 | 0.04914611 | 0.999753 |
| 381 | PTMAP2 | 0.18200957 | 0.04963155 | 0.999753 |
| 382 | ZW10 | −0.0832316 | 0.04969237 | 0.999753 |
| 383 | ADAM28 | 0.22059564 | 0.04971214 | 0.999753 |
| 384 | FAM175B | 0.06386437 | 0.04988883 | 0.999753 |
| 385 | ARHGAP45 | 0.09866914 | 0.04996179 | 0.999753 |
| 386 | TCEA1 | 0.05831703 | 0.04999775 | 0.999753 |
| 387 | NIPA2 | −0.1265798 | 0.05021501 | 0.999753 |
| 388 | PTMA | 0.10851123 | 0.05038825 | 0.999753 |
| 389 | MEF2D | 0.06287954 | 0.05041783 | 0.999753 |
| 390 | S100A8 | −0.1731034 | 0.05043263 | 0.999753 |
| 391 | UST | 0.19855501 | 0.05059008 | 0.999753 |
| 392 | TOP1 | 0.07870085 | 0.0506117 | 0.999753 |
| 393 | ZNF587 | 0.17157982 | 0.0506316 | 0.999753 |

### Example 22: Prediction of Pre-Term Birth (PTB) on Combined Multiple Cohorts Using an Effect Size

Features were identified from a training set comprising Log 2 RPM gene expression data from six cohorts (FIG. 44A), collected at about 25 weeks gestation). Seventy percent of the training data was split into a training set (38 cases and 186 controls), while the remaining 30% was used as a test set (18 cases and 79 controls) for feature engineering. Candidate genes were selected for an upregulated effect size in PTB greater than an effect size threshold. Principal component analysis (PCA) was trained on standardized Log 2 CPM counts from controls in the training set. The full training and test sets were then PCA transformed. A logistic model (L1 penalty) was trained on the PCA components calculated from the training data and then applied to principal components similarly calculated from the test dataset. The hyperparameters for the effect size threshold and the PCA variance threshold were optimized by a grid search based on optimizing the AUC on the test set. The effect size threshold was set to 0.3, yielding 837 high effect genes, and the PCA variance threshold was set to 0.6, obtaining an AUC of 0.56 in the test set using the aforementioned logistic regression model obtained from the training set.

Table 46 shows a set of top 50 genes contributing to 20% of the total PTB model weight. Table 47 shows the remaining 787 genes contributing to 80% of the model weight. Genes are sorted by total weight in the modeling, which is obtained as the matrix multiplication between PCA components and weights of the logistic regression model.

TABLE 46
Top 50 high effect genes identified using an effect size
threshold of 0.3 and contributing 20% of total PTB model
weight. Genes are sorted by total weight in the model.
Top 50 genes contribute to 20% of total model weight.

| # | Gene | Weight |
|---|---|---|
| 1 | EGFL7 | 0.03915196 |
| 2 | FAM65C | 0.03236397 |
| 3 | FAM212A | 0.03105369 |
| 4 | RNF8 | 0.02983798 |
| 5 | EPHX2 | 0.02916541 |
| 6 | SPCS2 | 0.02810884 |
| 7 | ACOT8 | 0.02800098 |
| 8 | RPS19BP1 | 0.02520334 |
| 9 | SMIM12 | 0.0245331 |
| 10 | TNFSF13 | 0.0243419 |
| 11 | SF3A2 | 0.02431467 |
| 12 | TRPM6 | 0.02420862 |
| 13 | C20orf96 | 0.02384787 |
| 14 | C1orf43 | 0.02382509 |
| 15 | SGMS1 | 0.02375853 |
| 16 | CCDC28B | 0.02329786 |
| 17 | DOLPP1 | 0.0223773 |
| 18 | TNFAIP8L1 | 0.0218296 |
| 19 | TRIP10 | 0.02178185 |
| 20 | SMIM1 | 0.02162177 |
| 21 | RER1 | 0.02157154 |
| 22 | ZNF429 | 0.02134285 |
| 23 | TATDN2 | 0.02073552 |
| 24 | FBXO18 | 0.02071262 |
| 25 | DNMT3B | 0.02065702 |
| 26 | VPS28 | 0.02052528 |
| 27 | FAM189B | 0.02015087 |
| 28 | BCL7B | 0.01989426 |
| 29 | OBSL1 | 0.01979065 |
| 30 | HERC6 | 0.01978811 |
| 31 | MYEF2 | 0.01938121 |
| 32 | APOC1 | 0.01933969 |
| 33 | TRA2B | 0.01901918 |
| 34 | ARAF | 0.01895693 |
| 35 | FGA | 0.01895179 |
| 36 | RNF181 | 0.01877974 |
| 37 | SERPINH1 | 0.01844746 |
| 38 | MAPK13 | 0.01829422 |
| 39 | RALY | 0.01829161 |
| 40 | RAB11FIP3 | 0.01819169 |
| 41 | NQO1 | 0.01815695 |
| 42 | ULK3 | 0.01806994 |
| 43 | C8orf76 | 0.01794826 |
| 44 | C1orf174 | 0.01780182 |
| 45 | BEND7 | 0.01764843 |
| 46 | AP1B1 | 0.01759565 |
| 47 | TRNAU1AP | 0.01749675 |
| 48 | ING2 | 0.01749674 |
| 49 | CHMP5 | 0.01733394 |
| 50 | SRSF3 | 0.01723014 |

TABLE 47
Remaining 787 high effect genes identified using
an effect size threshold of 0.3 and contributing
the remaining 80% of PTB model weight

| # | Gene | Weight |
|---|---|---|
| 1 | HEXIM1 | 0.01721642 |
| 2 | IFI44 | 0.01721479 |
| 3 | PIAS4 | 0.01712305 |
| 4 | SLC31A1 | 0.01692751 |
| 5 | ZDHHC12 | 0.01663261 |
| 6 | GTF2H5 | 0.01655058 |
| 7 | PAQR7 | 0.01628653 |
| 8 | UFD1L | 0.01623378 |
| 9 | RFESD | 0.01622693 |
| 10 | CDK16 | 0.01605331 |
| 11 | XPNPEP3 | 0.01599098 |
| 12 | SLC3A2 | 0.01592603 |
| 13 | ENSG00000281457 | 0.01589179 |
| 14 | FGFR1OP | 0.01573999 |
| 15 | MBIP | 0.01572768 |
| 16 | CNTROB | 0.01568919 |
| 17 | EPSTI1 | 0.01554056 |
| 18 | ANKRD9 | 0.01553828 |
| 19 | C11orf68 | 0.01553649 |
| 20 | PANX2 | 0.01550303 |
| 21 | KLC3 | 0.01542868 |
| 22 | RHOF | 0.01542195 |
| 23 | SURF4 | 0.01521329 |
| 24 | STUB1 | 0.01517591 |
| 25 | C12orf57 | 0.01515882 |
| 26 | ZC3H4 | 0.01506663 |
| 27 | SURF1 | 0.01501501 |
| 28 | FABP1 | 0.01491422 |
| 29 | NMI | 0.01490726 |
| 30 | TNNI3 | 0.01465785 |
| 31 | PRG4 | 0.01450515 |
| 32 | CYP 20.00 | 0.01438684 |
| 33 | APOH | 0.01435591 |
| 34 | MRVI1 | 0.01431809 |
| 35 | CDH5 | 0.01423431 |
| 36 | BSDC1 | 0.01422665 |
| 37 | SNED1 | 0.01412338 |
| 38 | ZNF470 | 0.01407822 |
| 39 | SEMA3D | 0.0140655 |
| 40 | KATNA1 | 0.01406457 |
| 41 | UCK1 | 0.01398802 |
| 42 | NEUROD2 | 0.0139867 |
| 43 | LZTS2 | 0.01388412 |
| 44 | TDRKH | 0.0138581 |
| 45 | TRMT2B | 0.01377213 |
| 46 | ZNF738 | 0.01375493 |
| 47 | FHOD1 | 0.01368045 |
| 48 | RSAD2 | 0.01365854 |
| 49 | ZNF235 | 0.01362804 |
| 50 | MYSM1 | 0.01360496 |
| 51 | ALB | 0.01360188 |
| 52 | NDUFB7 | 0.01347576 |
| 53 | HEXA | 0.01341841 |
| 54 | RNF7 | 0.01333575 |
| 55 | MT-TI | 0.01330716 |
| 56 | TCEA2 | 0.01326231 |
| 57 | GATA2 | 0.01325527 |
| 58 | TOR1A | 0.0131401 |
| 59 | CLP 1 | 0.01313316 |
| 60 | PLPP3 | 0.01308848 |
| 61 | NFE2 | 0.0130462 |
| 62 | FAM212B | 0.01288717 |
| 63 | PLB1 | 0.01282596 |
| 64 | TMEM126B | 0.01276746 |
| 65 | ZNF316 | 0.01269329 |
| 66 | TMEM173 | 0.01267247 |
| 67 | PFKP | 0.01259505 |
| 68 | SLC35A5 | 0.01246928 |
| 69 | SHARPIN | 0.01239333 |
| 70 | ZBED5 | 0.01238414 |
| 71 | MPST | 0.0123601 |
| 72 | INHBA | 0.01234872 |
| 73 | ZNF426 | 0.01226576 |
| 74 | FRRS1 | 0.01224469 |
| 75 | PTGIR | 0.01215383 |
| 76 | RERE | 0.01208942 |
| 77 | CHADL | 0.01204215 |
| 78 | GALNT14 | 0.01201084 |
| 79 | RNF103 | 0.01200383 |
| 80 | RFX1 | 0.0120024 |
| 81 | MT-TR | 0.01199505 |
| 82 | TSTA3 | 0.01194721 |
| 83 | TCEAL8 | 0.01192295 |
| 84 | GPS2 | 0.01189976 |
| 85 | ADGRG1 | 0.01189662 |
| 86 | ZNF688 | 0.01185935 |
| 87 | C16orf45 | 0.01185113 |
| 88 | PTS | 0.01178986 |
| 89 | APOB | 0.0117698 |
| 90 | NDUFB6 | 0.01173206 |
| 91 | TMEM241 | 0.01170914 |
| 92 | TCTA | 0.0116774 |
| 93 | DCTN3 | 0.01166422 |
| 94 | DPPA4 | 0.01166093 |
| 95 | WBP4 | 0.01162894 |
| 96 | SNX8 | 0.01162428 |
| 97 | SPTB | 0.01161443 |
| 98 | APBB1 | 0.01160381 |
| 99 | CACTIN | 0.01157742 |
| 100 | ABCB6 | 0.01152498 |
| 101 | SKI | 0.01151656 |
| 102 | BAHCC1 | 0.01148244 |
| 103 | MAFK | 0.01141461 |
| 104 | ORAI2 | 0.01130337 |
| 105 | ENG | 0.01126375 |
| 106 | CLPTM1L | 0.01125244 |
| 107 | EPHB1 | 0.01120639 |
| 108 | MT-TV | 0.01118425 |
| 109 | COL9A3 | 0.01115156 |
| 110 | FAM98C | 0.011115 |
| 111 | CHCHD2 | 0.01108176 |
| 112 | PSRC1 | 0.01108028 |
| 113 | RPTOR | 0.01106756 |
| 114 | AP5S1 | 0.01106511 |
| 115 | BPI | 0.01104209 |
| 116 | BAX | 0.01092365 |
| 117 | FKBP8 | 0.01087398 |
| 118 | RMND5B | 0.01083154 |
| 119 | RITA1 | 0.01080038 |
| 120 | PFN2 | 0.01074414 |
| 121 | C14orf37 | 0.01073079 |
| 122 | SCPEP1 | 0.01072412 |
| 123 | GLMP | 0.01069927 |
| 124 | LRRC23 | 0.01069669 |
| 125 | HHEX | 0.01069015 |
| 126 | ZNF790 | 0.01066268 |
| 127 | PIH1D1 | 0.01063902 |
| 128 | OIT3 | 0.01059278 |
| 129 | USP20 | 0.01056321 |
| 130 | WDR48 | 0.01054698 |
| 131 | BAG5 | 0.01053765 |
| 132 | MRPL41 | 0.01051548 |
| 133 | TACC3 | 0.01050731 |
| 134 | EBF1 | 0.01049728 |
| 135 | GLTSCR1 | 0.01048172 |
| 136 | CHMP6 | 0.0104744 |
| 137 | LRP3 | 0.01046161 |
| 138 | MT-TL2 | 0.01040473 |
| 139 | JAG1 | 0.01037697 |
| 140 | ZNF577 | 0.01030925 |
| 141 | UBA3 | 0.01029964 |
| 142 | ANKRD6 | 0.01027499 |
| 143 | EBAG9 | 0.01027133 |
| 144 | CDC37 | 0.01021894 |
| 145 | TCEAL9 | 0.01019624 |
| 146 | NUCKS1 | 0.01017028 |
| 147 | LRIG2 | 0.01016899 |
| 148 | TNNT1 | 0.01012428 |
| 149 | SPSB1 | 0.01005599 |
| 150 | CDC25A | 0.0099944 |
| 151 | FAM174A | 0.00991168 |
| 152 | CH507-9B2.3 | 0.00988169 |
| 153 | SNUPN | 0.00982907 |
| 154 | ARL5B | 0.00979701 |
| 155 | ASB16-AS1 | 0.00976137 |
| 156 | ACSL5 | 0.00974051 |
| 157 | SF3B6 | 0.00972095 |
| 158 | NDUFAF5 | 0.00970246 |
| 159 | RHAG | 0.00969147 |
| 160 | RILP | 0.00965655 |
| 161 | WDR34 | 0.00964694 |
| 162 | MRPL49 | 0.00955667 |
| 163 | PNRC2 | 0.00950779 |
| 164 | MAP3K9 | 0.00950116 |
| 165 | ATG9A | 0.00949969 |
| 166 | ATN1 | 0.00945919 |
| 167 | PRDM8 | 0.00945394 |
| 168 | SYT11 | 0.00944026 |
| 169 | ADH4 | 0.0094169 |
| 170 | BAIAP2-AS1 | 0.00936576 |
| 171 | SLC35B2 | 0.00934654 |
| 172 | BCORL1 | 0.00934404 |
| 173 | ZNF281 | 0.00928822 |
| 174 | MT-TS2 | 0.00927669 |
| 175 | IFNLR1 | 0.00927275 |
| 176 | CD163 | 0.0092677 |
| 177 | PGP | 0.00926172 |
| 178 | GNG7 | 0.00921657 |
| 179 | CSRP1 | 0.00919699 |
| 180 | C6orf106 | 0.009185 |
| 181 | CASP9 | 0.00918328 |
| 182 | ATP5S | 0.00918088 |
| 183 | RRNAD1 | 0.00917771 |
| 184 | ZNF221 | 0.00913142 |
| 185 | ACOX1 | 0.00910253 |
| 186 | SNX12 | 0.00909081 |
| 187 | PIGQ | 0.00907831 |
| 188 | SIRT3 | 0.00896525 |
| 189 | CCR7 | 0.0089525 |
| 190 | RBM25 | 0.00894769 |
| 191 | NIT2 | 0.00894521 |
| 192 | PTMS | 0.00893852 |
| 193 | ZNF563 | 0.00889911 |
| 194 | TRMT1 | 0.00889782 |
| 195 | RBM17 | 0.00889295 |
| 196 | B3GNT2 | 0.00887035 |
| 197 | SH2D4A | 0.00886797 |
| 198 | ZNF205 | 0.00884385 |
| 199 | HPD | 0.0088162 |
| 200 | RTFDC1 | 0.00880671 |
| 201 | ZNF267 | 0.00876904 |
| 202 | DLG3 | 0.00876036 |
| 203 | SRSF4 | 0.00872258 |
| 204 | UPP1 | 0.00871042 |
| 205 | TNFRSF10A | 0.00868123 |
| 206 | ZNF862 | 0.00867379 |
| 207 | SRBD1 | 0.00866858 |
| 208 | SCRIB | 0.00861318 |
| 209 | WASL | 0.0085974 |
| 210 | LIMA1 | 0.00857368 |
| 211 | SUMF1 | 0.00856865 |
| 212 | PHF13 | 0.00852661 |
| 213 | KMT5B | 0.00847853 |
| 214 | ZNF783 | 0.00842612 |
| 215 | ZNF668 | 0.00839873 |
| 216 | NINL | 0.00835549 |
| 217 | REXO1 | 0.00835175 |
| 218 | EXTL3 | 0.00834063 |
| 219 | FBXW4 | 0.00832495 |
| 220 | PCYT2 | 0.00831598 |
| 221 | NMT2 | 0.00828096 |
| 222 | F2RL3 | 0.00826484 |
| 223 | ARHGEF5 | 0.00825034 |
| 224 | ZFPM1 | 0.00819933 |
| 225 | FAM134A | 0.00814859 |
| 226 | CNPPD1 | 0.00814028 |
| 227 | MUC3A | 0.0081174 |
| 228 | ZNF76 | 0.00810961 |
| 229 | DONSON | 0.00808845 |
| 230 | ZNF35 | 0.00806021 |
| 231 | SOCS4 | 0.00797538 |
| 232 | ACADVL | 0.00795214 |
| 233 | 914K2A | 0.00792301 |
| 234 | HJURP | 0.00791244 |
| 235 | RHOC | 0.00789077 |
| 236 | AK1 | 0.00783309 |
| 237 | HIP1R | 0.00779878 |
| 238 | VPS39 | 0.00779387 |
| 239 | ZSCAN29 | 0.0077435 |
| 240 | KCNH2 | 0.00769522 |
| 241 | IQGAP3 | 0.00768821 |
| 242 | PAIP2B | 0.00768409 |
| 243 | KCNK6 | 0.00767881 |
| 244 | PDRG1 | 0.00767842 |
| 245 | TRAPPC3 | 0.00766951 |
| 246 | HMGN3 | 0.00766543 |
| 247 | CIRBP | 0.00762058 |
| 248 | EAPP | 0.00761623 |
| 249 | HBD | 0.00757263 |
| 250 | GARNL3 | 0.00756375 |
| 251 | ZNF71 | 0.00749732 |
| 252 | TRIM3 | 0.00749069 |
| 253 | FBXW5 | 0.00747122 |
| 254 | TRAPPC2B | 0.00746991 |
| 255 | FAM103A1 | 0.00745236 |
| 256 | VSIG10 | 0.00743924 |
| 257 | SNW1 | 0.00743495 |
| 258 | ST14 | 0.00742482 |
| 259 | PPP1R35 | 0.00737414 |
| 260 | CWC15 | 0.00736713 |
| 261 | DNAAF3 | 0.00733761 |
| 262 | CDH1 | 0.00733675 |
| 263 | PSMA7 | 0.00733262 |
| 264 | TOP 1.00 | 0.00721997 |
| 265 | IGHV3-30 | 0.00719987 |
| 266 | KATNB1 | 0.0071801 |
| 267 | ENTPD7 | 0.00717934 |
| 268 | TBC1D10B | 0.00717475 |
| 269 | CRACR2B | 0.00716528 |
| 270 | CAPN10 | 0.00713475 |
| 271 | HERC2 | 0.00708978 |
| 272 | CTC1 | 0.00701121 |
| 273 | ELMSAN1 | 0.00700645 |
| 274 | KCNQ4 | 0.00698507 |
| 275 | TONSL | 0.00698371 |
| 276 | PELP1 | 0.00695813 |
| 277 | ZNHIT3 | 0.00695297 |
| 278 | TRAM2 | 0.00693132 |
| 279 | SRSF10 | 0.00687069 |
| 280 | ANP32B | 0.00686986 |
| 281 | SAMD12 | 0.00684181 |
| 282 | KIN | 0.00683122 |
| 283 | ZNF257 | 0.00681605 |
| 284 | ATP6V0D1 | 0.00680417 |
| 285 | CKAP2L | 0.00680053 |
| 286 | TSPYL4 | 0.0067654 |
| 287 | EIF1AD | 0.00675332 |
| 288 | ZNF518B | 0.00675167 |
| 289 | HNRNPL | 0.00674865 |
| 290 | TNPO2 | 0.00672039 |
| 291 | MIER3 | 0.00671229 |
| 292 | C21orf2 | 0.00669982 |
| 293 | CNTNAP2 | 0.00665981 |
| 294 | SYNE3 | 0.00662893 |
| 295 | RACGAP1 | 0.00662596 |
| 296 | PEX16 | 0.00661942 |
| 297 | GPANK1 | 0.00661331 |
| 298 | SRGAP2C | 0.00660625 |
| 299 | IRF2BP1 | 0.00659663 |
| 300 | GFER | 0.00655544 |
| 301 | EPS8L2 | 0.00653381 |
| 302 | CBX4 | 0.00647188 |
| 303 | PPP1R26 | 0.00644835 |
| 304 | PIK3R6 | 0.00642804 |
| 305 | IFT122 | 0.00642399 |
| 306 | MRPL22 | 0.00638506 |
| 307 | PDAP1 | 0.00638494 |
| 308 | TTN | 0.00638015 |
| 309 | GABBR1 | 0.00637569 |
| 310 | LRRC59 | 0.00635053 |
| 311 | CAD | 0.00634658 |
| 312 | ABHD15 | 0.00632624 |
| 313 | P4HB | 0.00631207 |
| 314 | PATL1 | 0.00630895 |
| 315 | DCUN1D2 | 0.00630072 |
| 316 | ZNF394 | 0.00629403 |
| 317 | MORC2 | 0.00628119 |
| 318 | HIST1H2BB | 0.00626976 |
| 319 | ZCCHC6 | 0.00625588 |
| 320 | P2RX5 | 0.00625104 |
| 321 | DNAJB5 | 0.00624363 |
| 322 | ZNF629 | 0.00623278 |
| 323 | PTDSS2 | 0.00623102 |
| 324 | CCL3L3 | 0.00620529 |
| 325 | RRBP1 | 0.00618936 |
| 326 | RAB24 | 0.00616838 |
| 327 | UXT | 0.00614935 |
| 328 | NFATC1 | 0.00614695 |
| 329 | ZCWPW1 | 0.00612475 |
| 330 | ZNF678 | 0.00609963 |
| 331 | ADAM12 | 0.00607422 |
| 332 | WDR53 | 0.00599808 |
| 333 | CD19 | 0.00598854 |
| 334 | SMYD5 | 0.00598828 |
| 335 | FAM214B | 0.00597508 |
| 336 | CDC42SE1 | 0.0059579 |
| 337 | SLX4 | 0.00595597 |
| 338 | NEMP1 | 0.00595561 |
| 339 | HMGB2 | 0.00592168 |
| 340 | MRI1 | 0.00588256 |
| 341 | NAT6 | 0.00586786 |
| 342 | XRCC1 | 0.00585168 |
| 343 | IRF9 | 0.00583976 |
| 344 | OSGIN2 | 0.00583503 |
| 345 | MRNIP | 0.00582855 |
| 346 | RSRC2 | 0.0058153 |
| 347 | ZNF598 | 0.00577474 |
| 348 | PIK3IP1 | 0.00575823 |
| 349 | KIAA0922 | 0.00571143 |
| 350 | MRPL28 | 0.00567637 |
| 351 | ZNF326 | 0.00566734 |
| 352 | PDSS2 | 0.00566216 |
| 353 | ZC3H12A | 0.00565495 |
| 354 | MORN3 | 0.0056501 |
| 355 | RNF31 | 0.00561533 |
| 356 | KIAA1147 | 0.00560077 |
| 357 | CLCN7 | 0.00558628 |
| 358 | EVPL | 0.00557115 |
| 359 | CTSL | 0.00556813 |
| 360 | HP | 0.00556605 |
| 361 | HSPA1L | 0.00555607 |
| 362 | EMILIN1 | 0.00551661 |
| 363 | TSC22D4 | 0.00548898 |
| 364 | ORM1 | 0.00548706 |
| 365 | RASAL2-AS1 | 0.00546787 |
| 366 | APEX2 | 0.00546566 |
| 367 | CENPP | 0.00543941 |
| 368 | C7orf50 | 0.00543674 |
| 369 | MICAL3 | 0.00542727 |
| 370 | SNAPC4 | 0.00542409 |
| 371 | ZBTB39 | 0.00539849 |
| 372 | SELENOP | 0.00539036 |
| 373 | TBC1D25 | 0.00538649 |
| 374 | WDR73 | 0.00538553 |
| 375 | NPIPA5 | 0.0053847 |
| 376 | PARP6 | 0.0053542 |
| 377 | AHDC1 | 0.0053378 |
| 378 | PATJ | 0.00533587 |
| 379 | DHX37 | 0.00533578 |
| 380 | PPID | 0.00531605 |
| 381 | SMIM24 | 0.00531315 |
| 382 | ANKRD45 | 0.0053085 |
| 383 | TAF3 | 0.00528601 |
| 384 | POLM | 0.0052713 |
| 385 | DNAJB2 | 0.00525996 |
| 386 | GFAP | 0.00524745 |
| 387 | TOR1AIP2 | 0.00522342 |
| 388 | MICALL2 | 0.00520235 |
| 389 | GINS2 | 0.00516785 |
| 390 | CRHBP | 0.00516767 |
| 391 | MTIF2 | 0.00514099 |
| 392 | TRAF1 | 0.00513172 |
| 393 | HTRA2 | 0.0051272 |
| 394 | DUSP3 | 0.00511558 |
| 395 | NET1 | 0.00509752 |
| 396 | MEIS2 | 0.00508531 |
| 397 | ATG4D | 0.00503696 |
| 398 | CDADC1 | 0.00503346 |
| 399 | FBRSL1 | 0.00500885 |
| 400 | SWSAP1 | 0.00500631 |
| 401 | MTRNR2L8 | 0.00498493 |
| 402 | FTCDNL1 | 0.00498196 |
| 403 | PTGDS | 0.0049811 |
| 404 | ST3GAL1 | 0.00496821 |
| 405 | TRIM10 | 0.00496727 |
| 406 | NECTIN1 | 0.00494824 |
| 407 | NUF2 | 0.00494803 |
| 408 | SH3PXD2B | 0.00487005 |
| 409 | HNRNPH3 | 0.00485432 |
| 410 | TNFRSF21 | 0.00485095 |
| 411 | FBXL19 | 0.00482935 |
| 412 | C3orf38 | 0.00482822 |
| 413 | ERLEC1 | 0.00481757 |
| 414 | RAPGEF6 | 0.00481753 |
| 415 | FAM134B | 0.00476877 |
| 416 | NEK2 | 0.00476605 |
| 417 | PIGC | 0.00474254 |
| 418 | HDAC10 | 0.00467651 |
| 419 | RETN | 0.00467019 |
| 420 | AUNIP | 0.00465792 |
| 421 | CLSPN | 0.00463933 |
| 422 | SMC3 | 0.00463566 |
| 423 | TICRR | 0.00462759 |
| 424 | BCAR1 | 0.00455823 |
| 425 | TNK2 | 0.00451586 |
| 426 | NLRC3 | 0.00450598 |
| 427 | PGRMC2 | 0.0044856 |
| 428 | ITPKB | 0.00448118 |
| 429 | GAS8 | 0.00447802 |
| 430 | MFAP1 | 0.00445902 |
| 431 | KIAA1549 | 0.00445435 |
| 432 | STK36 | 0.0044393 |
| 433 | MSANTD2 | 0.00440631 |
| 434 | MID1IP1 | 0.00439898 |
| 435 | HLA-DQA2 | 0.00438787 |
| 436 | KIAA0232 | 0.00438699 |
| 437 | ZCCHC3 | 0.0043752 |
| 438 | ZDHHC5 | 0.00436213 |
| 439 | TCEAL1 | 0.00436064 |
| 440 | MCM7 | 0.00434985 |
| 441 | ZYG11B | 0.00432486 |
| 442 | HIST1H2BL | 0.00430363 |
| 443 | EMC7 | 0.0042997 |
| 444 | SOX12 | 0.00426019 |
| 445 | PSMC1 | 0.00425978 |
| 446 | PSENEN | 0.00424307 |
| 447 | FGFR1 | 0.00422946 |
| 448 | CIR1 | 0.00419353 |
| 449 | PLTP | 0.00418576 |
| 450 | CCNB2 | 0.00416864 |
| 451 | DOK1 | 0.00415016 |
| 452 | RNF145 | 0.00415008 |
| 453 | TBC1D22A | 0.00411891 |
| 454 | PLIN2 | 0.00408977 |
| 455 | P2RY8 | 0.00405717 |
| 456 | ROMO1 | 0.00403507 |
| 457 | HIST1H3F | 0.00403297 |
| 458 | MAD1L1 | 0.00402509 |
| 459 | DMTF1 | 0.0040051 |
| 460 | LONP1 | 0.00399071 |
| 461 | CMBL | 0.0039846 |
| 462 | METAP2 | 0.00398148 |
| 463 | BDH1 | 0.00397872 |
| 464 | CEP95 | 0.00397779 |
| 465 | SYS1 | 0.00397486 |
| 466 | BCDIN3D | 0.0039398 |
| 467 | NDC80 | 0.00391798 |
| 468 | SLC35F5 | 0.00390787 |
| 469 | ZNHIT6 | 0.00390234 |
| 470 | BNIP1 | 0.00390142 |
| 471 | PLIN3 | 0.00390095 |
| 472 | CHMP4A | 0.00389975 |
| 473 | SPHK2 | 0.00389825 |
| 474 | RALA | 0.00387198 |
| 475 | POMC | 0.00384375 |
| 476 | FXR2 | 0.00383397 |
| 477 | RRP15 | 0.00379515 |
| 478 | CNPY3 | 0.00379038 |
| 479 | FASTKD3 | 0.00378887 |
| 480 | RABL3 | 0.00376548 |
| 481 | SLC39A13 | 0.00374723 |
| 482 | ZBTB5 | 0.00374536 |
| 483 | SLC7A6OS | 0.0037395 |
| 484 | SNX21 | 0.00373102 |
| 485 | FAM171A1 | 0.00372713 |
| 486 | EHMT2 | 0.00367873 |
| 487 | GTPBP6 | 0.00367428 |
| 488 | 44258 | 0.00366069 |
| 489 | SCAF1 | 0.00365522 |
| 490 | ALDH18A1 | 0.00365454 |
| 491 | RABL2B | 0.00364771 |
| 492 | PCGF3 | 0.00364631 |
| 493 | FBRS | 0.00364104 |
| 494 | SFMBT1 | 0.00363168 |
| 495 | ZBTB41 | 0.00362658 |
| 496 | TMF1 | 0.00361566 |
| 497 | IRAK1BP1 | 0.00361537 |
| 498 | ZNF550 | 0.00359616 |
| 499 | RNF26 | 0.00356074 |
| 500 | ATRN | 0.0035562 |
| 501 | POLDIP3 | 0.00353106 |
| 502 | FAM32A | 0.0035253 |
| 503 | RBM19 | 0.00349255 |
| 504 | PLEKHA7 | 0.00349242 |
| 505 | BRF1 | 0.00349014 |
| 506 | EFTUD2 | 0.00348959 |
| 507 | ZDHHC13 | 0.00348433 |
| 508 | AKAP9 | 0.00346468 |
| 509 | DDRGK1 | 0.00338493 |
| 510 | ZBTB17 | 0.00338478 |
| 511 | C19orf43 | 0.00336635 |
| 512 | SUGP2 | 0.00334684 |
| 513 | CHID1 | 0.00331867 |
| 514 | MKL1 | 0.00330825 |
| 515 | IGLC3 | 0.00326331 |
| 516 | HOXB3 | 0.00325705 |
| 517 | PSMG1 | 0.00325184 |
| 518 | TRMT13 | 0.00324839 |
| 519 | GOLGA2 | 0.00324633 |
| 520 | RNASE3 | 0.00323686 |
| 521 | AXIN2 | 0.00323191 |
| 522 | GPAA1 | 0.00322351 |
| 523 | ZNF317 | 0.00321854 |
| 524 | HIST1H2AD | 0.00320508 |
| 525 | WRAP73 | 0.00320307 |
| 526 | NOD1 | 0.00319479 |
| 527 | HMGXB4 | 0.00318399 |
| 528 | ABL2 | 0.00314609 |
| 529 | SYNGAP1 | 0.00312749 |
| 530 | TSPAN31 | 0.00306728 |
| 531 | SLU7 | 0.0030589 |
| 532 | SPRED2 | 0.00302972 |
| 533 | FBXL15 | 0.00302544 |
| 534 | DNAJC14 | 0.00301706 |
| 535 | MAZ | 0.00301373 |
| 536 | AKT1 | 0.00300904 |
| 537 | EPS8L1 | 0.00298856 |
| 538 | ESPL1 | 0.00298083 |
| 539 | FAM50B | 0.00297548 |
| 540 | RLIM | 0.00296119 |
| 541 | SYMPK | 0.00294351 |
| 542 | DNHD1 | 0.00293687 |
| 543 | SDF2 | 0.00293563 |
| 544 | DUSP23 | 0.00292554 |
| 545 | C2CD2L | 0.0029136 |
| 546 | WHSC1 | 0.00290877 |
| 547 | NSRP1 | 0.00290313 |
| 548 | TSHZ2 | 0.00288423 |
| 549 | HIC1 | 0.00287728 |
| 550 | PLXNB2 | 0.0028503 |
| 551 | FOLR3 | 0.00283506 |
| 552 | CTB-50L17.10 | 0.0028331 |
| 553 | ZRSR2 | 0.0028224 |
| 554 | APBA2 | 0.00281752 |
| 555 | FEN1 | 0.00281398 |
| 556 | MAGEE1 | 0.00281389 |
| 557 | KLF16 | 0.0028058 |
| 558 | EPB41L5 | 0.00279834 |
| 559 | PPP4C | 0.00274163 |
| 560 | DCUN1D3 | 0.00273349 |
| 561 | GSDMB | 0.0027255 |
| 562 | AMY2B | 0.00271999 |
| 563 | FLT3 | 0.00271279 |
| 564 | MUT | 0.00269531 |
| 565 | FAM107B | 0.00269214 |
| 566 | CCDC88C | 0.00267412 |
| 567 | PPP1R12C | 0.00266498 |
| 568 | NAV2 | 0.00264828 |
| 569 | SH3GL1 | 0.00264045 |
| 570 | CEP83 | 0.00263927 |
| 571 | RANGAP1 | 0.00262376 |
| 572 | SIRT6 | 0.00262223 |
| 573 | SREK1 | 0.00261003 |
| 574 | CDCA2 | 0.00258655 |
| 575 | KAT2A | 0.00258023 |
| 576 | NUDCD3 | 0.00255822 |
| 577 | CSF1 | 0.00254994 |
| 578 | ZNF865 | 0.00253668 |
| 579 | TOB1 | 0.00251809 |
| 580 | BET1L | 0.00251733 |
| 581 | GJA4 | 0.00251321 |
| 582 | C11orf95 | 0.0024976 |
| 583 | ZNF182 | 0.00249399 |
| 584 | COQ5 | 0.00247868 |
| 585 | HIST1H4B | 0.00247098 |
| 586 | MR1 | 0.00247081 |
| 587 | MYO5A | 0.00246957 |
| 588 | DTX2P1-UPK3BP1- | 0.00243386 |
| | PMS2P11 | |
| 589 | GFOD1 | 0.00241489 |
| 590 | RINL | 0.00241422 |
| 591 | ING1 | 0.00241211 |
| 592 | SMARCC2 | 0.0023985 |
| 593 | ZBTB7A | 0.00238074 |
| 594 | MYCN | 0.00236136 |
| 595 | SHQ1 | 0.00235142 |
| 596 | CCDC3 | 0.00234966 |
| 597 | PDE2A | 0.00234651 |
| 598 | ERCC6L | 0.00233006 |
| 599 | DPH1 | 0.00231002 |
| 600 | NFKBIA | 0.0022911 |
| 601 | RP5-862P8.2 | 0.00227093 |
| 602 | ZDHHC6 | 0.00225623 |
| 603 | ZNF432 | 0.00225097 |
| 604 | CEP104 | 0.00224807 |
| 605 | ARRDC4 | 0.00224182 |
| 606 | H1FX | 0.00223116 |
| 607 | LMBR1L | 0.00222269 |
| 608 | USP8 | 0.0021974 |
| 609 | MED9 | 0.00219293 |
| 610 | TDP2 | 0.00217073 |
| 611 | DNTTIP1 | 0.00216686 |
| 612 | RILPL2 | 0.00214484 |
| 613 | SH3BP5 | 0.00214274 |
| 614 | MYO7A | 0.00212784 |
| 615 | NCOR2 | 0.00212433 |
| 616 | GTPBP8 | 0.00212003 |
| 617 | FO538757.1 | 0.00211862 |
| 618 | CXXC1 | 0.00211442 |
| 619 | AKAP8 | 0.00211194 |
| 620 | ZNRF1 | 0.00210383 |
| 621 | ULK1 | 0.0020961 |
| 622 | AVEN | 0.00209074 |
| 623 | ABCC10 | 0.00207338 |
| 624 | HIST2H2AC | 0.00203952 |
| 625 | FAN1 | 0.00203669 |
| 626 | OSBP | 0.00202982 |
| 627 | GOLM1 | 0.00202069 |
| 628 | P3H1 | 0.00201862 |
| 629 | CCDC71 | 0.00201133 |
| 630 | RPUSD1 | 0.00200975 |
| 631 | LZTR1 | 0.00197951 |
| 632 | NAPRT | 0.00196389 |
| 633 | EPN1 | 0.00196033 |
| 634 | LTB4R | 0.00194123 |
| 635 | PNKP | 0.0019049 |
| 636 | ZNF264 | 0.00189308 |
| 637 | GTSE1 | 0.00188309 |
| 638 | HIST1H2AL | 0.00188158 |
| 639 | IGLV1-47 | 0.00184976 |
| 640 | NAIF1 | 0.00184679 |
| 641 | TLE1 | 0.00183477 |
| 642 | CCDC96 | 0.00182908 |
| 643 | TFR2 | 0.00181797 |
| 644 | YTHDC1 | 0.00181123 |
| 645 | HDX | 0.00178841 |
| 646 | TAPT1 | 0.00178501 |
| 647 | SPA17 | 0.00177161 |
| 648 | FAM9C | 0.00176343 |
| 649 | FAM43A | 0.0017418 |
| 650 | ANKLE2 | 0.00173128 |
| 651 | ZNF496 | 0.00171209 |
| 652 | PARD6B | 0.00170735 |
| 653 | AKAP8L | 0.00169481 |
| 654 | LIAS | 0.00166417 |
| 655 | DBF4B | 0.00165354 |
| 656 | PLK1 | 0.00165293 |
| 657 | RAB3IL1 | 0.00163743 |
| 658 | OGG1 | 0.00162467 |
| 659 | FOXM1 | 0.00161892 |
| 660 | MT-RNR2 | 0.00160061 |
| 661 | GPIHBP1 | 0.00158073 |
| 662 | FOXO1 | 0.00157252 |
| 663 | ITGA9 | 0.00156769 |
| 664 | SDF4 | 0.00155878 |
| 665 | KLC2 | 0.00154916 |
| 666 | ANXA4 | 0.00153646 |
| 667 | CCHCR1 | 0.00152904 |
| 668 | ZNF282 | 0.00151814 |
| 669 | TSPYL1 | 0.00147807 |
| 670 | BAP1 | 0.0014725 |
| 671 | BBS10 | 0.00146978 |
| 672 | ZBTB48 | 0.00145997 |
| 673 | BRD9 | 0.00145826 |
| 674 | NLRX1 | 0.00142502 |
| 675 | YDJC | 0.00141928 |
| 676 | ZBTB7B | 0.00141311 |
| 677 | BRD1 | 0.00140997 |
| 678 | MNS1 | 0.00140356 |
| 679 | ABCD4 | 0.00139032 |
| 680 | MEX3C | 0.00138039 |
| 681 | ZNF219 | 0.00137284 |
| 682 | CCDC12 | 0.00136843 |
| 683 | SPATA2 | 0.00136746 |
| 684 | ZNF528 | 0.00135979 |
| 685 | SH3PXD2A | 0.00135844 |
| 686 | OLFML2B | 0.00133113 |
| 687 | C2orf49 | 0.00127454 |
| 688 | HMGN2 | 0.00125333 |
| 689 | POLE3 | 0.0012327 |
| 690 | MDM4 | 0.00119826 |
| 691 | INMT | 0.00117138 |
| 692 | MAN2C1 | 0.00114471 |
| 693 | PPARA | 0.00113824 |
| 694 | BPNT1 | 0.0011324 |
| 695 | IRS2 | 0.00112693 |
| 696 | TBC1D13 | 0.00109838 |
| 697 | SYF2 | 0.00109755 |
| 698 | RAPGEF3 | 0.00108811 |
| 699 | RPL41 | 0.00108174 |
| 700 | TMEM259 | 0.00108088 |
| 701 | CDK10 | 0.00107791 |
| 702 | ZNF420 | 0.00107789 |
| 703 | JAGN1 | 0.00107556 |
| 704 | SPRTN | 0.00106533 |
| 705 | CD79B | 0.00106206 |
| 706 | B3GAT3 | 0.00106058 |
| 707 | MYL4 | 0.00105931 |
| 708 | TCN1 | 0.00103934 |
| 709 | GNA12 | 0.00102483 |
| 710 | EFNB2 | 0.00102043 |
| 711 | OASL | 0.00100613 |
| 712 | SLC22A4 | 0.0009892 |
| 713 | TAF7 | 0.00096694 |
| 714 | ECHDC2 | 0.00095397 |
| 715 | CENPB | 0.0009517 |
| 716 | C15orf57 | 0.00094717 |
| 717 | PLCB3 | 0.00093872 |
| 718 | SYVN1 | 0.00092311 |
| 719 | TRIM62 | 0.00091832 |
| 720 | SMG9 | 0.00090996 |
| 721 | SCAPER | 0.00090709 |
| 722 | DMPK | 0.00089951 |
| 723 | DGKQ | 0.00089441 |
| 724 | NOC2L | 0.00088618 |
| 725 | ZNF341 | 0.0008737 |
| 726 | HDAC1 | 0.000863 |
| 727 | MZF1 | 0.00086231 |
| 728 | NT5C3B | 0.00085006 |
| 729 | GCHFR | 0.0008309 |
| 730 | RALB | 0.00082971 |
| 731 | TSGA10 | 0.00082398 |
| 732 | PPP6R1 | 0.00082136 |
| 733 | NBPF20 | 0.00081391 |
| 734 | ZNF595 | 0.00081372 |
| 735 | MROH1 | 0.00081248 |
| 736 | PPAT | 0.00081043 |
| 737 | KDM2B | 0.00080194 |
| 738 | CRISP3 | 0.00080069 |
| 739 | ZNF70 | 0.00077202 |
| 740 | PLP2 | 0.00076753 |
| 741 | IFT57 | 0.00075833 |
| 742 | HBQ1 | 0.00073992 |
| 743 | ZBTB4 | 0.00072527 |
| 744 | ASF1B | 0.0006931 |
| 745 | GNE | 0.00067357 |
| 746 | ODF3B | 0.00067249 |
| 747 | FAM184A | 0.00066331 |
| 748 | PDE12 | 0.00064095 |
| 749 | IL3RA | 0.00063461 |
| 750 | DIXDC1 | 0.00060502 |
| 751 | ANP32A | 0.00059486 |
| 752 | MAP3K12 | 0.00059293 |
| 753 | GOLGB1 | 0.00058282 |
| 754 | PPP4R2 | 0.00057197 |
| 755 | ENPP2 | 0.000558 |
| 756 | RPH3AL | 0.00055265 |
| 757 | ZNF791 | 0.00053816 |
| 758 | NPIPB4 | 0.00050393 |
| 759 | ZNF615 | 0.00048048 |
| 760 | CHAC2 | 0.00046328 |
| 761 | DDX43 | 0.00046102 |
| 762 | GMPPB | 0.0004581 |
| 763 | TNRC6A | 0.00045704 |
| 764 | LENG1 | 0.00045275 |
| 765 | TMEM218 | 0.00045032 |
| 766 | FUT4 | 0.00043039 |
| 767 | PRKCE | 0.00033648 |
| 768 | TMA7 | 0.00033279 |
| 769 | BTBD6 | 0.00031161 |
| 770 | ZFP30 | 0.00028603 |
| 771 | ATXN7L3 | 0.00028551 |
| 772 | FLVCR2 | 0.00028409 |
| 773 | P4HA2 | 0.00028193 |
| 774 | IP6K2 | 0.00027222 |
| 775 | CTSG | 0.00025912 |
| 776 | TMEM14A | 0.00024798 |
| 777 | RNF157 | 0.0002095 |
| 778 | ECD | 0.00020545 |
| 779 | KIF20A | 0.00018898 |
| 780 | MXD3 | 0.00018339 |
| 781 | SLC39A7 | 0.00017198 |
| 782 | ZNF787 | 0.00012374 |
| 783 | DUS3L | 5.1952E−05 |
| 784 | ALG3 | 3.8399E−05 |
| 785 | BCKDHB | 2.9225E−05 |
| 786 | CLN5 | 2.2305E−05 |
| 787 | DLGAP4 | 5.8398E−06 |

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-191. (canceled)

192. A method comprising:
(a) assaying a cell-free blood sample of a pregnant subject to determine at least one expression level of at least one pregnancy-associated gene, wherein said at least one pregnancy-associated gene is differentially expressed in a first population of subjects having a pregnancy-related hypertensive disorder as compared to a second population of subjects not having said pregnancy-related hypertensive disorder;
(b) computer processing said at least one expression level of said at least one pregnancy-associated gene determined in (a) (i) against at least one reference expression level of said at least one pregnancy-associated gene or (ii) with a trained machine learning algorithm;
(c) determining, based at least in part on said computer processing in (b), that said pregnant subject has an elevated risk of having said pregnancy-related hypertensive disorder; and
(d) based at least in part on said determining in (c), providing a treatment plan to said pregnant subject for said elevated risk of having said pregnancy-related hypertensive disorder.

193. The method of claim 192, wherein said treatment plan comprises a prophylactic intervention that reduces said elevated risk of having said pregnancy-related hypertensive disorder.

194. The method of claim 192, wherein said prophylactic intervention comprises providing medical monitoring to said pregnant subject.

195. The method of claim 194, wherein said medical monitoring comprises monitoring a blood pressure of said pregnant subject.

196. The method of claim 192, wherein said prophylactic intervention comprises providing a nutritional supplement to said pregnant subject.

197. The method of claim 196, wherein said nutritional supplement comprises calcium, vitamin D, vitamin B3, or docosahexaenoic acid (DHA).

198. The method of claim 192, wherein said prophylactic intervention comprises providing a lifestyle modification to said pregnant subject.

199. The method of claim 198, wherein said lifestyle modification comprises an exercise regimen, nutrition counseling, meditation, stress relief, weight loss or maintenance, or improving sleep quality.

200. The method of claim 192, further comprising performing a liver or renal dysfunction test on said pregnant subject.

201. The method of claim 192, wherein said treatment plan comprises a therapeutic intervention for said pregnancy-related hypertensive disorder or said elevated risk of having said pregnancy-related hypertensive disorder.

202. The method of claim 201, wherein said therapeutic intervention comprises administering a drug to said pregnant subject.

203. The method of claim 202, wherein said drug is selected from the group consisting of an antihypertensive drug, aspirin, progesterone, a corticosteroid, an antibiotic, a tocolytic drug, a cyclo-oxygenase inhibitor, an oxytocin antagonist, a betamimetic drug, magnesium sulfate, magnesium chloride, and magnesium oxide.

204. The method of claim 202, wherein said drug is selected from the group consisting of a cholesterol medication, a heartburn medication, an angiotensin II receptor antagonist, a calcium channel blocker, a diabetes medication, metformin, and an erectile dysfunction medication.

205. The method of claim 192, wherein (c) further comprises determining that said pregnant subject has an elevated risk of having a molecular subtype of said pregnancy-related hypertensive disorder, and wherein (d) further comprises providing said treatment plan to said pregnant subject for said molecular subtype of said pregnancy-related hypertensive disorder.

206. The method of claim 205, wherein said molecular subtype of said pregnancy-related hypertensive disorder is selected from the group consisting of: preeclampsia, mild preeclampsia, severe preeclampsia, preeclampsia determined at less than 34 weeks gestational age, preeclampsia determined at greater than 34 weeks gestational age, preeclampsia determined at less than 37 weeks gestational age, preeclampsia determined at greater than 37 weeks gestational age, preeclampsia with clinical indication of delivery at less than 34 weeks gestational age, preeclampsia with clinical indication of delivery at greater than 34 weeks gestational age, preeclampsia with clinical indication of delivery at less than 37 weeks gestational age, preeclampsia with clinical indication of delivery at greater than 37 weeks gestational age, eclampsia, chronic or pre-existing hypertension, gestational hypertension, and HELLP (hemolysis, elevated liver enzymes, and low platelets) syndrome.

207. The method of claim 206, wherein said molecular subtype of said pregnancy-related hypertensive disorder is preeclampsia.

208. The method of claim 192, wherein (a) further comprises determining at least one RNA level of said at least one pregnancy-associated gene, and wherein (b) further comprises computer processing said at least one RNA level of said at least one pregnancy-associated gene.

209. The method of claim 208, wherein (a) further comprises reverse transcribing ribonucleic acid (RNA) molecules from said cell-free blood sample to produce complementary deoxyribonucleic acid (cDNA) molecules; and assaying said cDNA molecules to determine said at least one RNA level of said at least one pregnancy-associated gene.

210. The method of claim 208, wherein said assaying further comprises nucleic acid sequencing.

211. The method of claim 208, wherein said assaying further comprises array hybridization.

212. The method of claim 208, wherein said assaying further comprises polymerase chain reaction (PCR).

213. The method of claim 212, wherein said PCR comprises digital PCR or digital droplet PCR.

214. The method of claim 208, wherein (a) further comprises selectively enriching nucleic acid molecules from said cell-free blood sample.

215. The method of claim 208, wherein (a) further comprises assaying nucleic acid molecules from said cell-free blood sample without selectively enriching said nucleic acid molecules.

216. The method of claim 192, wherein said cell-free blood sample comprises a plasma sample.

217. The method of claim 192, wherein said pregnant subject is asymptomatic for said pregnancy-related hypertensive disorder.

218. The method of claim 192, wherein said computer processing in (b) comprises said trained machine learning algorithm.

219. The method of claim 218, wherein said trained machine learning algorithm is selected from the group consisting of a linear regression, a logistic regression, an analysis of variance (ANOVA) model, a deep learning algorithm, a support vector machine (SVM), a neural network, a Random Forest, and a combination thereof.

220. The method of claim 192, further comprising monitoring said pregnant subject for risk of having said pregnancy-related hypertensive disorder, wherein said monitoring comprises determining whether said pregnant subject has an elevated risk of having said pregnancy-related hypertensive disorder at each of a plurality of time points.

221. The method of claim 220, wherein a difference in said determining whether said pregnant subject has said elevated risk of having said pregnancy-related hypertensive disorder at each of said plurality of time points is indicative of one or more clinical indications selected from the group consisting of: (i) a diagnosis of said pregnancy-related hypertensive disorder of said pregnant subject, (ii) a prognosis of said pregnancy-related hypertensive disorder of said pregnant subject, (iii) an efficacy or non-efficacy of a therapeutic intervention for treating said pregnancy-related hypertensive disorder of said pregnant subject, and (iv) an efficacy or non-efficacy of a prophylactic intervention for reducing said elevated risk of having said pregnancy-related hypertensive disorder of said pregnant subject.
